(19) United States
(12) Patent Application Publication
(10) Pub. No.: US 2008/0160116 A1
(43) Pub. Date: Jul. 3, 2008

(54) Compositions and Methods Comprising Zingiber Species

(75) Inventors: Dan Li, Singapore (CN); George W. Sypert, Naples, FL (US); Robert T. Gow, Naples, FL (US)

Correspondence Address: FOLEY HOAG, LLP;PATENT GROUP, WORLD TRADE CENTER WEST, 155 SEAPORT BLVD, BOSTON, MA, 02110, US

(73) Assignee: HerbalScience Singapore Pte. Ltd, Singapore (SG)

(21) Appl. No.: 11/951,948

(22) Filed: Dec. 6, 2007

Related U.S. Application Data
(60) Provisional application No. 60/873,320, filed on Dec. 7, 2006.

(51) Int. Cl. A61K 36/906; A61K 31/715; A61K 31/12; A61P 43/00

(52) U.S. Cl. 424/756; 514/678; 514/54

(57) ABSTRACT

An aspect of the present invention relates to compositions comprising a gingerol, for example, compositions comprising gingerol in an amount greater than about 2% by weight. In some aspects of the invention, the composition comprises 6-gingerol, 8-gingerol, 10-gingerol, 6-shagaol, or combinations thereof. Another aspect of the invention relates to a method for extracting a ginger species comprising, sequentially extracting a ginger species plant material to yield an essential oil fraction, a gingerol fraction, a phenolic fraction, and a polysaccharide fraction, wherein the essential oil and gingerol fractions are derived by extracting plant feedstock material by supercritical carbon dioxide extraction, the phenolic fraction is extracted from the plant feedstock material or from the remainder of the essential oil and gingerol extractions by hydroalcoholic extraction, and the polysaccharide fraction is derived by water extraction of the remainder of the phenolic extraction.

This application claims the benefit of priority of U.s. Provisional Application No. 60/873,320, filed on Dec. 7, 2006, the contents of which are incorporated by reference in their entirety.

### FIELD OF THE INVENTION

The disclosure relates to methods for making compositions derived from Zingiber species (ginger) having uniquely elevated volatile oil chemical constituents, gingerol chemical constituents (oleoresin), phenolic acid chemical constituents, and polysaccharide chemical constituents and compositions made by such methods, particularly oral delivery formulations, and methods for use of such compositions.

### BACKGROUND OF THE INVENTION

Plants of the ginger (Zingerber officinale Roscoe, Zingiberaceae) family are among the most heavily consumed dietary substances in the world. As an herb, ginger has been used as a food and medicine for more than 5000 years. Seeming to originate from Southern China, ginger is produced and exported in tropic and subtropic Asia, Brazil, Jamaica, and Nigeria. India, however, is the world's largest producer and exporter of ginger, harvesting greater than 50% of the world's supply. Ginger is used in food, drink, candy, cosmetics, perfumes, deodorants, and herbal medicine depending on the culture. Traditional medicine of many cultures primarily utilizes ginger as remedies for numerous ailments including nausea, sea or motion sickness, nausea related to pregnancy, vomiting, loss of appetite, stomach cramps, diarrhea, heartburn, colic, flatulence, indigestion, common cold, influenza, cough arthritis, rheumatic disorders, migraines, headaches, cardiac palpitations, hypertension, and impotence. It is reported to exhibit, stimulant, aphrodisiac, aromatic, and carminative properties when taken internally, while behaving as a sialogogue when chewed, a rubefacient when applied externally. Until recently, the medicinal, chemical, and pharmaceutical properties of ginger have not been verified with rigorous scientific methods.

To summarize briefly what is known regarding the therapeutic value of Zingiber species rhizome chemical constituents, scientific and clinical research studies have demonstrated the following therapeutic effects of the various chemical compounds, chemical fractions, and crude extracts of the ginger species which include the following: anti-nausea and vomiting related to pregnancy, motion sickness, anesthesia and surgery, and cancer chemotherapy, without drowsiness or fetal risk [gingerols, lipid soluble extract, crude extract] (1-9); anti-inflammatory, osteoarthritis, rheumatoid disorders, analgesia [gingerols, volatile oil extract, lipid soluble extract, water soluble extract, crude extract] (11-18); anti-oxidant, nitric oxide inhibition, and free radical scavenging [zingerone, volatile oil, lipid soluble extract, phenolic fraction, crude extract] (17, 19-23), hyperlipidemia, diabetes [gingerols, lipid soluble extract, crude extract] (24-27), anti-thrombosis [lipid soluble extract] (28), hypertension [aqueous extract, crude extract] (29, 30); vasodilation [aqueous extract, crude extract] (29, 30); cardiac palpitations [aqueous extract] (29); anti-atherosclerosis [ginger powder] (31); anti-obesity [aqueous extract] (32); cardiovascular disease [lipid soluble extract, water soluble extract, crude extract, ginger powder] (17, 19-32); cerebrovascular disease/stroke [lipid soluble extract, water soluble extract, crude extract, ginger powder] (17, 19-32); brain degenerative disease such as Alzheimer's and Parkinson's [zingerone, lipid soluble extract, crude extract] (19, 33, 34); headache and migraine [crude extract] (35); Immunomodulatory activity, anti-auto immune disease [volatile oil extract] (20, 36); radiation protection [hydroalcoholic extract] (37): anti-colic, anti-dyspepsia, anti-diarrhea [crude extract] (38, 39); antibacterial [volatile oil fraction, methanol extract] (20, 40-42); anti-viral [crude extract] (20); and anti-mutagenesis and cancer prevention and therapy [beta-elemene, gingerols, zerumbone, lipid soluble fraction] (17, 20, 22, 43-48). Moreover, ginger has been proven to be very safe taken in rather massive dosages such as 12 gm/day in humans and 1.5 gm/kg body weight in mice (20, 37). One caution would be taking large doses of ginger by patients who are also taking anti-coagulant pharmaceutical such as coumadin.

What is needed are novel and reproducible ginger extract compositions that combine purified volatile oil, purified gingerol, purified phenolic, and polysaccharide chemical constituent fractions that can be produced with standardized and reliable amounts of these synergistically (49) acting physiologically and medically beneficial ginger chemical constituents.

### SUMMARY OF THE INVENTION

Aspects of the disclosure relate to compositions of ginger (Zingerber species) and processes for the preparation and/or formulation thereof. In certain embodiments, the disclosure provides Zingerber species compositions with characteristics such as, but not limited to, elevated amounts of volatile oil chemical constituents, elevated amounts of gingerol chemical constituents, elevated amounts of phenolic acid chemical constituents with elevated 6-gingerol, and elevated amounts of polysaccharide chemical constituents by % mass weight compared to that found in the natural plant material or currently available Zingerber species extraction products. In certain embodiments, aspects of the disclosure relate to extraction of compounds such as volatile oil chemical constituents (essential oils), gingerol chemical constituents, phenolic acid chemical constituents, and polysaccharide chemical constituents from natural ginger plant material or from extracts of ginger plant material.

In one aspect, the disclosure features a composition comprising gingerol in an amount greater than 2% by weight.

Further embodiments feature the aforementioned composition, wherein the gingerol comprises 6-gingerol, 8-gingerol, 10-gingerol, or 6-shagaol.

Further embodiments feature the aforementioned composition, wherein the gingerol comprises 6-gingerol, 8-gingerol, 10-gingerol, and 6-shagaol.

Further embodiments feature any one of the compositions above, wherein the amount of gingerol is greater than 5, 10, 15, 20, 25, 30, 35, 40, 45, 50, 55, 60, 65, or 70% by weight.

Further embodiments feature any one of the compositions above, wherein the amount of gingerol is 50% to 70% by weight.

Further embodiments feature any one of the compositions above, wherein the amount of gingerol is 50% by weight.

Further embodiments feature any one of the compositions above, wherein the amount of gingerol is greater than 65% weight.

Further embodiments feature the aforementioned composition, further comprising an essential oil selected from the group consisting of beta-bisabolene, zingiberene, beta-sesquinhellandrene, arcurcumene, geranial, neral, champhene, phellandrene, cineol, citral, borneol, citronellol, linalool, limonene, zingiberol, betpinene, 2-undecanone, beta-elemene, beta-farnesene, cariophilene, cis-trans-alpha-farnesene, beta-sesquifel, elemol, nerolidol, beta-eudesmol, octanol, decenal, α-terpineol, and combinations thereof.

Further embodiments feature the aforementioned composition, further comprising the essential oil zingiberene.

Further embodiments feature either one of the compositions above, wherein the amount of essential oil is 5% to 20% by weight.

Further embodiments feature either one of the compositions above, wherein the gingerol comprises 6-gingerol, 8-gingerol, 10-gingerol, or 6-shagaol.

Further embodiments feature either one of the compositions above and any attendant definitions, wherein the gingerol comprises 6-gingerol, 8-gingerol, 10-gingerol, and 6-shagaol.

Further embodiments feature any one of the aforementioned compositions, wherein the amount of gingerol is greater than 5, 10, 15, 20, 25, 30, 35, 40, 45, 50, 55, 60, 65, or 70% by weight.

Further embodiments feature any one of the aforementioned compositions, wherein the amount of gingerol is 50% to 70% by weight.

Further embodiments feature any one of the aforementioned compositions, wherein the amount of gingerol is 50% by weight.

Further embodiments feature any one of the aforementioned compositions, wherein the amount of gingerol is greater than 65% by weight.

Further embodiments feature any one of the aforementioned compositions, wherein the amount of gingerol is 50% to 70% by weight, and the amount of essential oil is 5% to 20% by weight.

Further embodiments feature any one of the aforementioned compositions, wherein the amount of gingerol is greater than 65% by weight, and the amount of essential oil is greater than 10% by weight.

Further embodiments feature any one of the aforementioned compositions, wherein the amount of gingerol is 50% by weight, and the amount of essential oil is 5% by weight.

Further embodiments feature the composition first described above, further comprising a polysaccharide.

Further embodiments feature the aforementioned composition, wherein the polysaccharide comprises glucose, arabinose, galactose, rhamnose, xylose, or uronic acid.

Further embodiments feature the aforementioned composition, wherein the polysaccharide comprises glucose, arabinose, galactose, rhamnose, xylose, and uronic acid.

Further embodiments feature any one of the aforementioned compositions, wherein the amount of polysaccharide is greater than 5% to 30% by weight.

Further embodiments feature the aforementioned composition, wherein the gingerol comprises 6-gingerol, 8-gingerol, 10-gingerol, or 6-shagaol.

Further embodiments feature the aforementioned composition, wherein the gingerol comprises 6-gingerol, 8-gingerol, 10-gingerol, and 6-shagaol.

Further embodiments feature any one of the aforementioned compositions and any attendant definitions, wherein the amount of gingerol is greater than 5, 10, 15, 20, 25, 30, 35, 40, 45, 50, 55, 60, 65, or 70% by weight.

Further embodiments feature any one of the aforementioned compositions, wherein the amount of gingerol is 50% to 70% by weight.

Further embodiments feature any one of the aforementioned compositions, wherein the amount of gingerol is 50% by weight.

Further embodiments feature any one of the aforementioned compositions, wherein the amount of gingerol is greater than 65% by weight.

Further embodiments feature the aforementioned composition, wherein the amount of gingerol is 50% to 70% by weight, and the amount of polysaccharide is greater than 5% to 30% by weight.

Further embodiments feature the aforementioned composition, wherein the amount of gingerol is greater than 65% by weight, and the amount of polysaccharide is greater than 5% by weight.

Further embodiments feature the aforementioned composition, wherein the amount of gingerol is 50% by weight, and the amount of polysaccharide is 25% by weight.

Further embodiments feature any one of the aforementioned compositions, further comprising an essential oil selected from the group consisting of beta-bisabolene, zingiberene, beta-sesquinhellandrene, arcurcumene, geranial, neral, champhene, phellandrene, cineol, citral, borneol, citronellol, linalool, limonene, zingiberol, betpinene, 2-undecanone, beta-elemene, beta-farnesene, cariophilene, cis-trans-alpha-farnesene, beta-sesquifel, elemol, nerolidol, beta-eudesmol, octanol, decenal, α-terpineol, and combinations thereof.

Further embodiments feature any one of the aforementioned compositions, further comprising the essential oil zingiberene.

Further embodiments feature either of the compositions above, wherein the amount of essential oil is 5% to 20% by weight.

Further embodiments feature any one of the aforementioned compositions, further comprising phenolics.

Further embodiments feature the composition above, wherein the amount of phenolics is greater than 1% to 25% by weight.

In another aspect, the disclosure features a method for extracting a Ginger species comprising, sequentially extracting a Ginger species plant material to yield an essential oil fraction, a gingerol fraction, a phenolic fraction, and a polysaccharide fraction, wherein the essential oil and gingerol fractions are derived by extracting plant feedstock material by supercritical carbon dioxide extraction, the phenolic fraction is extracted from the plant feedstock material or from the remainder of the essential oil and gingerol extractions by hydroalcoholic extraction, and the polysaccharide fraction is derived by hot water extraction of the remainder of the phenolic extraction.

Further embodiments feature the aforementioned method, wherein phenolic extraction comprises: (a) contacting a plant feedstock material, or remainder thereof from an extraction of essential oil and gingerol fractions by supercritical carbon dioxide, with a hydroalcoholic mixture for a time sufficient to extract phenolics to form an aqueous solution of extracted phenolics; (b) passing the aqueous solution of extracted phenolics through an adsorbent resin column wherein the phenolics are adsorbed; and (c) eluting phenolics from adsorbent resin.

In another aspect, the disclosure features the aforementioned compositions and any attendant definitions, further comprising a pharmaceutical carrier. The compositions of the disclosure may comprise pastes, resins, oils, beverages, liquid infusion or decoction, powders, and dry flowable powders. Such products are processed for many different uses, including, but not limited to, a fast dissolve tablet or other oral delivery forms. The compositions of the disclosure may be used alone or in combination with other compositions such as other botanical extraction materials, herbal remedies, pharmacological agents, food, dietary supplements, or beverages. Compositions of the disclosure may be used for treatment of physiological and medical conditions.

The compositions of the disclosure are useful in providing physiological and medical effects including, but not limited to, anti-nausea and vomiting related to motion sickness, pregnancy, surgery, anesthesia, and cancer chemotherapy without drowsiness or fetal risk, anti-inflammatory, anti-arthritis, anti-rheumatic disorders, analgesia, anti-oxidant activity, oxygen free radical scavenging, nitrosation inhibition, anti-hyperlipidemia or hypercholesterolemia, anti-thrombosis, anti-hypertension, vasodilation, anti-cardiac palpitations, anti-atherosclerosis, anti-obesity, cardiovascular disease prevention and treatment, stroke prevention and treatment, anti-Alzheimer's disease, anti-Parkinson's disease, headache and migraine prevention and therapy, immunomodulation, anti-autoimmune disease, radiation protection, anti-colic and dyspepsia, anti-diarrhea, anti-heart burn, anti-flatulence, anti-indigestion, anti-mutagenic activity (cancer prevention), anti-carcinogenic activity (cancer therapy), skin protection, impotence, common cold, influenza, anti-bacterial activity, aphrodisiac, aromatic, and carminative.

These embodiments of the disclosure, other embodiments, and their features and characteristics, will be apparent from the description, drawings and claims that follow.

### BRIEF DESCRIPTION OF THE DRAWINGS

FIG. 1 depicts an exemplary method for the preparation of an essential oil fraction from plant feedstock.

FIG. 2 depicts an exemplary method for the preparation of purified and/or profiled essential oil sub-fractions.

FIG. 3 depicts an exemplary method for the preparation of phenolic fractions.

FIG. 4 depicts an exemplary method for the preparation of purified phenolic fractions using polymer adsorbent.

FIG. 5 depicts an exemplary method for the preparation of polysaccharide fractions.

FIG. 6 depicts AccuTOF-DART mass spectra for purified ginger polysaccharide fractions: (a) PS60 positive ion mode; and (b) PS60 negative ion mode.

FIG. 7 depicts AccuTOF-DART mass spectra for purified ginger polysaccharide fractions: (a) PS80 positive ion mode; and (b) PS80 negative ion mode.

FIGS. 8A-G depict AccuTOF-DART mass spectra for single stage SCCO₂ extracts.

FIGS. 9A-E depict AccuTOF-DART mass spectra for multi stage SCCO₂ extracts.

FIGS. 10A-B depict AccuTOF-DART mass spectra for fractional SCCO₂ separation of ginger essential oil.

FIGS. 11A-E depict AccuTOF-DART mass spectra for alcoholic leaching extracts of a residue SCCO₂ extraction.

### DETAILED DESCRIPTION OF THE INVENTION

### Definitions

Aspects of the disclosure relate to compositions of Zingerber species such as, but not limited, to its rhizome parts, and processes for the preparation and/or formulation thereof. As used herein, Ginger refers to the rhizome plant material derived from the Zingerber species botanical. The term “Ginger” is also used interchangeably with Zingerber species and means these plants, clones, variants, and sports, inter alia.

As used herein, the term “one or more compounds” means that at least one compound, such as, but not limited to, zingiberene (a lipid soluble volatile oil chemical constituent of Ginger species), or gingerol (an oleoresin of Ginger species) or 6-gingerol (a phenolic oleoresin of Ginger species), or a polysaccharide molecule of Ginger species is intended, or that more than one compound, for example, zingiberene and 6-gingerol is intended. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents.

As used herein, the term “fraction” means the extraction composition comprising a specific group of chemical compounds characterized by certain physical, chemical properties or physical or chemical properties.

As used herein, the term volatile oil fraction comprises lipid soluble, water insoluble compounds obtained or derived from Ginger and related species including, but not limited to, the chemical compound classified as zingiberene.

As used herein, the term volatile oil sub-fraction comprises lipid soluble, water insoluble compounds obtained or derived from Ginger and related species including, but not limited to, the chemical compound classified as zingiberene having enhanced concentrations of specific compounds found in the volatile oil of Ginger species.

As used herein, the term “gingerol” comprises the lipid soluble, water insoluble compounds obtained or derived from Ginger and related species including, but not limited to, the chemical compounds classified as gingerols such as 6-gingerol, 8-gingerol, 10-gingerol, gingerdiols, such as 6-gingerdiol, shagaols, such as 6-shagaol, and paradols, such as 6-paradol.

As used herein, the term “phenolic” comprises the water soluble and ethanol soluble polyphenolic acid compounds obtained or derived from Ginger and related species, further comprising, but not limited to, compounds such as 6-gingerol, 8-gingerol, and 10-gingerol.

As used herein, the term “polysaccharide” comprises water soluble-ethanol insoluble polysaccharide compounds obtained or derived from Ginger and related species.

As used herein, the term “purified” fraction or composition means a fraction or composition comprising a specific group of compounds characterized by certain physical-chemical properties or physical or chemical properties that are concentrated to greater than 20% of the fraction's or composition's chemical constituents. In other words, a purified fraction or composition comprises less than 80% chemical constituent compounds that are not characterized by certain desired physical-chemical properties or physical or chemical properties that define the fraction or composition.

As used herein, the term “profile” refers to the ratios by percent mass weight of the chemical compounds within an extraction fraction or sub-fraction or to the ratios of the percent mass weight of each of the four ginger fraction chemical constituents in a final ginger extraction composition. The term “profile” may also be used to refer to the ratios by percent mass weight of fractions or sub-fractions comprising compositions that contain more than one of the above ginger fractions.

As used herein, “feedstock” generally refers to raw plant material, comprising whole plants alone, or in combination with on or more constituent parts of a plant comprising leaves, roots, including, but not limited to, main roots, rhizomes, tail roots, and fiber roots, stems, bark, leaves, seeds, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to affected the size and physical integrity of the plant material. Occasionally, the term “feedstock” may be used to characterize an extraction product that is to be used as feed source for additional extraction processes.

As used herein, the term “Ginger constituents” shall mean chemical compounds found in Ginger species and shall include all such chemical compounds identified above as well as other compounds found in Ginger species, including but not limited to the volatile oil chemical constituents, the gingerol chemical constituents, phenolic chemical constituents, and polysaccharides.

### Compositions

Some of the component gingerols are depicted in Scheme 1 below. A summary of the known chemical constituents of Zingiber species rhizome is found Table 1.

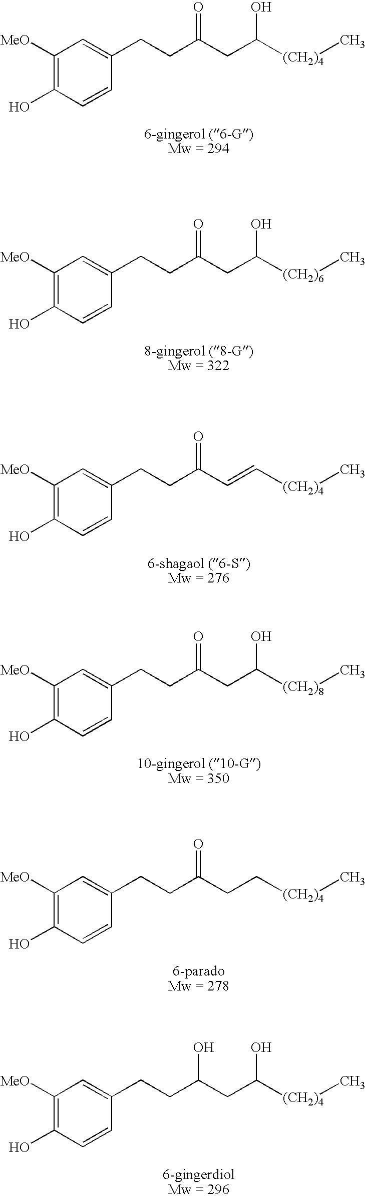

TABLE 1
Chemical constituents of Z. officianale rhizome based on literature.

| Constitiuents | | % mass weight |
|---|---|---|
| 1. | Volatile Oils | 1.0-3.3 (1.7) |

| | |
|---|---|
| a) | Monoterpenes |
| | betpinene |
| | neural |
| | geranial |
| | 2-undecanone |
| | beta-elemene |
| | beta-farnesene |
| b) | Sesquiterpenes (30-70% of volatile oils) |
| | (+)-ar-curcumene |
| | beta-bisabolene |
| | (−)-zingiberene |
| | beta-sequiphellandrene |
| | cariophilene |
| | alpha-zingiberene |
| | alpha-farnesene |
| | beta-sesquifel |
| | elemol |
| | cis-nerolidol |
| | trans-nerolidol |
| | beta-eudesmol |
| c) | Others |
| | cineol |
| | citral |
| | borneol |
| | citronellol |
| | linalool |
| | imonene |
| | zingerberol |

| | | |
|---|---|---|
| 2. | Non-volatile oils (oleoresin & gingerols) | 4.0-7.5 |

| | |
|---|---|
| gingerol (6-gingerol + 8-gingerol + | 0.6-1.4 (1.46) |
| 10-gingerol) | |
| palmitic acid | |
| farnesol | |
| m-linoleil | |
| cis-6-shogaol | |
| trans-6-shogaol | |
| trans-6-gingerol | |
| cis-m-gingerol | |
| m-8-gingerol | |
| trans-m-6-gingerol | |
| trans-8-shogaol | |
| syn-6-m-8-shogaol | |
| trans-10-shogaol | |
| trans-10-gingerol | |

| | | |
|---|---|---|
| 3. | Phenolics | (0.8) |
| 4. | Other Hydrocarbons | |
| 5. | Carbohydrate (mainly starch) | 40-60 |
| | Polysaccharides | (1.2) |
| 6. | Proteins | 9-10 |
| 7. | Lipids (triglycerides, phosphatitic acid, lecithins, | 6-10 |
| | fatty acids) | |
| 8. | Amino acids | |
| 9. | Others (Vit B6 & C, Ca, Mg, Phos, Potassium) | |

*bracketed (#) % mass weight value were measured on the ginger feedstock used in the disclosure.

Another chemical suspected having strong anti-nausea properties is galanolactone.

It is believed that synergistic bioactivity relationships exist among the bio-active chemical constituents.

The disclosure comprises compositions and methods for making and using Ginger and related species compositions, wherein the compositions comprise oral delivery dosage formulations, comprising the compositions taught herein. Such compositions include compositions that have predetermined amounts of at least one of the volatile oil, gingerol, phenolic, or polysaccharide fractions. Certain embodiments comprise compositions of Ginger and related species having at least one of a volatile oil, gingerol, phenolic, or polysaccharide fraction concentration that is in an amount greater than that found in the native Ginger and related species plant material or currently available Ginger species extract products. Certain embodiments also comprise compositions wherein one or more of the fractions, including volatile oils, gingerols, phenolics, or polysaccharides, are found in a concentration that is greater than that found in native Ginger species plant material. Certain embodiments also comprise compositions wherein one or more of the fractions, including volatile oils, gingerols, phenolics, or polysaccharides, are found in a concentration that is less than that found in native Ginger species.

For example, compositions of the disclosure comprise compositions wherein the concentration of volatile oils is from about 0.001 to about 60 times the concentration of native Ginger species, and/or compositions wherein the concentration of desired gingerols is from about 0.001 to about 50 times the concentration of native Ginger species, and/or the concentration of phenolics if from about 0.001 to about 40 times the concentration in native Ginger species, and/or compositions where the concentration of water soluble-ethanol insoluble polysaccharides is from about 0.001 to about 90 times the concentration of native Ginger species. Compositions of the disclosure comprise compositions wherein the concentration of volatile oils is from about 0.01 to about 60 times the concentration of native Ginger species, and/or compositions wherein the concentration of gingerols is from about 0.01 to about 50 times the concentration of native Ginger species, and/or phenolics is from about 0.01 to about 50 times the concentration of native Ginger species, and/or compositions wherein the concentration of polysaccharides is from about 0.01 to about 90 times the concentration of native Ginger species. Furthermore, compositions of the disclosure comprise sub-fractions of the volatile oil chemical constituents having at least one or more of chemical compounds present in the native plant material essential oil that is in amount greater or less than that found in native Ginger plant material volatile oil chemical constituents. For example, the chemical compound, zingiberene, may have its concentration increased in an essential oil sub-fraction to 13.7% by % mass weight of the sub-fraction or decreased to 4.7% by % mass weight of the total volatile oil chemical constituents in the sub-fraction. Compositions of the disclosure comprise compositions wherein the concentration of specific chemical compounds in such novel volatile oil sub-fractions is either increase by about 1.1 to about 3 times or decreased by about 0.1 to about 3 times that concentration found in the native Ginger volatile oil chemical constituents.

Compositions of the disclosure comprise combinations of one or more extraction compositions taught herein. In certain embodiments, a composition comprises Ginger volatile oil fractions and gingerol fraction compositions, optionally including the components: pentanal, 2-methyl-; α-thujene; camphene; hydroperoxide, hexyl; octanal; 4(10)-thujene; 5-hepten-2-one, 6-methyl-; borneol; α-terpineol; decanal; 1,3-di-tert-butylbenzene; 2-nonanone; α-linalool; 2-decenal, (E)-; 1,6,10-dodecatriene, 7,11-dimethyl-3-methylene-, (E)-; α-farnesene; β-caryphylline; β-cis-caryophylline; α-caryophylline; trans-α-bergamotene; α-zingiberene; germacrene D; β-bisabolene; α-cubebene; (Z,Z)-α-farnesene; (−)-α-panasinsen; β-sesquiphellandrene; 5,7-octadien-2-ol, 2,6-dimethyl-; butanoic acid, 3-hexenyl ester, (E)-; β-cis-farnesene; 4-((1E)-3-hydroxy-1-propenyl)-2-methoxyphenol; diethyl phthalate; 6,10-dodecadien-1-yn-3-ol, 3,7,11-trimethyl-; 2-furanmethanol, tetrahydro-, acetate; β-farnesene; stereoisomers of farnesene; gingerol; β-eudesmol; ledol; ledane; farnesol; caryophyllene oxide; 6,10-dodecadien-1-yn-3-ol, 3,7,11-trimethyl-; xanthorrhizol; (Z)-nerolidol; α-bisabolol; epi-α-bisabolol; levomenol; hexadecanoic acid, methyl ester; α-curcumene; 1-tetradecyne; methyl 2-hydroxydecanoate; 1-pentadecyne; methyl 2-hydroxydodecanoate; 2-pentenoic acid, 3-methyl-5-(2,6,6-trimethyl-1-cyclohexenyl); cis,cis-farnsol; hexadecanoic acid, 1,1-dimethylethyl ester; (+)-6-gingerol; 6-shagaol; octadecanoic acid, butyl ester; 8-shagaol; carinol; gingerol isomers; 6-gingerol; 8-gingerol; 6-shagaol; and 10-gingerol.

A further embodiment of a composition comprises a polysaccharide fraction composition, having a purity of about 350-590 mg/g 5K dextran equivalence, which may be determined by colormetric analytical methods.

In certain embodiments, a composition of the disclosure may comprise from about 5% to about 96% by mass weight of the volatile oil chemical constituents in the total composition. An embodiment of such compositions comprises a predetermined gingerol concentration that is greater than that which is present in natural ginger plant material or conventional ginger species extract products which can result from the extraction techniques taught herein. For example, a composition may comprise from about 5% to about 65% by mass weight of the gingerol chemical constituents in the total composition. Another embodiment of such compositions comprises a predetermined novel phenolic concentration in the extracted Ginger species composition wherein the phenolic acid concentration is greater than that found in the native plant material or conventional Ginger species extracts. For example, a composition may comprise phenolic acids at a concentration of about 2% to about 30% by mass weight of the total composition. A further embodiment of such compositions comprises a predetermined polysaccharide concentration substantially increased in relation to that found in natural Ginger species dried plant material or conventional Ginger species extract products. For example, an extract composition may comprise water soluble-ethanol insoluble polysaccharide chemical constituents of about 2% to about 90% by mass weight of the total composition.

In making a combined composition, from about 0.001 mg to about 1000 mg of a volatile oil fraction can be used. Moreover, from about 0.001 mg to about 1000 mg of a gingerol fraction can be used. Additionally, from about 0.001 mg to about 1000 mg of a phenolic fraction composition can be used. Further, from about 0.001 mg to about 1000 mg of the water-soluble ethanol insoluble polysaccharide fraction can be used.

An embodiment of such compositions comprise predetermined concentrations of the extracted and purified chemical constituent fractions wherein the Ginger species volatile oil/gingerol fraction, volatile oil fraction/phenolic fraction, volatile oil fraction/polysaccharide fraction, gingerol fraction/phenolic fraction, gingerol fraction/polysaccharide fraction, and phenolic fraction/polysaccharide fraction concentration (% dry weight) profiles (ratios) are greater or less than that found in the natural dried plant material or conventional Ginger species extraction products.

### Methods of Ginger Rhizome Extraction

Aspects of the disclosure also relates to processes for concentrating (purifying) and profiling the volatile oil and other lipid soluble compounds from Ginger plant material using SCCO₂ technology. The disclosure includes the fractionation of the lipid soluble chemical constituents of Ginger into, for example, a volatile oil fraction of high purity (high volatile oil chemical constituent concentration) and a gingerol fraction of high purity (high gingerol chemical constituent concentration). Moreover, the disclosure includes a SCCO₂ process wherein the individual chemical constituents within an extraction fraction may have their chemical constituent ratios or profiles altered. For example, SCCO₂ fractional separation of the chemical constituents within a volatile oil fraction permits the preferential extraction of certain volatile oil compounds relative to the other volatile oil compounds such that a volatile oil extract sub-fraction can be produced with a concentration of certain compounds greater than the concentration of other compounds. Extraction of the volatile oil and gingerol chemical constituents of the Ginger species with SCCO₂ as taught in the disclosure eliminates the use of toxic organic solvents and provides simultaneous fractionation of the extracts. Carbon dioxide is a natural and safe biological product and an ingredient in many foods and beverages.

The starting material for extraction is plant material from one or more Ginger species. The plant material may be any portion of the plant, though the rhizome is the most preferred starting material.

The Ginger species plant material may undergo pre-extraction steps to render the material into any particular form, and any form that is useful for extraction is contemplated by the disclosure. Such pre-extraction steps include, but are not limited to, those wherein the material is chopped, minced, shredded, ground, pulverized, cut, or torn, and the starting material, prior to pre-extraction steps, is dried or fresh plant material. A preferred pre-extraction step comprises grinding and/or pulverizing the Ginger species rhizome material into a fine powder. The starting material or material after the pre-extraction steps can be dried or have moisture added to it. Once the Ginger species plant material is in a form for extraction, methods of extraction are contemplated by the disclosure.

Methods of extraction of the disclosure comprise processes disclosed herein. In general, methods of the disclosure comprise, in part, methods wherein Ginger species plant material is extracted using supercritical fluid extraction (SFE) with carbon dioxide as the solvent (SCCO₂) that is followed by one or more solvent extraction steps, such as, but not limited to, water, hydroalcoholic, and affinity polymer absorbent extraction processes. Additional methods contemplated for the disclosure comprise extraction of Ginger species plant material using other organic solvents, refrigerant chemicals, compressible gases, sonification, pressure liquid extraction, high speed counter current chromatography, molecular imprinted polymers, and other known extraction methods. Such techniques are known to those skilled in the art.

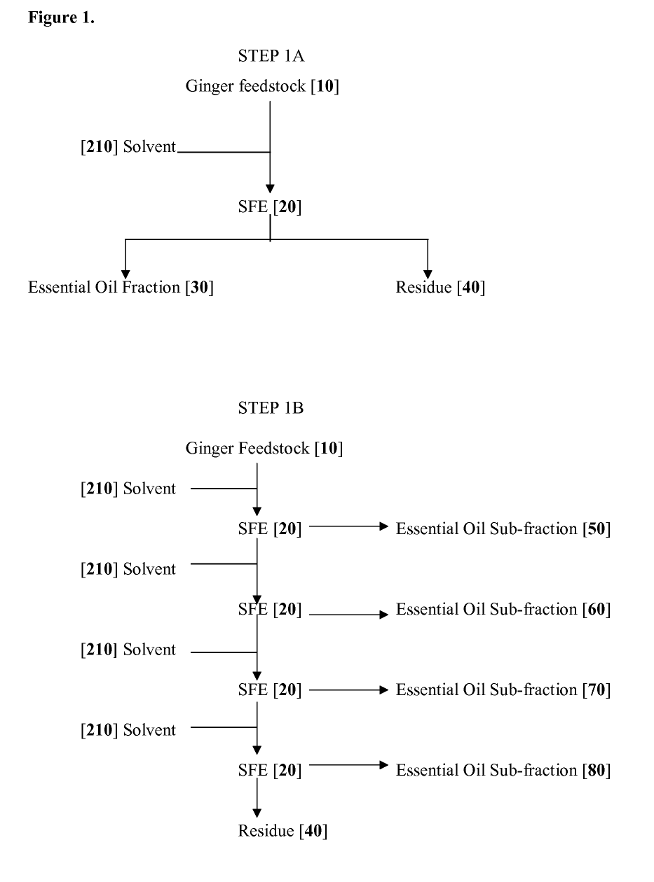

A schematic diagram of the methods of extraction of the biologically active chemical constituents of ginger is illustrated in FIGS. 1-5. The extraction process is typically, but not limited to, 4 steps. For reference in the text herein, when bold numbers appear in brackets [#], the numbers refer to the numbers in FIGS. 1-5.

### Step 1: Supercritical Fluid Carbon Dioxide Extraction of Ginger Essential Oil

Due to the hydrophobic nature of the essential oil, non-polar solvents, including, but not limited to SCCO₂, hexane, petroleum ether, and ethyl acetate may be used for this extraction process. Since some of the components of the essential oil are volatile, steam distillation may also be used as an extraction process. However, steam distillation cannot recovery the pungent components, since the dominant pungent components, the gingerols, are thermally degraded to produce volatile aldehydes or ketones. Some of the other aromatic components also have been shown to be degraded by heat. When extracted with organic solvent, the oleoresin or extracted essential oil lacks a strong aroma due the loss of volatile components during the evaporation process of the solvent. The extraction of ginger by SCCO₂ offers extracts with both aromatic and pungent components. SCCO₂ is carried out at relatively low temperature and solvent removal from the extract is quite easy, so that any alteration of heat sensitive components and the loss of volatile components are minimized. Furthermore, CO₂ as the solvent has high selectivity for the lipid soluble and volatile flavor components. There are numerous studies on the extraction of ginger with CO₂ (51-54).

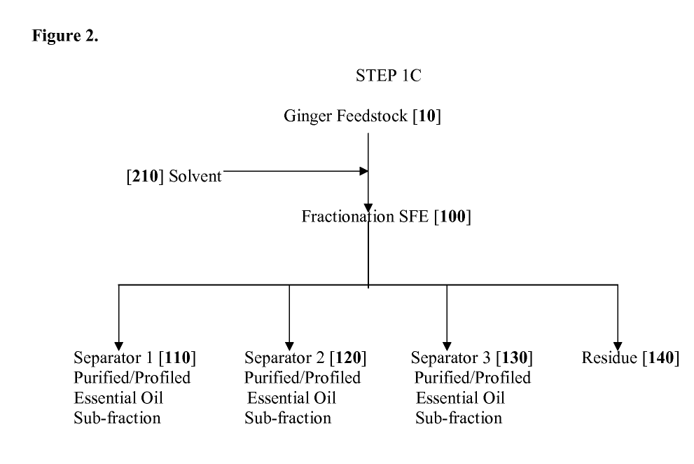

A generalized description of the extraction of the essential oil chemical constituents from the rhizome of the Ginger species using SCCO₂ is diagrammed in FIGS. 1 & 2—Steps 1A, 1B and 1C. The feedstock [10] is dried ground ginger bark (about 140 mesh). The extraction solvent [210] is pure carbon dioxide. Ethanol may be used as a co-solvent. The feedstock is loaded into a SCCO₂ extraction vessel [20]. After purge and leak testing, the process comprises liquefied CO₂ flowing from a storage vessel through a cooler to a CO₂ pump. The CO₂ is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO₂ extractions taught herein are preferably performed at pressures of at least 100 bar and a temperature of at least 35° C., and more preferably at a pressure of about 60 bar to 500 bar and at a temperature of about 40° C. to about 80° C. The time for extraction for a single stage of extraction range from about 30 minutes to about 2.5 hours, to about 1 hour. The solvent to feed ratio is typically about 60 to 1 for each of the SCCO₂ extractions. The CO₂ is recycled. The extracted, purified, and profiled essential oil chemical constituents [30] are then collected a collector or separator, saved in a light protective glass bottle, and stored in a dark refrigerator at 4° C. The Ginger feedstock [10] material may be extracted in a one step process (FIG. 1, Step 1A) wherein the resulting extracted and purified Ginger essential oil fraction [30] is collected in a one collector SFE or SCCO₂ system [20] or in multiple stages (FIG. 1, Step 1B) wherein the extracted purified and profiled Ginger essential oil (volatile oil and gingerol) sub-fractions [50, 60, 70, 80] are separately and sequentially collected in a one collector SFE system [20]. Alternatively, as in a fractional SFE system [100] (FIG. 2, Step 1C), the SCCO₂ extracted Ginger feedstock material may be segregated into collector vessels (separators) such that within each collector there is a differing relative percentage essential oil chemical constituent composition (profile) in each of the purified essential oil sub-fractions collected [110, 120, 130]. The residue (remainder) [40] is collected, saved and used for further processing to obtain purified fractions of the Ginger species phenolics and polysaccharides. An embodiment of the disclosure comprises extracting the Ginger species feedstock material using multi-stage SCCO₂ extraction at a pressure of 60 bar to 500 bar and at a temperature between 35° C. and 90° C. and collecting the extracted Ginger material after each stage. A second embodiment of the disclosure comprises extracting the Ginger species feedstock material using fractionation SCCO₂ extraction at pressures of 60 bar to 500 bar and at a temperature between 35° C. and 90° C. and collecting the extracted Ginger material in differing collector vessels at predetermined conditions (pressure, temperature, and density) and predetermined intervals (time). The resulting extracted Ginger purified essential oil sub-fraction compositions from each of the multi-stage extractors or in differing collector vessels (fractional system) can be retrieved and used independently or can be combined to form one or more Ginger essential oil compositions comprising a predetermined essential oil chemical constituent concentration that is higher or lower than that found in the native plant material or in conventional Ginger extraction products. Typically, the total yield of the essential oil fraction from ginger species using a single step maximal SCCO₂ extraction is about 0.2 to about 1.8% (>95% of the essential oil chemical constituents) by % weight having an essential oil chemical constituent purity of greater than 95% by mass weight of the extract.

In a single-step SFE maximal extraction and purification, the highest yield of the essential oil is obtained with SCCO₂ conditions of 40° C. and 300 bar. Using these optimum conditions (40° C., 300 bar), the chemical constituent composition of the extract is as follows: 35-38% gingerols, 33% sesquiterpenes, and 8-9% oxygenated sesquiterpenes (see Example 1, Tables 5 and 6). The gingerol chemical constituent purity is similar using both HPLC and GC-MS analytical methods supporting the conclusion that the essential oil extracts were of high purity (>95% by mass weight of the extract. The purity of the gingerol chemical constituents in the SCCO₂ extracts ranged from about 22% to 43%. Higher purity of the gingerols is achieved when the density of CO₂ is greater than 0.64 gm/ml. 6-gingerol makes up about 50% of the total gingerols in these extracts resembling the extracts obtained with organic solvents. In contrast with the organic solvent extracts, the SCCO₂ extracts exhibited a different profile of the chemical constituents. The monoterpene concentration was <1% by mass weight. The gingerol concentration increased with increasing SCCO₂ pressure and temperature. The sesquiterpene hydrocarbon concentration increased with decreasing pressure. These data indicate that low temperature favors sesquiterpene extraction and high pressure favors gingerol extraction indicating that SCCO₂ may be used to fractionate the essential oil and oleoresin into novel volatile oil fractions (sub-fractions) and novel gingerol fractions.

Data from multi-stage SCCO₂ extraction/fractionation confirm that multi-stage SCCO₂ can also fractionate ginger essential oil into purified volatile oil fractions (or sub-fractions) and purified gingerol fractions (or sub-fractions) using step increases in SCCO₂ pressure (see Example 2, Tables 7 and 8). The gingerol fraction purity can be about 55-68% by mass weight of the extract fraction (third and fourth stages). The volatile oil fraction contains less than 20% gingerols by mass weight of the extract fraction (first stage). The highest purity of sesquiterpenes is present in the first stage volatile oil fraction. Interestingly, oxygenated sesquiterpenes are found in high purity (23% by mass weight) as well as the compound 6-shogaol (25% by mass weight) in the second volatile oil extract fraction. The chemical constituent profiles of the gingerol fractions (third and fourth stages) are similar with low concentrations of sesquiterpenes and oxygenated sesquiterpenes by % mass weight of the gingerol extract fraction.

Based on a typical experiment utilizing a fractional SCCO₂ separation protocol, as well as others, approximately 85% of the gingerols in the native ginger rhizome feedstock are extracted in this single stage SCCO₂ extraction and fractionation of the essential oil chemical constituents (see Example 3, Tables 9 and 10). Moreover, the purity of the gingerols in separator 1 can be up to 65% by mass weight of the gingerol extract fraction (a 45 fold increase in concentration of the gingerols over that found in the native feedstock). In contrast the purity of the sesquiterpenes is separator 1 is only 15% by mass weight but the sesquiterpene purity in separator 2 can be up to 75% by mass weight of the volatile oil extract fraction (a 90-fold increase in the concentration of the sesquiterpenes over that found in the native feedstock). Comparing the multistage SCCO₂ fractionation to the single stage SCCO₂ fractional separation system, the single-stage fractionation separation system appears to be a more optimal process with respect to maximizing total yield and purity of the desired chemical constituents in the extract fractions.

### Step 2. Hydroalcoholic Leaching Process for Extraction of Crude Phenolic Fraction

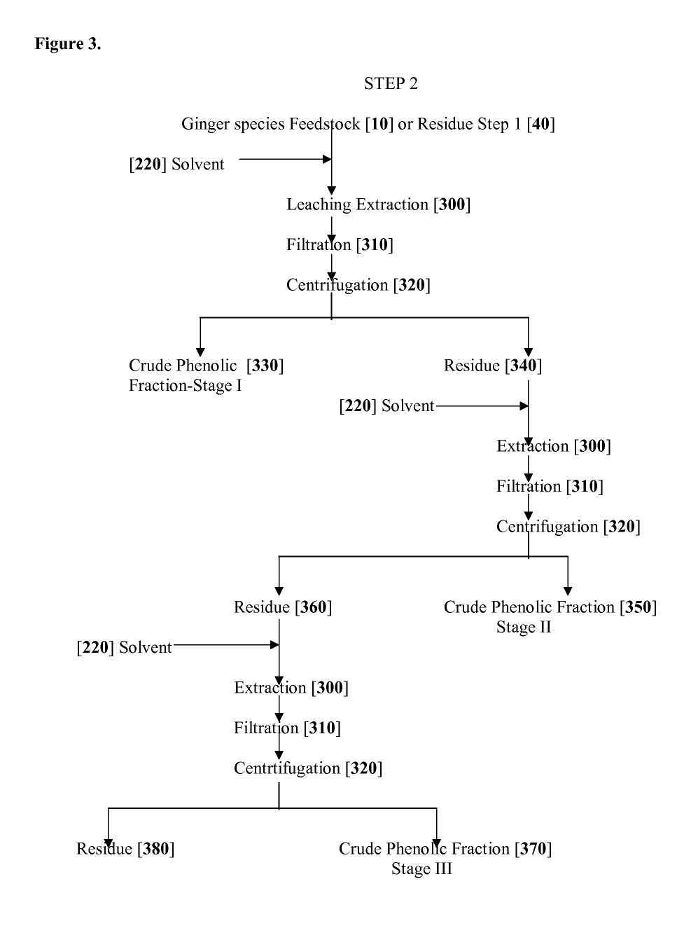

In one aspect, the disclosure comprises extraction and concentration of the bio-active phenolic chemical constituents. A generalized description of this step is diagrammed in FIG. 3. This Step 2 extraction process is a solvent leaching process. The feedstock for this extraction is either Ginger species ground rhizome material [10] or the residue [40] from the Step 1 SCCO₂ extraction-fractionation of the essential oil (volatile oil and gingerol) chemical constituents. The extraction solvent [220] is aqueous ethanol. The extraction solvent may be 10-95% aqueous alcohol, 25% aqueous ethanol is preferred. In this method, the Ginger feedstock material and the extraction solvent are loaded into an extraction vessel [300] that is heated and stirred. It may be heated to 100° C., to about 90° C., to about 80° C., to about 70° C., to about 60° C. or to about 30-50° C. The extraction is carried out for about 1-10 hours, for about 1-5 hours, for about 2 hours. The resultant extract solution is filtered [310] and centrifuged [320]. The filtrate (supernatant) [330, 350, 370] is collected as product, measured for volume and solid content dry mass after evaporation of the solvent. The extraction residue material [380] may be retained and saved for further processing or discarded. The extraction may be repeated as many times as is necessary or desired. It may be repeated 2 or more times, 3 or more times, 4 or more times, etc. FIG. 3—STEP 2 shows a three-stage process, where the second stage and third stage use the same methods and conditions.

### Step 3. Affinity Adsorbent Extraction Process

As taught herein, a purified phenolic fraction extract from Ginger and related species may be obtained by contacting a hydroalcoholic extract of Ginger feedstock with a solid affinity polymer adsorbent resin so as to adsorb the active phenolics contained in the hydro-alcoholic extract onto the affinity adsorbent. The bound chemical constituents are subsequently eluted by the methods taught herein. Prior to eluting the phenolic acid fraction chemical constituents, the affinity adsorbent with the desired chemical constituents adsorbed thereon may be separated from the remainder of the extract in any convenient manner, preferably, the process of contacting with the adsorbent and the separation is effected by passing the aqueous extract through an extraction column or bed of the adsorbent material.

A variety of affinity adsorbents can be utilized to purify the phenolic acid chemical constituents of Ginger species, such as, but not limited to “Amberlite XAD-2” (Rohm & Hass), “Duolite S-30” (Diamond Alkai Co.), “SP207” (Mitsubishi Chemical), ADS-5 (Nankai University, Tianjin, China), ADS-17 (Nankai University, Tianjin, China), Dialon HP 20 (Mitsubishi, Japan), and Amberlite XAD7 HP (Rohm & Hass). Amberlite XAD7 HP is preferably used due to the high affinity for the phenolic acid chemical constituents of Ginger and related species.

Although various eluants may be employed to recover the phenolic chemical constituents from the adsorbent, in one aspect of the disclosure, the eluant comprises low molecular weight alcohols, including, but not limited to, methanol, ethanol, or propanol. In a second aspect, the eluant comprises low molecular alcohol in an admixture with water. In another aspect, the eluant comprises low molecular weight alcohol, a second organic solvent, and water.

Preferably, the Ginger species feedstock has undergone a one or more preliminary purification process such as, but not limited to, the processes described in Step 1 and 2 prior to contacting the aqueous phenolic chemical constituent containing extract with the affinity adsorbent material.

Using affinity adsorbents as taught in the disclosure results in highly purified phenolic chemical constituents of the Ginger species that are remarkably free of other chemical constituents which are normally present in natural plant material or in available commercial extraction products. For example, the processes taught in the disclosure can result in purified phenolic extracts that contain total phenolic acid chemical constituents in excess of 30% by dry mass weight of the extract fraction.

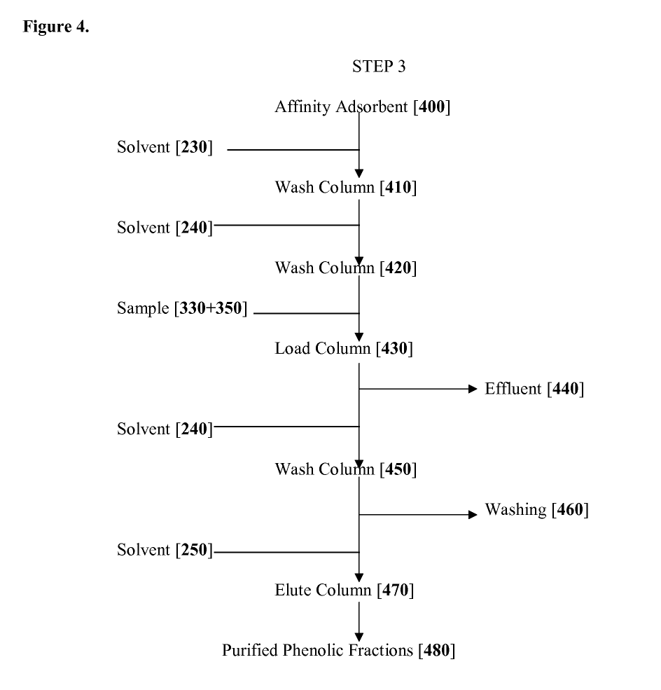

A generalized description of the extraction and purification of the phenolics from the roots of the Ginger species using polymer affinity adsorbent resin beads is diagrammed in FIG. 4. The feedstock for this extraction process may be the aqueous ethanol solution containing the phenolics from Step 2 Hydroalcoholic Leaching Extraction [330+/−350]. The appropriate weight of adsorbent resin beads (5 mg of phenolic acids per gm of adsorbent resin) is washed with 4-5 BV ethanol [230] and 4-5 BV distilled water [240] before and after being loaded into a column [410,420]. The phenolic containing aqueous solution [330+340] is then loaded onto the column [430] at a flow rate of 2.4 bed volume (BV)/hour. Once the column is fully loaded, the column is washed [450] with distilled water [240] at a flow rate of 3 BV/hour to remove any impurities from the adsorbed phenolics. The effluent residue [440] and washing residue [460] were collected, measured for mass content, phenolic acid, and discarded. Elution of the adsorbed phenolics [470] is accomplished in an isocratic fashion with 50-75% ethanol/water as an eluting solution [250] at a flow rate of 7 BV/hour and the elution curve was recorded for the eluate extract [480]. Elution volumes [480] may be collected at timed intervals and these samples are analyzed using HPLC and tested for solids content and purity.

In an exemplary experiment, the total yield of the hydro-alcoholic leaching crude phenolic extract was 12.4% by mass weight based on the original ginger rhizome feedstock with a phenolic acid purity of 5.9% by mass weight of the crude phenolic extract fraction (see Example 5, Table 11). Interestingly, the gingerols make up greater than 95% by mass weight of the total phenolic acids extracted. The gingerol yield was 0.71% by mass weight based on the original rhizome. These gingerols are the gingerols left in the residue after SCCO₂ processing. Remarkably, the profile of the gingerols in the leaching extract is different from that found in the original feedstock or the SCCO₂ gingerol extract with 6-gingerol now making up 62% by mass weight of the total gingerols. During the affinity adsorbent processing, no phenolics were detected in the effluent or washing samples. Greater than 80% of the loaded phenolics may be eluted using 75% ethanol. The purity of the total phenolics in the F3 and F4 fractions ranges from 26% to 30% by mass weight of the fraction. Combining F3 and F4 fractions results in a total yield of 0.7% based on the original ginger rhizome feedstock. Furthermore, 6-gingerol makes up about 90% of the total phenolics in these novel purified phenolic extract fractions.

### Step 4. Polysaccharide Fraction Extraction Processes

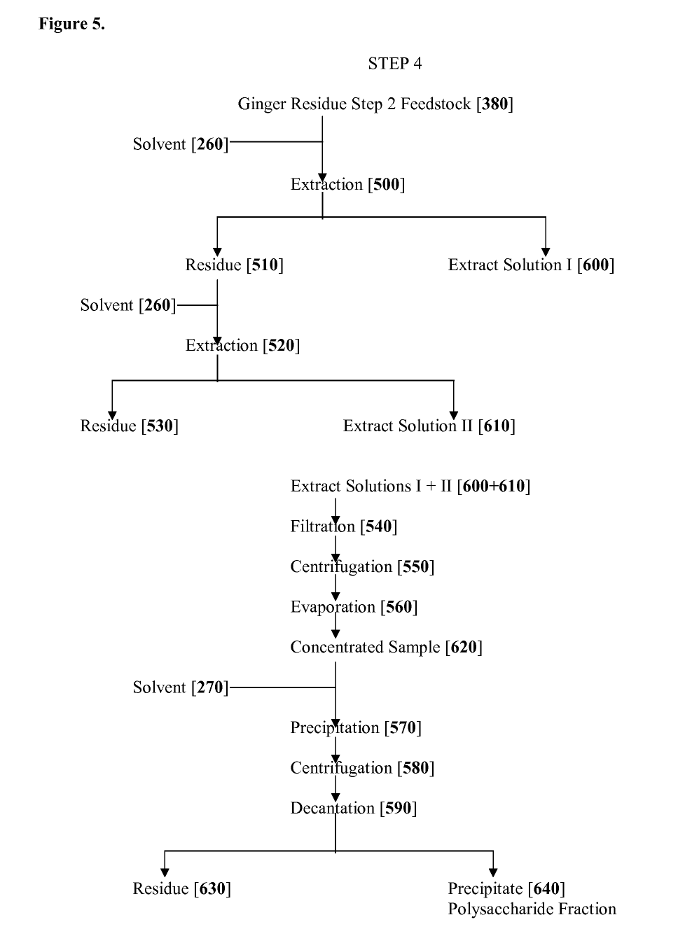

The polysaccharide extract fraction of the chemical constituents of Ginger species has been defined in the scientific literature as the “water soluble, ethanol insoluble extraction fraction”. A generalized description of the extraction of the polysaccharide fraction from extracts of Ginger species using water solvent leaching and ethanol precipitation processes is diagrammed in FIG. 5. The feedstock [380] is the solid residue from the hydro-alcoholic leaching extraction process of Step 2. This feedstock is leaching extracted in two stages. The solvent is distilled water [260]. In this method, the Ginger species residue [380, 510] and the extraction solvent 260 are loaded into an extraction vessel [500, 520] and heated and stirred. It may be heated to 100° C., to about 80° C., or to about 60-80° C. The extraction is carried out for about 1-5 hours, for about 2-4 hours, or for about 3 hours. The two stage extraction solutions [600+610] are combined and the slurry is filtered [540], centrifuged [550], and may be evaporated [560] to remove water until an about 8-fold increase in concentration of the chemicals in solution [620] to reduce the amount of alcohol required for the precipitation. Anhydrous ethanol [270] is then used to reconstitute the original volume of solution making the final ethanol concentration at 80%. A precipitate [570] is observed. The solution is centrifuged [580], decanted [590] and the supernatant residue [630] is discarded. The precipitate product [640] is the purified polysaccharide fraction that may be analyzed for polysaccharides using the colormetric method by using Dextran 5,000-410,000 molecular weight as reference standards. The purity of the extracted polysaccharide fraction is about 350 mg/g 5K dextran standard equivalent with a total yield of 1.15% by % mass weight of the original native Ginger leaf feedstock. Moreover, AccuTOF-DART mass spectrometry was used to further profile the molecular weights of the compounds comprising the purified polysaccharide fractions.

Following this procedure, the ginger purified polysaccharide yield was 1.15% (60% ethanol precipitation) and 1.16% (80% ethanol precipitation by mass weight of the original native ginger rhizome feedstock (see Example 6, Table 12). The purity of the polysaccharide fraction was 350-590 mg/gm 5K dextran standard equivalents indicating a polysaccharide purity of greater than 90% ginger polysaccharide chemical constituents in the fraction. Based on a large number and variety of experimental approaches, it is quite reasonable to conclude that 1.16% yield is almost 100% of the water soluble-ethanol insoluble polysaccharides in the natural ginger species rhizome feedstock material.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

### Summary

In performing the previously described extraction methods, it was found that greater than 90% yield by mass weight of the essential oil chemical constituents having greater than 95% purity of the essential oil chemical constituents in the original dried root feedstock of the Ginger species can be extracted in the essential oil SCCO₂ extract fraction (Step 1A). Moreover, greater than 85% of the gingerol chemical constituents can be extracted with the SCCO₂ processes of Step 1. Using the methods as taught in Step 1A and 1B, the essential oil yield may be reduced due to the sub-fractionation of the essential oil chemical constituents into highly purified volatile oil fractions and gingerol fractions having novel chemical constituent profiles. In addition, the SCCO₂ extraction and fractionation process as taught in this disclosure permits the ratios (profiles) of the individual chemical compounds comprising the essential oil chemical constituents to be altered such that unique volatile oil fraction and gingerol fraction profiles can be created for particular medicinal purposes. For example, the concentration of the gingerols may be increased while simultaneous reducing the concentration of the other essential oil chemical constituents such as, but not limited to, the monoterpenes, sesquiterpenes, and oxygenated sesquiterpenes or visa versa. Hence, single-stage, multi-stage fractionation, and single-stage fractionation SCCO₂ processes may be used to produce volatile oil fractions with total gingerols concentration ranging from about 8% to about 35% by mass weight of the volatile oil fraction and gingerol fractions with the gingerols concentration ranging from about 40% to about 69% by mass weight of the gingerol fraction.

Using the methods as taught in Step 2 of this disclosure, a hydroalcoholic leaching fraction is achieved with an about 12% mass weight yield from the original Ginger species feedstock having an about 6% concentration of total gingerols and an about 6% concentration of phenolic acids, a yield of about 0.7% of the gingerols while preserving the polysaccharides in the residue.

Using the methods as taught in Step 3 of this disclosure (Affinity Adsorbent Extraction Processes or Process Chromatography), phenolic fractions with total phenolic acid purities of about 26% to about 30% and total gingerol purities of about 25% to about 34% by mass weight of the extract fraction may be obtained. The total yield of this fraction is about 0.7% by mass weight based on the ginger root feedstock. Furthermore, this affinity adsorbent process can profile the gingerols resulting in a novel gingerol chemical composition with 6-gingerol making up about 90% of the gingerols in the phenolic acid fraction. The similarity of the concentration of the total phenolic acids and the total gingerols and the absence of other significant peaks in the HPLC chromatograms of these samples suggest that the gingerols are the predominant phenolic acid chemical constituents of ginger root.

Using the methods as taught in Step 4 of the disclosure (water leaching and ethanol precipitation, it appears that greater than about 90% yield by % mass weight of the water soluble-ethanol insoluble polysaccharide chemical constituents of the original dried Ginger species feedstock material can be extracted and purified in the polysaccharide fractions. Using 60-80% ethanol to precipitate the polysaccharides, purified polysaccharide fractions may be collected from the water leaching extract. The yield of a maximal polysaccharide fraction is about 1.1% by % mass weight based on the native Ginger plant material feedstock. Based on a colormetric analytical method using dextran as reference standards, a polysaccharide purity of about 0.35-0.59 mg/mg 5K dextran equivalent may be obtained.

Finally, the methods as taught in the disclosure permit the purification (concentration) of the Ginger species novel volatile oil chemical constituent fractions, novel gingerol fractions, novel phenolic fractions or, and novel polysaccharide fractions to be as high as about 90% by mass weight of the desired chemical constituents in the volatile oil fractions, as high as 69% by mass weight of the gingerols in a gingerol fraction, as high as about 30% by mass weight of the phenolic acids in the phenolic fraction, and as high as 90% polysaccharides by mass weight in a polysaccharide fraction. The specific extraction environments, rates of extraction, solvents, and extraction technology used depend on the starting chemical constituent profile of the source material and the level of purification desired in the final extraction products. Specific methods as taught in the disclosure can be readily determined by those skilled in the art using no more than routine experimentation typical for adjusting a process to account for sample variations in the attributes of starting materials that is processed to an output material that has specific attributes. For example, in a particular lot of Ginger species plant material, the initial concentrations of the essential oil chemical constituents, the gingerols, the phenolics, and the polysaccharides are determined using methods known to those skilled in the art as taught in the disclosure. One skilled in the art can determine the amount of change from the initial concentration of the essential oil chemical constituents, for instance, to the predetermined amounts or distribution (profile) of essential oil chemical constituents for the final extraction product using the extraction methods, as disclosed herein, to reach the desired concentration and/or chemical profile in the final Ginger species composition product.

In general, the methods and compositions of the disclosure comprise methods for making an extracted Ginger species composition having predetermined novel characteristics. Such an extracted Ginger species composition may comprise any one, two, three, or all four of the four concentrated extract fractions depending on the beneficial biological effect(s) desired for the given product. Typically, a composition containing all four Ginger species extraction fractions (chemical groups) is generally desired as such novel compositions represent the first highly purified Ginger species extraction products that contain all four of the principal biologically beneficial chemical constituent groups found in the native plant material Certain embodiments of the disclosure comprise methods wherein the predetermined characteristics comprise a predetermined selectively increased concentration of the Ginger species' essential oil chemical constituents, gingerols, phenolics, and polysaccharides in separate extraction fractions.

### Formulations and Pharmaceutical Compositions

Compositions of the disclosure comprise extracts of Ginger plant material and related species in forms such as a paste, powder, oils, liquids, suspensions, solutions, or other forms, comprising, one or more fractions or sub-fractions comprising volatile oils, gingerols, phenolics, or polysaccharides, to be used as dietary supplements, nutraceuticals, or pharmaceutical preparations and such compositions may be used to prevent or treat various human ailments. The extracts can be processed to produce such consumable items, for example, by mixing with them into a food product, in a capsule or tablet, or providing the paste itself for use as a dietary supplement, with sweeteners or flavors added as appropriate. Accordingly, such preparations may include, but are not limited to, compositions of Ginger and related species extract compositions for oral delivery in the form of tablets, capsules, lozenges, liquids, and emulsions. Other aspects of the compositions of the disclosure comprise Ginger species extract compositions in the form of a rapid dissolve tablet.

In certain embodiments, the disclosure comprises compositions comprising one or more chemical constituent fractions found in Ginger and related species. The disclosure also relates to ingestible products that comprise the Ginger and related species extraction compositions taught herein. For example, aspects of the disclosure relate to compositions comprising a rapid dissolve tablet, comprising an Ginger or related species extract composition wherein at least one of a volatile oil fraction, a volatile oil sub-fraction, a gingerol fraction, a phenolic fraction, or a polysaccharide fraction has been substantially increased in weight percent amount in relation to the weight percent amount of that found in the native plant material or to that currently found in known Ginger species extract compositions.

According to a further aspect of the disclosure, the novel extracted Ginger species plant material or a novel Ginger species extract composition can be further processed to dry, flowable powder. The powder can be used as a dietary supplement that can be added to various edible products. The powder or the final predetermined unique extract compositions of the Ginger species are also suitable for use in a rapid dissolve tablet.

According to a particular aspect of the disclosure, the extracted Ginger species compositions are produced to have a predetermined volatile oil, gingerols, phenolics, and polysaccharide concentrations that are greater than that found in the natural plant material or conventional Ginger species extract products and/or predetermined novel profiles of the four major bioactive chemical constituents of the Ginger species, wherein the ratios (profiles) of the amounts (% dry weight) of volatile oil/gingerols, volatile oil/phenolics, and/or volatile oil/polysaccharide, and/or gingerols/phenolics, and/or gingerols/polysaccharides, and/or phenolics/polysaccharides are greater or lesser than the chemical constituent profiles found in the natural Ginger species plant material or known Ginger species extraction products. Such novel compositions are particularly well suited for delivery in the oral cavity of human subjects, e.g., via a rapid dissolve tablet.

In one embodiment of a method for producing a Ginger species extraction powder, a dry extracted Ginger species composition is mixed with a suitable solvent, such as but not limited to water or ethyl alcohol, along with a suitable food-grade material using a high shear mixer and then spray air-dried using conventional techniques to produce a powder having grains of very small Ginger species extract particles combined with a food-grade carrier.

In a particular example, an extracted Ginger species composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spay air-drying. Typical amounts are in the range of between about 5 to about 10 liters per kilogram of extracted Ginger species material. The solution of extracted Ginger species composition, maltodextrin and ethyl alcohol is spray air-dried to generate a powder with an average particle size comparable to that of the starting carrier material.

In another embodiment, an extracted Ginger species composition and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extracted Ginger species composition material is combined with food grade material about one and one-half times by weight of the extracted Ginger species composition, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extracted Ginger species composition material is then heated until it flows like a heavy oil Preferably, it is heated to about 50° C. The heated extracted Ginger species composition is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extracted Ginger species composition material is introduced into a high shear mixer. The mixture of extracted Ginger species composition, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extracted Ginger species composition material, magnesium carbonate, silica and optional emulsifying agent and optional sweetener has an average particle size comparable to that of the starting carrier and a predetermined extraction Ginger species composition.

According to another embodiment, an extracted Ginger species composition material is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The Ginger species extraction composition material is heated until it flows like a heavy oil (preferably heated to about 50° C.). The heated Ginger species extraction composition is then added incrementally to the whey protein and inert carrier that is being mixed in the food processor-type mixer. The mixing of the Ginger species extraction composition and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of Ginger species extraction composition, whey protein, and inert carrier is introduced incrementally into the cold water containing high shear mixer while mixing. Sweetening agents or other taste additives of up to about 5% by weight can be added at this stage if desired. After mixing is completed, the mixture is dried using freeze drying or refractive window drying. The resulting dry flowable powder of Ginger species extraction composition, whey protein, inert carrier and optional sweetener has a particle size of about 150 to about 700 micrometers and an unique predetermined Ginger species extraction composition.

In a further embodiment, a predetermined Ginger species extraction composition is dissolved in a SFE CO₂ fluid that is then absorbed onto a suitable food-grade carrier such as maltodextrin, dextrose, or starch. Preferably, the SFE CO₂ is used as the solvent. Specific examples include starting with a novel extracted Ginger species composition and adding from one to one and a half times the extracted Ginger species material by weight of the food-grade carrier having a particle size of between about 100 to about 150 micrometers. This mixture is placed into a chamber containing mixing paddles and which can be pressurized and heated. The chamber is pressurized with CO₂ to a pressure in the range between about 1100 psi to about 8000 psi and set at a temperature in the range of between about 20° C. to about 100° C. The exact pressure and temperature are selected to place the CO₂ in a supercritical fluid state. Once the CO₂ in the chamber is in the supercritical state, the Ginger species extraction composition is dissolved. The mixing paddles agitate the carrier powder so that it has intimate contact with the supercritical CO₂ that contains the dissolved Ginger species extract material. The mixture of supercritical CO₂, dissolved Ginger species extraction material, and the carrier powder is then vented through an orifice in the chamber which is at a pressure and temperature that does not support the supercritical state for the CO₂. The CO₂ is thus dissipated as a gas. The resulting powder in the collection vessel is the carrier powder impregnated with the predetermined novel Ginger species extraction composition. The powder has an average particle size comparable to that of the starting carrier material. The resulting powder is dry and flowable. If needed, the flow characteristics can be improved by adding inert ingredients to the starting carrier powder such as silica up to about 2% by weight as previously discussed.

In the embodiments where the extract composition of the Ginger species with a predetermined composition or profile is to be included into a oral fast dissolve tablet as described in U.S. Pat. No. 5,298,261, the unique extract can be used “neat,” that is, without any additional components which are added later in the tablet forming process as described in the patent cited. This method obviates the necessity to take the unique Ginger species extract composition to a dry flowable powder that is then used to make the tablet.

Once a dry Ginger species extraction composition powder is obtained, such as by the methods discussed herein, it can be distributed for use, e.g., as a dietary supplement or for other uses. In a particular embodiment, the novel Ginger species extraction composition powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° C. to about 85° C. The dried tablet can then be packaged for distribution.

Methods and compositions of the disclosure comprise compositions comprising unique Ginger species extract compositions in the form of a paste, resin, oil, or powder. An aspect of the disclosure comprises compositions of liquid preparations of unique Ginger species extract compositions. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

An aspect of the disclosure comprises compositions of a dry powder Ginger species extraction composition. Such dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

Although the extraction techniques described herein are discussed in terms of Ginger species, it should be recognized that compositions of the disclosure can also comprise, in the form of a dry flowable powder or other forms, extracts from other plants such as, but not limited to, varieties of gymnemia, turmeric, boswellia, guarana, cherry, lettuce, Echinacia, piper betel leaf, Areca catechu, muira puama, ginger, willow, suma, kava, horny goat weed, ginko bilboa, mate, garlic, puncture vine, arctic root astragalus, eucommia, gastropodia, and uncaria, or pharmaceutical or nutraceutical agents.

The disclosure comprises compositions comprising unique Ginger species extract compositions in tablet formulations and methods for making such tablets. A tableting powder can be formed by adding about 1% to 40% by weight of the powdered Ginger species extract composition, with between 30% to about 80% by weight of a dry water-dispersible absorbent such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to about 100 mesh.

The disclosure comprises compositions comprising tablet formulations and methods for making such tablets. Preferably, the tablet has a formulation that results in a rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed in U.S. Pat. Nos. 5,464,632; 6,106,861; 6,221,392; 5,298,261; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1% and 1.5%. Additional components include maltodextrin (Maltrin, M-500) at between 1% and 5%. These amounts are solubilized in water and used as a starting mixture to which is added the Ginger species extraction composition, along with flavors, sweeteners such as Sucralose or Acesulfame K, and emulsifiers such as BeFlora and BeFloraPlus which are extracts of mung bean. A particularly preferred tableting composition or powder contains about 10% to 60% by of the Ginger species extract composition powder and about 30% to about 60% of a water-soluble diluent.

In a preferred implementation, the tableting powder is made by mixing in a dry powdered form the various components as described above, e.g., active ingredient (Ginger species extract composition), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the Ginger species extract composition in the form of a dry flowable powder in a single oral dosage is typically in the range of about 40 mg to about 1000 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 mg to about 1,500 mg. In addition to disk, wafer or coin shapes, the tablet can be in the form of a cylinder, sphere, cube, or other shapes. Although the tablet is preferably extract composition separated by non-Ginger species extract regions in periodic or non-periodic sequences, which can give the tablet a speckled appearance with different colors or shades of colors associated with the Ginger species extract regions and the non-Ginger species extract region.

Compositions of unique Ginger species extract compositions may also comprise Ginger species compositions in an amount between about 10 mg and about 2000 mg per dose. The volatile oil composition of the novel Ginger species extract composition can vary wherein the volatile oil fraction is in an amount between about 0.01 mg and about 1000.0 mg. The total gingerol fraction composition of the novel Ginger species extract composition can vary wherein the gingerol fraction is in an amount between 5 and about 1000 mg per dose wherein the % mass weight of the gingerol constituents in the novel Ginger species extract composition are greater in relation to the % mass weight than that found in natural Ginger plant material or conventional Ginger extraction products. The total phenolic fraction composition of the novel Ginger species extract compositions can vary between about 1 mg and about 1000 mg per dose wherein the % mass weight of the phenolic acid constituents in the unique Ginger species extraction composition are greater in relation to the % mass weight than that found in the natural Ginger species plant material or conventional Ginger species extracts and beverages. The Ginger species polysaccharide composition of the novel Ginger species extract composition can vary between about 1.0 mg and about 1000 mg wherein the % mass weight of the polysaccharide constituents are substantially increased in relation to the % mass weight of polysaccharides found in the natural Ginger species plant material or conventional Ginger species extracts or beverages. Furthermore, the % mass weight ratios of the four principal beneficial bioactive chemical constituent groups (volatile oil, gingerols, phenolics, and polysaccharides) derived from the Ginger species may be altered to yield additional novel Ginger species extract composition profiles for human oral delivery using the doses ranges mentioned previously. Finally, the % mass weight of the individual volatile oil or gingerol chemical constituent compounds may be altered (profiled) to yield novel volatile oil fraction composition and gingerol fraction composition profiles for human oral delivery using dose ranges as noted.

An exemplary 275 mg tablet contains about 150.0 mg powdered predetermined unique Ginger species extract composition, about 12.5 mg extract of Stevia, about 35.5 mg carboxymethylcellulose, and about 77.0 mg of lactose (see Example 7). An further exemplary formulation for 500 mg Ginger species extraction composition tablets is detailed in Example 8.

The disclosure comprises methods of using compositions comprising unique Ginger species extraction compositions disclosed herein. Methods of providing dietary supplementation are contemplated. Such compositions may further comprise vitamins, minerals and antioxidants. Compositions taught herein can also be used in the methods of treatment of various physiological, psychological, and medical conditions. The standardized, reliable and novel Ginger species extraction compositions of the disclosure are used to prevent and treat nausea and vomiting related to, but not limited to, pregnancy, motion sickness, vertigo, anesthesia, surgery, and cancer chemotherapy. The standardized, reliable, and novel Ginger species extraction composition can also be used to prevent and treat inflammatory disorders, arthritis, rheumatic diseases, and auto-immune diseases. The Ginger extract compositions may be used as an analgesic and for the management of headache and migraine. Cardiovascular and cerebrovascular disease benefits include anti-artery damage, anti-oxidant activity, reduction of oxygen free radicals, anti-arteriosclerosis, anti-hyerlipidemia, anti-thrombosis, anti-hypertension, vasodilation, anti-cardiac arrhythmias, and anti-diabetes. Ginger extraction compositions of the disclosure may be used to prevent and treat obesity. Alzheimer's disease and Parkinson's disease as well as other brain degenerative disease may benefit from the use of the novel high quality, standardized, and reliable Ginger extract compositions. Ginger extract compositions have immunomodulatory activity and protect from ionizing radiation Ginger extract compositions also have anti-colic, anti-dyspepsia, and anti-diarrhea activity. Other properties include anti-viral disease and anti-bacterial diseases. Moreover, the Ginger species extraction compositions of the disclosure are used to prevent and treat cancer. These and other related pathologies are prevented or treated by administering an effective amount of the novel Ginger species extraction compositions of the disclosure.

The novel Ginger species extraction compositions may be administered daily, for one or more times, for the effective treatment of acute or chronic conditions. One method of the disclosure comprises administering at least one time a day a composition comprising Ginger species constituent compounds. Methods also comprise administering such compositions more than one time per day, more than two times per day, more than three times per day and in a range from 1 to about 15 times per day. Such administration may be continuously, as in every day for a period of days, weeks, months, or years, or may occur at specific times to treat or prevent specific conditions. For example, a person may be administered Ginger species extract compositions at least once a day for years to treat chronic nausea, vomiting, and disequilibrium inflammatory disorders, arthritis, rheumatoid disease, and auto-immune disease, to prevent or treat cardiovascular disease and stroke, obesity, diabetes, hypertension, cardiac arrhythmias, Alzheimer's disease, Parkinson's disease, other brain degenerative disease and cancer.

A pharmaceutical composition comprising the Ginger composition of the disclosure may be administered to a subject by known procedures, including, without limitation, oral administration, parenteral administration, transdermal administration, and by way of catheter. For example, the Ginger composition may be administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual injection. The pharmaceutical composition may be provided in an amount effective to treat a pathological condition (e.g., a menopausal disorder) in a subject to whom the composition is administered. As used herein, the phrase “effective to treat a disorder” means effective to eliminate, ameliorate, or minimize the clinical impairment or symptoms resulting from the disorder. As used herein, the term “subject” refers to an animal, including, without limitation, a human, cow, dog, monkey, mouse, pig, rat, chicken, or fish. Preferably, the subject is a human.

For oral administration, a formulation comprising the Ginger composition may be presented as capsules, tablets, powders, granules, or as a suspension. The formulation may have conventional additives, such as, lactose, mannitol, corn starch, or potato starch. The formulation also may be presented with binders, such as, crystalline cellulose, cellulose derivatives, acacia, corn starch, and gelatins. Additionally, the formulation may be presented with disintegrators, such as, corn starch, potato starch, and sodium carboxymethylcellulose. The formulation also may be presented with dibasic calcium phosphate anhydrous or sodium starch glycolate. Moreover, the formulation may be presented with lubricants, such as talc and magnesium stearate.

For parenteral administration (i.e., administration by injection through a route other than the alimentary canal), the Ginger composition may be combined with a sterile aqueous solution that may be isotonic with the blood of the subject. Such a formulation may be prepared by dissolving the Ginger composition in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, and sublingual.

For transdermal administration, the Ginger composition may be combined with skin penetration enhancers, such as propylene glycol, polyethylene glycol, isopropanol, ethanol, oleic acid, N-methylpyrrolidone, and the like, which increase the permeability of the skin to the Ginger composition, and permit the Ginger composition to penetrate through the skin and into the bloodstream. The Ginger composition may be further combined with a polymeric substance, such as ethylcellulose, hydroxypropyl cellulose, ethylene/vinylacetate, polyvinyl pyrrolidone, and the like, to provide the composition in gel form which may be dissolved in a solvent, such as methylene chloride, evaporated to the desired viscosity, and then applied to backing material to provide a patch.

In accordance with the method of the disclosure, the Ginger composition also may be administered to a subject by way of a pharmaceutical composition for use in treating or preventing a pathological condition. The pharmaceutical composition of the disclosure comprises a pharmacological effective amount of the Ginger composition and a pharmaceutically-acceptable carrier. The pharmaceutically-acceptable carrier may be “acceptable” in the sense of being compatible with the other ingredients of the composition, and not deleterious to the recipient thereof. The pharmaceutically-acceptable carrier employed herein may be selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the disclosure may be prepared by methods well-known in the pharmaceutical arts, such as, using methods disclosed in Remington's Pharmaceutical Sciences (18^{th} ed, Mack Publishing Company, Easton, Pa. (1990)). For example, the composition may be brought into association with a carrier or diluent, as a suspension or solution, such as, dissolution or suspension of the Ginger extract in a vehicle, e.g., water or naturally occurring vegetable oil like sesame, peanut, or cottonseed oil or a synthetic fatty vehicle like ethyl oleate or the like. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which may be administered orally, topically, or by injection, including, without limitation, intravenous, intraperitoneal, subcutaneous, and intramuscular injection.

The pharmaceutical composition of the disclosure may be in an instant-release or sustained-release form Suitable sustained-release preparations include, without limitation, semipermeable matrices of solid hydrophobic polymers containing the Curcuma extracts in the form of shaped articles, films, or microcapsules. Examples of sustained-release matrices include, for instance, polyesters, hydrogels (e.g., poly(2-hydroxyethyl-methacrylate) as described by Langer et al., J Biomed Mater. Res., 15:167-277 (1981) and Langer, Chem. Tech., 12:98-105 (1982), or poly(vinylalcohol)), polylactides (U.S. Pat. No. 3,773,919, EP 58,481), copolymers of L-glutamic acid and gamma ethyl-L-glutamate (Sidman et al, Biopolymers, 22:547-556 (1983)), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

Aspects of the disclosure also relate to methods for treatment and prevention of human disorders with novel Ginger compositions. For example, a novel Ginger species composition for prevention or treatment of nausea and vomiting may have an increased gingerol and phenolic fraction composition concentration and reduced volatile oil and polysaccharide fraction composition concentrations, by % weight, than that found in the Ginger species native plant material or conventional known extraction products. A novel Ginger species composition for anti-inflammatory, anti-arthritis, anti-rheumatoid diseases, anti-autoimmune diseases and analgesia may have an increased volatile oil, gingerol, phenolic and polysaccharide fraction composition concentrations. A novel Ginger species composition for anti-oxidant, anti-blood vessel damage, and ischemic cerebrovascular and cardiovascular disease may have an increased volatile oil, gingerol and phenolic fraction composition and a reduced polysaccharide fraction composition, by % weight, than that found in the native Ginger species plant material or conventional known extraction products. Another example of a novel Ginger species composition, for prevention and treatment of allergic Alzheimer's and Parkinson's disease comprises a composition having an increased volatile oil fraction composition concentration, an increased gingerol fraction concentration, a reduced phenolic fraction concentration, and a reduced polysaccharide fraction composition than that found in native Ginger species plant material or known conventional extraction products.

Alteration of the concentration relationships (chemical profiles) of the beneficial chemical constituents of the individual Ginger species permits the formulation of unique or novel Ginger species extract composition products designed for specific human conditions or ailments. For example, a novel and powerful Ginger composition for nausea and vomiting related to pregnancy, motion sickness, anesthesia, surgery, and cancer chemotherapy prevention and treatment could have a greater purified gingerol composition and phenolic composition and a reduced volatile oil composition and polysaccharide composition by % mass weight than that found in the Ginger native plant material or conventional known extraction products. In contrast, a novel Ginger composition for anti-inflammatory activity, arthritis, rheumatic diseases and analgesia activity could have a greater purified volatile oil composition, gingerol composition, phenolic composition and polysaccharide composition by % mass weight than that found in the Ginger native plant material or conventional known extraction products. Another example of a novel Ginger composition profile for anti-oxidant and reactive oxygen species scavenging activity could be a composition profile with greater purified volatile oil composition and phenolic composition and a reduced purified gingerol composition and a reduced purified polysaccharide composition than that found in native Ginger plant material or known conventional Ginger extraction products. An additional example of a novel Ginger composition profile for prevention and treatment of obesity could be a composition profile with a greater purified phenolic composition and polysaccharide composition and a reduced volatile oil composition and gingerol composition than that found in native Ginger plant material or known conventional Ginger extraction products.

A further embodiment of the disclosure is compositions comprising novel sub-fractions of the volatile oil chemical constituents wherein the concentration of specific chemical groups or compounds such as, but not limited to, sesquiterpenes or zingiberene having their respective concentrations increased for decreased in novel extraction composition products.

Another embodiment of the disclosure is compositions comprising novel fractions of the purified gingerol chemical constituents wherein the concentration of specific chemical compounds such as, but not limited to, the 6-gingerol or 6-shagaol have their respective concentrations increased or decreased in novel extraction compositions.

### EXEMPLIFICATION

The disclosure now being generally described, it will be more readily understood by reference to the following examples, which are included merely for purposes of illustration of certain aspects and embodiments of the disclosure, and are not intended to limit the disclosure.

### Materials and Methods

Botanical: Ginger root was purchased from Kalyx Co. (Camden, N.Y., USA). The ground powder for was in a particle size of 100 μm The moisture content of this feedstock was 7.21%.
Organic Solvents Acetonitrile (75-05-8), for HPLC, gradient grade≧99.9% (GC) (000687); Ethanol, denatured with 4.8% isopropanol (02853); Ethanol (64-17-5), absolute, (02883); Methanol (67-56-1), 99.93%, ACS HPLC grade, (4391993); and Water (7732-18-5), HPLC grade, (95304). All were purchased from Sigma-Aldrich Co (St. Louis, Mo., USA).
Acids and Bases: Phosphoric acid (7664-38-2), 85% was purchased from Merck Co. (Whitehouse Station, N.J., USA); and Hydrochloric acid (045603), 36.5% in water; Sodium hydroxide solution (023196-24), 50% solution; Sulfuric acid (7664-93-9), ACS reagent, 9597% (44719); Phenol (108-95-2) (P3653), Folin-Ciocalteu phenol reagent (2N) (47641); Sulfuric acid (7664-93-9), all were purchased from Sigma-Aldric Co. (St. Louis, Mo.); and Sodium carbonate (S263-1, Lot #: 037406) were all purchased from Fisher Co (Hampton, N.J., USA).
Chemical Reference Standards: Dextran standards 5,000 (00269), 50,000 (00891), 410,000 (00895) certified according to DIN were purchased from Fluka Co. (St. Louis, Mo.) Gingerol standard kit (ASB-00030290) was purchased from ChromaDex Co. (Santa Ana, Calif.).
Polymer Affinity Adsorbent: Amberlite XAD 7HP (Rohm & Haas, France), macroreticular aliphatic acrylic cross-linked polymer used as white translucent beads with particle size of 500-710 nm and surface area is 380 m²/gm. ADS-5 (Nankai University, Tianjin, China), ester group modified polystyrene with particle size of 300-1200 nm and surface area is 500-600 m²/gm.

### High Performance Liquid Chromatography (HPLC) Methods:

[Chromatographic system]: Shimadzu high Performance Liquid Chromatographic LC-10AVP system equipped with LC10ADVP pump with SPD-M 10AVP photo diode array detector.

[HPLC Method]: The extraction products obtained were measured on a reversed phase Synergi Max-RP column (150×4.6 mm I. D., 4μ, 80 Å) (Phenomenex, Part No. 00F-4337-E0, serial No.: 328492-20). The injection volume was 10 μl, the flow rate of mobile phase was 1 ml/min and the column temperature was 40° C. The mobile phase consisted of A (0.05% aqueous phosphoric acid, v/v) and B (0.05% phosphoric acid in acetonitrile). The gradient was programmed as follows: mobile B increased linearly from 40% to 90% over 20 min, followed by 90% B for 10 min. Detection: 210 nm.

Methanol stock solutions of 4 standards were prepared by dissolving weighted quantities of standard compounds into methanol at 5 mg/ml. One milliliter aliquots of 4 reference standards were transferred into a 10 ml volumetric flask to yield a mixed standard solution. The mixed reference standard solution was then diluted step by step to yield a series of solutions at final concentrations of 2, 1, 0.5, 0.1, and 0.05 mg/ml, respectively. All the stock solutions and working solution were used within 7 days and stored in +4° C. chiller and brought to room temperature before use. The solutions were used to identify and quantify the compounds in Ginger. Retention times of 6-gingerol, 8-gingerol, 6-shagaol and 10-gingerol were about 8.44, 12.99, 14.28 and 17.54 min, respectively. A linear fit ranging from 0.1 to 20 μg was found. The regression equations and correlation coefficients were as follows: 6-gingerol: peak area/100=16391×C (μg) 431.42, R²=0.9976 (N=6); 8-gingerol: peak area/100=15576×C (μg)+687.16, R²=0.9995 (N=6); 6-shagaol: peak area/100=3456.6×C (μg)+289.59, R²=0.9988 (N=6); 10-gingerol: peak area/100=10423×C (μg)+951.57, R²=0.9987 (N=6). HPLC results are shown in Table 2. The contents of the reference standards in each sample were calculated by interpolation from the corresponding calibration curves based on the peak area.

TABLE 2
HPLC analysis results on Ginger reference standards [concentration 1 mg/ml in MeOH].

| | Retention time | Area | Height | Width | Start time | Stop time | Theoretical |
|---|---|---|---|---|---|---|---|
| ID | (min) | (mAu · min) | (mAu) | (min) | (min) | (min) | plate* |
| 6-gingerol | 8.448 | 4029187 | 512190 | 0.79 | 8.21 | 9.00 | 1830 |
| 8-gingerol | 12.992 | 4015329 | 469072 | 0.73 | 12.59 | 13.31 | 5068 |
| 6-shoraol | 14.283 | 7168524 | 831008 | 0.52 | 13.97 | 14.50 | 12071 |
| 10-gingerol | 17.536 | 2625268 | 280960 | 0.92 | 17.18 | 18.10 | 5813 |

*Theoretical plates was calculated by: N = 16 × (t_{R}/w)², wherein t_{R} is retention time and w is width of the peak.See: https://www.mn-net.com/web%5CMNWEBHPLCKatalog.nsf/WebE/GRUNDLAGEN

### Gas Chromatography-Mass Spectrometry (GC-MS) Methods:

GC-MS analysis was performed at Shimadzu GCMS-QP2010 system. The system includes high-performance gas chromatograph, direct coupled GC/MS interface, electro impact (EI) ion source with independent temperature control, quadrupole mass filter et al. The system is controlled with GCMS solution Ver. 2 software for data acquisition and post run analysis. Separation was carried out on a Agilent J&W DB-5 fused silica capillary column (30 m×0.25 mm i.d., 0.25 μm film (5% phenyl, 95% dimethylsiloxane) thickness) (catalog: 1225032, serial No: US5285774H) using the following temperature program. The initial temperature was 60° C., held for 2 min, then it increased to 80° C. at rate of 4° C./min and hold for 2 min, then it increased to 240° C. at rate of 3° C./min, held for 15 min. The total run time was approximately 78 minutes. The sample injection temperature was 240° C. and 1 μl of sample was injected by auto injector at splitless mode in 1 minute. The sample concentration were 200 ppm in dichloromethane. The carrier gas was helium and flow rate was controlled by pressure at 55 KPa. Under such pressure, the flow rate was 0.97 ml/min and linear velocity was 35.9 cm/min and total flow was 33.3 ml/min. MS ion source temperature was 250° C., and GC/MS interface temperature was 250° C. MS detector was scanned between m/z of 35 and 500 at scan speed of 1000 AMU/second with an ionizing voltage at 70 eV. Solvent cutoff temperature was 3.5 min. Volatile oil constituents were identified by matching their fragmentation pattern in mass spectra with those of NIST27, NIST147 library and literature.

### Folin-Ciocalteu Method for Total Phenolic Acid (Markar 1988):

[Instruments]: Shimazu UV-Vis spectrophotometer (UV 1700 with UV probe S/N:A1102421982LP).
[Reference Standard]: Make stock Gallic acid/water solution at concentration of 1 mg/ml. Take suitable amount of Gallic acid solution in test tubes, make up the volume to 0.5 ml with distilled water, add 0.25 ml of the Folin Ciocalteu reagent and then 1.25 ml of the 20 wt % sodium carbonate solution. Shake the tube well in an ultrasonic bath for 40 min and record absorbance at 725 mm. The data are shown in Table 3.

TABLE 3
Preparations of calibration curve for Gallic acid.

| | | | | | Sodium | |
|---|---|---|---|---|---|---|
| | Gallic acid solution | Gallic acid | Distilled | Folin | carbonate | Absorbance |
| Tube | (0.1 mg/ml) (ml) | (μg) | water (ml) | reagent (ml) | solution (ml) | at 725 mm* |
| Blank | 0.00 | 0 | 0.50 | 0.25 | 1.25 | 0.000 |
| 1 | 0.02* | 2 | 0.48* | 0.25 | 1.25 | 0.111 |
| 2 | 0.04 | 4 | 0.46 | 0.25 | 1.25 | 0.226 |
| 3 | 0.06 | 6 | 0.44 | 0.25 | 1.25 | 0.324 |
| 4 | 0.08 | 8 | 0.42 | 0.25 | 1.25 | 0.464 |
| 5 | 0.1 | 10 | 0.40 | 0.25 | 1.25 | 0.608 |

*Amount of gallic acid by % weight in solution is directly dependent on the absorbance.

[Unknown Sample]: Take suitable aliquots of the tannin-containing extract in test tubes, make up the volume to 0.5 ml with distilled water, add 0.25 ml of the Folin-Ciocalteu reagent, and then 1.25 ml of the sodium carbonate solution. Vortex the tubes and record the absorbance at 725 nm after 40 min. Calculate the amount of total phenolic acids as gallic acid equivalent from the above calibration curve.

### Polysaccharide Analysis:

[Spectrophotometer system]: Shimadzu U-1700 ultraviolet visible spectrophotometer (190-1100 nm, 1 mm resolution) has been used in this study.

Colorimetric method (50) was used for ginger polysaccharide analysis. Make 0.1 mg/ml stock dextran (Mw=5000, 50,000 and 410,000) solutions. Take 0.08, 0.16, 0.24, 0.32, 0.40 ml of stock solution and make up volume to 0.4 ml with distilled water. Then add in 0.2 ml 5% phenol solution and 1 ml concentrated sulfuric acid. The mixtures were allowed to stand for 10 minutes prior to performing UV scanning. The maximum absorbance was found at 488 nm. Then set the wavelength at 488 nm and measure absorbance for each sample. The results are shown in Table 4. The standard calibration curves were obtained for each of the dextran solutions as follows: Dextan 5000, Absorbance=0.01919+0.027782 C (μg), R²=0.97 (N=5); Dextan 50,000, Absorbance=0.0075714+0.032196 C (μg), R²=0.96 (N=5); and Dextan 410,000, Absorbance=0.03481+0.036293C (μg), R²=0.98 (N=5).

TABLE 4
Colorimetric analysis of dextran reference standards.

| | Dextran | Distill | 5% phenol | Sulfuric | Absorb. | Absorb. | Absorb. |
|---|---|---|---|---|---|---|---|
| Tube | solution (ml) | water (ml) | (ml) | acid (ml) | (Mw = 5K) | (Mw = 50K) | (Mw = 410K) |
| Blank | 0 | 0.40 | 0.2 | 1 | 0 | 0 | 0 |
| 1 | 0.08 | 0.32 | 0.2 | 1 | 0.238 | 0.301 | 0.335 |
| 2 | 0.16 | 0.24 | 0.2 | 1 | 0.462 | 0.504 | 0.678 |
| 3 | 0.24 | 0.16 | 0.2 | 1 | 0.744 | 0.752 | 0.854 |
| 4 | 0.32 | 0.08 | 0.2 | 1 | 0.907 | 1.045 | 1.247 |
| 5 | 0.40 | 0.00 | 0.2 | 1 | 1.098 | 1.307 | 1.450 |

### Direct Analysis in Real Time (DART) Mass Spectrometry for Polysaccharide Analysis.

Instruments: JOEL AccuTOF DART LC time of flight mass spectrometer (Joel USA, Inc., Peabody, Mass., USA). This Time of Flight (TOF) mass spectrometer technology does not require any sample preparation and yields masses with accuracies to 0.00001 mass units.
Methods: The instrument settings utilized to capture and analyze polysaccharide fractions are as follows: For cationic mode, the DART needle voltage is 3000 V, heating element at 250° C., Electrode 1 at 100 V, Electrode 2 at 250 V, and helium gas flow of 7.45 liters/minute (L/min). For the mass spectrometer, orifice 1 is 10 V, ring lens is 5 V, and orifice 2 is 3 V. The peaks voltage is set to 600 V in order to give resolving power starting at approximately 60 m/z, yet allowing sufficient resolution at greater mass ranges. The micro-channel plate detector (MCP) voltage is set at 2450 V. Calibrations are performed each morning prior to sample introduction using a 0.5 M caffeine solution standard (Sigma-Aldrich Co., St. Louis, USA). Calibration tolerances are held to ≦5 mmu.

The samples are introduced into the DART helium plasma with sterile forceps ensuring that a maximum surface area of the sample is exposed to the helium plasma beam. To introduce the sample into the beam, a sweeping motion is employed. This motion allows the sample to be exposed repeatedly on the forward and back stroke for approximately 0.5 sec/swipe and prevented pyrolysis of the sample. This motion is repeated until an appreciable Total Ion Current (TIC) signal is observed at the detector, then the sample is removed, allowing for baseline/background normalization.

For anionic mode, the DART and AccuTOF MS are switched to negative ion mode. The needle voltage is 3000 V, heating element 250° C., Electrode 1 at 100 V, Electrode 2 at 250 V, and helium gas flow at 7.45 L/min. For the mass spectrometer, orifice 1 is −20 V, ring lens is −13 V, and orifice 2 is −5 V. The peak voltage is 200 V. The MCP voltage is set at 2450 V. Samples are introduced in the exact same manner as cationic mode. All data analysis is conducted using MassCenterMain Suite software provided with the instrument.

### Dart Mass Spectrometry for Ginger Extracts Prepared Using SCCO₂.

In all extracts analyzed by DART TOF-MS, 20-55% of all peaks present in the mass spectra are accurately identified. Of the remaining unassigned peaks, approximately 20-30% are isotopes of identified chemicals or fragments of higher molecular weight chemicals. Therefore, in most extracts, 40-85% of the total chemicals present in each extract can be identified in a few minutes using DART TOF-MS without adulteration (i.e. sample preparation, sample derivatizing, etc.) of the sample.

The DART settings were loaded as follows: DART Needle voltage=3000 V; Electrode 1 voltage=150 V; Electrode 2 voltage=250 V; Temperature=250° C.; He Flow Rate=1.20-2.42 LPM. The following AccuTOF mass spectrometer settings were loaded: Ring Lens voltage=5 V; Orifice 1 voltage=10 V; Orifice 2 voltage=5 V; Peaks voltage=1000 V (for resolution between 100-1000 amu); Orifice 1 temperature was turned off.

The samples were introduced by placing the closed end of a borosilicate glass capillary tube into the Zingiber extracts, and the coated tip capillary tube was passed through the He plasma until signal was observed in the total-ion-chromatogram (TIC). The sample was removed and the TIC was brought down to baseline levels before the next sample was introduced. A polyethylene glycol 600 (Ultra Chemicals, Kingston RI) was used as an internal calibration standard giving mass peaks throughout the desired range of 100-1000 amu.

The DART mass spectra of each extract was searched against a proprietary chemical database and used to identify chemicals present in the Zingiber extracts. Search criteria were held to the [M+H]⁺ ions to within 10 mmu of the calculated exact masses of each chemical. The identified chemistries are reported with greater than 90% confidence.

### Example 1

### Example of Step 1A (FIG. 1)

### Single-Step SCCO₂ Maximal Extraction and Purification of Ginger Essential Oil

All SFE extractions were performed on SFT 250 (Supercritical Fluid Technologies, Inc., Newark, Del., USA) designed for pressures and temperatures up to 690 bar and 200° C., respectively. This apparatus allows simple and efficient extractions at supercritical conditions with flexibility to operate in either dynamic or static modes. This apparatus consists of mainly three modules: an oven, a pump and control, and collection module. The oven has one preheat column and one 100 ml extraction vessel. The pump module is equipped with a compressed air-driven pump with constant flow capacity of 300 ml/min. The collection module is a glass vial of 40 ml, sealed with caps and septa for the recovery of extracted products. The equipment is provided with micrometer valves and a flow meter. The extraction vessel pressure and temperature are monitored and controlled within ±3 bar and ±1° C.

In typical experimental examples, 15 grams of ginger rhizome powder with size above 105 μm sieved by 140 mesh screen was loaded into a 100 ml extraction vessels for each experiment. Glass wool was placed at the two ends of the column to avoid any possible carry over of solid material. The oven was preheated to the desired temperature before the packed vessel was loaded. After the vessel was connected into the oven, the extraction system was tested for leakage by pressurizing the system with CO₂ (˜850 psig), and purged. The system was closed and pressurized to desired extraction pressure using the air-driven liquid pump. The system was then left for equilibrium for ˜3 min. A sampling vial (40 ml) was weighed and connected to the sampling port. The extraction was started by flowing CO₂ at a rate of ˜5 SLPM (9.8 g/min), which is controlled by a meter valve. The solvent/feed ratio, defined as the weight ratio of total CO₂ used to the weight of loaded raw material, was calculated. During the extraction process, the extracted sample was weighed every 5 min. Extraction was presumed to be finished when the weight of the sample did not change more than 5% between two weighing measurements. The yield was defined to be the weight percentage of the essential oil extracted with respect to the initial total weight of the feedstock material loaded into the extraction vessel. A full factorial extraction design was adopted varying the temperature from 40-60° C. to 80-500 bar. The extracts obtained at each SCCO₂ condition were dissolved in methanol at 2 mg/ml for HPLC analysis and in dichloromethane at 0.2 mg/ml for GC-MS analysis. The HPLC results are shown in Table 5 and the GC-MS results are shown in Table 6. The extraction time ranged from 50 to 80 minutes and the solvent/feed ratio ranged from 33 to 75.

TABLE 5
Ginger SFE extraction yield and gingerol purity and yield based on HPLC analysis.

| T | P | Density | Purity (%) | | | | | 6-G ratio | Yield (%) | |
|---|---|---|---|---|---|---|---|---|---|---|
| (° C.) | (bar) | (g/cc) | 6-G | 8-G | 10-G | 6-S | total | (%) | total | gingerol |
| 40 | 100 | 0.64 | 19.16 | 3.87 | 8.72 | 7.56 | 39.31 | 48.7 | 1.79 | 0.70 |
| 40 | 300 | 0.915 | 19.46 | 4.02 | 9.71 | 5.52 | 38.71 | 50.3 | 3.01 | 1.17 |
| 40 | 500 | 0.996 | 17.51 | 3.65 | 8.53 | 4.60 | 34.28 | 51.1 | 0.82 | 0.28 |
| 60 | 100 | 0.297 | 9.91 | 1.96 | 4.86 | 4.94 | 21.67 | 45.7 | 0.58 | 0.13 |
| 60 | 300 | 0.834 | 17.23 | 3.40 | 7.62 | 5.66 | 33.91 | 50.8 | 1.68 | 0.57 |
| 60 | 500 | 0.938 | 15.32 | 3.10 | 7.31 | 4.54 | 30.27 | 50.6 | 0.63 | 0.19 |

TABLE 6
GC-MS analysis results of peak area % of essential oil extracted
at different conditions.

| | T = 40° C. | | | T = 60° C. | | |
|---|---|---|---|---|---|---|
| P (bar) | 100 | 300 | 500 | 100 | 300 | 500 |
| Peak No. | Peak percentage (%) | | | | | |
| 1 | 0.21 | | | | | |
| 2 | | | | | | |
| 3 | | | | | | |
| 4 | 0.17 | | | | | |
| 5 | 2.52 | 3.35 | 3.37 | 0.91 | 3.52 | 3.73 |
| 6 | | | | | | |
| 7 | | | | | | |
| 8 | 0.18 | 0.6 | 0.19 | | 0.4 | |
| 9 | 0.16 | 0.21 | 0.15 | | 0.21 | |
| 10 | 5.52 | 7.7 | 7.86 | 2.88 | 7.48 | 8.55 |
| 11 | 0.29 | 0.73 | 0.68 | 0.5 | 0.43 | 0.59 |
| 12 | 0.25 | 0.44 | 0.33 | | 0.36 | 0.33 |
| 13 | | 0.16 | | | 0.21 | |
| 14 | | 0.38 | 0.14 | 0.19 | 0.35 | 0.32 |
| 15 | 0.23 | 0.23 | 0.09 | 0.32 | 0.23 | |
| 16 | | 0.13 | | 0.16 | | |
| 17 | 1.01 | | 0.28 | | | 0.23 |
| 18 | 0.51 | | | | | |
| 19 | 0.53 | | | | | |
| 20 | 0.77 | 0.22 | 0.17 | 0.32 | | |
| 21 | 13.66 | 10.68 | 10.14 | 12.77 | 8.69 | 4.66 |
| 22 | 5.83 | 7.06 | 8.48 | 7.2 | 6.87 | 9.51 |
| 23 | 1.02 | 1.17 | 0.98 | 1.21 | 0.81 | 0.46 |
| 24 | 5.85 | 5.24 | 5.52 | 6.74 | 4.82 | 3.81 |
| 25 | 0.14 | 0.2 | 0.19 | 0.38 | 0.18 | |
| 26 | 0.24 | | | 0.23 | | |
| 27 | 0.34 | 0.13 | | 0.26 | 0.15 | |
| 28 | 9.15 | 6.99 | 7.5 | 11.13 | 6.46 | 5.62 |
| 29 | 0.14 | 0.27 | | 0.59 | 0.14 | |
| 30 | 0.14 | | | 0.64 | 0.13 | |
| 31 | 0.79 | 0.48 | 0.43 | 1.72 | 0.49 | 0.36 |
| 32 | 0.11 | 0.52 | 0.54 | 0.3 | 0.44 | 1 |
| 33 | 0.16 | | | 0.4 | | |
| 34 | 0.26 | 0.12 | 0.14 | 1.05 | 0.27 | |
| 35 | | 1.08 | 0.38 | | | |
| 36 | 0.47 | 0.21 | 0.23 | 1.55 | 0.4 | 0.29 |
| 37 | 0.24 | 0.13 | 0.18 | 1.09 | 0.16 | |
| 38 | 0.4 | 0.27 | 0.34 | 1.36 | 0.19 | 0.19 |
| 39 | 20.67 | 23.4 | 25.61 | 11.94 | 25.32 | 27.3 |
| 40 | 0.81 | 0.74 | 0.57 | 1.75 | 0.39 | 0.51 |
| 41 | 0.47 | 0.42 | 0.34 | 1.26 | 0.28 | 0.45 |
| 42 | 0.65 | 0.64 | 0.58 | 0.8 | 0.46 | 0.41 |
| 43 | 0.76 | 0.89 | 0.8 | 0.62 | 0.7 | 0.92 |
| 44 | | 0.09 | | | | |
| 45 | 1.33 | 0.87 | 0.75 | 2.43 | 1.08 | 0.64 |
| 46 | 3.03 | 2.84 | 2.64 | 4.33 | 2.32 | 2.42 |
| 47 | 0.35 | 0.29 | 0.28 | 0.51 | 0.32 | 0.32 |
| 48 | 2.21 | 1.78 | 1.68 | 2.54 | 2.59 | 2.34 |
| 49 | | 0.13 | | | 0.33 | |
| 50 | 0.41 | 0.48 | 0.39 | 0.61 | 0.81 | 0.9 |
| 51 | 0.26 | 0.35 | 0.27 | 0.45 | 0.46 | 0.45 |
| 52 | 0.14 | 0.15 | 0.18 | 0.18 | 0.22 | 0.11 |
| 53 | 0.64 | | | | | |
| 54 | 0.57 | 0.34 | 0.23 | 1.02 | 0.3 | 0.17 |
| 55 | | 0.05 | | 0.23 | | |
| 56 | 0.14 | | | 0.4 | | |
| 57 | 0.21 | | | 0.25 | | |
| 58 | 0.31 | | | | | |
| 59 | 0.2 | | | | | |
| 60 | 0.33 | 0.21 | | 0.48 | | 0.24 |
| 61 | 0.35 | 0.2 | 0.22 | 0.63 | 0.24 | 0.24 |
| 62 | 0.19 | 1.23 | 1.3 | 0.36 | 0.4 | 0.44 |
| 63 | 0.61 | 0.54 | 0.5 | 0.33 | 0.61 | 0.49 |
| 64 | 0.32 | 0.27 | 0.29 | 0.38 | 0.47 | 0.43 |
| 65 | 9.86 | 9.85 | 9.84 | 8.23 | 14.17 | 14.88 |
| 66 | 0.35 | | | 0.3 | | 1.31 |
| 67 | 0.46 | 2.96 | 3.2 | 0.76 | 1.62 | 0.8 |
| 68 | 0.66 | 0.82 | 0.94 | 0.6 | 1.38 | 1.74 |
| 69 | 0.41 | 0.11 | | 0.34 | 0.49 | 0.42 |
| 70 | 0.35 | 0.87 | 0.79 | 0.4 | 1.21 | 1.49 |
| Total | 98.13 | 99.22 | 99.71 | 96.03 | 99.56 | 99.07 |
| Monoterpene | 0.34 | 0.97 | 0.34 | 0 | 0.82 | 0 |
| Sesquiterpene | 41.18 | 33.14 | 34.53 | 46.44 | 29.45 | 25.13 |
| Oxygenated Sesquiterpene | 9.42 | 8.66 | 7.62 | 14.4 | 8.85 | 8.74 |
| Gingerol | 32.21 | 35.21 | 37.47 | 21.85 | 42.55 | 47.15 |

### Accu-TOF DART Analysis of Single Stage SCCO₂ Fractions

Compounds in Single Stage SCCO₂ Extraction at 40° C. and 100 Bar

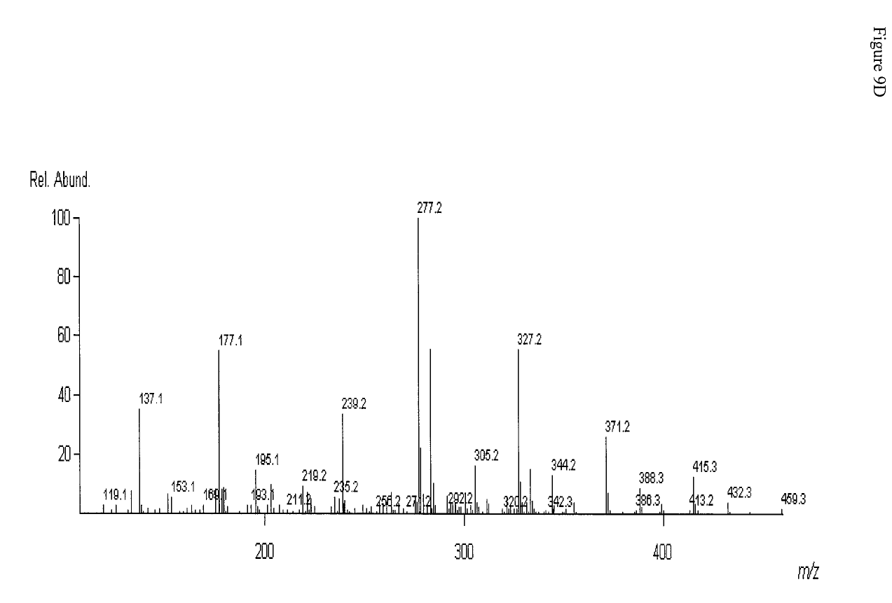

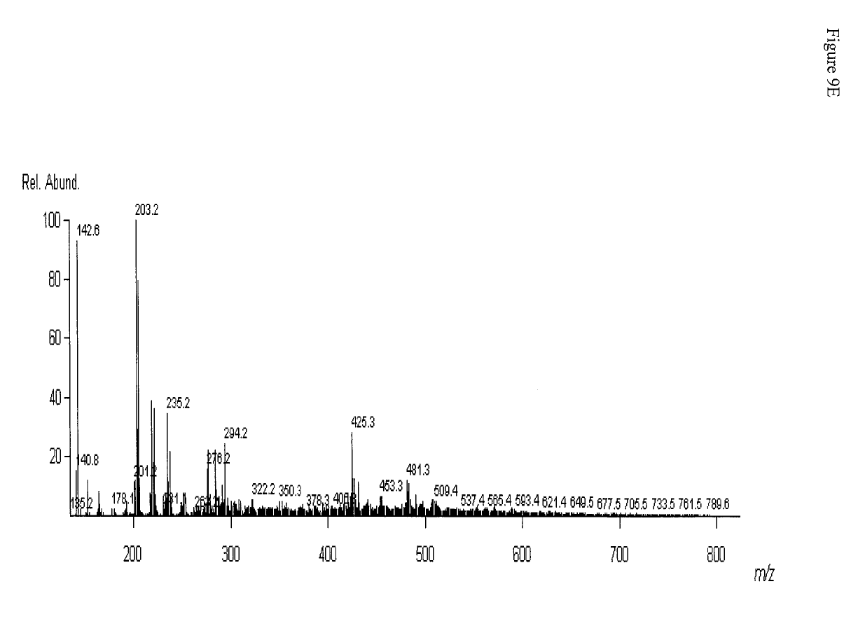

Shogaols, paradols, gingerols, and gingerdiols were present in the extract Amino acids, vitamins, fatty acids, saccharides, phenolic acids, phenols, sterols, capsaicins, gymnemagins and hydrocarbons were also present in this extract. 109 out of 326 (33%) unique chemicals have been directly identified in this extract using the DART TOF-MS coupled with the HerbalScience DART Database. Table 7 shows the compounds identified in the extracts along with their relative abundance. FIG. 8A shows the DART Spectrum

TABLE 7
Compounds in Single Stage SCCO₂ extraction at 40° C. and 100 bar.

| Compounds | Meas. | Calc. | Diff(u) | Abund. |
|---|---|---|---|---|
| 2-acetylpyrrole | 110.0646 | 110.0606 | 0.0039 | 14.867 |
| catechol/resorcinol/hydroquinone | 111.0512 | 111.0446 | 0.0066 | 16.652 |
| 2-methoxypyrazine | 111.0512 | 111.0558 | −0.0046 | 16.652 |
| povidone | 112.0862 | 112.0762 | 0.01 | 23.85 |
| histamine | 112.0862 | 112.0874 | −0.0012 | 23.85 |
| creatinine | 114.0609 | 114.0667 | −0.0058 | 5.2439 |
| butyl isothiocyanate | 116.0567 | 116.0534 | 0.0032 | 11.628 |
| levulinic acid | 117.0558 | 117.0551 | 0.0006 | 86.319 |
| indole | 118.0628 | 118.0656 | −0.0029 | 4.5397 |
| propyl sulfide | 119.0847 | 119.0894 | −0.0047 | 9.6793 |
| L-threonine | 120.075 | 120.066 | 0.009 | 5.0792 |
| 2,3,5-trimethylpyrazine | 123.0913 | 123.0922 | −0.0009 | 2.1557 |
| 2-ethyl-3-methylpyrazine | 123.0913 | 123.0922 | −0.0009 | 2.1557 |
| 4-dimethylaminopyridine | 123.0913 | 123.0922 | −0.0009 | 2.1557 |
| pyrogallol/phlorglucinol/maltol | 127.0405 | 127.0395 | 0.0009 | 100 |
| azulene | 129.0636 | 129.0704 | −0.0068 | 24.349 |
| leucine | 132.0955 | 132.1024 | −0.0069 | 5.8202 |
| glutaric acid | 133.0579 | 133.0501 | 0.0078 | 5.2865 |
| 2-indolinone | 133.0579 | 133.0527 | 0.0052 | 5.2865 |
| asparagine | 133.0579 | 133.0613 | −0.0034 | 5.2865 |
| cinnamaldehyde/methylbenzofuran | 133.0579 | 133.0653 | −0.0074 | 5.2865 |
| p-cymene | 135.1201 | 135.1174 | 0.0027 | 4.4355 |
| adenine | 136.0693 | 136.0623 | 0.007 | 17.114 |
| anisaldehyde/formic acid benzoate | 137.0614 | 137.0602 | 0.0012 | 70.628 |
| 2,3-dimethylhydroquinone | 139.0855 | 139.0759 | 0.0096 | 7.107 |
| 4-ethylquinol | 139.0855 | 139.0759 | 0.0096 | 7.107 |
| tyrosol | 139.0855 | 139.0759 | 0.0096 | 7.107 |
| furfuryl acetate | 141.0611 | 141.0551 | 0.006 | 18.412 |
| 3-hydroxy-2,3 dihydromaltol | 145.0504 | 145.0501 | 0.0003 | 85.787 |
| myosmine | 147.1 | 147.0922 | 0.0078 | 10.662 |
| 4-hydroxyisoleucine | 148.0991 | 148.0973 | 0.0017 | 5.53 |
| 4-OH Ile | 148.0991 | 148.0974 | 0.0017 | 5.53 |
| chitosan | 150.0857 | 150.0766 | 0.0091 | 7.3256 |
| 2-methoxy-4-vinylphenol | 151.0757 | 151.0759 | −0.0002 | 12.415 |
| benzoic acid ethyl ester | 151.0757 | 151.0759 | −0.0002 | 12.415 |
| cresyl acetate | 151.0757 | 151.0759 | −0.0002 | 12.415 |
| hydrocinnamic acid | 151.0757 | 151.0759 | −0.0002 | 12.415 |
| acetaminophen | 152.079 | 152.0711 | 0.0079 | 12.752 |
| anthranilic acid methyl ester | 152.079 | 152.0711 | 0.0079 | 12.752 |
| decadienal/santolina epoxide | 153.1274 | 153.1279 | −0.0006 | 21.572 |
| pinene oxide/piperitone pule | 153.1274 | 153.1279 | −0.0006 | 21.572 |
| diphenyl | 155.0823 | 155.0861 | −0.0038 | 21.825 |
| betonicine/acetyl valine | 160.0876 | 160.0973 | −0.0097 | 3.5724 |
| methylcoumarin | 161.063 | 161.0602 | 0.0028 | 3.3727 |
| L-2-aminoadipic acid | 162.0839 | 162.0766 | 0.0073 | 9.8416 |
| methyl cinnamic acid | 163.0764 | 163.0759 | 0.0005 | 45.877 |
| safrole | 163.0764 | 163.0759 | 0.0004 | 45.877 |
| cinnamaldehyde, o-methoxy | 163.0764 | 163.0759 | 0.0004 | 45.877 |
| deoxyglucose | 165.0857 | 165.0763 | 0.0094 | 18.715 |
| 2-deoxy-D-galactose/6-deoxyglucose | 165.0857 | 165.0763 | 0.0094 | 18.715 |
| fucose | 165.0857 | 165.0763 | 0.0094 | 18.715 |
| methyl-B-arabinopyranoside | 165.0857 | 165.0763 | 0.0094 | 18.715 |
| rhamnose | 165.0857 | 165.0763 | 0.0094 | 18.715 |
| 4-hydroxyphenyl-2-butanone | 165.0857 | 165.0915 | −0.0058 | 18.715 |
| acetic acid phenethyl ester | 165.0857 | 165.0915 | −0.0058 | 18.715 |
| 2-tert-butyl-p-quinone | 165.0857 | 165.0915 | −0.0058 | 18.715 |
| eugenol | 165.0857 | 165.0915 | −0.0058 | 18.715 |
| isoeugenol | 165.0857 | 165.0915 | −0.0058 | 18.715 |
| phenylacetic acid ethylester | 165.0857 | 165.0915 | −0.0058 | 18.715 |
| 2-methoxy-1-(1-propenyl)-phe | 165.0857 | 165.0915 | −0.0058 | 18.715 |
| eugenol | 165.0857 | 165.0916 | −0.0059 | 18.715 |
| iridol | 169.0869 | 169.0864 | 0.0005 | 21.529 |
| vitamin B6 | 170.0915 | 170.0817 | 0.0098 | 9.2136 |
| vitamin B6(pyridoxine) | 170.0915 | 170.0817 | 0.0098 | 9.2136 |
| L-methylhistidine | 170.0915 | 170.0929 | −0.0014 | 9.2136 |
| diphenylamine | 170.0915 | 170.0969 | −0.0054 | 9.2136 |
| 2,3-dimethoxyhydroquione | 171.0705 | 171.0657 | 0.0048 | 4.089 |
| 2-ketopimelic acid | 175.0685 | 175.0606 | 0.0079 | 2.8044 |
| shikimic acid | 175.0685 | 175.0606 | 0.0079 | 2.8044 |
| DL-a aminopimelic acid | 176.1 | 176.0923 | 0.0077 | 7.5639 |
| citrulline | 176.1 | 176.1035 | −0.0035 | 7.5639 |
| 4-dimethylaminocinnamaldehyde | 176.1 | 176.1075 | −0.0075 | 7.5639 |
| 4-methylumbelliferone | 177.056 | 177.0551 | 0.0008 | 2.3502 |
| herniarin | 177.056 | 177.0551 | 0.0008 | 2.3502 |
| hydroxymethylcoumarin | 177.056 | 177.0551 | 0.0008 | 2.3502 |
| methoxycoumarin | 177.056 | 177.0551 | 0.0008 | 2.3502 |
| 3-phenylpropyl isothiocyanat | 178.0658 | 178.069 | −0.0032 | 1.213 |
| coniferaldehyde | 179.0716 | 179.0708 | 0.0008 | 57.196 |
| methoxycinnamic acid | 179.0716 | 179.0708 | 0.0008 | 57.196 |
| D-mannosamine | 180.0969 | 180.0872 | 0.0096 | 34.927 |
| galacgtosamine | 180.0969 | 180.0872 | 0.0096 | 34.927 |
| glucosamine | 180.0969 | 180.0872 | 0.0096 | 34.927 |
| homophenylalanine | 180.0969 | 180.1024 | −0.0056 | 34.927 |
| salsolinol | 180.0969 | 180.1024 | −0.0056 | 34.927 |
| stilbene | 181.0979 | 181.1017 | −0.0039 | 24.053 |
| carbahcol | 183.0995 | 183.09 | 0.0095 | 15.508 |
| difluoromethylornithine | 183.0995 | 183.0945 | 0.005 | 15.508 |
| dihydroconiferyl alcohol | 183.0995 | 183.1021 | −0.0026 | 15.508 |
| epinephrine | 184.0988 | 184.0973 | 0.0015 | 11.967 |
| L-adrenaline | 184.0988 | 184.0973 | 0.0015 | 11.967 |
| n-acetyl-L-glutamine | 189.0922 | 189.0875 | 0.0047 | 2.8001 |
| castanospermine | 190.1034 | 190.1079 | −0.0045 | 8.9677 |
| ligustilide | 191.1136 | 191.1072 | 0.0064 | 10.978 |
| 5-methoxytryptamine | 191.1136 | 191.1184 | −0.0048 | 10.978 |
| cytisine | 191.1136 | 191.1184 | −0.0048 | 10.978 |
| myristicin | 193.0878 | 193.0864 | 0.0013 | 17.455 |
| dehydrozingerone | 193.0878 | 193.0865 | 0.0013 | 17.455 |
| a-phenylindol | 194.0961 | 194.0969 | −0.0008 | 13.263 |
| ferulic acid | 195.073 | 195.0657 | 0.0073 | 5.6639 |
| isoferulic acid | 195.073 | 195.0657 | 0.0073 | 5.6639 |
| methyl caffeate | 195.073 | 195.0657 | 0.0073 | 5.6639 |
| scytalone | 195.073 | 195.0657 | 0.0073 | 5.6639 |
| ferulic acid | 195.073 | 195.0657 | 0.0073 | 5.6639 |
| dehydrocurcumene | 201.1625 | 201.1643 | −0.0018 | 14.519 |
| curcumene/cuparene/calamenen | 203.1794 | 203.18 | −0.0006 | 66.723 |
| zingiberene/farnesene | 205.1951 | 205.1956 | −0.0005 | 87.596 |
| alloaromadendrene/elemene | 205.1951 | 205.1956 | −0.0005 | 87.596 |
| bicyclo[5,3,0] decane, 2-met | 205.1951 | 205.1956 | −0.0005 | 87.596 |
| cycloheptane, 4-methylene-1- | 205.1951 | 205.1956 | −0.0005 | 87.596 |
| aromadendrene, (+) | 205.1951 | 205.1956 | −0.0005 | 87.596 |
| caryophyllene | 205.1951 | 205.1956 | −0.0005 | 87.596 |
| cedrene | 205.1951 | 205.1956 | −0.0005 | 87.596 |
| farnesene | 205.1951 | 205.1956 | −0.0005 | 87.596 |
| humulene | 205.1951 | 205.1956 | −0.0005 | 87.596 |
| isocaryophyllene | 205.1951 | 205.1956 | −0.0005 | 87.596 |
| isolongifolene | 205.1951 | 205.1956 | −0.0005 | 87.596 |
| longicyclene/longifolene | 205.1951 | 205.1956 | −0.0005 | 87.596 |
| thujopsen | 205.1951 | 205.1956 | −0.0005 | 87.596 |
| valencene | 205.1951 | 205.1956 | −0.0005 | 87.596 |
| beta-gualene/cis-gamma-bisabollene | 205.1951 | 205.1956 | −0.0005 | 87.596 |
| copaene | 205.1951 | 205.1956 | −0.0005 | 87.596 |
| germacrene D | 205.1951 | 205.1956 | −0.0005 | 87.596 |
| a-cubebene | 205.1951 | 205.1956 | −0.0005 | 87.596 |
| a-muurolene | 205.1951 | 205.1956 | −0.0005 | 87.596 |
| β-farnesene | 205.1951 | 205.1956 | −0.0005 | 87.596 |
| caryophyllene | 205.1951 | 205.1956 | −0.0005 | 87.596 |
| trans-a-bergamotene | 205.1951 | 205.1956 | −0.0005 | 87.596 |
| a-zingiberene | 205.1951 | 205.1956 | −0.0005 | 87.596 |
| (−)-a-panasinsen | 205.1951 | 205.1956 | −0.0005 | 87.596 |
| β-sesquiphellandrene | 205.1951 | 205.1956 | −0.0005 | 87.596 |
| cedrene/valencene/β-guainene | 205.1951 | 205.1956 | −0.0005 | 87.596 |
| cycloheptane, 4-methylene-1- | 205.1951 | 205.1956 | −0.0005 | 87.596 |
| humulene | 205.1951 | 205.1956 | −0.0005 | 87.596 |
| (−)-zingiberene/sesquiphella | 205.1951 | 205.1956 | −0.0005 | 87.596 |
| caryophyllene | 205.1951 | 205.1956 | −0.0005 | 87.596 |
| bisabolene | 205.1951 | 205.1956 | −0.0005 | 87.596 |
| zingiberene | 205.1951 | 205.1956 | −0.0005 | 87.596 |
| β-sesquiphellandrene | 205.1951 | 205.1956 | −0.0005 | 87.596 |
| kynurenine | 209.0949 | 209.0926 | 0.0023 | 13.435 |
| chalcone | 209.0949 | 209.0966 | −0.0017 | 13.435 |
| flavan | 211.1099 | 211.1123 | −0.0025 | 6.6915 |
| harmine | 213.112 | 213.1028 | 0.0092 | 9.5389 |
| n-acetyl-DL-arginne | 217.1265 | 217.13 | −0.0035 | 11.138 |
| furanoeremophilane | 219.1749 | 219.1749 | 0 | 25 |
| nootkatone | 219.1749 | 219.1749 | 0 | 25 |
| valerenal | 219.1749 | 219.1749 | 0 | 25 |
| xanthorrhizol | 219.1749 | 219.1749 | 0 | 25 |
| curlone | 219.1749 | 219.1749 | 0 | 25 |
| turmerone/ar-turmerol | 219.1749 | 219.1749 | 0 | 25 |
| caryophellene oxide | 221.1917 | 221.1905 | 0.0012 | 20.892 |
| β-caryophyllene epoxide | 221.1917 | 221.1905 | 0.0012 | 20.892 |
| 6-isopropenyl-4,8a-dimethyl- | 221.1917 | 221.1905 | 0.0012 | 20.892 |
| caryophyllene oxide | 221.1917 | 221.1905 | 0.0012 | 20.892 |
| 6,10-dodecadien-1-yn-3-ol, 3 | 221.1917 | 221.1905 | 0.0012 | 20.892 |
| bergamotol, Z-a-trans- | 221.1917 | 221.1905 | 0.0012 | 20.892 |
| spathulenol/9-cedranone/lanceol | 221.1917 | 221.1905 | 0.0012 | 20.892 |
| 6-isopropenyl-4,8a-dimethyl- | 221.1917 | 221.1905 | 0.0012 | 20.892 |
| 6-benzylaminopurine | 226.1179 | 226.1092 | 0.0087 | 4.5744 |
| carnosine | 227.1171 | 227.1144 | 0.0027 | 7.9565 |
| terphenyl | 231.1174 | 231.1174 | 0 | 7.3927 |
| costunolide | 233.1525 | 233.1541 | −0.0016 | 16.294 |
| eremophilanlactone | 235.1622 | 235.1698 | −0.0076 | 16.474 |
| 2-octyl benzoate | 235.1622 | 235.1698 | −0.0077 | 16.474 |
| valerenic acid | 235.1622 | 235.1698 | −0.0077 | 16.474 |
| proposed compound 4/6-(3-hyd | 235.1622 | 235.1698 | −0.0077 | 16.474 |
| vellerdiol | 237.1783 | 237.1854 | −0.0071 | 12.789 |
| 3-methyl-but-2-enoic acid, 1 | 237.1783 | 237.1854 | −0.0071 | 12.789 |
| 2-pentenoic acid, 3-methyl-5 | 237.1783 | 237.1854 | −0.0071 | 12.789 |
| bicyclo[4.4.0]dec-2-ene-4-ol | 237.1783 | 237.1854 | −0.0071 | 12.789 |
| a-ionyl acetate | 237.1783 | 237.1854 | −0.0071 | 12.789 |
| phosphatidylcholine | 243.1191 | 243.123 | −0.0039 | 2.0074 |
| osthole | 245.1214 | 245.1177 | 0.0036 | 5.9698 |
| 3,3′-di-inoylmethane | 247.1257 | 247.1235 | 0.0021 | 9.9559 |
| ellpticine | 247.1257 | 247.1235 | 0.0021 | 9.9559 |
| santonin | 247.1257 | 247.1334 | −0.0077 | 9.9559 |
| 4-shogaol | 249.137 | 249.1291 | 0.0079 | 5.4547 |
| 6-paradol | 251.1556 | 251.1647 | −0.0091 | 7.1153 |
| hydroxyvalerenic acid | 251.1556 | 251.1647 | −0.0091 | 7.1153 |
| 1,6-dimethyl-9-(1-methylethy | 251.1556 | 251.1647 | −0.0091 | 7.1153 |
| cimetidine | 253.13 | 253.1235 | 0.0065 | 4.662 |
| panaxydol/octanoic acid, 3-p | 261.1861 | 261.1854 | 0.0007 | 26.437 |
| abscisic acid | 265.1437 | 265.144 | −0.0003 | 6.1304 |
| taxol side chain diol | 272.1359 | 272.1286 | 0.0073 | 3.5549 |
| 3,6-epoxy-1-(4-hydroxy-3-met | 275.1734 | 275.1647 | 0.0087 | 17.935 |
| podocarpic acid | 275.1734 | 275.1647 | 0.0087 | 17.935 |
| eserine | 276.1744 | 276.1712 | 0.0032 | 34.578 |
| galanthamine | 288.1631 | 288.1599 | 0.0031 | 5.0341 |
| hyoscyamine | 290.1732 | 290.1756 | −0.0024 | 11.686 |
| 6-dehydrogingerdione | 291.1664 | 291.1596 | 0.0067 | 28.079 |
| N-octyl-B-D-glucopyranoside | 293.1897 | 293.1964 | −0.0067 | 11.86 |
| cinchonidine/cinchonine | 295.1785 | 295.181 | −0.0025 | 6.0972 |
| eburnamonine | 295.1785 | 295.181 | −0.0025 | 6.0972 |
| retinoic acid | 301.2214 | 301.2167 | 0.0047 | 5.6157 |
| abietic acid | 303.2263 | 303.2324 | −0.0062 | 13.392 |
| eicosapentaenoic acid | 303.2263 | 303.2324 | −0.0062 | 13.392 |
| 8-shogaol | 305.2128 | 305.2117 | 0.0011 | 31.205 |
| bioallethrin | 313.2751 | 313.2742 | 0.0008 | 1.3618 |
| homocapsaicin | 320.2137 | 320.2226 | −0.0089 | 5.5596 |
| 8-gingerol/rapanone | 323.2257 | 323.2222 | 0.0035 | 4.5601 |
| hydroxyprogesterone/DHEA acetate | 331.2242 | 331.2273 | −0.0032 | 11.296 |
| 10-shogaol | 333.245 | 333.243 | 0.002 | 67.777 |
| pregnanetriol | 337.2836 | 337.2742 | 0.0094 | 7.3797 |
| 10-dehydrogingerdione | 347.2294 | 347.2222 | 0.0072 | 15.409 |
| calycanthine | 347.2294 | 347.2235 | 0.0059 | 15.409 |
| tetrahydrocorticosterone | 351.257 | 351.2535 | 0.0035 | 5.0889 |
| 10-gingerdiol | 353.2792 | 353.2692 | 0.01 | 1.2072 |
| rubrocyanin | 354.2135 | 354.2096 | 0.0039 | 1.8111 |
| tetrahydropalmatine/glaucine | 356.1956 | 356.1862 | 0.0093 | 5.0211 |
| cafestol acetate | 359.22 | 359.2222 | −0.0022 | 11.031 |
| diacetyl-6-gingerdiol | 381.2218 | 381.2277 | −0.0059 | 2.1247 |
| resibufogenin | 385.2355 | 385.2379 | −0.0024 | 8.1143 |
| dehydrocholic acid | 403.2399 | 403.2484 | −0.0085 | 1.5196 |
| cholic acid | 409.2958 | 409.2954 | 0.0004 | 1.2576 |
| spironolactone | 417.2119 | 417.2099 | 0.002 | 15.168 |
| neoruscogenin | 429.3092 | 429.3005 | 0.0087 | 3.0249 |
| schisandrin | 433.2156 | 433.2226 | −0.007 | 1.8743 |
| gymnemagenin | 492.3486 | 492.3451 | 0.0035 | 2.7927 |

Compounds in Single Stage SCCO₂ Extraction at 40° C. and 500 Bar

6-shogoal and galanolactone were present in this extract in 48.6 and 2.5% relative abundance, respectively. Other shogaols, paradols, gingerols, and gingerdiols are present in the extract Amino acids, vitamins, fatty acids, saccharides, phenolic acids, phenols, sterols, capsaicins, gymnemagins, quinines, alkaloids, terpenoids, boswellic acids, saponins and hydrocarbons were also present in this extract. 99 out of 474 (21%) unique chemicals have been directly identified in this extract using the DART TOF-MS. Table 8 shows the compounds identified in the extracts along with their relative abundance. FIG. 8B shows the DART Spectrum.

TABLE 8
Compounds in Single Stage SCCO₂ extraction at 40° C. and 500 bar.

| Compound | Meas. | Calc. | Diff(u) |
|---|---|---|---|
| ethylbenzene | 107.0861 | 107.0861 | 0 |
| norcamphor/heptadienal | 111.0838 | 111.081 | 0.0028 |
| propyl sulfide | 119.0863 | 119.0894 | −0.0031 |
| pseudocumene/propynylcyclohexene | 121.1019 | 121.1017 | 0.0002 |
| 5-hepten-2-one, 6-methyl- | 127.1118 | 127.1123 | −0.0005 |
| leucine | 132.0932 | 132.1024 | −0.0092 |
| ornithine | 133.1035 | 133.0977 | 0.0058 |
| dicyclopentadiene | 133.1035 | 133.1017 | 0.0018 |
| p-cymene | 135.1185 | 135.1174 | 0.0011 |
| anisaldehyde/formic acid benoate | 137.0617 | 137.0602 | 0.0015 |
| 2-acetyl-3-methypyrazine | 137.0617 | 137.0715 | −0.0098 |
| trigonelline/vitamin H | 138.064 | 138.0555 | 0.0085 |
| octalactone | 143.1094 | 143.1072 | 0.0022 |
| crotonylbetaine | 145.1027 | 145.1103 | −0.0077 |
| lysine | 147.1179 | 147.1133 | 0.0046 |
| carvacrol/thymol/cymenol | 151.1169 | 151.1123 | 0.0046 |
| 2-butyl-3-methylpyrazine | 151.1169 | 151.1235 | −0.0066 |
| norpseudophedrine | 152.11 | 152.1075 | 0.0024 |
| decadienal/santolina epoxide | 153.1278 | 153.1279 | −0.0001 |
| pinene oxide/piperitone pule | 153.1278 | 153.1279 | −0.0001 |
| methylcholine | 161.1332 | 161.1416 | −0.0084 |
| acetylthiocholine | 163.1024 | 163.1031 | −0.0007 |
| N-phenylmorpholine | 164.1168 | 164.1075 | 0.0093 |
| jasmone | 165.1185 | 165.1279 | −0.0094 |
| ephedrine | 166.1229 | 166.1232 | −0.0003 |
| hordenine | 166.1229 | 166.1232 | −0.0003 |
| pseudoephedrine | 166.1229 | 166.1232 | −0.0003 |
| camphorquinone | 167.107 | 167.1072 | −0.0002 |
| perillic acid | 167.107 | 167.1072 | −0.0002 |
| 3-(phenylmethoxy)-1-propanol | 167.107 | 167.1072 | −0.0002 |
| (3Z)-3-hexenyl-2-butenoate | 169.1236 | 169.1228 | 0.0007 |
| chrysanthemolactone | 169.1236 | 169.1228 | 0.0007 |
| a-Limonene diepoxide | 169.1236 | 169.1228 | 0.0007 |
| decalactone | 171.1371 | 171.1385 | −0.0015 |
| linalool oxide | 171.1371 | 171.1385 | −0.0015 |
| butanoic acid, 3-hexenyl ester | 171.1371 | 171.1385 | −0.0015 |
| 3,7-octadiene-2,6-diol, 2,6- | 171.1371 | 171.1385 | −0.0015 |
| 1,7-octadiene-3,6-diol, 2,6- | 171.1371 | 171.1385 | −0.0015 |
| arcaine | 173.1453 | 173.1514 | −0.0061 |
| n-octyl acetate | 173.1453 | 173.1541 | −0.0088 |
| capric acid | 173.1453 | 173.1541 | −0.0088 |
| caprylic acid ethyl ester | 173.1453 | 173.1541 | −0.0088 |
| n-decanoic acid/1,3-dioxolane | 173.1453 | 173.1541 | −0.0088 |
| cinnamyl acetate | 177.0927 | 177.0915 | 0.0012 |
| canavanine | 177.0927 | 177.0987 | −0.006 |
| eugenol methyl ether | 179.111 | 179.1072 | 0.0038 |
| 4-(p-methoxyphenyl)-2-butanone | 179.111 | 179.1072 | 0.0038 |
| anisylacetone | 179.111 | 179.1072 | 0.0038 |
| eugenol methylether | 179.111 | 179.1072 | 0.0038 |
| 2(4H)-benzofuranone, 5,6,7,7 | 181.1239 | 181.1228 | 0.001 |
| chamazulen | 185.1293 | 185.133 | −0.0037 |
| 1,3-di-tert-butylbenzene | 191.183 | 191.18 | 0.003 |
| damascone | 193.1615 | 193.1592 | 0.0023 |
| ionone | 193.1615 | 193.1592 | 0.0023 |
| β-pinene, 3-(acetylmethyl)- | 193.1615 | 193.1592 | 0.0023 |
| guaiazulene | 199.144 | 199.1487 | −0.0048 |
| naphthalene, 1,6-dimethyl-4- | 199.144 | 199.1487 | −0.0048 |
| dehydrocurcumene | 201.165 | 201.1643 | 0.0007 |
| curcumene/cuparene/calamenene | 203.1794 | 203.18 | −0.0007 |
| zingiberene/(Z,E)-a-farnesene | 205.195 | 205.1956 | −0.0007 |
| alloaromadendrene/elemene | 205.195 | 205.1956 | −0.0007 |
| cycloheptane, 4-methylene-1- | 205.195 | 205.1956 | −0.0007 |
| aromadendrene, (+) | 205.195 | 205.1956 | −0.0007 |
| caryophyllene | 205.195 | 205.1956 | −0.0007 |
| cedrene | 205.195 | 205.1956 | −0.0007 |
| farnesene | 205.195 | 205.1956 | −0.0007 |
| humulene | 205.195 | 205.1956 | −0.0007 |
| isocaryophyllene | 205.195 | 205.1956 | −0.0007 |
| isolongifolene | 205.195 | 205.1956 | −0.0007 |
| longicyclene/longifolene | 205.195 | 205.1956 | −0.0007 |
| thujopsen | 205.195 | 205.1956 | −0.0007 |
| valencene | 205.195 | 205.1956 | −0.0007 |
| beta-caryophyllene | 205.195 | 205.1956 | −0.0007 |
| beta-gualene/cis-gamma-bisabollene | 205.195 | 205.1956 | −0.0007 |
| copaene | 205.195 | 205.1956 | −0.0007 |
| germacrene D | 205.195 | 205.1956 | −0.0007 |
| a-cubebene | 205.195 | 205.1956 | −0.0007 |
| a-muurolene | 205.195 | 205.1956 | −0.0007 |
| trans-a-bergamotene | 205.195 | 205.1956 | −0.0007 |
| (−)-a-panasinsen | 205.195 | 205.1956 | −0.0007 |
| β-sesquiphellandrene | 205.195 | 205.1956 | −0.0007 |
| cedrene/valencene/β-guainene | 205.195 | 205.1956 | −0.0007 |
| humulene | 205.195 | 205.1956 | −0.0007 |
| (−)-zingiberene/sesquiphelladrene | 205.195 | 205.1956 | −0.0007 |
| 3,5-bis(1,1-dimethylethyl)-p | 207.1776 | 207.1749 | 0.0027 |
| carvylacetate | 209.1564 | 209.1541 | 0.0023 |
| epoxy-a-terpenyl acetate | 213.1576 | 213.149 | 0.0086 |
| hexylcinnamaldehyde | 217.1597 | 217.1592 | 0.0005 |
| ar-tumerone | 217.1597 | 217.1592 | 0.0005 |
| furanoeremophilane | 219.1752 | 219.1749 | 0.0003 |
| nootkatone | 219.1752 | 219.1749 | 0.0003 |
| valerenal | 219.1752 | 219.1749 | 0.0003 |
| xanthorrhizol | 219.1752 | 219.1749 | 0.0003 |
| curlone | 219.1752 | 219.1749 | 0.0003 |
| turmerone/ar-turmerol/xanthorrhizol | 219.1752 | 219.1749 | 0.0003 |
| caryophellene oxide | 221.1908 | 221.1905 | 0.0003 |
| bergamotol, Z-a-trans- | 221.1908 | 221.1905 | 0.0003 |
| spathulenol/9-cedranone/lanceol | 221.1908 | 221.1905 | 0.0003 |
| 6-isopropenyl-4,8a-dimethyl- | 221.1908 | 221.1905 | 0.0003 |
| undec-2-ene-8,10-diynoic acid | 232.1731 | 232.1701 | 0.003 |
| costunolide | 233.161 | 233.1541 | 0.0069 |
| panthenol | 234.1746 | 234.1705 | 0.0041 |
| eremophilanlactone | 235.1693 | 235.1698 | −0.0005 |
| 2-octyl benzoate | 235.1693 | 235.1698 | −0.0005 |
| valerenic acid | 235.1693 | 235.1698 | −0.0005 |
| vellerdiol | 237.1843 | 237.1854 | −0.0011 |
| 3-methyl-but-2-enoic acid, 1 | 237.1843 | 237.1854 | −0.0011 |
| 2-pentenoic acid, 3-methyl-5 | 237.1843 | 237.1854 | −0.0011 |
| bicyclo[4.4.0]dec-2-ene-4-ol | 237.1843 | 237.1854 | −0.0011 |
| a-ionyl acetate | 237.1843 | 237.1854 | −0.0011 |
| isobornyl isovalerate | 239.2059 | 239.2011 | 0.0048 |
| linalyl iso-valerate | 239.2059 | 239.2011 | 0.0048 |
| 3-hydroxymyristic acid | 245.2041 | 245.2116 | −0.0075 |
| 6-paradol | 251.1662 | 251.1647 | 0.0014 |
| hydroxyvalerenic acid | 251.1662 | 251.1647 | 0.0014 |
| palmitic acid | 257.2487 | 257.248 | 0.0006 |
| oxymatrine | 265.1935 | 265.1916 | 0.0019 |
| C20H32/biformene/kaur-16-ene | 273.2546 | 273.2582 | −0.0036 |
| eserine | 276.178 | 276.1712 | 0.0068 |
| 6-shogaol | 277.1802 | 277.1803 | −0.0001 |
| menthyl salicylate | 277.1802 | 277.1803 | −0.0001 |
| cyclohexanecarboxylic acid, | 277.1802 | 277.1803 | −0.0001 |
| 6-shogaol | 277.1802 | 277.1804 | −0.0002 |
| stearolic acid | 281.2426 | 281.248 | −0.0055 |
| linoleic acid | 281.2426 | 281.248 | −0.0055 |
| 9,12-octadecadienoic acid | 281.2426 | 281.248 | −0.0055 |
| stearolic acid/linoelaidic acid | 281.2426 | 281.248 | −0.0055 |
| 9,12-octadecadienoic acid | 281.2426 | 281.248 | −0.0055 |
| linoleic acid | 281.2426 | 281.248 | −0.0055 |
| oleic acid | 283.2614 | 283.2637 | −0.0023 |
| elaidic acid | 283.2614 | 283.2637 | −0.0023 |
| petroselaidic acid | 283.2614 | 283.2637 | −0.0023 |
| vaccenic acid | 283.2614 | 283.2637 | −0.0023 |
| vitamin A(retinol) | 287.2406 | 287.2375 | 0.003 |
| abieta-8,11,13-trien-18-ol | 287.2406 | 287.2375 | 0.003 |
| atropine | 291.1766 | 291.1834 | −0.0068 |
| N-octyl-B-D-glucopyranoside | 293.1941 | 293.1964 | −0.0023 |
| 9,12-octadecadienoyl chloride | 299.2222 | 299.2141 | 0.0081 |
| aleuritic acid | 305.2238 | 305.2328 | −0.009 |
| 10-paradol | 307.2349 | 307.2273 | 0.0076 |
| dihydrocapsaicin | 308.2177 | 308.2225 | −0.0048 |
| galanolactone/aframodial/galanal | 319.2334 | 319.2273 | 0.0061 |
| 2-chloroethyl palmitate | 319.2334 | 319.2404 | −0.0071 |
| homodihydrocapsaicin | 322.2406 | 322.2382 | 0.0024 |
| docosahexenoic acid | 329.2493 | 329.248 | 0.0013 |
| hydroxyprogesterone/DHEA acetate | 331.2352 | 331.2273 | 0.0079 |
| 10-shogaol | 333.2481 | 333.243 | 0.0052 |
| incensole acetate | 349.2672 | 349.2742 | −0.0071 |
| ginkgolic acid II | 375.2967 | 375.2899 | 0.0068 |
| mogroside backbone-4H2O | 405.3577 | 405.3522 | 0.0055 |
| benzethonium | 413.3294 | 413.3294 | 0 |
| tomatidine | 416.361 | 416.3528 | 0.0082 |
| calcitriol/sarsapogenin | 417.34 | 417.3368 | 0.0032 |
| jervine | 426.2919 | 426.3008 | −0.0089 |
| hecogenin/ruscogenin | 431.321 | 431.3161 | 0.0049 |
| ursonic acid/dehydroboswellic acid | 455.3452 | 455.3525 | −0.0074 |
| ursolic/oleanolic/boswellic acids | 457.3701 | 457.3682 | 0.0019 |
| Diepoxydammar diol | 459.3408 | 459.3474 | −0.0067 |
| ganoderic acid D/M | 469.3395 | 469.3318 | 0.0077 |
| keto boswellic acid/glycyrrhizol | 471.344 | 471.3474 | −0.0035 |
| jujubogenin/bacoside A | 473.362 | 473.3631 | −0.0011 |
| Gymnemasaponin II - 2 Glc | 475.3733 | 475.3787 | −0.0055 |
| 18-glycyrrhetinic acid methyl ester | 485.3644 | 485.3631 | 0.0013 |
| keto boswellic acid/ganodermol | 487.374 | 487.3787 | −0.0048 |
| 23-Hydroxylongispinogenin | 491.3747 | 491.3736 | 0.001 |
| gymnemagenin | 492.3497 | 492.3451 | 0.0046 |
| 3-O-acetyl-9,11-dehydro BA | 497.3661 | 497.3631 | 0.003 |
| 3-O-acetyl-11-hydroxy boswellic acid | 515.3749 | 515.3737 | 0.0012 |
| betulin diacetate | 527.4039 | 527.41 | −0.0062 |
| adhyperforin | 551.4041 | 551.41 | −0.006 |
| gymnemic acid IV/XIV - GlcA | 589.4142 | 589.4104 | 0.0038 |

Compounds in Single Stage SCCO₂ Extraction at 60° C. and 100 Bar

6-shogoal and galanolactone were present in this extract in 30.1 and 0.9% relative abundance, respectively. Other shogaols, paradols, gingerols, and gingerdiols were present in the extract. Amino acids, alkaloids, quinones, tumerones, vitamins, fatty acids, saccharides, phenolic acids, phenols, sterols, capsaicins, gymnemagins, saponins and hydrocarbons were also present in this extract. 92 out of 276 (33%) unique chemicals have been directly identified in this extract using the DART TOF-MS. Table 9 shows the compounds identified in the extracts along with their relative abundance. FIG. 8C shows the DART Spectrum.

TABLE 9
Compounds in Single Stage SCCO₂ extraction at 60° C. and 100 bar

| Compound | Meas. | Calc. | Diff(u) | Abund. |
|---|---|---|---|---|
| ethylbenzene | 107.0863 | 107.0861 | 0.0002 | 1.5333 |
| propyl sulfide | 119.0858 | 119.0894 | −0.0036 | 10.859 |
| pseudocumene/propynylcyclohe | 121.1014 | 121.1017 | −0.0003 | 11.726 |
| 5-hepten-2-one, 6-methyl- | 127.1143 | 127.1123 | 0.002 | 0.7487 |
| ornithine | 133.1031 | 133.0977 | 0.0054 | 2.8522 |
| dicyclopentadiene | 133.1031 | 133.1017 | 0.0014 | 2.8522 |
| p-cymene | 135.1184 | 135.1174 | 0.001 | 6.3634 |
| anisaldehyde/formic acid benzoate | 137.0601 | 137.0602 | −0.0001 | 14.533 |
| trigonelline/vitamin H | 138.0643 | 138.0555 | 0.0087 | 0.3485 |
| octalactone | 143.1081 | 143.1072 | 0.0009 | 0.847 |
| crotonylbetaine | 145.1021 | 145.1103 | −0.0083 | 3.0053 |
| lysine | 147.1171 | 147.1133 | 0.0038 | 8.368 |
| 1-methyl-3-phenylpropylamine | 150.1371 | 150.1282 | 0.0088 | 1.086 |
| 4-phenylbutylamine | 150.1371 | 150.1282 | 0.0088 | 1.086 |
| carvacrol/thymol/cymenol | 151.1178 | 151.1123 | 0.0055 | 3.117 |
| 2-butyl-3-methylpyrazine | 151.1178 | 151.1235 | −0.0058 | 3.117 |
| norpseudophedrine | 152.1119 | 152.1075 | 0.0044 | 0.2933 |
| decadienal/santolina epoxide | 153.1275 | 153.1279 | −0.0005 | 6.4149 |
| pinene oxide/piperitone pule | 153.1275 | 153.1279 | −0.0005 | 6.4149 |
| pseudopelletierine | 154.1313 | 154.1232 | 0.008 | 0.4655 |
| cineole/borneol | 155.1456 | 155.1436 | 0.002 | 0.2191 |
| methone/pinocampheol/pulegol | 155.1456 | 155.1436 | 0.002 | 0.2191 |
| methylcholine | 161.1327 | 161.1416 | −0.0089 | 6.4216 |
| jasmone | 165.1325 | 165.1279 | 0.0045 | 1.5611 |
| ephedrine | 166.128 | 166.1232 | 0.0048 | 0.2228 |
| hordenine | 166.128 | 166.1232 | 0.0048 | 0.2228 |
| pseudoephedrine | 166.128 | 166.1232 | 0.0048 | 0.2228 |
| camphorquinone | 167.1061 | 167.1072 | −0.0011 | 4.1549 |
| perillic acid | 167.1061 | 167.1072 | −0.0011 | 4.1549 |
| 3-(phenylmethoxy)-1-propanol | 167.1061 | 167.1072 | −0.0011 | 4.1549 |
| (3Z)-3-hexenyl-2-butenoate | 169.1225 | 169.1228 | −0.0003 | 1.1254 |
| chrysanthemolactone | 169.1225 | 169.1228 | −0.0003 | 1.1254 |
| a-Limonene diepoxide | 169.1225 | 169.1228 | −0.0003 | 1.1254 |
| decalactone | 171.1365 | 171.1385 | −0.002 | 1.021 |
| linalool oxide | 171.1365 | 171.1385 | −0.002 | 1.021 |
| butanoic acid, 3-hexenyl ester | 171.1365 | 171.1385 | −0.002 | 1.021 |
| 3,7-octadiene-2,6-diol, 2,6- | 171.1365 | 171.1385 | −0.002 | 1.021 |
| 1,7-octadiene-3,6-diol, 2,6- | 171.1365 | 171.1385 | −0.002 | 1.021 |
| arcaine | 173.1463 | 173.1514 | −0.0052 | 1.466 |
| n-octyl acetate | 173.1463 | 173.1541 | −0.0079 | 1.466 |
| capric acid | 173.1463 | 173.1541 | −0.0079 | 1.466 |
| caprylic acid ethyl ester | 173.1463 | 173.1541 | −0.0079 | 1.466 |
| n-decanoic acid/1,3-dioxolane | 173.1463 | 173.1541 | −0.0079 | 1.466 |
| cinnamyl acetate | 177.0925 | 177.0915 | 0.0009 | 9.8921 |
| canavanine | 177.0925 | 177.0987 | −0.0063 | 9.8921 |
| 2(4H)-benzofuranone, 5,6,7,7 | 181.1263 | 181.1228 | 0.0035 | 1.2848 |
| pinonic acid | 185.1275 | 185.1177 | 0.0098 | 1.1723 |
| 3-methyl-2-butenoic acid, 2- | 185.1275 | 185.1177 | 0.0098 | 1.1723 |
| chamazulen | 185.1275 | 185.133 | −0.0055 | 1.1723 |
| 1,3-di-tert-butylbenzene | 191.1824 | 191.18 | 0.0024 | 3.2908 |
| damascone | 193.1614 | 193.1592 | 0.0022 | 1.3571 |
| ionone | 193.1614 | 193.1592 | 0.0022 | 1.3571 |
| β-pinene, 3-(acetylmethyl)- | 193.1614 | 193.1592 | 0.0022 | 1.3571 |
| a-phenylindol | 194.0946 | 194.0969 | −0.0023 | 1.4029 |
| guaiazulene | 199.1457 | 199.1487 | −0.003 | 1.1177 |
| naphthalene, 1,6-dimethyl-4- | 199.1457 | 199.1487 | −0.003 | 1.1177 |
| dehydrocurcumene | 201.1644 | 201.1643 | 0.0001 | 17.337 |
| curcumene/cuparene/calamenene | 203.1789 | 203.18 | −0.0011 | 100 |
| zingiberene/(Z,E)-a-farnesene | 205.1946 | 205.1956 | −0.001 | 81.558 |
| alloaromadendrene/elemene | 205.1946 | 205.1956 | −0.001 | 81.558 |
| cycloheptane, 4-methylene-1- | 205.1946 | 205.1956 | −0.001 | 81.558 |
| aromadendrene, (+) | 205.1946 | 205.1956 | −0.001 | 81.558 |
| caryophyllene | 205.1946 | 205.1956 | −0.001 | 81.558 |
| cedrene | 205.1946 | 205.1956 | −0.001 | 81.558 |
| farnesene | 205.1946 | 205.1956 | −0.001 | 81.558 |
| humulene | 205.1946 | 205.1956 | −0.001 | 81.558 |
| isocaryophyllene | 205.1946 | 205.1956 | −0.001 | 81.558 |
| isolongifolene | 205.1946 | 205.1956 | −0.001 | 81.558 |
| longicyclene/longifolene | 205.1946 | 205.1956 | −0.001 | 81.558 |
| thujopsen | 205.1946 | 205.1956 | −0.001 | 81.558 |
| valencene | 205.1946 | 205.1956 | −0.001 | 81.558 |
| beta-gualene/cis-gamma-bisab | 205.1946 | 205.1956 | −0.001 | 81.558 |
| copaene | 205.1946 | 205.1956 | −0.001 | 81.558 |
| germacrene D | 205.1946 | 205.1956 | −0.001 | 81.558 |
| a-cubebene | 205.1946 | 205.1956 | −0.001 | 81.558 |
| a-muurolene | 205.1946 | 205.1956 | −0.001 | 81.558 |
| (−)-a-panasinsen | 205.1946 | 205.1956 | −0.001 | 81.558 |
| β-sesquiphellandrene | 205.1946 | 205.1956 | −0.001 | 81.558 |
| cedrene/valencene/β-guainene | 205.1946 | 205.1956 | −0.001 | 81.558 |
| cycloheptane, 4-methylene-1- | 205.1946 | 205.1956 | −0.001 | 81.558 |
| humulene | 205.1946 | 205.1956 | −0.001 | 81.558 |
| 3,5-bis(1,1-dimethylethyl)-p | 207.1736 | 207.1749 | −0.0013 | 11.414 |
| carvylacetate | 209.158 | 209.1541 | 0.0039 | 1.1113 |
| hexylcinnamaldehyde | 217.1595 | 217.1592 | 0.0003 | 9.2758 |
| ar-tumerone | 217.1595 | 217.1592 | 0.0003 | 9.2758 |
| furanoeremophilane | 219.1744 | 219.1749 | −0.0005 | 31.303 |
| nootkatone | 219.1744 | 219.1749 | −0.0005 | 31.303 |
| valerenal | 219.1744 | 219.1749 | −0.0005 | 31.303 |
| xanthorrhizol | 219.1744 | 219.1749 | −0.0005 | 31.303 |
| curlone | 219.1744 | 219.1749 | −0.0005 | 31.303 |
| turmerone/ar-turmerol | 219.1744 | 219.1749 | −0.0005 | 31.303 |
| caryophellene oxide | 221.1901 | 221.1905 | −0.0004 | 25.267 |
| β-caryophyllene epoxide | 221.1901 | 221.1905 | −0.0004 | 25.267 |
| spathulenol/caryophyllene oxide | 221.1901 | 221.1905 | −0.0004 | 25.267 |
| 6,10-dodecadien-1-yn-3-ol, 3 | 221.1901 | 221.1905 | −0.0004 | 25.267 |
| caryophyllene oxide | 221.1901 | 221.1905 | −0.0004 | 25.267 |
| bergamotol, Z-a-trans- | 221.1901 | 221.1905 | −0.0004 | 25.267 |
| spathulenol/9-cedranone/lanceol | 221.1901 | 221.1905 | −0.0004 | 25.267 |
| caryophyllene oxide/(−)-spat | 221.1901 | 221.1905 | −0.0004 | 25.267 |
| undec-2-ene-8,10-diynoic aci | 232.1755 | 232.1701 | 0.0054 | 4.3332 |
| costunolide | 233.1629 | 233.1541 | 0.0088 | 5.6157 |
| panthenol | 234.177 | 234.1705 | 0.0065 | 2.1038 |
| eremophilanlactone | 235.1685 | 235.1698 | −0.0013 | 12.339 |
| 2-octyl benzoate | 235.1685 | 235.1698 | −0.0013 | 12.339 |
| valerenic acid | 235.1685 | 235.1698 | −0.0013 | 12.339 |
| vellerdiol | 237.1826 | 237.1854 | −0.0028 | 9.8963 |
| 3-methyl-but-2-enoic acid, 1 | 237.1826 | 237.1854 | −0.0028 | 9.8963 |
| 2-pentenoic acid, 3-methyl-5 | 237.1826 | 237.1854 | −0.0028 | 9.8963 |
| a-ionyl acetate | 237.1826 | 237.1854 | −0.0028 | 9.8963 |
| 3-hydroxymyristic acid | 245.2117 | 245.2116 | 0.0001 | 0.5613 |
| 6-paradol | 251.1706 | 251.1647 | 0.0059 | 3.2657 |
| hydroxyvalerenic acid | 251.1706 | 251.1647 | 0.0058 | 3.2657 |
| 1,6-dimethyl-9-(1-methylethy | 251.1706 | 251.1647 | 0.0058 | 3.2657 |
| palmitic acid | 257.2488 | 257.248 | 0.0008 | 2.2584 |
| C20H32/biformene/kaur-16-ene | 273.2553 | 273.2582 | −0.0029 | 5.8918 |
| eserine | 276.1743 | 276.1712 | 0.003 | 8.2442 |
| 6-shogaol | 277.1791 | 277.1803 | −0.0012 | 30.134 |
| menthyl salicylate | 277.1791 | 277.1803 | −0.0012 | 30.134 |
| cyclohexanecarboxylic acid, | 277.1791 | 277.1803 | −0.0012 | 30.134 |
| 6-shogaol | 277.1791 | 277.1804 | −0.0012 | 30.134 |
| stearolic acid | 281.2426 | 281.248 | −0.0054 | 0.4987 |
| linoleic acid | 281.2426 | 281.248 | −0.0054 | 0.4987 |
| 9,12-octadecadienoic acid | 281.2426 | 281.248 | −0.0054 | 0.4987 |
| stearolic acid/linoelaidic a | 281.2426 | 281.248 | −0.0054 | 0.4987 |
| 9,12-octadecadienoic acid | 281.2426 | 281.248 | −0.0054 | 0.4987 |
| linoleic acid | 281.2426 | 281.248 | −0.0054 | 0.4987 |
| vitamin A(retinol) | 287.2413 | 287.2375 | 0.0038 | 3.4986 |
| abieta-8,11,13-trien-18-ol | 287.2413 | 287.2375 | 0.0038 | 3.4986 |
| 7-shogaol | 291.1936 | 291.196 | −0.0025 | 3.0488 |
| N-octyl-B-D-glucopyranoside | 293.2018 | 293.1964 | 0.0054 | 1.3715 |
| aleuritic acid | 305.2244 | 305.2328 | −0.0084 | 4.0136 |
| dihydrocapsaicin | 308.217 | 308.2225 | −0.0055 | 1.2157 |
| kahweol | 315.2041 | 315.196 | 0.0081 | 0.3245 |
| allopregnendione | 317.2418 | 317.248 | −0.0062 | 1.0414 |
| pregnenolone | 317.2418 | 317.248 | −0.0062 | 1.0414 |
| galanolactone/aframodial/galanal | 319.2341 | 319.2273 | 0.0068 | 0.8834 |
| 2-chloroethyl palmitate | 319.2341 | 319.2404 | −0.0063 | 0.8834 |
| homodihydrocapsaicin | 322.2377 | 322.2382 | −0.0005 | 2.0007 |
| hydroxyprogesterone/DHEA acetate | 331.2245 | 331.2273 | −0.0029 | 0.5192 |
| 10-shogaol | 333.2519 | 333.243 | 0.0089 | 2.3223 |
| kauran-18-al, 17-(acetyloxy) | 347.2641 | 347.2586 | 0.0055 | 0.4534 |
| mogroside backbone-4H2O | 405.3608 | 405.3522 | 0.0087 | 0.5658 |
| jervine | 426.2976 | 426.3008 | −0.0032 | 0.5259 |
| hecogenin/ruscogenin | 431.3158 | 431.3161 | −0.0003 | 0.5012 |

Compounds in Single Stage SCCO₂ Extraction at 60° C. and 300 Bar

6-shogoal and galanolactone were present in this extract in 39.3 and 1.7% relative abundance, respectively. Other shogaols, paradols, gingerols, and gingerdiols were present in the extract. Amino acids, alkaloids, tumerones, ganoderols, vitamins, fatty acids, saccharides, phenolic acids, phenols, sterols, capsaicins, gymnemagins, boswellic acids, saponins and hydrocarbons were also present in this extract. 103 out of 527 (20%) unique chemicals have been directly identified in this extract using the DART TOF-MS. Table 10 shows the compounds identified in the extracts along with their relative abundance. FIG. 8D shows the DART Spectrum of this extract.

TABLE 10
Compounds in Single Stage SCCO₂ extraction at 60° C. and 300 bar

| Name | Meas. | Calc. | Diff(u) | Abund. |
|---|---|---|---|---|
| ethylbenzene | 107.0864 | 107.0861 | 0.0003 | 1.6143 |
| norcamphor/heptadienal | 111.0822 | 111.081 | 0.0012 | 0.3417 |
| 2-methylcyclohexanone | 113.1011 | 113.0966 | 0.0044 | 0.3225 |
| propyl sulfide | 119.0861 | 119.0894 | −0.0033 | 14.59 |
| pseudocumene/propynylcyclohexene | 121.1018 | 121.1017 | 0 | 14.222 |
| 5-hepten-2-one, 6-methyl- | 127.113 | 127.1123 | 0.0007 | 1.2392 |
| leucine | 132.0937 | 132.1024 | −0.0088 | 0.6502 |
| ornithine | 133.1029 | 133.0977 | 0.0052 | 4.5079 |
| dicyclopentadiene | 133.1029 | 133.1017 | 0.0012 | 4.5079 |
| p-cymene | 135.118 | 135.1174 | 0.0006 | 6.4116 |
| anisaldehyde/formic acid benzoate | 137.0605 | 137.0602 | 0.0003 | 15.43 |
| trigonelline/vitamin H | 138.0639 | 138.0555 | 0.0084 | 1.2486 |
| tropine | 142.1171 | 142.1232 | −0.0061 | 0.2123 |
| octalactone | 143.1085 | 143.1072 | 0.0013 | 3.2087 |
| baogongteng B | 144.1105 | 144.1024 | 0.0081 | 0.3472 |
| crotonylbetaine | 145.1042 | 145.1103 | −0.0061 | 3.0818 |
| lysine | 147.1176 | 147.1133 | 0.0043 | 6.6514 |
| 1-methyl-3-phenylpropylamine | 150.1379 | 150.1282 | 0.0097 | 1.8568 |
| 4-phenylbutylamine | 150.1379 | 150.1282 | 0.0097 | 1.8568 |
| carvacrol/thymol/cymenol | 151.1148 | 151.1123 | 0.0025 | 4.5627 |
| 2-butyl-3-methylpyrazine | 151.1148 | 151.1235 | −0.0087 | 4.5627 |
| norpseudophedrine | 152.1121 | 152.1075 | 0.0046 | 0.9576 |
| decadienal/santolina epoxide | 153.1276 | 153.1279 | −0.0003 | 8.6639 |
| pinene oxide | 153.1276 | 153.1279 | −0.0003 | 8.6639 |
| arecoline/hydroxytropinone | 156.1021 | 156.1024 | −0.0003 | 0.3468 |
| methylcholine | 161.1331 | 161.1416 | −0.0085 | 5.7597 |
| jasmone | 165.1198 | 165.1279 | −0.0081 | 2.2783 |
| ephedrine | 166.1242 | 166.1232 | 0.0009 | 0.756 |
| hordenine | 166.1242 | 166.1232 | 0.0009 | 0.756 |
| pseudoephedrine | 166.1242 | 166.1232 | 0.0009 | 0.756 |
| camphorquinone | 167.1064 | 167.1072 | −0.0008 | 4.125 |
| perillic acid | 167.1064 | 167.1072 | −0.0008 | 4.125 |
| 3-(phenylmethoxy)-1-propanol | 167.1064 | 167.1072 | −0.0008 | 4.125 |
| (3Z)-3-hexenyl-2-butenoate | 169.1228 | 169.1228 | 0 | 1.9413 |
| chrysanthemolactone | 169.1228 | 169.1228 | 0 | 1.9413 |
| a-Limonene diepoxide | 169.1228 | 169.1228 | 0 | 1.9413 |
| lupinine | 170.1451 | 170.1545 | −0.0095 | 0.3701 |
| decalactone | 171.1414 | 171.1385 | 0.0029 | 1.4512 |
| linalool oxide | 171.1414 | 171.1385 | 0.0029 | 1.4512 |
| butanoic acid, 3-hexenyl ester | 171.1414 | 171.1385 | 0.0029 | 1.4512 |
| 3,7-octadiene-2,6-diol, 2,6- | 171.1414 | 171.1385 | 0.0029 | 1.4512 |
| 1,7-octadiene-3,6-diol, 2,6- | 171.1414 | 171.1385 | 0.0029 | 1.4512 |
| arcaine | 173.1466 | 173.1514 | −0.0048 | 1.4156 |
| n-octyl acetate | 173.1466 | 173.1541 | −0.0075 | 1.4156 |
| capric acid | 173.1466 | 173.1541 | −0.0075 | 1.4156 |
| caprylic acid ethyl ester | 173.1466 | 173.1541 | −0.0075 | 1.4156 |
| n-decanoic acid/1,3-dioxolane | 173.1466 | 173.1541 | −0.0075 | 1.4156 |
| cinnamyl acetate | 177.0927 | 177.0915 | 0.0012 | 15.995 |
| canavanine | 177.0927 | 177.0987 | −0.006 | 15.995 |
| eugenol methyl ether | 179.1164 | 179.1072 | 0.0092 | 4.2184 |
| 4-(p-methoxyphenyl)-2-butanone | 179.1164 | 179.1072 | 0.0092 | 4.2184 |
| anisylacetone | 179.1164 | 179.1072 | 0.0092 | 4.2184 |
| eugenol methylether | 179.1164 | 179.1072 | 0.0092 | 4.2184 |
| 2(4H)-benzofuranone, 5,6,7,7 | 181.128 | 181.1228 | 0.0052 | 1.6434 |
| chamazulen | 185.131 | 185.133 | −0.002 | 1.1947 |
| 1,3-di-tert-butylbenzene | 191.1826 | 191.18 | 0.0026 | 3.5088 |
| damascone | 193.155 | 193.1592 | −0.0042 | 1.8663 |
| ionone | 193.155 | 193.1592 | −0.0042 | 1.8663 |
| β-pinene, 3-(acetylmethyl)- | 193.155 | 193.1592 | −0.0042 | 1.8663 |
| sedanolide | 195.1344 | 195.1385 | −0.0041 | 2.3625 |
| trans-chrysanthenyl acetate | 195.1344 | 195.1385 | −0.0041 | 2.3625 |
| (−)-myrtenyl acetate | 195.1344 | 195.1385 | −0.0041 | 2.3625 |
| 2,6-octadien-1-ol, 3,7-dimet | 197.146 | 197.1541 | −0.0081 | 0.9299 |
| dihydrocarvylacetate | 197.146 | 197.1541 | −0.0081 | 0.9299 |
| geranyl acetate | 197.146 | 197.1541 | −0.0081 | 0.9299 |
| isobornyl acetate | 197.146 | 197.1541 | −0.0081 | 0.9299 |
| isopulegyl acetate | 197.146 | 197.1541 | −0.0081 | 0.9299 |
| lavandulyl acetate | 197.146 | 197.1541 | −0.0081 | 0.9299 |
| L-bornyl acetate | 197.146 | 197.1541 | −0.0081 | 0.9299 |
| linalyl acetate | 197.146 | 197.1541 | −0.0081 | 0.9299 |
| neryl acetate | 197.146 | 197.1541 | −0.0081 | 0.9299 |
| terpinyl acetate | 197.146 | 197.1541 | −0.0081 | 0.9299 |
| acetic acid, bornyl ester | 197.146 | 197.1541 | −0.0081 | 0.9299 |
| butane, 1-cyclopropylidene-5 | 197.146 | 197.1541 | −0.0081 | 0.9299 |
| bornyl acetate | 197.146 | 197.1541 | −0.0081 | 0.9299 |
| bornyl acetate/linalyl acetatate | 197.146 | 197.1541 | −0.0081 | 0.9299 |
| guaiazulene | 199.1472 | 199.1487 | −0.0015 | 1.8486 |
| naphthalene, 1,6-dimethyl-4- | 199.1472 | 199.1487 | −0.0015 | 1.8486 |
| dehydrocurcumene | 201.1651 | 201.1643 | 0.0008 | 14.031 |
| curcumene/cuparene/calamenen | 203.1793 | 203.18 | −0.0007 | 86.331 |
| elemene | 205.195 | 205.1956 | −0.0007 | 100 |
| cedrene | 205.195 | 205.1956 | −0.0007 | 100 |
| isocaryophyllene | 205.195 | 205.1956 | −0.0007 | 100 |
| isolongifolene | 205.195 | 205.1956 | −0.0007 | 100 |
| longicyclene/longifolene | 205.195 | 205.1956 | −0.0007 | 100 |
| thujopsen | 205.195 | 205.1956 | −0.0007 | 100 |
| valencene | 205.195 | 205.1956 | −0.0007 | 100 |
| copaene | 205.195 | 205.1956 | −0.0007 | 100 |
| germacrene D | 205.195 | 205.1956 | −0.0007 | 100 |
| a-cubebene | 205.195 | 205.1956 | −0.0007 | 100 |
| a-muurolene | 205.195 | 205.1956 | −0.0007 | 100 |
| trans-a-bergamotene | 205.195 | 205.1956 | −0.0007 | 100 |
| germacrene D | 205.195 | 205.1956 | −0.0007 | 100 |
| a-cubebene | 205.195 | 205.1956 | −0.0007 | 100 |
| (−)-a-panasinsen | 205.195 | 205.1956 | −0.0007 | 100 |
| cedrene/valencene/β-guainene | 205.195 | 205.1956 | −0.0007 | 100 |
| cycloheptane, 4-methylene-1- | 205.195 | 205.1956 | −0.0007 | 100 |
| beta-gualene/cis-gamma-bisabollene | 205.195 | 205.1956 | −0.0007 | 100 |
| aromadendrene | 205.195 | 205.1956 | −0.0007 | 100 |
| humulene | 205.195 | 205.1956 | −0.0007 | 100 |
| sesquiphellandrene | 205.195 | 205.1956 | −0.0007 | 100 |
| caryophyllene | 205.195 | 205.1956 | −0.0007 | 100 |
| bisabolene | 205.195 | 205.1956 | −0.0007 | 100 |
| zingiberene | 205.195 | 205.1956 | −0.0007 | 100 |
| β-sesquiphellandrene | 205.195 | 205.1956 | −0.0007 | 100 |
| 3,5-bis(1,1-dimethylethyl)-p | 207.1773 | 207.1749 | 0.0024 | 8.3263 |
| carvylacetate | 209.1584 | 209.1541 | 0.0042 | 1.5141 |
| isobornyl propionate | 211.1608 | 211.1698 | −0.009 | 0.9643 |
| hexylcinnamaldehyde | 217.1609 | 217.1592 | 0.0017 | 8.2271 |
| ar-tumerone | 217.1609 | 217.1592 | 0.0017 | 8.2271 |
| furanoeremophilane | 219.175 | 219.1749 | 0.0001 | 26.615 |
| nootkatone | 219.175 | 219.1749 | 0 | 26.615 |
| valerenal | 219.175 | 219.1749 | 0 | 26.615 |
| curlone | 219.175 | 219.1749 | 0 | 26.615 |
| turmerone/ar-turmerol/xanthorrhizol | 219.175 | 219.1749 | 0 | 26.615 |
| spathulenol/caryophyllene | 221.1907 | 221.1905 | 0.0001 | 19.718 |
| oxide/lancelol | | | | |
| 6-isopropenyl-4,8a-dimethyl- | 221.1907 | 221.1905 | 0.0001 | 19.718 |
| 6,10-dodecadien-1-yn-3-ol | 221.1907 | 221.1905 | 0.0001 | 19.718 |
| caryophyllene oxide | 221.1907 | 221.1905 | 0.0001 | 19.718 |
| bergamotol, Z-a-trans- | 221.1907 | 221.1905 | 0.0001 | 19.718 |
| undec-2-ene-8,10-diynoic acid | 232.1728 | 232.1701 | 0.0026 | 4.3978 |
| costunolide | 233.1614 | 233.1541 | 0.0073 | 5.7806 |
| panthenol | 234.1766 | 234.1705 | 0.0061 | 2.7698 |
| eremophilanlactone | 235.1692 | 235.1698 | −0.0006 | 12.394 |
| 2-octyl benzoate | 235.1692 | 235.1698 | −0.0007 | 12.394 |
| valerenic acid | 235.1692 | 235.1698 | −0.0007 | 12.394 |
| vellerdiol | 237.1839 | 237.1854 | −0.0015 | 10.638 |
| 2-pentenoic acid, 3-methyl-5 | 237.1839 | 237.1854 | −0.0015 | 10.638 |
| a-ionyl acetate | 237.1839 | 237.1854 | −0.0015 | 10.638 |
| 3-hydroxymyristic acid | 245.211 | 245.2116 | −0.0006 | 1.5855 |
| 6-paradol | 251.1671 | 251.1647 | 0.0024 | 3.6762 |
| hydroxyvalerenic acid | 251.1671 | 251.1647 | 0.0024 | 3.6762 |
| palmitic acid | 257.25 | 257.248 | 0.002 | 4.1935 |
| C20H32/biformene/kaur-16-ene | 273.2554 | 273.2582 | −0.0029 | 5.2754 |
| eserine | 276.1761 | 276.1712 | 0.0049 | 5.586 |
| 6-shogaol | 277.1797 | 277.1803 | −0.0006 | 39.3 |
| menthyl salicylate | 277.1797 | 277.1803 | −0.0006 | 39.3 |
| cyclohexanecarboxylic acid, | 277.1797 | 277.1803 | −0.0006 | 39.3 |
| 6-shogaol | 277.1797 | 277.1804 | −0.0007 | 39.3 |
| stearolic acid | 281.247 | 281.248 | −0.001 | 2.2052 |
| linoleic acid | 281.247 | 281.248 | −0.001 | 2.2052 |
| 9,12-octadecadienoic acid | 281.247 | 281.248 | −0.001 | 2.2052 |
| stearolic acid/linoelaidic a | 281.247 | 281.248 | −0.001 | 2.2052 |
| 9,12-octadecadienoic acid | 281.247 | 281.248 | −0.001 | 2.2052 |
| linoleic acid | 281.247 | 281.248 | −0.001 | 2.2052 |
| 16-oxokahweol | 283.1785 | 283.1698 | 0.0087 | 5.324 |
| miltirone | 283.1785 | 283.1698 | 0.0087 | 5.324 |
| vitamin A(retinol) | 287.241 | 287.2375 | 0.0034 | 4.1517 |
| abieta-8,11,13-trien-18-ol | 287.241 | 287.2375 | 0.0034 | 4.1517 |
| atropine | 291.1853 | 291.1834 | 0.0019 | 4.7098 |
| N-octyl-B-D-glucopyranoside | 293.1969 | 293.1964 | 0.0005 | 2.5037 |
| aleuritic acid | 305.2249 | 305.2328 | −0.0079 | 5.1869 |
| dihydrocapsaicin | 308.2174 | 308.2226 | −0.0051 | 1.8857 |
| progesterone | 315.235 | 315.2324 | 0.0026 | 1.0936 |
| allopregnendione | 317.239 | 317.248 | −0.0091 | 1.5621 |
| pregnenolone | 317.239 | 317.246 | −0.0091 | 1.5621 |
| galanolactone/aframodial/galanal | 319.2363 | 319.2273 | 0.009 | 1.6737 |
| 2-chloroethyl palmitate | 319.2363 | 319.2404 | −0.0041 | 1.6737 |
| homodihydrocapsaicin | 322.2411 | 322.2382 | 0.0029 | 2.3517 |
| ajmaline | 327.2025 | 327.2072 | −0.0047 | 4.5029 |
| 10-shogaol | 333.25 | 333.243 | 0.007 | 3.1887 |
| chenodeoxycholic acid | 345.2913 | 345.3005 | −0.0093 | 0.9795 |
| kauran-18-al, 17-(acetyloxy) | 347.2594 | 347.2586 | 0.0008 | 1.0118 |
| incensole acetate | 349.274 | 349.2742 | −0.0002 | 0.7688 |
| lithocholic acid | 377.2985 | 377.3055 | −0.0071 | 0.5347 |
| delta-tocotrienol | 397.3048 | 397.3107 | −0.0058 | 0.8288 |
| mogroside backbone-4H2O | 405.3598 | 405.3522 | 0.0076 | 1.4023 |
| beta/gamma-tocotrienol | 411.3204 | 411.3263 | −0.0059 | 0.7663 |
| benzethonium | 413.3223 | 413.3294 | −0.0071 | 0.837 |
| calcitriol/sarsapogenin | 417.3382 | 417.3368 | 0.0014 | 0.8041 |
| jervine | 426.2937 | 426.3008 | −0.0072 | 2.2778 |
| hecogenin/ruscogenin | 431.3208 | 431.3161 | 0.0047 | 1.5114 |
| mogroside backbone- | 441.3657 | 441.3733 | −0.0076 | 1.0943 |
| 2H2O/ganoderol | | | | |
| vitamin K1(phytonadione) | 451.351 | 451.3576 | −0.0066 | 0.428 |
| ursonic acid/dehydroboswellic acid | 455.3492 | 455.3525 | −0.0033 | 1.2916 |
| Diepoxydammar diol | 459.3541 | 459.3474 | 0.0067 | 0.6523 |
| ganoderic acid D/M | 469.3389 | 469.3318 | 0.0071 | 0.3131 |
| keto boswellic acid/glycyrrhizol | 471.3529 | 471.3474 | 0.0055 | 0.285 |
| Gymnemasaponin II - 2 Glc | 475.3698 | 475.3787 | −0.009 | 0.1532 |
| psychosine | 478.343 | 478.338 | 0.005 | 0.2112 |
| 18-glycyrrhetinic acid methyl ester | 485.3707 | 485.3631 | 0.0076 | 0.272 |
| cholesteryl benzoate | 491.3986 | 491.3889 | 0.0097 | 0.2616 |
| 3-O-acetyl-9,11-dehydro BA | 497.3684 | 497.3631 | 0.0054 | 0.6837 |
| 3-O-acetyl-11-hydroxy BA | 515.3825 | 515.3737 | 0.0088 | 0.1532 |
| vitamin E succinate | 531.4122 | 531.4049 | 0.0073 | 0.1175 |
| adhyperforin | 551.4163 | 551.41 | 0.0063 | 0.0367 |
| echinenone | 551.4163 | 551.4253 | −0.009 | 0.0367 |
| ganodermic acids R/S | 571.3923 | 571.3999 | −0.0075 | 0.1642 |

Compounds in Single Stage SCCO₂ Extraction at 60° C. and 500 Bar

6-shogoal and galanolactone were present in this extract in 100 and 4.5% relative abundance, respectively. Other shogaols, paradols, gingerols, and gingerdiols were present in the extract. Amino acids, terpenoids, quinones, tumerones, ganoderols, vitamins, fatty acids, saccharides, phenolic acids, phenols, sterols, capsaicins, gymnemagins, boswellic acids, saponins and hydrocarbons were also present in this extract. 109 out of 485 (22%) unique chemicals have been directly identified in this extract using the DART TOF-MS. Table 11 shows the compounds identified in the extracts along with their relative abundance. FIG. 8E shows the DART Spectrum of this extract.

TABLE 11
Compounds in Single Stage SCCO₂ extraction at 60° C. and 500 bar

| Compound | Meas. | Calc. | Diff(u) | Abund. |
|---|---|---|---|---|
| ethylbenzene | 107.0857 | 107.0861 | −0.0004 | 1.3959 |
| propyl sulfide | 119.0855 | 119.0894 | −0.0039 | 5.7415 |
| pseudocumene/propynylcyclohexene | 121.1028 | 121.1017 | 0.0011 | 2.0497 |
| 5-hepten-2-one, 6-methyl- | 127.1109 | 127.1123 | −0.0014 | 0.5563 |
| DL-mevalonic acid lactone | 131.0734 | 131.0708 | 0.0026 | 0.5563 |
| ornithine | 133.1042 | 133.0977 | 0.0065 | 2.7934 |
| dicyclopentadiene | 133.1042 | 133.1017 | 0.0025 | 2.7934 |
| p-cymene | 135.12 | 135.1174 | 0.0026 | 3.8795 |
| adenine | 136.0673 | 136.0623 | 0.0049 | 0.989 |
| anisaldehyde/formic acid benzoate | 137.0599 | 137.0602 | −0.0004 | 56.476 |
| trigonelline/vitamin H | 138.0632 | 138.0555 | 0.0076 | 4.1484 |
| octalactone | 143.1082 | 143.1072 | 0.001 | 1.21 |
| crotonylbetaine | 145.1037 | 145.1103 | −0.0066 | 2.5553 |
| lysine | 147.1183 | 147.1133 | 0.005 | 3.7442 |
| carvacrol/thymol/cymenol | 151.1026 | 151.1123 | −0.0097 | 4.3318 |
| decadienal/santolina epoxide | 153.1279 | 153.1279 | 0 | 6.1565 |
| pinene oxide | 153.1279 | 153.1279 | 0 | 6.1565 |
| cineole/borneol | 155.1352 | 155.1436 | −0.0084 | 0.694 |
| methone/pinocampheol/pulegol | 155.1352 | 155.1436 | −0.0084 | 0.694 |
| citronellol/dihdroymercenol | 157.159 | 157.1592 | −0.0002 | 1.4661 |
| methylcholine | 161.1333 | 161.1416 | −0.0083 | 3.8554 |
| methyl cinnamic acid | 163.0759 | 163.0759 | 0 | 16.073 |
| safrole | 163.0759 | 163.0759 | 0 | 16.073 |
| cinnamaldehyde, o-methoxy- | 163.0759 | 163.0759 | 0 | 16.073 |
| camphorquinone | 167.105 | 167.1072 | −0.0023 | 2.6284 |
| perillic acid | 167.105 | 167.1072 | −0.0023 | 2.6284 |
| 3-(phenylmethoxy)-1-propanol | 167.105 | 167.1072 | −0.0023 | 2.6284 |
| (3Z)-3-hexenyl-2-butenoate | 169.1206 | 169.1228 | −0.0022 | 1.6839 |
| chrysanthemolactone | 169.1206 | 169.1228 | −0.0022 | 1.6839 |
| a-Limonene diepoxide | 169.1206 | 169.1228 | −0.0022 | 1.6839 |
| decalactone | 171.1359 | 171.1385 | −0.0026 | 0.3103 |
| linalool oxide | 171.1359 | 171.1385 | −0.0026 | 0.3103 |
| butanoic acid, 3-hexenyl ester | 171.1359 | 171.1385 | −0.0026 | 0.3103 |
| 3,7-octadiene-2,6-diol, 2,6- | 171.1359 | 171.1385 | −0.0026 | 0.3103 |
| 1,7-octadiene-3,6-diol, 2,6- | 171.1359 | 171.1385 | −0.0026 | 0.3103 |
| arcaine | 173.1508 | 173.1514 | −0.0006 | 1.5209 |
| n-octyl acetate | 173.1508 | 173.1541 | −0.0033 | 1.5209 |
| capric acid | 173.1508 | 173.1541 | −0.0033 | 1.5209 |
| caprylic acid ethyl ester | 173.1508 | 173.1541 | −0.0033 | 1.5209 |
| n-decanoic acid/1,3-dioxolane | 173.1508 | 173.1541 | −0.0033 | 1.5209 |
| cinnamyl acetate | 177.0915 | 177.0915 | 0 | 42.096 |
| canavanine | 177.0915 | 177.0987 | −0.0072 | 42.096 |
| coniferaldehyde | 179.0711 | 179.0708 | 0.0002 | 14.082 |
| methoxycinnamic acid | 179.0711 | 179.0708 | 0.0002 | 14.082 |
| D-mannosamine | 180.0929 | 180.0872 | 0.0057 | 2.3863 |
| galacgtosamine | 180.0929 | 180.0872 | 0.0057 | 2.3863 |
| glucosamine | 180.0929 | 180.0872 | 0.0057 | 2.3863 |
| homophenylalanine | 180.0929 | 180.1024 | −0.0095 | 2.3863 |
| salsolinol | 180.0929 | 180.1024 | −0.0095 | 2.3863 |
| stilbene | 181.1069 | 181.1017 | 0.0051 | 2.5818 |
| difluoromethylornithine | 183.1024 | 183.0945 | 0.0079 | 0.2022 |
| dihydroconiferyl alcohol | 183.1024 | 183.1021 | 0.0003 | 0.2022 |
| chamazulen | 185.1309 | 185.133 | −0.0021 | 0.5722 |
| 1,3-di-tert-butylbenzene | 191.1845 | 191.18 | 0.0045 | 3.8285 |
| myristicin | 193.0964 | 193.0864 | 0.01 | 4.3365 |
| dehydrozingerone | 193.0964 | 193.0865 | 0.0099 | 4.3365 |
| a-phenylindol | 194.0938 | 194.0969 | −0.0032 | 1.902 |
| guaiazulene | 199.141 | 199.1487 | −0.0077 | 0.8855 |
| naphthalene, 1,6-dimethyl-4- | 199.141 | 199.1487 | −0.0077 | 0.8855 |
| dehydrocurcumene | 201.1656 | 201.1643 | 0.0013 | 9.9673 |
| curcumene/cuparene/calamenene | 203.1797 | 203.18 | −0.0003 | 31.702 |
| zingiberene/(Z,E)-a-farnesene | 205.1951 | 205.1956 | −0.0005 | 19.326 |
| alloaromadendrene/elemene | 205.1951 | 205.1956 | −0.0005 | 19.326 |
| cycloheptane, 4-methylene-1- | 205.1951 | 205.1956 | −0.0005 | 19.326 |
| aromadendrene, (+) | 205.1951 | 205.1956 | −0.0005 | 19.326 |
| caryophyllene | 205.1951 | 205.1956 | −0.0005 | 19.326 |
| cedrene | 205.1951 | 205.1956 | −0.0005 | 19.326 |
| humulene | 205.1951 | 205.1956 | −0.0005 | 19.326 |
| isocaryophyllene | 205.1951 | 205.1956 | −0.0005 | 19.326 |
| isolongifolene | 205.1951 | 205.1956 | −0.0005 | 19.326 |
| longicyclene/longifolene | 205.1951 | 205.1956 | −0.0005 | 19.326 |
| thujopsen | 205.1951 | 205.1956 | −0.0005 | 19.326 |
| valencene | 205.1951 | 205.1956 | −0.0005 | 19.326 |
| 1,3,6,10-dodecatetraene, 3,7 | 205.1951 | 205.1956 | −0.0005 | 19.326 |
| copaene | 205.1951 | 205.1956 | −0.0005 | 19.326 |
| germacrene D | 205.1951 | 205.1956 | −0.0005 | 19.326 |
| a-cubebene | 205.1951 | 205.1956 | −0.0005 | 19.326 |
| a-muurolene | 205.1951 | 205.1956 | −0.0005 | 19.326 |
| beta-gualene/cis-gamma-bisabollene | 205.1951 | 205.1956 | −0.0005 | 19.326 |
| trans-a-bergamotene | 205.1951 | 205.1956 | −0.0005 | 19.326 |
| (−)-a-panasinsen | 205.1951 | 205.1956 | −0.0005 | 19.326 |
| β-sesquiphellandrene | 205.1951 | 205.1956 | −0.0005 | 19.326 |
| cedrene/valencene/β-guainene | 205.1951 | 205.1956 | −0.0005 | 19.326 |
| cycloheptane, 4-methylene-1- | 205.1951 | 205.1956 | −0.0005 | 19.326 |
| isopilocarpine | 209.1355 | 209.129 | 0.0065 | 0.4944 |
| philocarpine | 209.1355 | 209.129 | 0.0065 | 0.4944 |
| epoxy-a-terpenyl acetate | 213.1542 | 213.149 | 0.0052 | 0.5272 |
| hexylcinnamaldehyde | 217.1607 | 217.1592 | 0.0014 | 7.5022 |
| ar-tumerone | 217.1607 | 217.1592 | 0.0014 | 7.5022 |
| furanoeremophilane | 219.1757 | 219.1749 | 0.0008 | 18.534 |
| nootkatone | 219.1757 | 219.1749 | 0.0008 | 18.534 |
| valerenal | 219.1757 | 219.1749 | 0.0008 | 18.534 |
| xanthorrhizol | 219.1757 | 219.1749 | 0.0008 | 18.534 |
| curlone | 219.1757 | 219.1749 | 0.0008 | 18.534 |
| turmerone/ar-turmerol | 219.1757 | 219.1749 | 0.0008 | 18.534 |
| caryophellene oxide | 221.192 | 221.1905 | 0.0015 | 13.147 |
| 6,1 0-dodecadien-1-yn-3-ol, 3 | 221.192 | 221.1905 | 0.0015 | 13.147 |
| caryophyllene oxide | 221.192 | 221.1905 | 0.0015 | 13.147 |
| bergamotol, Z-a-trans- | 221.192 | 221.1905 | 0.0015 | 13.147 |
| spathulenol/9-cedranone/lanceol | 221.192 | 221.1905 | 0.0015 | 13.147 |
| 6-isopropenyl-4,8a-dimethyl- | 221.192 | 221.1905 | 0.0015 | 13.147 |
| caryophyllene oxide/(−)-spat | 221.192 | 221.1905 | 0.0015 | 13.147 |
| N-isobutylundeca-(2E,4E)-dienoic acid | 230.1625 | 230.1545 | 0.008 | 0.5942 |
| undec-2-ene-8,10-diynoic acid | 232.1667 | 232.1701 | −0.0034 | 0.2558 |
| costunolide | 233.164 | 233.1541 | 0.0099 | 5.4688 |
| eremophilanlactone | 235.169 | 235.1698 | −0.0008 | 9.7551 |
| 2-octyl benzoate | 235.169 | 235.1698 | −0.0008 | 9.7551 |
| valerenic acid | 235.169 | 235.1698 | −0.0008 | 9.7551 |
| vellerdiol | 237.1838 | 237.1854 | −0.0016 | 8.7509 |
| 3-methyl-but-2-enoic acid, 1 | 237.1838 | 237.1854 | −0.0016 | 8.7509 |
| 2-pentenoic acid, 3-methyl-5 | 237.1838 | 237.1854 | −0.0016 | 8.7509 |
| a-ionyl acetate | 237.1838 | 237.1854 | −0.0016 | 8.7509 |
| 6-paradol | 251.1705 | 251.1647 | 0.0058 | 4.205 |
| hydroxyvalerenic acid | 251.1705 | 251.1647 | 0.0058 | 4.205 |
| palmitic acid | 257.2528 | 257.248 | 0.0048 | 3.9292 |
| panaxydol | 261.1873 | 261.1854 | 0.0018 | 11.394 |
| oxymatrine | 265.1887 | 265.1916 | −0.0029 | 2.0769 |
| hydroxypalmitic acid | 273.2487 | 273.2429 | 0.0058 | 2.7563 |
| C20H32/biformene/kaur-16-ene | 273.2487 | 273.2582 | −0.0095 | 2.7563 |
| 1,6-octadien-3-ol, 3,7-dimet | 274.18 | 274.1807 | −0.0008 | 0.4155 |
| podocarpic acid | 275.1727 | 275.1647 | 0.008 | 8.8397 |
| eserine | 276.1761 | 276.1712 | 0.0049 | 6.7483 |
| 6-shogaol | 277.1792 | 277.1803 | −0.0011 | 100 |
| menthyl salicylate | 277.1792 | 277.1803 | −0.0011 | 100 |
| cyclohexanecarboxylic acid | 277.1792 | 277.1803 | −0.0011 | 100 |
| 6-shogacl | 277.1792 | 277.1804 | −0.0011 | 100 |
| stearolic acid | 281.2481 | 281.248 | 0.0001 | 2.6299 |
| linoleic acid | 281.2481 | 281.248 | 0.0001 | 2.6299 |
| stearolic acid/linoelaidic acid | 281.2481 | 281.248 | 0.0001 | 2.6299 |
| 9,12-octadecadienoic acid | 281.2481 | 281.248 | 0.0001 | 2.6299 |
| linoleic acid | 281.2481 | 281.248 | 0.0001 | 2.6299 |
| lynestrenol | 285.2201 | 285.2218 | −0.0017 | 2.5849 |
| vitamin A(retinol) | 287.2276 | 287.2375 | −0.01 | 3.3689 |
| abieta-8,11,13-trien-18-ol | 287.2276 | 287.2375 | −0.01 | 3.3689 |
| 17a-methyl-19-nortestosterone | 289.2249 | 289.2167 | 0.0081 | 3.8056 |
| androstanedione | 289.2249 | 289.2167 | 0.0081 | 3.8056 |
| dehydroisoandosterone(DHEA) | 289.2249 | 289.2167 | 0.0081 | 3.8056 |
| testosterone | 289.2249 | 289.2167 | 0.0081 | 3.8056 |
| N-octyl-B-D-glucopyranoside | 293.1904 | 293.1964 | −0.0061 | 4.5593 |
| 6-gingerdiol | 297.2102 | 297.2066 | 0.0036 | 3.4666 |
| 9,12-octadecadienoyl chloride | 299.2187 | 299.2141 | 0.0045 | 1.1592 |
| retinoic acid | 301.2256 | 301.2167 | 0.0089 | 3.2289 |
| C20H28O2 | 301.2256 | 301.2167 | 0.0089 | 3.2289 |
| abietic acid | 303.2279 | 303.2324 | −0.0045 | 5.4508 |
| eicosapentaenoic acid | 303.2279 | 303.2324 | −0.0045 | 5.4508 |
| 8-shogaol | 305.2136 | 305.2117 | 0.002 | 21.379 |
| 10-paradol | 307.2209 | 307.2273 | −0.0064 | 2.6032 |
| dihydrocapsaicin | 308.2177 | 308.2225 | −0.0048 | 1.2639 |
| galanolactone/aframodial/galanal | 319.2243 | 319.2273 | −0.003 | 4.5194 |
| homocapsaicin | 320.2321 | 320.2226 | 0.0095 | 1.5724 |
| homodihydrocapsaicin | 322.2291 | 322.2382 | −0.0091 | 2.7408 |
| 8-gingerdiol | 325.2372 | 325.2379 | −0.0007 | 1.4433 |
| ajmaline | 327.2043 | 327.2072 | −0.0029 | 4.5409 |
| hydroxyprogesterone/DHEA acetate | 331.2265 | 331.2273 | −0.0009 | 6.5012 |
| 10-shogaol | 333.244 | 333.243 | 0.001 | 31.394 |
| pregnanetriol | 337.2689 | 337.2742 | −0.0053 | 1.7575 |
| yohimbic acid | 341.1933 | 341.1865 | 0.0067 | 3.7719 |
| menisperine | 341.1933 | 341.1985 | −0.0053 | 3.7719 |
| 10-dehydrogingerdione | 347.2277 | 347.2222 | 0.0055 | 6.4402 |
| calycanthine | 347.2277 | 347.2235 | 0.0042 | 6.4402 |
| 10-gingerdione | 349.2461 | 349.2379 | 0.0082 | 3.0647 |
| 10-gingerdiol | 353.2711 | 353.2692 | 0.0019 | 2.3326 |
| 12-shogaol | 361.278 | 361.2743 | 0.0037 | 3.1802 |
| cinobufotalin | 363.2668 | 363.2688 | −0.002 | 0.9159 |
| odorigenin/digitoxigenin | 375.2622 | 375.2535 | 0.0087 | 4.5295 |
| vitexilactone | 381.2592 | 381.2641 | −0.0049 | 0.9199 |
| benzethonium | 413.3234 | 413.3294 | −0.006 | 3.6383 |
| diosgenin | 415.318 | 415.3212 | −0.0032 | 2.5276 |
| spironolactone | 417.2026 | 417.2099 | −0.0073 | 10.678 |
| cholic acid methyl ester | 423.3124 | 423.311 | 0.0014 | 3.2026 |
| 4-methylumbelliferyl elaidate | 441.299 | 441.3005 | −0.0015 | 3.3733 |
| soyasapogenol A | 474.3676 | 474.3709 | −0.0033 | 0.8559 |
| hovenolactone/trevoagenin D | 489.3633 | 489.358 | 0.0054 | 1.3633 |
| gymnemagenin | 492.3401 | 492.3451 | −0.0051 | 2.0558 |
| 3-O-acetyl-9,11-dehydro BA | 497.3594 | 497.3631 | −0.0037 | 3.293 |
| acetylboswellic acid/ganoder | 499.3791 | 499.3787 | 0.0003 | 2.2434 |
| 3-O-acetyl-11-hydroxy boswellic acid | 515.3663 | 515.3737 | −0.0073 | 1.171 |
| fusic acid/phytolaccinic acid | 517.3595 | 517.3529 | 0.0066 | 0.6054 |
| hyperforin | 537.3911 | 537.3944 | −0.0033 | 0.7422 |
| adhyperforin | 551.4124 | 551.41 | 0.0024 | 1.381 |
| lutein/zeaxanthin | 569.4297 | 569.4359 | −0.0061 | 0.7047 |

Compounds in Single Stage SCCO₂ Extraction at 40° C. and 300 Bar at 5 Minutes

6-shogoal, 6 gingerol and galanolactone were present in this extract in 47.5, 4.2 and 1.0% relative abundance, respectively. Other shogaols, paradols, gingerols, and gingerdiols were present in the extract. Amino acids, vitamins, fatty acids, alkaloids, tumerones, ganoderols, saccharides, phenolic acids, phenols, sterols, capsaicins, gymnemagins, boswellic acids, saponins and hydrocarbons were also present in this extract. 90 oout 384 (23%) unique chemicals have been directly identified in this extract using the DART TOF-MS. Table 12 shows the compounds identified in the extracts along with their relative abundance. FIG. 8F shows the DART Spectrum of this extract.

TABLE 12
Compounds in Single Stage SCCO₂ extraction at 40° C. and 300 bar at 5 minutes

| Compounds | Meas. | Calc. | Diff(u) | Abund. |
|---|---|---|---|---|
| ethylbenzene | 107.0875 | 107.0861 | 0.0014 | 0.1988 |
| propyl sulfide | 119.0857 | 119.0894 | −0.0037 | 13.361 |
| pseudocumene/propynylcyclohexene | 121.1017 | 121.1017 | −0.0001 | 11.88 |
| 5-hepten-2-one, 6-methyl- | 127.1153 | 127.1123 | 0.0029 | 0.4689 |
| leucine | 132.0939 | 132.1024 | −0.0086 | 0.7597 |
| 2-indolinone | 133.0615 | 133.0527 | 0.0088 | 0.2498 |
| asparagine | 133.0615 | 133.0613 | 0.0002 | 0.2498 |
| cinnamaldehyde/methylbenzofuran | 133.0615 | 133.0653 | −0.0038 | 0.2498 |
| ornithine | 133.1037 | 133.0977 | 0.006 | 3.1418 |
| dicyclopentadiene | 133.1037 | 133.1017 | 0.002 | 3.1418 |
| p-cymene | 135.1182 | 135.1174 | 0.0008 | 5.2477 |
| anisaldehyde/formic acid benzoate | 137.0611 | 137.0602 | 0.0008 | 10.575 |
| trigonelline/vitamin H | 138.0631 | 138.0555 | 0.0075 | 0.7353 |
| octalactone | 143.1077 | 143.1072 | 0.0005 | 1.5193 |
| crotonylbetaine | 145.1021 | 145.1103 | −0.0082 | 2.3052 |
| lysine | 147.1178 | 147.1133 | 0.0045 | 5.4988 |
| 1-methyl-3-phenylpropylamine | 150.137 | 150.1282 | 0.0088 | 1.3702 |
| 4-phenylbutylamine | 150.137 | 150.1282 | 0.0088 | 1.3702 |
| carvacrol/thymol/cymenol | 151.1162 | 151.1123 | 0.0039 | 2.2486 |
| 2-butyl-3-methylpyrazine | 151.1162 | 151.1235 | −0.0073 | 2.2486 |
| norpseudophedrine | 152.1138 | 152.1075 | 0.0062 | 0.2169 |
| decadienal/santolina epoxide | 153.1282 | 153.1279 | 0.0003 | 4.2416 |
| pinene oxide/piperitone pule | 153.1282 | 153.1279 | 0.0003 | 4.2416 |
| cineole/borneol | 155.1422 | 155.1436 | −0.0014 | 0.2894 |
| methone/pinocampheol/pulegol | 155.1422 | 155.1436 | −0.0015 | 0.2894 |
| methylcholine | 161.1333 | 161.1416 | −0.0084 | 4.9628 |
| jasmone | 165.1238 | 165.1279 | −0.0042 | 1.2017 |
| ephedrine | 166.125 | 166.1232 | 0.0018 | 0.1824 |
| hordenine | 166.125 | 166.1232 | 0.0018 | 0.1824 |
| pseudoephedrine | 166.125 | 166.1232 | 0.0018 | 0.1824 |
| camphorquinone | 167.1065 | 167.1072 | −0.0007 | 2.8811 |
| perillic acid | 167.1065 | 167.1072 | −0.0007 | 2.8811 |
| 3-(phenylmethoxy)-1-propanol | 167.1065 | 167.1072 | −0.0007 | 2.8811 |
| (3Z)-3-hexenyl-2-butenoate | 169.1233 | 169.1228 | 0.0004 | 1.1041 |
| chrysanthemolactone | 169.1233 | 169.1228 | 0.0004 | 1.1041 |
| a-Limonene diepoxide | 169.1233 | 169.1228 | 0.0004 | 1.1041 |
| lupinine | 170.156 | 170.1545 | 0.0015 | 0.0811 |
| decalactone | 171.1361 | 171.1385 | −0.0024 | 0.7356 |
| linalool oxide | 171.1361 | 171.1385 | −0.0024 | 0.7356 |
| butanoic acid, 3-hexenyl ester | 171.1361 | 171.1385 | −0.0024 | 0.7356 |
| 3,7-octadiene-2,6-diol, 2,6- | 171.1361 | 171.1385 | −0.0024 | 0.7356 |
| 1,7-octadiene-3,6-diol, 2,6- | 171.1361 | 171.1385 | −0.0024 | 0.7356 |
| arcaine | 173.1507 | 173.1514 | −0.0007 | 0.915 |
| n-octyl acetate | 173.1507 | 173.1541 | −0.0034 | 0.915 |
| capric acid | 173.1507 | 173.1541 | −0.0034 | 0.915 |
| caprylic acid ethyl ester | 173.1507 | 173.1541 | −0.0034 | 0.915 |
| n-decanoic acid/1,3-dioxolane | 173.1507 | 173.1541 | −0.0034 | 0.915 |
| cinnamyl acetate | 177.0919 | 177.0915 | 0.0003 | 14.243 |
| canavanine | 177.0919 | 177.0987 | −0.0069 | 14.243 |
| 2(4H)-benzofuranone, 5,6,7,7 | 181.1273 | 181.1228 | 0.0045 | 1.3532 |
| pinonic acid | 185.1275 | 185.1177 | 0.0098 | 0.5279 |
| 3-methyl-2-butenoic acid, 2- | 185.1275 | 185.1177 | 0.0098 | 0.5279 |
| chamazulen | 185.1275 | 185.133 | −0.0055 | 0.5279 |
| 1,3-di-tert-butylbenzene | 191.1829 | 191.18 | 0.0029 | 2.8767 |
| damascone | 193.1661 | 193.1592 | 0.0069 | 1.4657 |
| ionone | 193.1661 | 193.1592 | 0.0069 | 1.4657 |
| β-pinene, 3-(acetylmethyl)- | 193.1661 | 193.1592 | 0.0069 | 1.4657 |
| D-glucosaminic acid | 196.0921 | 196.0821 | 0.0099 | 0.1605 |
| DL-a-methyl-m-tyrosine | 196.0921 | 196.0973 | −0.0053 | 0.1605 |
| guaiazulene | 199.1439 | 199.1487 | −0.0048 | 0.746 |
| naphthalene, 1,6-dimethyl-4- | 199.1439 | 199.1487 | −0.0048 | 0.746 |
| dehydrocurcumene | 201.1654 | 201.1643 | 0.0011 | 12.69 |
| curcumene/cuparene/calamenene | 203.1793 | 203.18 | −0.0007 | 70.063 |
| cycloheptane, 4-methylene-1- | 205.1948 | 205.1956 | −0.0008 | 100 |
| cedrene | 205.1948 | 205.1956 | −0.0008 | 100 |
| isocaryophyllene | 205.1948 | 205.1956 | −0.0008 | 100 |
| isolongifolene | 205.1948 | 205.1956 | −0.0008 | 100 |
| longicyclene/longifolene | 205.1948 | 205.1956 | −0.0008 | 100 |
| thujopsen | 205.1948 | 205.1956 | −0.0008 | 100 |
| valencene | 205.1948 | 205.1956 | −0.0008 | 100 |
| copaene | 205.1948 | 205.1956 | −0.0008 | 100 |
| a-muurolene | 205.1948 | 205.1956 | −0.0008 | 100 |
| 1,6,10-dodecatriene, 7,11-di | 205.1948 | 205.1956 | −0.0008 | 100 |
| farnesene | 205.1948 | 205.1956 | −0.0008 | 100 |
| trans-a-bergamotene | 205.1948 | 205.1956 | −0.0008 | 100 |
| a-zingiberene | 205.1948 | 205.1956 | −0.0008 | 100 |
| germacrene D | 205.1948 | 205.1956 | −0.0008 | 100 |
| a-cubebene | 205.1948 | 205.1956 | −0.0008 | 100 |
| (−)-a-panasinsen | 205.1948 | 205.1956 | −0.0008 | 100 |
| cedrene/valencene/β-guainene | 205.1948 | 205.1956 | −0.0008 | 100 |
| cycloheptane, 4-methylene-1- | 205.1948 | 205.1956 | −0.0008 | 100 |
| beta-gualene/cis-gamma-bisabollene | 205.1948 | 205.1956 | −0.0008 | 100 |
| aromadendrene | 205.1948 | 205.1956 | −0.0008 | 100 |
| humulene | 205.1948 | 205.1956 | −0.0008 | 100 |
| caryophyllene | 205.1948 | 205.1956 | −0.0008 | 100 |
| bisabolene | 205.1948 | 205.1956 | −0.0008 | 100 |
| zingiberene | 205.1948 | 205.1956 | −0.0008 | 100 |
| β-sesquiphellandrene | 205.1948 | 205.1956 | −0.0008 | 100 |
| hexylcinnamaldehyde | 217.1634 | 217.1592 | 0.0042 | 6.5534 |
| ar-tumerone | 217.1634 | 217.1592 | 0.0042 | 6.5534 |
| furanoeremophilane | 219.1759 | 219.1749 | 0.001 | 20.92 |
| nootkatone | 219.1759 | 219.1749 | 0.0009 | 20.92 |
| valerenal | 219.1759 | 219.1749 | 0.0009 | 20.92 |
| xanthorrhizol | 219.1759 | 219.1749 | 0.0009 | 20.92 |
| curlone | 219.1759 | 219.1749 | 0.0009 | 20.92 |
| turmerone/ar-turmerol | 219.1759 | 219.1749 | 0.0009 | 20.92 |
| caryophellene oxide/spathulenol/bergamotol | 221.1916 | 221.1905 | 0.0011 | 17.16 |
| spathulenol/9-cedranone/lanceol | 221.1916 | 221.1905 | 0.0011 | 17.16 |
| undec-2-ene-8,10-diynoic aci | 232.1752 | 232.1701 | 0.0051 | 2.582 |
| costunolide | 233.1619 | 233.1541 | 0.0078 | 3.9941 |
| panthenol | 234.175 | 234.1705 | 0.0045 | 1.6686 |
| eremophilanlactone | 235.1698 | 235.1698 | 0 | 8.0883 |
| 2-octyl benzoate | 235.1698 | 235.1698 | 0 | 8.0883 |
| valerenic acid | 235.1698 | 235.1698 | 0 | 8.0883 |
| vellerdiol | 237.1842 | 237.1854 | −0.0012 | 9.1467 |
| 3-methyl-but-2-enoic acid, 1 | 237.1842 | 237.1854 | −0.0012 | 9.1467 |
| 2-pentenoic acid, 3-methyl-5 | 237.1842 | 237.1854 | −0.0012 | 9.1467 |
| a-ionyl acetate | 237.1842 | 237.1854 | −0.0012 | 9.1467 |
| 3-hydroxymyristic acid | 245.2193 | 245.2116 | 0.0076 | 0.6104 |
| 6-paradol | 251.1707 | 251.1647 | 0.006 | 2.2288 |
| hydroxyvalerenic acid | 251.1707 | 251.1647 | 0.006 | 2.2288 |
| palmitic acid | 257.2517 | 257.248 | 0.0036 | 1.6911 |
| farnesyl acetate | 265.2094 | 265.2167 | −0.0073 | 0.8614 |
| C20H32/biformene/kaur-16-ene | 273.2547 | 273.2582 | −0.0035 | 3.9459 |
| podocarpic acid | 275.1744 | 275.1647 | 0.0097 | 2.7248 |
| eserine | 276.1755 | 276.1712 | 0.0043 | 3.9929 |
| 6-shogaol | 277.1801 | 277.1803 | −0.0003 | 47.523 |
| menthyl salicylate | 277.1801 | 277.1803 | −0.0003 | 47.523 |
| cyclohexanecarboxylic acid | 277.1801 | 277.1803 | −0.0003 | 47.523 |
| 6-shogaol | 277.1801 | 277.1804 | −0.0003 | 47.523 |
| stearolic acid | 281.2483 | 281.248 | 0.0003 | 0.5256 |
| linoleic acid | 281.2483 | 281.248 | 0.0003 | 0.5256 |
| 9,12-octadecadienoic acid | 281.2483 | 281.248 | 0.0003 | 0.5256 |
| stearolic acid/linoelaidic acid | 281.2483 | 281.248 | 0.0003 | 0.5256 |
| 9,12-octadecadienoic acid | 281.2483 | 281.248 | 0.0003 | 0.5256 |
| linoleic acid | 281.2483 | 281.248 | 0.0003 | 0.5256 |
| vitamin A(retinol) | 287.241 | 287.2375 | 0.0034 | 2.014 |
| abieta-8,11,13-trien-18-ol | 287.241 | 287.2375 | 0.0034 | 2.014 |
| atropine | 291.1797 | 291.1834 | −0.0037 | 4.8426 |
| N-octyl-B-D-glucopyranoside | 293.1952 | 293.1964 | −0.0012 | 1.9744 |
| 6-gingerol | 295.2004 | 295.1909 | 0.0095 | 4.167 |
| embelin | 295.2004 | 295.1909 | 0.0095 | 4.167 |
| 6-gingerol | 295.2004 | 295.1909 | 0.0095 | 4.167 |
| abietic acid | 303.2422 | 303.2324 | 0.0098 | 1.5836 |
| eicosapentaenoic acid | 303.2422 | 303.2324 | 0.0098 | 1.5836 |
| 8-shogaol | 305.2177 | 305.2117 | 0.006 | 6.1055 |
| dihydrocapsaicin | 308.2162 | 308.2225 | −0.0064 | 1.0241 |
| progesterone | 315.2253 | 315.2324 | −0.0071 | 0.7179 |
| galanolactone/aframodial/galanal | 319.2354 | 319.2273 | 0.0081 | 0.9716 |
| 2-chloroethyl palmitate | 319.2354 | 319.2404 | −0.005 | 0.9716 |
| homodihydrocapsaicin | 322.2408 | 322.2382 | 0.0026 | 1.5194 |
| hydroxyprogesterone/DHEA acetate | 331.232 | 331.2273 | 0.0047 | 0.6114 |
| 10-shogaol | 333.2477 | 333.243 | 0.0047 | 4.7715 |
| chenodeoxycholic acid | 345.3027 | 345.3005 | 0.0022 | 0.1657 |
| incensole acetate | 349.2673 | 349.2742 | −0.0069 | 0.5485 |
| lithocholic acid | 377.2974 | 377.3055 | −0.0082 | 0.1939 |
| mogroside backbone-4H2O | 405.3597 | 405.3522 | 0.0076 | 1.0739 |
| beta/gamma-tocotrienol | 411.3168 | 411.3263 | −0.0095 | 0.3107 |
| benzethonium | 413.3268 | 413.3294 | −0.0026 | 0.4962 |
| calcitriol/sarsapogenin | 417.34 | 417.3368 | 0.0032 | 0.7499 |
| jervine | 426.2959 | 426.3008 | −0.005 | 1.4351 |
| hecogenin/ruscogenin | 431.3197 | 431.3161 | 0.0036 | 0.5419 |
| deoxymogroside backbone-2H | 443.341 | 443.3425 | −0.0015 | 0.7832 |
| ursonic acid/dehydroboswellic acid | 455.3501 | 455.3525 | −0.0025 | 1.1013 |
| ursolic/oleanolic/boswellic acids | 457.3728 | 457.3682 | 0.0046 | 0.5082 |
| ganoderic acid D/M | 469.334 | 469.3318 | 0.0022 | 0.1322 |
| ganodermadiol | 485.3902 | 485.3995 | −0.0093 | 0.5699 |
| 3-O-acetyl-9,11-dehydro BA | 497.3699 | 497.3631 | 0.0068 | 0.9493 |
| a-boswellic acid | 499.4099 | 499.4151 | −0.0053 | 1.059 |
| 3-O-acetyl-11-hydroxy boswellic acid | 515.3796 | 515.3737 | 0.0059 | 0.2249 |
| vitamin E succinate | 531.4136 | 531.4049 | 0.0087 | 0.3516 |
| hyperforin | 537.403 | 537.3944 | 0.0086 | 0.1584 |
| gymnemic acid IV/XIV - GlcA | 589.4186 | 589.4104 | 0.0082 | 0.2646 |

Compounds in Single Stage SCCO₂ Extraction at 40° C. and 300

6-shogoal and galanolactone were present in this extract in 52.2 and 2.8% relative abundance, respectively. Other shogaols, paradols, gingerols, and gingerdiols were present in the extract. Amino acids, vitamins, fatty acids, alkaloids, tumerones, terpenoids, ganoderols, gymnemic acids, ginsenosides, phenolic acids, phenols, sterols, capsaicins, gymnemagins, boswellic acids, saponins and hydrocarbons were also present in this extract. 104 out 564 (18%) unique chemicals have been directly identified in this extract using the DART TOF-MS. Table 13 shows the compounds identified in the extracts along with their relative abundance. FIG. 8G shows the DART Spectrum of this extract.

TABLE 13
Compounds in Single Stage SCCO₂ extraction at 40° C. and 300 bar

| Compound | Meas. | Calc. | Diff(u) | Abund. |
|---|---|---|---|---|
| ethylbenzene | 107.0872 | 107.0861 | 0.0011 | 0.6923 |
| propyl sulfide | 119.0863 | 119.0894 | −0.0031 | 7.7348 |
| pseudocumene/propynylcyclohexene | 121.1024 | 121.1017 | 0.0006 | 8.9814 |
| 5-hepten-2-one, 6-methyl- | 127.1133 | 127.1123 | 0.001 | 0.7543 |
| leucine | 132.0934 | 132.1024 | −0.009 | 0.3163 |
| ornithine | 133.1033 | 133.0977 | 0.0056 | 2.975 |
| dicyclopentadiene | 133.1033 | 133.1017 | 0.0016 | 2.975 |
| p-cymene | 135.1185 | 135.1174 | 0.0011 | 4.7417 |
| anisaldehyde/formic acid benzoate | 137.0606 | 137.0602 | 0.0004 | 7.8226 |
| octalactone | 143.1089 | 143.1072 | 0.0017 | 1.8029 |
| baogongteng B | 144.1087 | 144.1024 | 0.0063 | 0.0611 |
| crotonylbetaine | 145.1032 | 145.1103 | −0.0071 | 1.9648 |
| lysine | 147.1178 | 147.1133 | 0.0044 | 5.3976 |
| carvacrol/thymol/cymenol | 151.1173 | 151.1123 | 0.005 | 2.8056 |
| 2-butyl-3-methylpyrazine | 151.1173 | 151.1235 | −0.0062 | 2.8056 |
| norpseudophedrine | 152.1131 | 152.1075 | 0.0056 | 0.3637 |
| decadienal/santolina epoxide | 153.1283 | 153.1279 | 0.0004 | 3.9006 |
| pinene oxide | 153.1283 | 153.1279 | 0.0004 | 3.9006 |
| methylcholine | 161.1337 | 161.1416 | −0.0079 | 5.0524 |
| acetylthiocholine | 163.1096 | 163.1031 | 0.0065 | 5.7484 |
| jasmone | 165.1193 | 165.1279 | −0.0086 | 1.9368 |
| ephedrine | 166.1254 | 166.1232 | 0.0022 | 0.417 |
| hordenine | 166.1254 | 166.1232 | 0.0022 | 0.417 |
| pseudoephedrine | 166.1254 | 166.1232 | 0.0022 | 0.417 |
| camphorquinone | 167.1075 | 167.1072 | 0.0003 | 1.9858 |
| perillic acid | 167.1075 | 167.1072 | 0.0003 | 1.9858 |
| 3-(phenylmethoxy)-1-propanol | 167.1075 | 167.1072 | 0.0003 | 1.9858 |
| (3Z)-3-hexenyl-2-butenoate | 169.1303 | 169.1228 | 0.0075 | 1.0858 |
| chrysanthemolactone | 169.1303 | 169.1228 | 0.0075 | 1.0858 |
| a-Limonene diepoxide | 169.1303 | 169.1228 | 0.0075 | 1.0858 |
| lupinine | 170.1471 | 170.1545 | −0.0074 | 0.057 |
| decalactone | 171.1409 | 171.1385 | 0.0023 | 0.3885 |
| linalool oxide | 171.1409 | 171.1385 | 0.0023 | 0.3885 |
| butanoic acid, 3-hexenyl ester | 171.1409 | 171.1385 | 0.0023 | 0.3885 |
| 3,7-octadiene-2,6-diol, 2,6- | 171.1409 | 171.1385 | 0.0023 | 0.3885 |
| 1,7-octadiene-3,6-diol, 2,6- | 171.1409 | 171.1385 | 0.0023 | 0.3885 |
| arcaine | 173.1421 | 173.1514 | −0.0093 | 0.9527 |
| cinnamyl acetate | 177.0921 | 177.0915 | 0.0006 | 15.172 |
| canavanine | 177.0921 | 177.0987 | −0.0066 | 15.172 |
| eugenol methyl ether | 179.11 | 179.1072 | 0.0027 | 3.469 |
| 4-(p-methoxyphenyl)-2-butanonone | 179.11 | 179.1072 | 0.0027 | 3.469 |
| anisylacetone | 179.11 | 179.1072 | 0.0027 | 3.469 |
| eugenol methylether | 179.11 | 179.1072 | 0.0027 | 3.469 |
| chamazulen | 185.1301 | 185.133 | −0.0029 | 0.7601 |
| 1,3-di-tert-butylbenzene | 191.1821 | 191.18 | 0.002 | 3.6119 |
| damascone | 193.1596 | 193.1592 | 0.0004 | 1.7493 |
| ionone | 193.1596 | 193.1592 | 0.0004 | 1.7493 |
| β-pinene, 3-(acetylmethyl)- | 193.1596 | 193.1592 | 0.0004 | 1.7493 |
| sedanolide | 195.1378 | 195.1385 | −0.0007 | 1.1973 |
| trans-chrysanthenyl acetate | 195.1378 | 195.1385 | −0.0007 | 1.1973 |
| (−)-myrtenyl acetate | 195.1378 | 195.1385 | −0.0007 | 1.1973 |
| guaiazulene | 199.1455 | 199.1487 | −0.0032 | 1.6029 |
| naphthalene, 1,6-dimethyl-4- | 199.1455 | 199.1487 | −0.0032 | 1.6029 |
| dehydrocurcumene | 201.1651 | 201.1643 | 0.0008 | 17.07 |
| curcumene/cuparene/calamenene | 203.18 | 203.18 | 0 | 100 |
| zingiberene/(Z,E)-a-farnesene | 205.1955 | 205.1956 | −0.0001 | 81.075 |
| alloaromadendrene/elemene | 205.1955 | 205.1956 | −0.0001 | 81.075 |
| cycloheptane, 4-methylene-1- | 205.1955 | 205.1956 | −0.0001 | 81.075 |
| caryophyllene | 205.1955 | 205.1956 | −0.0001 | 81.075 |
| cedrene | 205.1955 | 205.1956 | −0.0001 | 81.075 |
| humulene | 205.1955 | 205.1956 | −0.0001 | 81.075 |
| isocaryophyllene | 205.1955 | 205.1956 | −0.0001 | 81.075 |
| isolongifolene | 205.1955 | 205.1956 | −0.0001 | 81.075 |
| longicyclene/longifolene | 205.1955 | 205.1956 | −0.0001 | 81.075 |
| thujopsen | 205.1955 | 205.1956 | −0.0001 | 81.075 |
| valencene | 205.1955 | 205.1956 | −0.0001 | 81.075 |
| beta-gualene/cis-gamma-bisabolene | 205.1955 | 205.1956 | −0.0001 | 81.075 |
| copaene | 205.1955 | 205.1956 | −0.0001 | 81.075 |
| germacrene D | 205.1955 | 205.1956 | −0.0001 | 81.075 |
| a-cubebene | 205.1955 | 205.1956 | −0.0001 | 81.075 |
| a-muurolene | 205.1955 | 205.1956 | −0.0001 | 81.075 |
| trans-a-bergamotene | 205.1955 | 205.1956 | −0.0001 | 81.075 |
| (−)-a-panasinsen | 205.1955 | 205.1956 | −0.0001 | 81.075 |
| β-sesquiphellandrene | 205.1955 | 205.1956 | −0.0001 | 81.075 |
| cedrene/valencene/β-guainene | 205.1955 | 205.1956 | −0.0001 | 81.075 |
| carvylacetate | 209.1568 | 209.1541 | 0.0026 | 1.2319 |
| epoxy-a-terpenyl acetate | 213.1584 | 213.149 | 0.0094 | 0.9335 |
| hexylcinnamaldehyde | 217.161 | 217.1592 | 0.0018 | 10.369 |
| ar-tumerone | 217.161 | 217.1592 | 0.0018 | 10.369 |
| furanoeremophilane | 219.1754 | 219.1749 | 0.0005 | 31.609 |
| nootkatone | 219.1754 | 219.1749 | 0.0005 | 31.609 |
| valerenal | 219.1754 | 219.1749 | 0.0005 | 31.609 |
| xanthorrhizol | 219.1754 | 219.1749 | 0.0005 | 31.609 |
| curlone | 219.1754 | 219.1749 | 0.0005 | 31.609 |
| turmerone/ar-turmerol | 219.1754 | 219.1749 | 0.0005 | 31.609 |
| caryophellene oxide | 221.1909 | 221.1905 | 0.0003 | 20.984 |
| spathulenol | 221.1909 | 221.1905 | 0.0003 | 20.984 |
| bergamotol, Z-a-trans- | 221.1909 | 221.1905 | 0.0003 | 20.984 |
| spathulenol/9-cedranone/lanceol | 221.1909 | 221.1905 | 0.0003 | 20.984 |
| 2,2,6-trimethyl-1-(3-methylb | 223.1787 | 223.1698 | 0.0089 | 3.9139 |
| undec-2-ene-8,10-diynoic aci | 232.174 | 232.1701 | 0.0038 | 6.1055 |
| costunolide | 233.1614 | 233.1541 | 0.0073 | 7.132 |
| panthenol | 234.1768 | 234.1705 | 0.0063 | 3.6014 |
| eremophilanlactone | 235.1698 | 235.1698 | −0.0001 | 18.113 |
| 2-octyl benzoate | 235.1698 | 235.1698 | −0.0001 | 18.113 |
| valerenic acid | 235.1698 | 235.1698 | −0.0001 | 18.113 |
| vellerdiol | 237.1845 | 237.1854 | −0.0009 | 12.955 |
| 3-methyl-but-2-enoic acid, 1 | 237.1845 | 237.1854 | −0.0009 | 12.955 |
| 2-pentenoic acid, 3-methyl-5 | 237.1845 | 237.1854 | −0.0009 | 12.955 |
| a-ionyl acetate | 237.1845 | 237.1854 | −0.0009 | 12.955 |
| 3-hydroxymyristic acid | 245.2046 | 245.2116 | −0.007 | 1.9742 |
| 6-paradol | 251.1669 | 251.1647 | 0.0022 | 5.0224 |
| hydroxyvalerenic acid | 251.1669 | 251.1647 | 0.0021 | 5.0224 |
| palmitic acid | 257.2469 | 257.248 | −0.0011 | 2.4987 |
| oxymatrine | 265.1989 | 265.1916 | 0.0073 | 2.3646 |
| C20H32/biformene/kaur-16-ene | 273.255 | 273.2582 | −0.0032 | 6.3804 |
| eserine | 276.18 | 276.1712 | 0.0088 | 5.7987 |
| 6-shogaol | 277.1801 | 277.1803 | −0.0002 | 52.231 |
| menthyl salicylate | 277.1801 | 277.1803 | −0.0002 | 52.231 |
| cyclohexanecarboxylic acid, | 277.1801 | 277.1803 | −0.0002 | 52.231 |
| 6-shogaol | 277.1801 | 277.1804 | −0.0003 | 52.231 |
| stearolic acid | 281.243 | 281.248 | −0.005 | 1.8718 |
| linoleic acid | 281.243 | 281.248 | −0.005 | 1.8718 |
| 9,12-octadecadienoic acid | 281.243 | 281.248 | −0.005 | 1.8718 |
| stearolic acid/linoelaidic acid | 281.243 | 281.248 | −0.005 | 1.8718 |
| 9,12-octadecadienoic acid (Z | 281.243 | 281.248 | −0.005 | 1.8718 |
| linoleic acid | 281.243 | 281.248 | −0.005 | 1.8718 |
| vitamin A(retinol) | 287.2404 | 287.2375 | 0.0029 | 6.5772 |
| abieta-8,11,13-trien-18-ol | 287.2404 | 287.2375 | 0.0029 | 6.5772 |
| atropine | 291.1778 | 291.1834 | −0.0057 | 7.4642 |
| N-octyl-B-D-glucopyranoside | 293.1946 | 293.1964 | −0.0018 | 3.5232 |
| abietic acid | 303.2422 | 303.2324 | 0.0098 | 5.6102 |
| eicosapentaenoic acid | 303.2422 | 303.2324 | 0.0098 | 5.6102 |
| 8-shogaol | 305.2194 | 305.2117 | 0.0077 | 8.3405 |
| dihydrocapsaicin | 308.2253 | 308.2225 | 0.0028 | 2.5435 |
| progesterone | 315.2296 | 315.2324 | −0.0029 | 1.6945 |
| galanolactone/aframodial/galanal | 319.2359 | 319.2273 | 0.0086 | 2.7601 |
| 2-chloroethyl palmitate | 319.2359 | 319.2404 | −0.0046 | 2.7601 |
| homodihydrocapsaicin | 322.2399 | 322.2382 | 0.0017 | 3.4923 |
| docosahexenoic acid | 329.2538 | 329.248 | 0.0058 | 2.0997 |
| 10-shogaol | 333.2468 | 333.243 | 0.0038 | 6.7516 |
| kauran-18-al, 17-(acetyloxy) | 347.2523 | 347.2586 | −0.0063 | 1.8247 |
| incensole acetate | 349.269 | 349.2742 | −0.0052 | 1.6397 |
| lithocholic acid | 377.299 | 377.3055 | −0.0066 | 1.1237 |
| delta-tocotrienol | 397.307 | 397.3107 | −0.0036 | 1.3727 |
| mogroside backbone-4H2O | 405.3586 | 405.3522 | 0.0065 | 1.9204 |
| beta/gamma-tocotrienol | 411.318 | 411.3263 | −0.0083 | 1.4743 |
| benzethonium | 413.3289 | 413.3294 | −0.0005 | 1.2704 |
| tomatidine | 416.3588 | 416.3528 | 0.0059 | 1.9041 |
| calcitriol/sarsapogenin | 417.3395 | 417.3368 | 0.0027 | 1.0793 |
| isopomiferin | 421.1653 | 421.1651 | 0.0002 | 0.1403 |
| pomiferin | 421.1653 | 421.1651 | 0.0002 | 0.1403 |
| jervine | 426.2927 | 426.3008 | −0.0081 | 5.1766 |
| hecogenin/ruscogenin | 431.3206 | 431.3161 | 0.0045 | 2.9993 |
| deoxymogroside backbone-2H | 443.3383 | 443.3425 | −0.0042 | 1.6474 |
| ursonic acid/dehydroboswellic acid | 455.3503 | 455.3525 | −0.0022 | 2.7522 |
| ursolic/oleanolic/boswellic acids | 457.3781 | 457.3682 | 0.0099 | 1.4344 |
| ganoderic acid D/M | 469.3404 | 469.3318 | 0.0086 | 0.6453 |
| Gymnemasaponin II - 2 Glc | 475.374 | 475.3787 | −0.0047 | 0.5451 |
| 18-glycyrrhetinic acid methyl ester | 485.3729 | 485.3631 | 0.0098 | 0.662 |
| keto boswellic acid/ganodermol | 487.3848 | 487.3787 | 0.006 | 0.712 |
| 3-O-acetyl-9,11-dehydro BA | 497.3688 | 497.3631 | 0.0058 | 1.4628 |
| 3-O-acetyl-11-hydroxy boswellic acid | 515.3801 | 515.3737 | 0.0065 | 0.2704 |
| vitamin E succinate | 531.4044 | 531.4049 | −0.0005 | 0.1699 |
| adhyperforin | 551.4146 | 551.41 | 0.0045 | 0.1352 |
| gymnemic acid III/XIII - Glc | 591.4243 | 591.4261 | −0.0018 | 0.1973 |
| ginsenoside M1 | 609.4337 | 609.4367 | −0.003 | 0.153 |

### Example 2

### Example of Step 1B (FIG. 1)

### Multi-Stage SCCO₂ Fractionation of Ginger Essential Oil

Multi-stage SCCO₂ extraction/fractionation was performed using a SFT 250 (Supercritical Fluid Technology, Inc., Newark, Del., USA). In typical multi-stage extractions, 19 gm ground ginger rhizome, particle size greater than 105 μm, was loaded into an extraction vessel with an internal volume of 100 ml. The extraction solution was collected in a 40 ml collector vessel connected to the exit of the extraction vessel. The flow rate of CO₂ was set at 19 g/min. The first extraction step was performed at a pressure of 70 bar and a temperature of 40° C. (CO₂ density 0.206 gm/ml). This extraction step was carried out for 30 minutes. The second extraction step was performed at a pressure of 80 bar and a temperature of 40° C. (CO₂ density 0.293 gm/ml). The second extraction step lasted for 30 minutes. The third extraction step was performed at a pressure of 90 bar and a temperature of 40° C. for 30 minutes (CO₂ density 0.524 gm/ml). Another two extraction stages at a temperature of 40° C. and a pressure of 100 bar (CO₂ density 0.640) and 120 bar (CO₂ density 0.723 gm/ml) was then sequentially performed for 30 minutes each. The analytical results are reported in Table 14 (HPLC) and Table 15 (GC-MS).

TABLE 14
Multi-stage extraction yield and HPLC analysis results for each stage.

| | | | | | | | | | | 6-G | | |
|---|---|---|---|---|---|---|---|---|---|---|---|---|
| | T | P | | density | Purity (%) | | | | | ratio | Yield (%) | |
| stage | (° C.) | (bar) | S/F | (g/cc) | 6-G | 8-G | 10-G | 6-S | total | (%) | total | gingerol |
| 1 | 40 | 70 | 38 | 0.206 | 9.00 | 1.72 | 4.89 | 2.58 | 18.19 | 49.5 | 0.34 | 0.06 |
| 2 | 40 | 80 | 38 | 0.293 | 19.30 | 2.19 | 3.77 | 25.81 | 51.07 | 37.8 | 0.27 | 0.14 |
| 3 | 40 | 90 | 38 | 0.523 | 29.07 | 5.63 | 10.74 | 8.78 | 54.22 | 53.6 | 0.82 | 0.44 |
| 4 | 40 | 100 | 38 | 0.64 | 27.44 | 6.46 | 16.40 | 5.72 | 56.02 | 49.0 | 0.5 | 0.28 |
| 5 | 40 | 120 | 38 | 0.723 | 7.83 | 1.72 | 5.22 | 1.43 | 16.19 | 48.4 | 0.34 | 0.06 |
| | | total: | 190 | | | | | | | | 2.27 | 0.98 |

TABLE 15
Multi-stage extraction GC-MS analysis results.

| | Stages | | | | |
|---|---|---|---|---|---|
| | 1 | 2 | 3 | 4 | 5 |
| Peak No. | Peak area percentage (%) | | | | |
| 1 | | | 0.27 | 0.27 | |
| 2 | | | | | |
| 3 | | | | | |
| 4 | | | | | |
| 5 | 0.67 | 0.43 | 5.25 | 6.37 | 6.27 |
| 6 | | | | | |
| 7 | | | | | |
| 8 | | | | | |
| 9 | 0.18 | | | | |
| 10 | 1.99 | 0.72 | 11.41 | 14.72 | 13.31 |
| 11 | 0.57 | 0.39 | 0.47 | 1.01 | 3.44 |
| 12 | | | 0.39 | 0.59 | 0.23 |
| 13 | | | | | |
| 14 | | | 0.3 | 0.6 | |
| 15 | 0.63 | 0.16 | | | |
| 16 | 0.38 | | | | |
| 17 | | | | 0.6 | |
| 18 | | | | | |
| 19 | | | | | |
| 20 | 0.46 | 0.45 | | | |
| 21 | 28.62 | 3.29 | 1.36 | 1.84 | 3.93 |
| 22 | 10.9 | 4.48 | 1.06 | 1.99 | 6.42 |
| 23 | 2.74 | 0.35 | | | |
| 24 | 13.82 | 2.09 | 0.29 | 0.79 | 2.02 |
| 25 | 0.46 | 0.23 | 0.74 | | |
| 26 | | | | | |
| 27 | 0.25 | 0.17 | | | |
| 28 | 18.18 | 3.85 | | 1.29 | 3.8 |
| 29 | 0.39 | 0.3 | | | |
| 30 | 0.34 | 0.2 | | | |
| 31 | 1.64 | 0.56 | | | |
| 32 | | | 0.41 | 0.69 | 0.76 |
| 33 | | | | | |
| 34 | 0.32 | 0.39 | | | |
| 35 | | 0.17 | 0.54 | 0.34 | 2.77 |
| 36 | 0.75 | 0.52 | | | |
| 37 | 0.29 | 0.59 | | | |
| 38 | 0.3 | 0.74 | | 0.16 | 0.4 |
| 39 | 6.17 | 7.53 | 42.79 | 39.7 | 31.21 |
| 40 | 0.71 | 1.33 | | | |
| 41 | 1.02 | 1.01 | | | |
| 42 | | 1.12 | 0.33 | 0.29 | 0.23 |
| 43 | 0.37 | 1.23 | 0.83 | 0.87 | 0.72 |
| 44 | | 0.36 | | 0.34 | |
| 45 | 0.98 | 1.66 | 0.45 | 0.42 | 0.26 |
| 46 | 2.5 | 5.21 | 1.55 | 1.72 | 1.86 |
| 47 | | 1.36 | 0.14 | | |
| 48 | 0.45 | 8.45 | 1.53 | 1.06 | 0.47 |
| 49 | | 0.85 | | | |
| 50 | | 1.17 | 0.52 | 0.42 | |
| 51 | | 1.49 | 0.38 | 0.2 | |
| 52 | | 0.74 | | | |
| 53 | | 0.56 | | | 0.26 |
| 54 | 0.6 | 1.02 | | | |
| 55 | | 0.51 | | | |
| 56 | | 1.12 | | | |
| 57 | | 0.89 | | | |
| 58 | | | | | |
| 59 | | | | | |
| 60 | | 1.42 | | | |
| 61 | | 1.66 | | | |
| 62 | 0.42 | 0.52 | 0.62 | 1.06 | 4.32 |
| 63 | | 1.74 | 1.37 | 0.85 | |
| 64 | | 2.08 | 0.53 | 0.09 | 0.39 |
| 65 | 1.52 | 20.53 | 19.86 | 14.22 | 5.76 |
| 66 | | | 0.86 | 0.44 | |
| 67 | 0.92 | 1.37 | 1.11 | 2.02 | 7.88 |
| 68 | | 1.01 | 1.99 | 1.93 | 0.39 |
| 69 | | 2.2 | 0.48 | 0.24 | |
| 70 | | 0.42 | 1.78 | 1.97 | |
| Total | 99.54 | 90.64 | 99.34 | 98.83 | 97.1 |
| Monoterpene | 0.18 | 0 | 0 | 0 | 0 |
| Sesquiterpene | 79.42 | 17.48 | 3.45 | 6.67 | 16.57 |
| Oxygenated Sesquiterpene | 6.03 | 23.16 | 5.26 | 5.03 | 3.31 |
| Gingerol | 7.69 | 31.57 | 67.81 | 58.35 | 37.75 |

Compounds in Multi Stage SCCO₂ Extraction Stage 1: 40° C. and 70 Bar

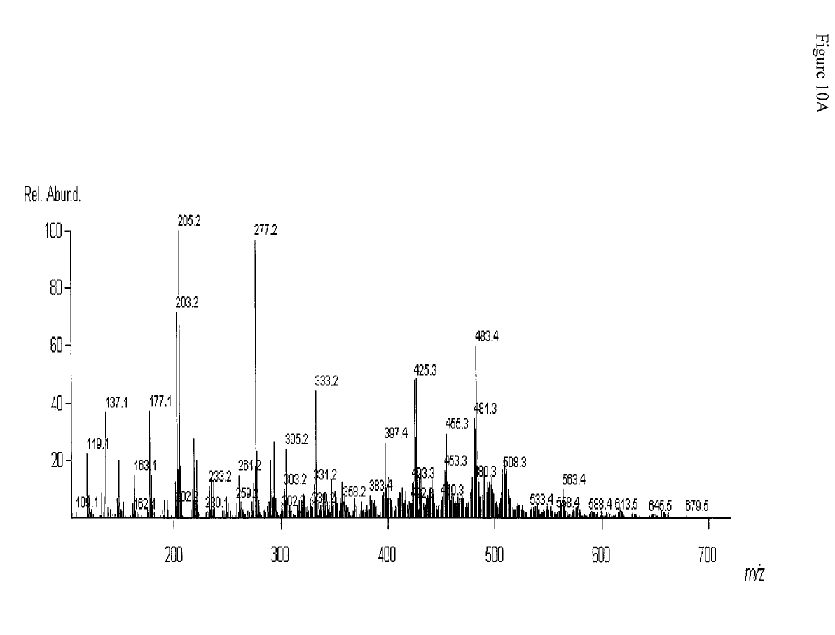

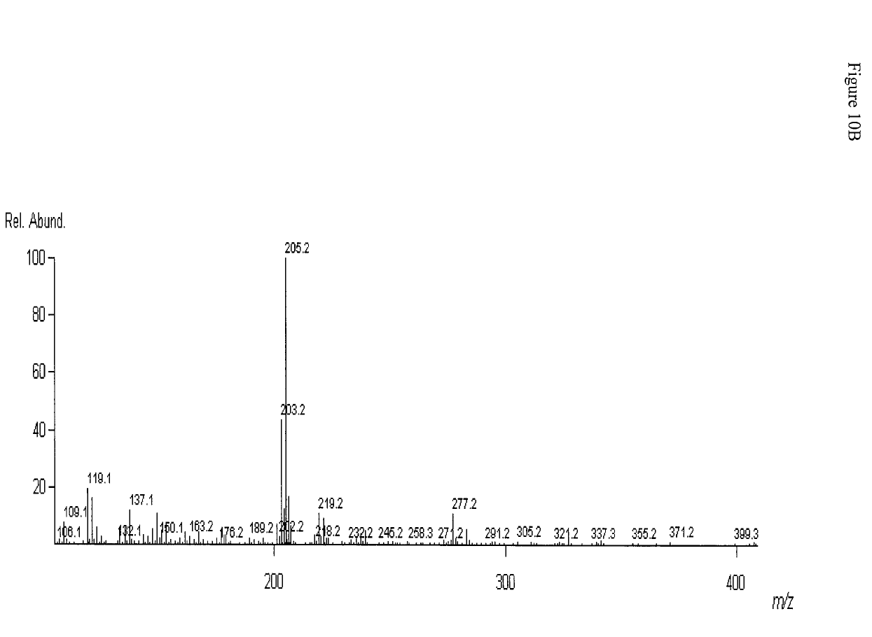

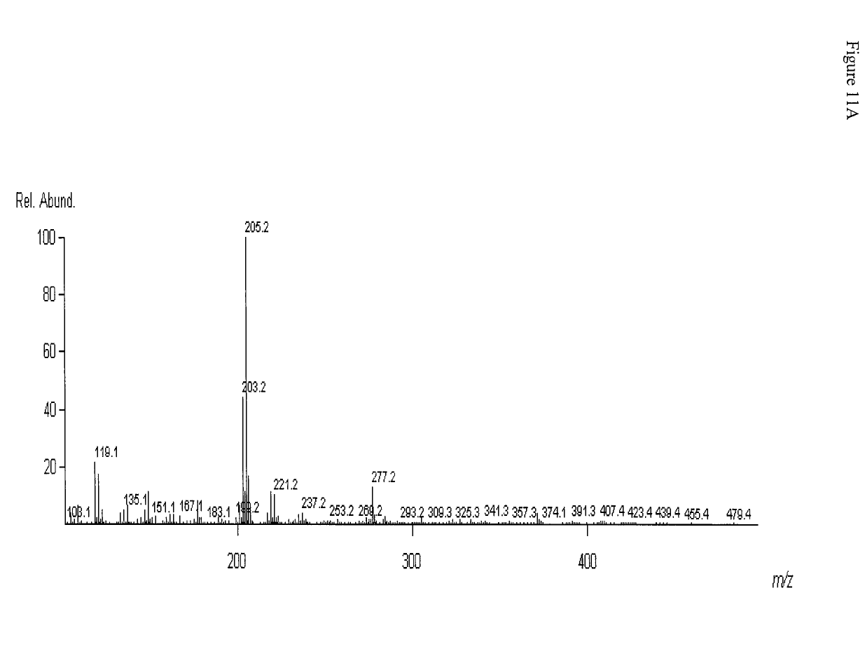

6-shogoal, 6 gingerol, and galanolactone were present in this extract in 48.0, 3.4, and 2.3% relative abundance, respectively. Other shogaols, paradols, gingerols, and gingerdiols were present in the extract. Amino acids, vitamins, fatty acids, saccharides, phenolic acids, phenols, sterols, capsaicins, alkaloids, quinones, terpenoids, xanthines, boswellic acids, saponins and hydrocarbons were also present in this extract. 109 out of 570 (19%) unique chemicals have been directly identified in this extract using the DART TOF-MS. Table 16 shows the compounds identified in the extracts along with their relative abundance. FIG. 9A shows the DART Spectrum of this extract.

TABLE 16
Compounds in Multi Stage SCCO₂ extraction stage 1: 40° C. and 70 bar

| Compounds | Meas. | Calc. | Diff(u) | Abund. |
|---|---|---|---|---|
| ethylbenzene | 107.0862 | 107.0861 | 0.0001 | 2.0116 |
| norcamphor/heptadienal | 111.0817 | 111.081 | 0.0007 | 0.4232 |
| povidone | 112.0829 | 112.0762 | 0.0067 | 0.0436 |
| histamine | 112.0829 | 112.0874 | −0.0045 | 0.0436 |
| 2-methylcyclohexanone | 113.0962 | 113.0966 | −0.0004 | 0.2763 |
| propyl sulfide | 119.0857 | 119.0894 | −0.0038 | 11.765 |
| pseudocumene/propynylcyclohexene | 121.1013 | 121.1017 | −0.0004 | 14.703 |
| 2,6-dimethylanilene/conyrin | 122.1064 | 122.0969 | 0.0095 | 1.4348 |
| 5-hepten-2-one, 6-methyl- | 127.1121 | 127.1123 | −0.0002 | 1.2195 |
| leucine | 132.093 | 132.1024 | −0.0094 | 0.1937 |
| ornithine | 133.1027 | 133.0977 | 0.005 | 3.1928 |
| dicyclopentadiene | 133.1027 | 133.1017 | 0.001 | 3.1928 |
| p-cymene | 135.1172 | 135.1174 | −0.0001 | 5.9449 |
| anisaldehyde/formic acid benzoate | 137.0596 | 137.0602 | −0.0007 | 14.446 |
| trigonelline/vitamin H | 138.0631 | 138.0555 | 0.0075 | 1.1369 |
| tropine | 142.1324 | 142.1232 | 0.0092 | 0.1603 |
| octalactone | 143.1077 | 143.1072 | 0.0004 | 1.7023 |
| baogongteng B | 144.1082 | 144.1024 | 0.0058 | 0.1493 |
| crotonylbetaine | 145.1022 | 145.1103 | −0.0081 | 5.1879 |
| lysine | 147.1169 | 147.1133 | 0.0035 | 9.0739 |
| 1-methyl-3-phenylpropylamine | 150.1373 | 150.1282 | 0.0091 | 1.6607 |
| 4-phenylbutylamine | 150.1373 | 150.1282 | 0.0091 | 1.6607 |
| carvacrol/thymol/cymenol | 151.1154 | 151.1123 | 0.0031 | 3.856 |
| 2-butyl-3-methylpyrazine | 151.1154 | 151.1235 | −0.0082 | 3.856 |
| norpseudophedrine | 152.1133 | 152.1075 | 0.0058 | 0.5075 |
| decadienal/santolina epoxide | 153.1268 | 153.1279 | −0.0011 | 5.364 |
| pinene oxide | 153.1268 | 153.1279 | −0.0011 | 5.364 |
| methylcholine | 161.1321 | 161.1416 | −0.0095 | 6.871 |
| jasmone | 165.124 | 165.1279 | −0.0039 | 2.6091 |
| ephedrine | 166.1262 | 166.1232 | 0.003 | 0.5565 |
| hordenine | 166.1262 | 166.1232 | 0.003 | 0.5565 |
| pseudoephedrine | 166.1262 | 166.1232 | 0.003 | 0.5565 |
| camphorquinone | 167.1061 | 167.1072 | −0.0011 | 4.4069 |
| perillic acid | 167.1061 | 167.1072 | −0.0011 | 4.4069 |
| 3-(phenylmethoxy)-1-propanol | 167.1061 | 167.1072 | −0.0011 | 4.4069 |
| (3Z)-3-hexenyl-2-butenoate | 169.1319 | 169.1228 | 0.0091 | 1.7546 |
| chrysanthemolactone | 169.1319 | 169.1228 | 0.0091 | 1.7546 |
| a-Limonene diepoxide | 169.1319 | 169.1228 | 0.0091 | 1.7546 |
| lupinine | 170.15 | 170.1545 | −0.0046 | 0.3492 |
| decalactone | 171.1362 | 171.1385 | −0.0023 | 1.1212 |
| linalool oxide | 171.1362 | 171.1385 | −0.0023 | 1.1212 |
| butanoic acid, 3-hexenyl ester | 171.1362 | 171.1385 | −0.0023 | 1.1212 |
| 3,7-octadiene-2,6-diol, 2,6- | 171.1362 | 171.1385 | −0.0023 | 1.1212 |
| 1,7-octadiene-3,6-diol, 2,6- | 171.1362 | 171.1385 | −0.0023 | 1.1212 |
| arcaine | 173.142 | 173.1514 | −0.0094 | 2.5979 |
| cinnamyl acetate | 177.0909 | 177.0915 | −0.0007 | 23.071 |
| canavanine | 177.0909 | 177.0987 | −0.0079 | 23.071 |
| 2(4H)-benzofuranone, 5,6,7,7 | 181.1276 | 181.1228 | 0.0048 | 2.0066 |
| chamazulen | 185.1278 | 185.133 | −0.0052 | 1.0573 |
| 1,3-di-tert-butylbenzene | 191.1789 | 191.18 | −0.0011 | 4.7933 |
| sedanolide | 195.1316 | 195.1385 | −0.0069 | 1.9736 |
| trans-chrysanthenyl acetate | 195.1316 | 195.1385 | −0.0069 | 1.9736 |
| (−)-myrtenyl acetate | 195.1316 | 195.1385 | −0.0069 | 1.9736 |
| 2,6-octadien-1-ol, 3,7-dimethyl | 197.1469 | 197.1541 | −0.0072 | 1.2923 |
| dihydrocarvylacetate | 197.1469 | 197.1541 | −0.0072 | 1.2923 |
| geranyl acetate | 197.1469 | 197.1541 | −0.0072 | 1.2923 |
| isobornyl acetate | 197.1469 | 197.1541 | −0.0072 | 1.2923 |
| isopulegyl acetate | 197.1469 | 197.1541 | −0.0072 | 1.2923 |
| lavandulyl acetate | 197.1469 | 197.1541 | −0.0072 | 1.2923 |
| L-bornyl acetate | 197.1469 | 197.1541 | −0.0072 | 1.2923 |
| linalyl acetate | 197.1469 | 197.1541 | −0.0072 | 1.2923 |
| neryl acetate | 197.1469 | 197.1541 | −0.0072 | 1.2923 |
| terpinyl acetate | 197.1469 | 197.1541 | −0.0072 | 1.2923 |
| acetic acid, bornyl ester | 197.1469 | 197.1541 | −0.0072 | 1.2923 |
| butane, 1-cyclopropylidene-5 | 197.1469 | 197.1541 | −0.0072 | 1.2923 |
| bornyl acetate | 197.1469 | 197.1541 | −0.0072 | 1.2923 |
| bornyl acetate/linalyl acetate | 197.1469 | 197.1541 | −0.0072 | 1.2923 |
| guaiazulene | 199.1472 | 199.1487 | −0.0015 | 3.4173 |
| naphthalene, 1,6-dimethyl-4- | 199.1472 | 199.1487 | −0.0015 | 3.4173 |
| dehydrocurcumene | 201.1638 | 201.1643 | −0.0006 | 22.984 |
| curcumene/cuparene/calamenene | 203.1788 | 203.18 | −0.0012 | 100 |
| zingiberene/farnesene/bisabolene | 205.1943 | 205.1956 | −0.0014 | 92.721 |
| alloaromadendrene/elemene/gualene | 205.1943 | 205.1956 | −0.0014 | 92.721 |
| cycloheptane, 4-methylene-1- | 205.1943 | 205.1956 | −0.0014 | 92.721 |
| aromadendrene | 205.1943 | 205.1956 | −0.0014 | 92.721 |
| caryophyllene | 205.1943 | 205.1956 | −0.0014 | 92.721 |
| cedrene | 205.1943 | 205.1956 | −0.0014 | 92.721 |
| farnesene | 205.1943 | 205.1956 | −0.0014 | 92.721 |
| humulene | 205.1943 | 205.1956 | −0.0014 | 92.721 |
| isocaryophyllene | 205.1943 | 205.1956 | −0.0014 | 92.721 |
| isolongifolene | 205.1943 | 205.1956 | −0.0014 | 92.721 |
| longicyclene/longifolene | 205.1943 | 205.1956 | −0.0014 | 92.721 |
| thujopsen | 205.1943 | 205.1956 | −0.0014 | 92.721 |
| valencene | 205.1943 | 205.1956 | −0.0014 | 92.721 |
| copaene | 205.1943 | 205.1956 | −0.0014 | 92.721 |
| germacrene D | 205.1943 | 205.1956 | −0.0014 | 92.721 |
| a-cubebene | 205.1943 | 205.1956 | −0.0014 | 92.721 |
| a-muurolene | 205.1943 | 205.1956 | −0.0014 | 92.721 |
| trans-a-bergamotene | 205.1943 | 205.1956 | −0.0014 | 92.721 |
| (−)-a-panasinsen | 205.1943 | 205.1956 | −0.0014 | 92.721 |
| β-sesquiphellandrene | 205.1943 | 205.1956 | −0.0014 | 92.721 |
| 3,5-bis(1,1-dimethylethyl)-p | 207.1738 | 207.1749 | −0.0011 | 14.585 |
| carvylacetate | 209.1589 | 209.1541 | 0.0048 | 1.646 |
| epoxy-a-terpenyl acetate/hyd | 213.1557 | 213.149 | 0.0067 | 1.1863 |
| hexylcinnamaldehyde | 217.1594 | 217.1592 | 0.0002 | 13.56 |
| ar-tumerone | 217.1594 | 217.1592 | 0.0002 | 13.56 |
| furanoeremophilane | 219.174 | 219.1749 | −0.0009 | 37.678 |
| nootkatone | 219.174 | 219.1749 | −0.0009 | 37.678 |
| valerenal | 219.174 | 219.1749 | −0.0009 | 37.678 |
| curlone | 219.174 | 219.1749 | −0.0009 | 37.678 |
| turmerone/ar-turmerol/xanthorrhizol | 219.174 | 219.1749 | −0.0009 | 37.678 |
| caryophellene oxide | 221.1893 | 221.1905 | −0.0012 | 27.467 |
| spathulenol | 221.1893 | 221.1905 | −0.0012 | 27.467 |
| bergamotol | 221.1893 | 221.1905 | −0.0012 | 27.467 |
| spathulenol/9-cedranone/lanceol | 221.1893 | 221.1905 | −0.0012 | 27.467 |
| undec-2-ene-8,10-diynoic acid | 232.1726 | 232.1701 | 0.0025 | 3.0178 |
| costunolide | 233.1568 | 233.1541 | 0.0027 | 6.6904 |
| panthenol | 234.1736 | 234.1705 | 0.0031 | 2.3819 |
| eremophilanlactone | 235.1684 | 235.1698 | −0.0014 | 15.523 |
| 2-octyl benzoate | 235.1684 | 235.1698 | −0.0015 | 15.523 |
| valerenic acid | 235.1684 | 235.1698 | −0.0015 | 15.523 |
| vellerdiol | 237.1833 | 237.1854 | −0.0021 | 11.896 |
| 3-methyl-but-2-enoic acid, 1 | 237.1833 | 237.1854 | −0.0021 | 11.896 |
| 2-pentenoic acid, 3-methyl-5 | 237.1833 | 237.1854 | −0.0021 | 11.896 |
| a-ionyl acetate | 237.1833 | 237.1854 | −0.0021 | 11.896 |
| isobornyl isovalerate | 239.1986 | 239.2011 | −0.0025 | 2.2028 |
| linalyl iso-valerate | 239.1986 | 239.2011 | −0.0025 | 2.2028 |
| 6-paradol | 251.1647 | 251.1647 | −0.0001 | 4.6301 |
| hydroxyvalerenic acid | 251.1647 | 251.1647 | −0.0001 | 4.6301 |
| palmitic acid | 257.2469 | 257.248 | −0.0011 | 2.1186 |
| panaxydol | 261.1884 | 261.1854 | 0.0029 | 5.9937 |
| oxymatrine | 266.1868 | 265.1916 | −0.0048 | 1.6215 |
| C20H32/biformene/kaur-16-ene | 273.255 | 273.2582 | −0.0032 | 4.5309 |
| podocarpic acid | 275.1734 | 275.1647 | 0.0087 | 4.5942 |
| eserine | 276.1796 | 276.1712 | 0.0083 | 2.5919 |
| 6-shogaol | 277.1785 | 277.1803 | −0.0018 | 48.026 |
| menthyl salicylate | 277.1785 | 277.1803 | −0.0018 | 48.026 |
| cyclohexanecarboxylic acid | 277.1785 | 277.1803 | −0.0018 | 48.026 |
| 6-shogaol | 277.1785 | 277.1804 | −0.0019 | 48.026 |
| 8-paradol | 279.2003 | 279.196 | 0.0043 | 5.0122 |
| lynestrenol | 285.2309 | 285.2218 | 0.0091 | 4.4387 |
| vitamin A(retinol) | 287.237 | 287.2375 | −0.0005 | 4.0953 |
| abieta-8,11,13-trien-18-ol | 287.237 | 287.2375 | −0.0005 | 4.0953 |
| atropine | 291.1823 | 291.1834 | −0.0011 | 5.0016 |
| N-octyl-B-D-glucopyranoside | 293.1911 | 293.1964 | −0.0053 | 3.0687 |
| 6-gingerol | 295.1999 | 295.1909 | 0.009 | 3.3567 |
| embelin | 295.1999 | 295.1909 | 0.009 | 3.3567 |
| 6-gingerol | 295.1999 | 295.1909 | 0.009 | 3.3567 |
| 6-gingerdiol | 297.2131 | 297.2066 | 0.0065 | 2.0639 |
| abietic acid | 303.2359 | 303.2324 | 0.0035 | 3.6598 |
| eicosapentaenoic acid | 303.2359 | 303.2324 | 0.0035 | 3.6598 |
| 8-shogaol | 305.2138 | 305.2117 | 0.0022 | 8.9048 |
| 10-paradol | 307.2318 | 307.2273 | 0.0045 | 1.5846 |
| dihydrocapsaicin | 308.2246 | 308.2225 | 0.0021 | 1.0071 |
| galanolactone/aframodial/galanal | 319.2308 | 319.2273 | 0.0035 | 2.305 |
| 2-chloroethyl palmitate | 319.2308 | 319.2404 | −0.0096 | 2.305 |
| homodihydrocapsaicin | 322.2381 | 322.2382 | −0.0001 | 2.0374 |
| incensole oxide | 323.2598 | 323.2586 | 0.0011 | 2.0488 |
| hydroxyprogesterone/DHEA acetate | 331.228 | 331.2273 | 0.0006 | 2.2684 |
| 10-shogaol | 333.2434 | 333.243 | 0.0005 | 9.8764 |
| pregnanetriol | 337.2683 | 337.2742 | −0.0059 | 1.7693 |
| C23H34O2 | 343.2681 | 343.2637 | 0.0044 | 1.2761 |
| 10-gingerdiol | 353.277 | 353.2692 | 0.0079 | 1.3799 |
| incensole oxide acetate | 365.2741 | 365.2692 | 0.0049 | 0.5913 |
| ginkgolic acid II | 375.2867 | 375.2899 | −0.0032 | 1.1948 |
| mogroside backbone - 4H2O | 405.354 | 405.3522 | 0.0018 | 1.6428 |
| diosgenin | 415.3239 | 415.3212 | 0.0027 | 1.0047 |
| mogroside backbone - 3H2O | 423.3661 | 423.3627 | 0.0034 | 1.9577 |
| jervine | 426.2992 | 426.3008 | −0.0016 | 1.8096 |
| hecogenin/ruscogenin | 431.3076 | 431.3161 | −0.0085 | 1.6629 |
| ursonic acid/dehydroboswellic acid | 455.3459 | 455.3525 | −0.0066 | 2.0491 |
| ursolic/oleanolic/boswellic acids | 457.366 | 457.3682 | −0.0022 | 1.2155 |
| ganoderic acid D/M | 469.3337 | 469.3318 | 0.0019 | 1.0287 |
| keto boswellic acid/glycyrrhizol | 471.3455 | 471.3474 | −0.0019 | 0.8512 |
| jujubogenin/bacoside A | 473.3586 | 473.3631 | −0.0045 | 0.6672 |
| soyasapogenol A | 474.3782 | 474.3709 | 0.0073 | 0.4791 |
| psychosine | 478.3396 | 478.338 | 0.0015 | 0.8087 |
| 23-Hydroxylongispinogenin | 491.3708 | 491.3736 | −0.0028 | 0.619 |
| gymnemagenin | 492.3536 | 492.3451 | 0.0084 | 0.8566 |
| 3-O-acetyl-9,11-dehydro BA | 497.3652 | 497.3631 | 0.0021 | 1.1927 |
| acetylboswellic acid/ganoderol | 499.3797 | 499.3787 | 0.001 | 0.7873 |
| gymnestrogenin/gymnemagenin | 507.3739 | 507.3686 | 0.0053 | 0.8647 |
| 3-O-acetyl-11-hydroxy boswellic acid | 515.3734 | 515.3737 | −0.0003 | 0.4959 |
| 3-acetyl-a-boswellic acid | 541.4286 | 541.4257 | 0.0029 | 0.3287 |
| adhyperforin | 551.4084 | 551.41 | −0.0016 | 0.2365 |
| lutein/zeaxanthin | 569.4382 | 569.4359 | 0.0023 | 0.1646 |
| ganodermic acids R/S | 571.3939 | 571.3999 | −0.006 | 0.478 |
| antheraxanthin/capsanthin | 585.4228 | 585.4307 | −0.0079 | 0.0571 |
| neoxanthin/violaxanthin | 601.4279 | 601.4257 | 0.0022 | 0.1942 |
| ginsenoside Rh1 | 627.4491 | 627.4472 | 0.0019 | 0.1619 |

Compounds in Multi Stage SCCO₂ Extraction Stage 2: 40° C. and 80 Bar

6-shogoal, 6 gingerol, and galanolactone were present in this extract in 19.7, 1.6, and 1.0% relative abundance, respectively. Other shogaols, paradols, gingerols, and gingerdiols were present in the extract. Amino acids, vitamins, fatty acids, saccharides, phenolic acids, phenols, sterols, capsaicins, gymnemagins, alkaloids, quinones, terpenoids, xanthines, boswellic acids, saponins and hydrocarbons were also present in this extract. 121 out of 672 (18%) unique chemicals have been directly identified in this extract using the DART TOF-MS. Table 17 shows the compounds identified in the extracts along with their relative abundance. FIG. 9B shows the DART Spectrum of this extract.

TABLE 17
Compounds in Multi Stage SCCO₂ extraction stage 2: 40° C. and 80 bar

| Compounds | Meas. | Calc. | Diff(u) | Abund. |
|---|---|---|---|---|
| p-cymene | 135.1167 | 135.1174 | −0.0007 | 1.1597 |
| ocimene/camphene/adamantane | 137.129 | 137.133 | −0.004 | 3.1942 |
| octalactone | 143.1047 | 143.1072 | −0.0026 | 2.7262 |
| carvacrol/thymol/cymenol | 151.1108 | 151.1123 | −0.0015 | 0.8188 |
| norpseudophedrine | 152.1107 | 152.1075 | 0.0032 | 0.3077 |
| decadienal/santolina epoxide | 153.1248 | 153.1279 | −0.0031 | 3.7976 |
| pinene oxide | 153.1248 | 153.1279 | −0.0031 | 3.7976 |
| pseudopelletierine | 154.1291 | 154.1232 | 0.0059 | 0.2021 |
| cineole/borneol | 155.1337 | 155.1436 | −0.0099 | 0.0876 |
| methone/pinocampheol/pulegol | 155.1337 | 155.1436 | −0.0099 | 0.0876 |
| methylcholine | 161.1449 | 161.1416 | 0.0032 | 0.1615 |
| 1,9-nonanediol | 161.1449 | 161.1541 | −0.0093 | 0.1615 |
| N-phenylmorpholine | 164.1054 | 164.1075 | −0.0021 | 0.2706 |
| ephedrine | 166.1176 | 166.1232 | −0.0057 | 0.1015 |
| hordenine | 166.1176 | 166.1232 | −0.0057 | 0.1015 |
| pseudoephedrine | 166.1176 | 166.1232 | −0.0057 | 0.1015 |
| camphorquinone | 167.1065 | 167.1072 | −0.0007 | 2.3624 |
| perillic acid | 167.1065 | 167.1072 | −0.0007 | 2.3624 |
| 3-(phenylmethoxy)-1-propanol | 167.1065 | 167.1072 | −0.0007 | 2.3624 |
| synephrine | 168.1107 | 168.1024 | 0.0083 | 0.1618 |
| (3Z)-3-hexenyl-2-butenoate | 169.122 | 169.1228 | −0.0008 | 0.8105 |
| chrysanthemolactone | 169.122 | 169.1228 | −0.0008 | 0.8105 |
| a-Limonene diepoxide | 169.122 | 169.1228 | −0.0008 | 0.8105 |
| eugenol methyl ether | 179.1037 | 179.1072 | −0.0036 | 1.1355 |
| 4-(p-methoxyphenyl)-2-butanone | 179.1037 | 179.1072 | −0.0036 | 1.1355 |
| anisylacetone | 179.1037 | 179.1072 | −0.0036 | 1.1355 |
| eugenol methylether | 179.1037 | 179.1072 | −0.0036 | 1.1355 |
| homophenylalanine | 180.1026 | 180.1024 | 0.0001 | 0.4382 |
| salsolinol | 180.1026 | 180.1024 | 0.0001 | 0.4382 |
| damascone | 193.1523 | 193.1592 | −0.007 | 0.3501 |
| ionone | 193.1523 | 193.1592 | −0.007 | 0.3501 |
| β-pinene, 3-(acetylmethyl)- | 193.1523 | 193.1592 | −0.007 | 0.3501 |
| sedanolide | 195.137 | 195.1385 | −0.0015 | 0.1106 |
| trans-chrysanthenyl acetate | 195.137 | 195.1385 | −0.0015 | 0.1106 |
| (−)-myrtenyl acetate | 195.137 | 195.1385 | −0.0015 | 0.1106 |
| dehydrocurcumene | 201.1643 | 201.1643 | 0 | 8.8815 |
| curcumene/cuparene/calamenene | 203.1784 | 203.18 | −0.0016 | 100 |
| zingiberene/farnesene/bisabolene | 205.1939 | 205.1956 | −0.0017 | 97.9606 |
| alloaromadendrene/elemene/cedrene | 205.1939 | 205.1956 | −0.0017 | 97.9606 |
| aromadendrene/humulene/thujopsen | 205.1939 | 205.1956 | −0.0017 | 97.9606 |
| isocaryophyllene/isolongifolene | 205.1939 | 205.1956 | −0.0017 | 97.9606 |
| longicyclene/longifolene | 205.1939 | 205.1956 | −0.0017 | 97.9606 |
| valencene/gualene/guainene/copaene | 205.1939 | 205.1956 | −0.0017 | 97.9606 |
| germacrene D/cubebene/muurolene | 205.1939 | 205.1956 | −0.0017 | 97.9606 |
| bergamotene/panasinsen/sesquiphellandrene | 205.1939 | 205.1956 | −0.0017 | 97.9606 |
| xanthurenic acid | 206.055 | 206.0453 | 0.0097 | 0.0256 |
| salsolidine | 208.1398 | 208.1337 | 0.0061 | 0.8336 |
| carvylacetate | 209.1518 | 209.1541 | −0.0023 | 0.5132 |
| hexylcinnamaldehyde | 217.159 | 217.1592 | −0.0002 | 3.6504 |
| ar-tumerone | 217.159 | 217.1592 | −0.0002 | 3.6504 |
| furanoeremophilane/nootkatone/valerenal | 219.1734 | 219.1749 | −0.0015 | 27.5323 |
| xanthorrhizol/curlone | 219.1734 | 219.1749 | −0.0015 | 27.5323 |
| turmerone/ar-turmerol | 219.1734 | 219.1749 | −0.0015 | 27.5323 |
| caryophellene oxide/spathulenol/bergamotol | 221.1888 | 221.1905 | −0.0017 | 28.5684 |
| 9-cedranone/lanceol | 221.1888 | 221.1905 | −0.0017 | 28.5684 |
| undec-2-ene-8,10-diynoic aci | 232.1691 | 232.1701 | −0.001 | 3.2578 |
| costunolide | 233.1595 | 233.1541 | 0.0054 | 3.3894 |
| panthenol | 234.1721 | 234.1705 | 0.0015 | 1.7176 |
| eremophilanlactone | 235.1687 | 235.1698 | −0.0011 | 8.6985 |
| 2-octyl benzoate | 235.1687 | 235.1698 | −0.0011 | 8.6985 |
| valerenic acid | 235.1687 | 235.1698 | −0.0011 | 8.6985 |
| vellerdiol | 237.1844 | 237.1854 | −0.001 | 8.5326 |
| a-ionyl acetate | 237.1844 | 237.1854 | −0.001 | 8.5326 |
| isobornyl isovalerate | 239.197 | 239.2011 | −0.0041 | 1.9453 |
| linalyl iso-valerate | 239.197 | 239.2011 | −0.0041 | 1.9453 |
| atractylenolide III | 249.1563 | 249.149 | 0.0073 | 1.5465 |
| parthenolide | 249.1563 | 249.149 | 0.0073 | 1.5465 |
| 6-paradol | 251.1651 | 251.1647 | 0.0003 | 1.8065 |
| hydroxyvalerenic acid | 251.1651 | 251.1647 | 0.0003 | 1.8065 |
| palmitic acid | 257.2511 | 257.248 | 0.0031 | 0.5545 |
| estrone | 271.1676 | 271.1698 | −0.0022 | 0.0292 |
| C20H32/biformene/kaur-16-ene | 273.2585 | 273.2582 | 0.0003 | 5.2881 |
| 3,6-epoxy-1-(4-hydroxy-3-met | 275.1682 | 275.1647 | 0.0035 | 2.1229 |
| podocarpic acid | 275.1682 | 275.1647 | 0.0035 | 2.1229 |
| eserine | 276.1732 | 276.1712 | 0.002 | 7.6599 |
| 6-shogaol | 277.1792 | 277.1803 | −0.0011 | 19.6639 |
| menthyl salicylate | 277.1792 | 277.1803 | −0.0011 | 19.6639 |
| cyclohexanecarboxylic acid, | 277.1792 | 277.1803 | −0.0011 | 19.6639 |
| 6-shogaol | 277.1792 | 277.1804 | −0.0011 | 19.6639 |
| 8-paradol | 279.2045 | 279.196 | 0.0084 | 1.5105 |
| stearolic acid/linoleic acid | 281.2439 | 281.248 | −0.0042 | 1.1839 |
| 9,12-octadecadienoic acid/linoleidic acid | 281.2439 | 281.248 | −0.0042 | 1.1839 |
| vitamin A(retinol) | 287.2384 | 287.2375 | 0.0008 | 4.3513 |
| abieta-8,11,13-trien-18-ol | 287.2384 | 287.2375 | 0.0008 | 4.3513 |
| atropine | 291.1832 | 291.1834 | −0.0002 | 1.8907 |
| N-octyl-B-D-glucopyranoside | 293.1919 | 293.1964 | −0.0046 | 1.0427 |
| 6-gingerol | 295.196 | 295.1909 | 0.005 | 1.563 |
| embelin | 295.196 | 295.1909 | 0.005 | 1.563 |
| 6-gingerol | 295.196 | 295.1909 | 0.005 | 1.563 |
| 10-heneicosene | 295.3432 | 295.3365 | 0.0067 | 0.0868 |
| abietic acid | 303.2357 | 303.2324 | 0.0033 | 1.9544 |
| eicosapentaenoic acid | 303.2357 | 303.2324 | 0.0033 | 1.9544 |
| aleuritic acid | 305.2229 | 305.2328 | −0.0099 | 2.3835 |
| averionol D | 313.2673 | 313.2733 | −0.0059 | 0.7544 |
| bioallethrin | 313.2673 | 313.2742 | −0.0069 | 0.7544 |
| allopregnendione | 317.2408 | 317.248 | −0.0072 | 0.7907 |
| pregnenolone | 317.2408 | 317.248 | −0.0072 | 0.7907 |
| galanolactone/aframodial/galanal | 319.2321 | 319.2273 | 0.0048 | 1.0205 |
| 2-chloroethyl palmitate | 319.2321 | 319.2404 | −0.0083 | 1.0205 |
| homodihydrocapsaicin | 322.2339 | 322.2382 | −0.0043 | 1.4553 |
| incensole oxide | 323.2676 | 323.2586 | 0.009 | 2.3531 |
| 10-shogaol | 333.2434 | 333.243 | 0.0005 | 1.5939 |
| kauran-18-al, 17-(acetyloxy) | 347.2617 | 347.2586 | 0.0031 | 0.861 |
| incensole acetate | 349.2643 | 349.2742 | −0.0099 | 0.8296 |
| docosahexenoic acid ethyl ester | 358.2909 | 358.2872 | 0.0037 | 0.6314 |
| ginkgolic acid II | 375.2981 | 375.2899 | 0.0082 | 0.7162 |
| vitexilactone | 381.2689 | 381.2641 | 0.0048 | 1.1467 |
| lithocholic acid methylester | 391.3249 | 391.3212 | 0.0037 | 0.734 |
| delta-tocotrienol | 397.3173 | 397.3107 | 0.0066 | 0.7689 |
| beta/gamma-tocotrienol | 411.3284 | 411.3263 | 0.0021 | 0.8515 |
| diosgenin | 415.3178 | 415.3212 | −0.0034 | 0.4735 |
| mogroside backbone - 3H2O | 423.36 | 423.3627 | −0.0027 | 2.2727 |
| hecogenin/ruscogenin | 431.319 | 431.3161 | 0.0029 | 1.4648 |
| mogroside backbone - 2H2O | 441.3641 | 441.3733 | −0.0091 | 1.478 |
| ursonic acid/dehydroboswelli | 455.3508 | 455.3525 | −0.0018 | 1.4344 |
| ursolic/oleanolic/boswellic/ | 457.3649 | 457.3682 | −0.0033 | 1.3444 |
| keto boswellic acid/glycyrrh | 471.3522 | 471.3474 | 0.0047 | 0.9253 |
| jujubogenin/bacoside A/caulo | 473.3601 | 473.3631 | −0.003 | 0.8016 |
| Gymnemasaponin II - 2 Glc | 475.3779 | 475.3787 | −0.0008 | 0.6448 |
| 18-glycyrrhetinic acid methy | 485.3705 | 485.3631 | 0.0074 | 0.7159 |
| keto boswellic acid/ganodermol | 487.3884 | 487.3787 | 0.0097 | 0.7089 |
| 3-O-acetyl-9,11-dehydro BA | 497.3697 | 497.3631 | 0.0066 | 1.6139 |
| acetylboswellic acid/ganoder | 499.3804 | 499.3787 | 0.0017 | 1.0594 |
| acetylketoboswellic acid | 513.3677 | 513.358 | 0.0096 | 1.2341 |
| 3-O-acetyl-11-hydroxy boswellic acid | 515.3757 | 515.3737 | 0.002 | 0.7516 |
| adhyperforin | 551.4189 | 551.41 | 0.0088 | 0.2965 |
| echinenone | 551.4189 | 551.4253 | −0.0064 | 0.2965 |
| diatoxanthin | 567.4266 | 567.4202 | 0.0063 | 0.3723 |
| eleutheroside A/sitosterol g | 577.4476 | 577.4469 | 0.0007 | 0.2576 |
| diadinoxanthin | 583.4204 | 583.4151 | 0.0053 | 0.2594 |
| gymnemic acid IV/XIV - GlcA | 589.4148 | 589.4104 | 0.0044 | 0.4188 |
| neoxanthin/violaxanthin | 601.4355 | 601.4257 | 0.0098 | 0.2227 |
| ginsenoside M1 | 609.4286 | 609.4367 | −0.0081 | 0.2736 |

Compounds in Multi Stage SCCO₂ Extraction Stage 3: 40° C. and 90 Bar

6-shogoal, 6 gingerol, and galanolactone were present in this extract in 100, 8.2 and 5.1% relative abundance, respectively. Other shogaols, paradols, gingerols, and gingerdiols were present in the extract. Amino acids, vitamins, fatty acids, saccharides, phenolic acids, phenols, sterols, capsaicins, gymnemagins, boswellic acids, alkaloids, quinones, tumerones, xanthins, saponins and hydrocarbons were also present in this extract. 104 out of 481 (22%) unique chemicals have been directly identified in this extract using the DART TOF-MS. Table 18 shows the compounds identified in the extracts along with their relative abundance. FIG. 9C shows the DART Spectrum of this extract.

TABLE 18
Compounds in Multi Stage SCCO₂ extraction stage 3: 40° C. and 90 bar

| Compound | Meas. | Calc. | Diff(u) | Abund. |
|---|---|---|---|---|
| propyl sulfide | 119.0864 | 119.0894 | −0.0031 | 2.7538 |
| pseudocumene/propynylcyclohexene | 121.1044 | 121.1017 | 0.0027 | 1.925 |
| ornithine | 133.1069 | 133.0977 | 0.0091 | 0.9358 |
| dicyclopentadiene | 133.1069 | 133.1017 | 0.0051 | 0.9358 |
| p-cymene | 135.1199 | 135.1174 | 0.0025 | 2.8125 |
| anisaldehyde/formic acid benzoate | 137.0604 | 137.0602 | 0.0001 | 33.901 |
| trigonelline/vitamin H | 138.0642 | 138.0555 | 0.0086 | 2.5332 |
| octalactone | 143.1089 | 143.1072 | 0.0016 | 1.0144 |
| crotonylbetaine | 145.1009 | 145.1103 | −0.0094 | 1.4016 |
| lysine | 147.1187 | 147.1133 | 0.0053 | 3.8512 |
| nornicotine | 149.1128 | 149.1078 | 0.0049 | 3.0568 |
| 2-acetyl-3-ethylpyrazine | 151.097 | 151.0871 | 0.0098 | 2.2263 |
| norpseudophedrine | 152.0994 | 152.1075 | −0.0081 | 0.309 |
| decadienal/santolina epoxide | 153.1292 | 153.1279 | 0.0013 | 3.2603 |
| pinene oxide | 153.1292 | 153.1279 | 0.0013 | 3.2603 |
| pseudopelletierine | 154.1331 | 154.1232 | 0.0098 | 0.2835 |
| methylcholine | 161.1337 | 161.1416 | −0.0079 | 2.796 |
| N-phenylmorpholine | 164.1108 | 164.1075 | 0.0033 | 1.2637 |
| jasmone | 165.1183 | 165.1279 | −0.0096 | 1.1475 |
| ephedrine | 166.1272 | 166.1232 | 0.0039 | 0.5033 |
| hordenine | 166.1272 | 166.1232 | 0.0039 | 0.5033 |
| pseudoephedrine | 166.1272 | 166.1232 | 0.0039 | 0.5033 |
| camphorquinone | 167.1171 | 167.1072 | 0.0099 | 0.3835 |
| perillic acid | 167.1171 | 167.1072 | 0.0099 | 0.3835 |
| 3-(phenylmethoxy)-1-propanol | 167.1171 | 167.1072 | 0.0099 | 0.3835 |
| 2-undecanal | 169.1613 | 169.1592 | 0.0021 | 0.6851 |
| undec-4-enal | 169.1613 | 169.1592 | 0.0021 | 0.6851 |
| arcaine | 173.1427 | 173.1514 | −0.0087 | 0.5079 |
| cinnamyl acetate | 177.0918 | 177.0915 | 0.0003 | 44.9824 |
| canavanine | 177.0918 | 177.0987 | −0.0069 | 44.9824 |
| coniferaldehyde | 179.0766 | 179.0708 | 0.0058 | 6.2364 |
| methoxycinnamic acid | 179.0766 | 179.0708 | 0.0058 | 6.2364 |
| homophenylalanine | 180.1107 | 180.1024 | 0.0083 | 1.6082 |
| salsolinol | 180.1107 | 180.1024 | 0.0083 | 1.6082 |
| 2(4H)-benzofuranone, 5,6,7,7 | 181.1245 | 181.1228 | 0.0016 | 0.3018 |
| 1,3-di-tert-butylbenzene | 191.1717 | 191.18 | −0.0083 | 2.8853 |
| a-phenylindol | 194.0951 | 194.0969 | −0.0018 | 2.1565 |
| caffeine | 195.0979 | 195.0882 | 0.0097 | 0.5043 |
| zingerone | 195.0979 | 195.1021 | −0.0042 | 0.5043 |
| dihydromyristicin | 195.0979 | 195.1021 | −0.0042 | 0.5043 |
| sedanolide | 195.1419 | 195.1385 | 0.0033 | 0.243 |
| trans-chrysanthenyl acetate | 195.1419 | 195.1385 | 0.0033 | 0.243 |
| (−)-myrtenyl acetate | 195.1419 | 195.1385 | 0.0033 | 0.243 |
| guaiazulene | 199.139 | 199.1487 | −0.0097 | 0.3305 |
| naphthalene, 1,6-dimethyl-4- | 199.139 | 199.1487 | −0.0097 | 0.3305 |
| dehydrocurcumene | 201.1655 | 201.1643 | 0.0012 | 9.9826 |
| curcumene/cuparene/calamenene | 203.1799 | 203.18 | −0.0001 | 47.6359 |
| zingiberene/farnesene/bisabolene | 205.1959 | 205.1956 | 0.0003 | 20.9022 |
| alloaromadendrene/elemene/caryophyllene | 205.1959 | 205.1956 | 0.0003 | 20.9022 |
| cycloheptane, 4-methylene-1- | 205.1959 | 205.1956 | 0.0003 | 20.9022 |
| cedrene | 205.1959 | 205.1956 | 0.0003 | 20.9022 |
| humulene | 205.1959 | 205.1956 | 0.0003 | 20.9022 |
| isocaryophyllene | 205.1959 | 205.1956 | 0.0003 | 20.9022 |
| isolongifolene | 205.1959 | 205.1956 | 0.0003 | 20.9022 |
| longicyclene/longifolene | 205.1959 | 205.1956 | 0.0003 | 20.9022 |
| thujopsen | 205.1959 | 205.1956 | 0.0003 | 20.9022 |
| valencene | 205.1959 | 205.1956 | 0.0003 | 20.9022 |
| copaene | 205.1959 | 205.1956 | 0.0003 | 20.9022 |
| germacrene D | 205.1959 | 205.1956 | 0.0003 | 20.9022 |
| a-cubebene | 205.1959 | 205.1956 | 0.0003 | 20.9022 |
| a-muurolene | 205.1959 | 205.1956 | 0.0003 | 20.9022 |
| trans-a-bergamotene | 205.1959 | 205.1956 | 0.0003 | 20.9022 |
| (−)-a-panasinsen | 205.1959 | 205.1956 | 0.0003 | 20.9022 |
| β-sesquiphellandrene/gualene/guainene | 205.1959 | 205.1956 | 0.0003 | 20.9022 |
| carvylacetate | 209.1522 | 209.1541 | −0.002 | 0.3267 |
| epoxy-a-terpenyl acetate | 213.1512 | 213.149 | 0.0022 | 0.5727 |
| hexylcinnamaldehyde | 217.1597 | 217.1592 | 0.0005 | 7.0005 |
| ar-tumerone | 217.1597 | 217.1592 | 0.0005 | 7.0005 |
| furanoeremophilane | 219.1764 | 219.1749 | 0.0015 | 17.208 |
| nootkatone | 219.1764 | 219.1749 | 0.0014 | 17.208 |
| valerenal | 219.1764 | 219.1749 | 0.0014 | 17.208 |
| curlone | 219.1764 | 219.1749 | 0.0014 | 17.208 |
| turmerone/ar-turmerol/xanthorrhizol | 219.1764 | 219.1749 | 0.0014 | 17.208 |
| caryophellene oxide | 221.1924 | 221.1905 | 0.0019 | 11.7786 |
| bergamotol, Z-a-trans- | 221.1924 | 221.1905 | 0.0019 | 11.7786 |
| spathulenol/9-cedranone/lanceol | 221.1924 | 221.1905 | 0.0019 | 11.7786 |
| 2,2,6-trimethyl-1-(3-methylb | 223.1769 | 223.1698 | 0.0071 | 1.3592 |
| neostigmine | 224.1554 | 224.1525 | 0.0029 | 1.0495 |
| undec-2-ene-8,10-diynoic aci | 232.1731 | 232.1701 | 0.003 | 4.557 |
| costunolide | 233.1626 | 233.1541 | 0.0085 | 6.7463 |
| panthenol | 234.1777 | 234.1705 | 0.0072 | 3.7803 |
| eremophilanlactone | 235.1697 | 235.1698 | −0.0001 | 15.096 |
| 2-octyl benzoate | 235.1697 | 235.1698 | −0.0001 | 15.096 |
| valerenic acid | 235.1697 | 235.1698 | −0.0001 | 15.096 |
| vellerdiol | 237.1843 | 237.1854 | −0.0011 | 9.1157 |
| 3-methyl-but-2-enoic acid, 1 | 237.1843 | 237.1854 | −0.0011 | 9.1157 |
| 2-pentenoic acid, 3-methyl-5 | 237.1843 | 237.1854 | −0.0011 | 9.1157 |
| a-ionyl acetate | 237.1843 | 237.1854 | −0.0011 | 9.1157 |
| isobornyl isovalerate | 239.2061 | 239.2011 | 0.005 | 0.3923 |
| linalyl iso-valerate/4,4,8-t | 239.2061 | 239.2011 | 0.005 | 0.3923 |
| dodec-2,4-diene-8,10-diynoic | 244.1767 | 244.1701 | 0.0066 | 1.5589 |
| 3-hydroxymyristic acid | 245.2074 | 245.2116 | −0.0042 | 2.7277 |
| 6-paradol | 251.166 | 251.1647 | 0.0013 | 4.6942 |
| hydroxyvalerenic acid | 251.166 | 251.1647 | 0.0013 | 4.6942 |
| palmitic acid | 257.2526 | 257.248 | 0.0046 | 4.7618 |
| panaxydol | 261.1919 | 261.1854 | 0.0064 | 9.9765 |
| eserine | 276.175 | 276.1712 | 0.0037 | 17.5269 |
| 6-shogaol | 277.1801 | 277.1803 | −0.0002 | 100 |
| menthyl salicylate | 277.1801 | 277.1803 | −0.0002 | 100 |
| cyclohexanecarboxylic acid | 277.1801 | 277.1803 | −0.0002 | 100 |
| 6-shogaol | 277.1801 | 277.1804 | −0.0002 | 100 |
| stearolic acid | 281.2466 | 281.248 | −0.0015 | 2.2684 |
| linoleic acid | 281.2466 | 281.248 | −0.0015 | 2.2684 |
| 9,12-octadecadienoic acid | 281.2466 | 281.248 | −0.0015 | 2.2684 |
| stearolic acid/linoelaidic acid | 281.2466 | 281.248 | −0.0015 | 2.2684 |
| 9,12-octadecadienoic acid | 281.2466 | 281.248 | −0.0015 | 2.2684 |
| linoleic acid | 281.2466 | 281.248 | −0.0015 | 2.2684 |
| oleic acid | 283.2695 | 283.2637 | 0.0058 | 1.527 |
| elaidic acid | 283.2695 | 283.2637 | 0.0058 | 1.527 |
| petroselaidic acid | 283.2695 | 283.2637 | 0.0058 | 1.527 |
| vaccenic acid | 283.2695 | 283.2637 | 0.0058 | 1.527 |
| vitamin A(retinol) | 287.2393 | 287.2375 | 0.0018 | 5.5193 |
| abieta-8,11,13-trien-18-ol | 287.2393 | 287.2375 | 0.0018 | 5.5193 |
| atropine | 291.1743 | 291.1834 | −0.0092 | 18.3401 |
| N-octyl-B-D-glucopyranoside | 293.1932 | 293.1964 | −0.0032 | 7.0394 |
| 6-gingerol | 295.2001 | 295.1909 | 0.0091 | 8.1706 |
| embelin | 295.2001 | 295.1909 | 0.0091 | 8.1706 |
| 6-gingerol | 295.2001 | 295.1909 | 0.0091 | 8.1706 |
| 6-gingerdiol | 297.2143 | 297.2066 | 0.0077 | 1.8724 |
| 9,12-octadecadienoyl chloride | 299.2166 | 299.2141 | 0.0024 | 1.6362 |
| abietic acid | 303.2414 | 303.2324 | 0.0089 | 8.0316 |
| eicosapentaenoic acid | 303.2414 | 303.2324 | 0.0089 | 8.0316 |
| 8-shogaol | 305.2154 | 305.2117 | 0.0037 | 19.827 |
| 10-paradol | 307.2274 | 307.2273 | 0.0001 | 2.0535 |
| galanolactone/aframodial/galanal | 319.2295 | 319.2273 | 0.0022 | 5.1438 |
| homocapsaicin | 320.2314 | 320.2226 | 0.0088 | 3.8363 |
| homodihydrocapsaicin | 322.2314 | 322.2382 | −0.0068 | 6.8543 |
| hydroxyprogesterone/DHEA acetate | 331.2297 | 331.2273 | 0.0024 | 4.7579 |
| 10-shogaol | 333.2457 | 333.243 | 0.0027 | 23.8262 |
| pregnanetriol | 337.2748 | 337.2742 | 0.0006 | 2.8376 |
| C23H34O2 | 343.262 | 343.2637 | −0.0017 | 2.8267 |
| docosahexenoic acid ethyl ester | 358.2837 | 358.2872 | −0.0035 | 1.4662 |
| incensole oxide acetate | 365.2749 | 365.2692 | 0.0056 | 0.5165 |
| ginkgolic acid II | 375.2863 | 375.2899 | −0.0036 | 1.393 |
| mogroside backbone - 4H2O | 405.3497 | 405.3522 | −0.0025 | 1.5177 |
| benzethonium | 413.3293 | 413.3294 | −0.0002 | 2.9694 |
| jervine | 426.292 | 426.3008 | −0.0088 | 12.5271 |
| hecogenin/ruscogenin | 431.3213 | 431.3161 | 0.0052 | 7.7582 |
| vitamin K1(phytonadione) | 451.3507 | 451.3576 | −0.0069 | 1.8265 |
| ursolic/oleanolic/boswellic acids | 457.3593 | 457.3682 | −0.0088 | 2.1786 |
| ganoderic acid D/M | 469.3348 | 469.3318 | 0.003 | 1.0469 |
| keto boswellic acid/glycyrrhizol | 471.3389 | 471.3474 | −0.0085 | 0.8018 |
| jujubogenin/bacoside A | 473.3657 | 473.3631 | 0.0026 | 0.6163 |
| psychosine | 478.3472 | 478.338 | 0.0091 | 0.8807 |
| 18-glycyrrhetinic acid methyl ester | 485.3631 | 485.3631 | 0 | 1.1833 |
| keto boswellic acid/ganodermol | 487.3815 | 487.3787 | 0.0028 | 1.1373 |
| cholesteryl benzoate | 491.396 | 491.3889 | 0.0071 | 1.0829 |
| 3-O-acetyl-9,11-dehydro BA | 497.3719 | 497.3631 | 0.0088 | 2.2094 |
| 3-O-acetyl-11-hydroxy boswellic acid | 515.3729 | 515.3737 | −0.0008 | 0.8221 |
| vitamin E succinate | 531.4055 | 531.4049 | 0.0006 | 0.0932 |
| adhyperforin | 551.4056 | 551.41 | −0.0045 | 0.2053 |
| lutein/zeaxanthin | 569.4366 | 569.4359 | 0.0007 | 0.1696 |
| ganodermic acids R/S | 571.3966 | 571.3999 | −0.0032 | 1.1024 |

Compounds in Multi Stage SCCO₂ Extraction Stage 4: 40° C. and 100 Bar

6-shogoal, 6 gingerol, and galanolactone were present in this extract in 100, 3.6, and 1.3% relative abundance, respectively. Other shogaols, paradols, gingerols, and gingerdiols were present in the extract. Amino acids, vitamins, fatty acids, saccharides, phenolic acids, phenols, sterols, capsaicins, gymnemagins, quinones, tumerones, alkaloids, xanthenes, and hydrocarbons were also present in this extract. 104 out of 187 (56%) unique chemicals have been directly identified in this extract using the DART TOF-MS. Table 19 shows the compounds identified in the extracts along with their relative abundance. FIG. 9D shows the DART Spectrum of this extract.

TABLE 19
Compounds in Multi Stage SCCO₂ extraction stage 4: 40° C. and 100 bar

| Compound | Meas. | Calc. | Diff(u) | Abund. |
|---|---|---|---|---|
| ethylbenzene | 107.0871 | 107.0861 | 0.001 | 0.8871 |
| propyl sulfide | 119.0832 | 119.0894 | −0.0062 | 2.4747 |
| benzoic acid/salicylaldehyde | 123.0401 | 123.0446 | −0.0045 | 0.3586 |
| guaiacol | 125.0575 | 125.0602 | −0.0027 | 2.2536 |
| methoxyphenol, para- | 125.0575 | 125.0602 | −0.0027 | 2.2536 |
| methylcatechol | 125.0575 | 125.0602 | −0.0027 | 2.2536 |
| salicyl alcohol | 125.0575 | 125.0602 | −0.0027 | 2.2536 |
| 2-methoxyphenol | 125.0575 | 125.0603 | −0.0027 | 2.2536 |
| DL-mevalonic acid lactone | 131.0729 | 131.0708 | 0.0021 | 0.7946 |
| anisaldehyde/formic acid benzoate | 137.0597 | 137.0602 | −0.0005 | 34.7725 |
| trigonelline/vitamin H | 138.0619 | 138.0555 | 0.0064 | 2.2572 |
| 4-hydroxybenzoic acid | 139.0406 | 139.0395 | 0.0011 | 0.315 |
| 2,3-dimethylhydroquinone | 139.075 | 139.0759 | −0.0009 | 0.3755 |
| 4-ethylquinol | 139.075 | 139.0759 | −0.0009 | 0.3755 |
| tyrosol | 139.075 | 139.0759 | −0.0009 | 0.3755 |
| 2-furanmethanol, tetrahydro- | 145.085 | 145.0864 | −0.0014 | 1.0097 |
| lysine | 147.1166 | 147.1133 | 0.0033 | 1.2416 |
| dihydroxyacetophenone | 153.0546 | 153.0551 | −0.0005 | 5.478 |
| methyl cinnamic acid | 163.0828 | 163.0759 | 0.0069 | 2.6722 |
| safrole | 163.0828 | 163.0759 | 0.0069 | 2.6722 |
| methoxycinnamaldehyde | 163.0828 | 163.0759 | 0.0069 | 2.6722 |
| 4-hydroxyphenyl-2-butanone | 165.1001 | 165.0915 | 0.0086 | 0.9972 |
| acetic acid phenethyl ester | 165.1001 | 165.0915 | 0.0086 | 0.9972 |
| butyl-p-quinone; 2-tert- | 165.1001 | 165.0915 | 0.0086 | 0.9972 |
| eugenol | 165.1001 | 165.0915 | 0.0086 | 0.9972 |
| isoeugenol | 165.1001 | 165.0915 | 0.0086 | 0.9972 |
| phenylacetic acid ethylester | 165.1001 | 165.0915 | 0.0086 | 0.9972 |
| eugenol | 165.1001 | 165.0916 | 0.0085 | 0.9972 |
| camphorquinone | 167.1051 | 167.1072 | −0.0022 | 0.9212 |
| perillic acid | 167.1051 | 167.1072 | −0.0022 | 0.9212 |
| 3-(phenylmethoxy)-1-propanol | 167.1051 | 167.1072 | −0.0022 | 0.9212 |
| vanillic acid/homogentisic acid | 169.0504 | 169.0501 | 0.0003 | 2.3965 |
| theanine | 175.1004 | 175.1083 | −0.0079 | 7.8317 |
| cinnamyl acetate | 177.0926 | 177.0915 | 0.0011 | 55.0139 |
| canavanine | 177.0926 | 177.0987 | −0.0061 | 55.0139 |
| coniferaldehyde | 179.0797 | 179.0708 | 0.0089 | 8.5777 |
| methoxycinnamic acid | 179.0797 | 179.0708 | 0.0089 | 8.5777 |
| D-mannosamine | 180.088 | 180.0872 | 0.0008 | 0.6063 |
| galacgtosamine | 180.088 | 180.0872 | 0.0008 | 0.6063 |
| glucosamine | 180.088 | 180.0872 | 0.0008 | 0.6063 |
| 10-hydroxy-2-decenoic acid | 187.1363 | 187.1334 | 0.0029 | 0.5556 |
| 1,2-isopropylidene-D-xylofur | 191.1009 | 191.0919 | 0.0089 | 2.7066 |
| 2,6-diaminopimelic acid | 191.1009 | 191.1032 | −0.0023 | 2.7066 |
| ligustilide | 191.1009 | 191.1072 | −0.0063 | 2.7066 |
| dehydrocurcumene | 201.1625 | 201.1643 | −0.0018 | 2.4567 |
| curcumene/cuparene/calamenene | 203.1772 | 203.18 | −0.0029 | 9.5511 |
| valeric acid phenylethylester | 207.1474 | 207.1385 | 0.0089 | 2.2791 |
| neocuprone | 209.1171 | 209.1078 | 0.0093 | 1.0454 |
| asarone | 209.1171 | 209.1177 | −0.0006 | 1.0454 |
| 7-tetradecenal, (Z)- | 211.203 | 211.2062 | −0.0032 | 1.0387 |
| E-11,13-tetradecadien-1-ol | 211.203 | 211.2062 | −0.0032 | 1.0387 |
| hexylcinnamaldehyde | 217.1557 | 217.1592 | −0.0035 | 0.9901 |
| ar-tumerone | 217.1557 | 217.1592 | −0.0035 | 0.9901 |
| benzene, 1-(3-cyclopentylpro | 217.2026 | 217.1956 | 0.007 | 0.3825 |
| furanoeremophilane | 219.1738 | 219.1749 | −0.0011 | 9.1316 |
| nootkatone | 219.1738 | 219.1749 | −0.0011 | 9.1316 |
| valerenal | 219.1738 | 219.1749 | −0.0011 | 9.1316 |
| xanthorrhizol | 219.1738 | 219.1749 | −0.0011 | 9.1316 |
| curlone | 219.1738 | 219.1749 | −0.0011 | 9.1316 |
| turmerone/ar-turmerol | 219.1738 | 219.1749 | −0.0011 | 9.1316 |
| bergamotol/caryophyllene oxide | 221.1899 | 221.1905 | −0.0006 | 6.6399 |
| spathulenol/9-cedranone/lanceol | 221.1899 | 221.1905 | −0.0006 | 6.6399 |
| propanoic acid, 3-hdroxy-3-p | 223.1415 | 223.1334 | 0.0081 | 4.6205 |
| costunolide | 233.155 | 233.1541 | 0.0009 | 2.0051 |
| eremophilanlactone | 235.1643 | 235.1698 | −0.0055 | 5.173 |
| 2-octyl benzoate | 235.1643 | 235.1698 | −0.0056 | 5.173 |
| valerenic acid | 235.1643 | 235.1698 | −0.0056 | 5.173 |
| vellerdiol | 237.1792 | 237.1854 | −0.0062 | 4.8974 |
| 3-methyl-but-2-enoic acid, 1 | 237.1792 | 237.1854 | −0.0062 | 4.8974 |
| 2-pentenoic acid, 3-methyl-5 | 237.1792 | 237.1854 | −0.0062 | 4.8974 |
| a-ionyl acetate | 237.1792 | 237.1854 | −0.0062 | 4.8974 |
| atractylenolide III | 249.1452 | 249.149 | −0.0039 | 2.3361 |
| parthenolide | 249.1452 | 249.149 | −0.0039 | 2.3361 |
| 6-paradol | 251.1637 | 251.1647 | −0.001 | 1.494 |
| hydroxyvalerenic acid | 251.1637 | 251.1647 | −0.0011 | 1.494 |
| palmitic acid | 257.2486 | 257.248 | 0.0005 | 3.7679 |
| panaxydol/octanoic acid, 3-p | 261.1888 | 261.1854 | 0.0034 | 3.5078 |
| oxymatrine | 265.2012 | 265.1916 | 0.0095 | 0.4641 |
| honokiol | 267.1457 | 267.1385 | 0.0071 | 3.0372 |
| magnolol | 267.1457 | 267.1385 | 0.0071 | 3.0372 |
| 3,6-epoxy-1-(4-hydroxy-3-met | 275.1666 | 275.1647 | 0.0018 | 5.1176 |
| podocarpic acid | 275.1666 | 275.1647 | 0.0018 | 5.1176 |
| eserine | 276.172 | 276.1712 | 0.0008 | 3.6944 |
| 6-shogaol | 277.1785 | 277.1803 | −0.0018 | 100 |
| menthyl salicylate | 277.1785 | 277.1803 | −0.0018 | 100 |
| cyclohexanecarboxylic acid, | 277.1785 | 277.1803 | −0.0018 | 100 |
| 6-shogaol | 277.1785 | 277.1804 | −0.0019 | 100 |
| stearolic acid | 281.2485 | 281.248 | 0.0005 | 2.2267 |
| linoleic acid | 281.2485 | 281.248 | 0.0005 | 2.2267 |
| 9,12-octadecadienoic acid | 281.2485 | 281.248 | 0.0005 | 2.2267 |
| stearolic acid/linoelaidic acid | 281.2485 | 281.248 | 0.0005 | 2.2267 |
| 16-oxokahweol | 283.1751 | 283.1698 | 0.0053 | 55.355 |
| miltirone | 283.1751 | 283.1698 | 0.0053 | 55.355 |
| 16-oxocafestol | 285.1829 | 285.1854 | −0.0025 | 2.5489 |
| atropine | 291.1883 | 291.1834 | 0.0049 | 5.7022 |
| 7-shogaol | 291.1883 | 291.196 | −0.0077 | 5.7022 |
| N-octyl-B-D-glucopyranoside | 293.1886 | 293.1964 | −0.0078 | 2.8425 |
| 6-gingerol | 295.195 | 295.1909 | 0.004 | 3.5935 |
| embelin | 295.195 | 295.1909 | 0.004 | 3.5935 |
| 6-gingerol | 295.195 | 295.1909 | 0.004 | 3.5935 |
| retinoic acid | 301.2144 | 301.2167 | −0.0023 | 1.4143 |
| C20H28O2 | 301.2144 | 301.2167 | −0.0023 | 1.4143 |
| abietic acid | 303.2259 | 303.2324 | −0.0065 | 2.2524 |
| eicosapentaenoic acid | 303.2259 | 303.2324 | −0.0065 | 2.2524 |
| 8-shogaol | 305.2138 | 305.2117 | 0.0021 | 15.9379 |
| sarpagine | 311.1813 | 311.1759 | 0.0054 | 4.7307 |
| galanolactone/aframodial/galanal | 319.2325 | 319.2273 | 0.0052 | 1.3035 |
| 2-chloroethyl palmitate | 319.2325 | 319.2404 | −0.0079 | 1.3035 |
| homocapsaicin | 320.2231 | 320.2226 | 0.0005 | 0.5161 |
| homodihydrocapsaicin | 322.2327 | 322.2382 | −0.0055 | 1.2803 |
| 8-gingerol/rapanone | 323.2204 | 323.2222 | −0.0018 | 1.8401 |
| 8-gingerdiol | 325.2307 | 325.2379 | −0.0072 | 1.5607 |
| ajmaline | 327.202 | 327.2072 | −0.0052 | 55.576 |
| 10-shogaol | 333.2438 | 333.243 | 0.0008 | 14.8363 |
| pregnanetriol | 337.2678 | 337.2742 | −0.0064 | 0.4874 |
| menisperine | 341.2002 | 341.1985 | 0.0017 | 0.9965 |
| 10-gingerdione | 349.2433 | 349.2379 | 0.0054 | 0.2881 |
| tetrahydrocorticosterone | 351.2622 | 351.2535 | 0.0087 | 1.5133 |
| corynanthine/vincamine/yohimbine | 355.198 | 355.2021 | −0.0041 | 3.6252 |
| tamoxifen | 372.2314 | 372.2327 | −0.0014 | 6.8502 |
| mitragynine picrate | 399.2306 | 399.2284 | 0.0022 | 2.8416 |

Compounds in Multi Stage SCCO₂ Extraction Stage 5: 40° C. and 120 Bar

6-shogoal, 6 gingerol, and galanolactone were present in this extract in 22.2, 3.2 and 2.8% relative abundance, respectively. Other shogaols, paradols, gingerols, and gingerdiols were present in the extract. Amino acids, vitamins, fatty acids, saccharides, phenolic acids, phenols, sterols, capsaicins, gymnemagins, quinones, tumerones, alkaloids, xanthenes, and hydrocarbons were also present in this extract. 119 out of 842 (14%) unique chemicals have been directly identified in this extract using the DART TOF-MS. Table 20 shows the compounds identified in the extracts along with their relative abundance. FIG. 9E shows the DART Spectrum of this extract.

TABLE 20
Compounds in Multi Stage SCCO₂ extraction stage 5: 40° C. and 120 bar

| Compound | Meas. | Calc. | Diff(u) | Abund. |
|---|---|---|---|---|
| p-cymene | 135.1173 | 135.1174 | −0.0001 | 0.897 |
| octalactone | 143.1077 | 143.1072 | 0.0005 | 1.6015 |
| carvacrol/thymol/cymenol | 151.1116 | 151.1123 | −0.0007 | 0.9128 |
| norpseudophedrine | 152.1167 | 152.1075 | 0.0091 | 0.2756 |
| dihydroxyacetophenone/anisic acid | 153.0513 | 153.0551 | −0.0038 | 0.5088 |
| decadienal/santolina epoxide | 153.1254 | 153.1279 | −0.0025 | 11.4926 |
| pinene oxide | 153.1254 | 153.1279 | −0.0025 | 11.4926 |
| pseudopelletierine | 154.1287 | 154.1232 | 0.0054 | 0.9998 |
| N-phenylmorpholine | 164.1006 | 164.1075 | −0.0069 | 8.1613 |
| 4-hydroxyphenyl-2-butanone | 165.0992 | 165.0915 | 0.0077 | 0.7885 |
| acetic acid phenethyl ester | 165.0992 | 165.0915 | 0.0077 | 0.7885 |
| butyl-p-quinone; 2-tert- | 165.0992 | 165.0915 | 0.0077 | 0.7885 |
| eugenol | 165.0992 | 165.0915 | 0.0077 | 0.7885 |
| isoeugenol | 165.0992 | 165.0915 | 0.0077 | 0.7885 |
| phenylacetic acid ethylester | 165.0992 | 165.0915 | 0.0077 | 0.7885 |
| eugenol | 165.0992 | 165.0916 | 0.0076 | 0.7885 |
| camphorquinone | 167.1053 | 167.1072 | −0.0019 | 1.9249 |
| perillic acid | 167.1053 | 167.1072 | −0.0019 | 1.9249 |
| 3-(phenylmethoxy)-1-propanol | 167.1053 | 167.1072 | −0.0019 | 1.9249 |
| (3Z)-3-hexenyl-2-butenoate | 169.1198 | 169.1228 | −0.0031 | 0.727 |
| chrysanthemolactone | 169.1198 | 169.1228 | −0.0031 | 0.727 |
| a-Limonene diepoxide | 169.1198 | 169.1228 | −0.0031 | 0.727 |
| 5-bromouracil | 178.9516 | 178.9456 | 0.006 | 0.0256 |
| homophenylalanine | 180.1017 | 180.1024 | −0.0007 | 1.7704 |
| salsolinol | 180.1017 | 180.1024 | −0.0007 | 1.7704 |
| acetoveratrone | 181.0899 | 181.0864 | 0.0034 | 0.692 |
| coniferyl alcohol | 181.0899 | 181.0864 | 0.0034 | 0.692 |
| 4-((1E)-3-hydroxy-1-propenyl | 181.0899 | 181.0864 | 0.0034 | 0.692 |
| carvacryl acetate | 193.1291 | 193.1228 | 0.0063 | 2.1864 |
| sedanolide | 195.139 | 195.1385 | 0.0005 | 0.3114 |
| trans-chrysanthenyl acetate | 195.139 | 195.1385 | 0.0005 | 0.3114 |
| (−)-myrtenyl acetate | 195.139 | 195.1385 | 0.0005 | 0.3114 |
| D-glucosaminic acid | 196.0917 | 196.0821 | 0.0096 | 0.7094 |
| DL-a-methyl-m-tyrosine | 196.0917 | 196.0973 | −0.0056 | 0.7094 |
| guaiazulene | 199.1395 | 199.1487 | −0.0092 | 0.4745 |
| naphthalene, 1,6-dimethyl-4- | 199.1395 | 199.1487 | −0.0092 | 0.4745 |
| dehydrocurcumene | 201.1648 | 201.1643 | 0.0005 | 11.0642 |
| curcumene/cuparene/calamenene | 203.1788 | 203.18 | −0.0013 | 100 |
| zingiberene/farnesene/bisabolene | 205.1944 | 205.1956 | −0.0012 | 79.5541 |
| alloaromadendrene/elemene | 205.1944 | 205.1956 | −0.0012 | 79.5541 |
| cycloheptane, 4-methylene-1- | 205.1944 | 205.1956 | −0.0012 | 79.5541 |
| aromadendrene/cedrene/humulene | 205.1944 | 205.1956 | −0.0012 | 79.5541 |
| isocaryophyllene/isolongifolene | 205.1944 | 205.1956 | −0.0012 | 79.5541 |
| longicyclene/longifolene | 205.1944 | 205.1956 | −0.0012 | 79.5541 |
| thujopsen/valencene/gualene | 205.1944 | 205.1956 | −0.0012 | 79.5541 |
| copaene/guainene/germacrene D | 205.1944 | 205.1956 | −0.0012 | 79.5541 |
| cubebene/muurolene/bergamotene | 205.1944 | 205.1956 | −0.0012 | 79.5541 |
| panasinsen/sesquiphellandrene | 205.1944 | 205.1956 | −0.0012 | 79.5541 |
| xanthurenic acid | 206.0372 | 206.0453 | −0.0081 | 0.1215 |
| 3,5-bis(1,1-dimethylethyl)-p | 207.181 | 207.1749 | 0.0061 | 1.267 |
| carvylacetate | 209.15 | 209.1541 | −0.0041 | 0.8376 |
| hexylcinnamaldehyde | 217.1589 | 217.1592 | −0.0003 | 7.39 |
| ar-tumerone | 217.1589 | 217.1592 | −0.0003 | 7.39 |
| furanoeremophilane | 219.1735 | 219.1749 | −0.0014 | 38.826 |
| nootkatone | 219.1735 | 219.1749 | −0.0014 | 38.826 |
| valerenal | 219.1735 | 219.1749 | −0.0014 | 38.826 |
| xanthorrhizol | 219.1735 | 219.1749 | −0.0014 | 38.826 |
| curlone | 219.1735 | 219.1749 | −0.0014 | 38.826 |
| turmerone/ar-turmerol | 219.1735 | 219.1749 | −0.0014 | 38.826 |
| caryophellene oxide | 221.1893 | 221.1905 | −0.0012 | 35.9348 |
| β-caryophyllene epoxide | 221.1893 | 221.1905 | −0.0012 | 35.9348 |
| spathulenol | 221.1893 | 221.1905 | −0.0012 | 35.9348 |
| caryophyllene oxide | 221.1893 | 221.1905 | −0.0012 | 35.9348 |
| bergamotol/9-cedranone/lanceol | 221.1893 | 221.1905 | −0.0012 | 35.9348 |
| neostigmine | 224.1555 | 224.1525 | 0.003 | 1.4957 |
| undec-2-ene-8,10-diynoic acid | 232.1647 | 232.1701 | −0.0054 | 3.8093 |
| costunolide | 233.1568 | 233.1541 | 0.0026 | 7.2976 |
| panthenol | 234.1644 | 234.1705 | −0.0061 | 5.1587 |
| eremophilanlactone | 235.1691 | 235.1698 | −0.0007 | 34.4972 |
| 2-octyl benzoate | 235.1691 | 235.1698 | −0.0007 | 34.4972 |
| valerenic acid | 235.1691 | 235.1698 | −0.0007 | 34.4972 |
| vellerdiol | 237.1849 | 237.1854 | −0.0005 | 21.4749 |
| a-ionyl acetate | 237.1849 | 237.1854 | −0.0005 | 21.4749 |
| osthole | 245.1141 | 245.1177 | −0.0036 | 0.2091 |
| santonin | 247.1343 | 247.1334 | 0.0009 | 0.7518 |
| atractylenolide III | 249.1541 | 249.149 | 0.0051 | 4.2187 |
| parthenolide | 249.1541 | 249.149 | 0.0051 | 4.2187 |
| 6-paradol | 251.1652 | 251.1647 | 0.0005 | 7.6119 |
| hydroxyvalerenic acid | 251.1652 | 251.1647 | 0.0004 | 7.6119 |
| panaxydol/octanoic acid, 3-p | 261.1854 | 261.1854 | 0 | 2.3991 |
| C20H32/biformene/kaur-16-ene | 273.2592 | 273.2582 | 0.001 | 4.4012 |
| 3,6-epoxy-1-(4-hydroxy-3-met | 275.1682 | 275.1647 | 0.0035 | 4.3966 |
| podocarpic acid | 275.1682 | 275.1647 | 0.0035 | 4.3966 |
| eserine | 276.1741 | 276.1712 | 0.0029 | 15.4662 |
| 6-shogaol | 277.1801 | 277.1803 | −0.0003 | 22.1905 |
| menthyl salicylate | 277.1801 | 277.1803 | −0.0003 | 22.1905 |
| cyclohexanecarboxylic acid, | 277.1801 | 277.1803 | −0.0003 | 22.1905 |
| vitamin A(retinol) | 287.2363 | 287.2375 | −0.0012 | 6.4724 |
| abieta-8,11,13-trien-18-ol | 287.2363 | 287.2375 | −0.0012 | 6.4724 |
| 6-dehydrogingerdione | 291.1663 | 291.1596 | 0.0066 | 10.2183 |
| 6-gingerdione | 293.1844 | 293.1753 | 0.0091 | 5.3638 |
| acetoxyvalerenic acid | 293.1844 | 293.1753 | 0.009 | 5.3638 |
| cinchonidine/cinchonine | 295.188 | 295.181 | 0.0069 | 3.1967 |
| eburnamonine | 295.188 | 295.181 | 0.0069 | 3.1967 |
| 6-gingerol | 295.188 | 295.1909 | −0.003 | 3.1967 |
| embelin | 295.188 | 295.1909 | −0.003 | 3.1967 |
| 6-gingerdiol | 297.2101 | 297.2066 | 0.0035 | 3.0747 |
| 9,12-octadecadienoyl chloride | 299.2062 | 299.2141 | −0.0079 | 1.3243 |
| retinoic acid | 301.2238 | 301.2167 | 0.0071 | 3.2831 |
| C20H28O2 | 301.2238 | 301.2167 | 0.0071 | 3.2831 |
| abietic acid | 303.2327 | 303.2324 | 0.0003 | 4.7988 |
| eicosapentaenoic acid | 303.2327 | 303.2324 | 0.0003 | 4.7988 |
| aleuritic acid | 305.223 | 305.2328 | −0.0098 | 3.7036 |
| 10-paradol | 307.2205 | 307.2273 | −0.0068 | 1.3189 |
| galanolactone/aframodial/galanal | 319.2241 | 319.2273 | −0.0032 | 2.7756 |
| homocapsaicin | 320.2326 | 320.2226 | 0.01 | 2.5421 |
| incensole oxide | 323.2523 | 323.2586 | −0.0063 | 2.3701 |
| hydroxyprogesterone/DHEA acetate | 331.2294 | 331.2273 | 0.0021 | 2.1075 |
| 10-shogaol | 333.2347 | 333.243 | −0.0083 | 2.5172 |
| deoxy-andrographolide | 335.2318 | 335.2222 | 0.0096 | 2.6911 |
| C23H34O2 | 343.2636 | 343.2637 | −0.0001 | 2.6758 |
| 10-dehydrogingerdione | 347.2278 | 347.2222 | 0.0056 | 3.1156 |
| calycanthine | 347.2278 | 347.2235 | 0.0043 | 3.1156 |
| tetrahydrocorticosterone | 351.2487 | 351.2535 | −0.0048 | 1.4769 |
| 10-gingerdiol | 353.2605 | 353.2692 | −0.0087 | 1.7901 |
| 17B-estradiol-17-valerate | 357.2332 | 357.2429 | −0.0098 | 3.9169 |
| 12-shogaol | 361.2761 | 361.2743 | 0.0018 | 1.5684 |
| cinobufotalin | 363.2639 | 363.2688 | −0.0049 | 1.308 |
| incensole oxide acetate | 365.2719 | 365.2692 | 0.0026 | 1.2216 |
| vitexilactone | 381.2714 | 381.2641 | 0.0073 | 1.7585 |
| deoxycholic acid | 393.2935 | 393.3005 | −0.007 | 0.8399 |
| spironolactone | 417.2193 | 417.2099 | 0.0094 | 4.7545 |
| schisandrin A/lucidone A | 417.2193 | 417.2277 | −0.0084 | 4.7545 |
| neoruscogenin | 429.302 | 429.3005 | 0.0015 | 2.5616 |
| hecogenin/ruscogenin | 431.3153 | 431.3161 | −0.0008 | 11.05 |
| 4-methylumbelliferyl elaidate | 441.299 | 441.3005 | −0.0015 | 5.0687 |
| vitamin K2(menaquinone) | 445.3172 | 445.3106 | 0.0066 | 2.7364 |
| ganoderic acid D/M | 469.3301 | 469.3318 | −0.0018 | 2.336 |
| hovenolactone/trevoagenin D | 489.3536 | 489.358 | −0.0044 | 2.008 |
| gymnemagenin | 492.3476 | 492.3451 | 0.0024 | 2.6385 |
| 3-O-acetyl-9,11-dehydro BA | 497.361 | 497.3631 | −0.0021 | 4.433 |
| acetylketoboswellic acid | 513.3641 | 513.358 | 0.006 | 2.9936 |
| hyperforin | 537.3921 | 537.3944 | −0.0023 | 1.5347 |
| adhyperforin | 551.4061 | 551.41 | −0.004 | 1.7794 |
| alloxanthin | 565.4012 | 565.4046 | −0.0033 | 1.1733 |
| canthaxanthin | 565.4012 | 565.4046 | −0.0033 | 1.1733 |
| carbenoxolone | 571.3727 | 571.3635 | 0.0092 | 3.395 |
| diadinoxanthin | 583.4117 | 583.4151 | −0.0034 | 0.9621 |
| neoxanthin/violaxanthin | 601.429 | 601.4257 | 0.0033 | 0.9094 |
| ginsenoside Rh1 | 627.4395 | 627.4472 | −0.0077 | 1.5309 |
| mogroside V - 4glc | 639.444 | 639.4472 | −0.0032 | 0.5528 |
| gymnemic acid XV - GlcA | 673.4751 | 673.468 | 0.0071 | 0.4554 |

### Example 3

### Example of Step 1C (FIG. 2)

### Fractional SCCO₂ Separation of Ginger Essential Oil Chemical Constituents

SCCO₂ fractional separation was carried out on a proprietary HerbalScience designed 1 L laboratory scale SFE equipment. The apparatus consists essentially of solvent delivery, extraction and phase separation sections. Carbon dioxide is the solvent in the present work. It is contacted with the bed of solid feedstock in the extraction section and the amount of solute dissolved in it during the operation is determined in the phase separation section. The carbon dioxide entering the extraction section is brought to the pressure and temperature, at which the extraction is to be carried out. In the solvent delivery section, the desired pressure is reached by compressing liquid carbon dioxide from the supply cylinders using a compressed air driven pump, fine control being achieved by using a back pressure regulator and compressed air used to activate the pump. The required temperature is reached by passing the compressed carbon dioxide stream through a pre-heating element. Upon reaching the desired pressure and temperature, the CO₂ stream enters the pressure vessel used for the extraction. The temperature of the extraction vessel was controlled using heating bands that are controlled by a temperature controller. When carrying out an extraction, carbon dioxide from the solvent delivery section is fed continually to the foot of the bed of feedstock, passes up through the bed and exits at the top bearing solute material from the bed in solution. Then the carbon dioxide stream passes to the separation section where the pressure is reduced and the solute is precipitated in a series of separators. The solute free carbon dioxide leaving these collectors is vented from the laboratory via a flow meter. Pressure reduction of the carbon dioxide stream, initially at the extraction pressure, is achieved by passing it through a pressure-reducing valve. This valve provides an intermediate pressure reduction stage. Because the reduction in pressure is accompanied by pronounced cooling, the pressure reduction valve system is enclosed with an electrical heating tape that is used to warm both the valve and the piping leading into the middle pressure separator 1. The temperature of heating tape is adjusted to be high enough to ensure that dry ice formation (and hence unsteady flow) is avoided.

The equipment described above was used to perform extraction experiment from the herb Ginger, which was ground into powder with particle size above 100 μm and placed inside the extractor vessel. After 300 g of feedstock packing the bed, a plug of glass wool was added on the top to prevent flotation of fine particles from the bed. Leak testing was performed on the apparatus at various intervals or when the apparatus underwent a configuration change. Leak testing was discontinued when the apparatus held the working pressures for a sufficient period of time. Having prepared the equipment and having waited for all temperatures to reach steady state values, the extraction was started. A constant stream of carbon dioxide 40 (g/min) was passed through sample, at constant pressure of 300 bar and temperature of 40° C. The condition of separator 1 (SP1) was set at 60° C. and 100 bar; the condition of separator 2 (SP2) was set at 45° C. and 45 bar. After 3 hours processing (solvent to feed ratio=24), isolate two separator valves and shut down heating power. The extracts in both separators were collected for calculation yield, HPLC (Table 21) and GC-MS (Table 22) analysis. The feedstock and residue was extracted by methanol for active component analysis.

TABLE 21
HPLC analysis results on feedstock, SFE residue and extracts
in two separators.

| | | | | | | 6-G | | |
|---|---|---|---|---|---|---|---|---|
| | Purity (%) | | | | | ratio | Yield (%) | |
| Sample | 6-G | 8-G | 10-G | 6-S | total | (%) | total | gingerol |
| Feedstock | 7.9 | 2.0 | 4.7 | 2.5 | 17.0 | 46.2 | 8.6 | 1.46 |
| Residue | 2.36 | 0.25 | 0.71 | 0.06 | 3.4 | 69.8 | 6.6 | 0.22 |
| SP1 | 33.2 | 7.7 | 19.3 | 5.4 | 65.5 | 50.7 | 2.5 | 1.66 |
| SP2 | 19.6 | 3.5 | 8.8 | 7.4 | 39.3 | 49.8 | 0.8 | 0.33 |

TABLE 22
GC-MS analysis results on extracts in two separators.

| | |
|---|---|
| SP1 | SP2 |

| | |
|---|---|
| | Peak area |
| Peak No. | percentage (%) |

| | | |
|---|---|---|
| 1 | 0.14 | |
| 2 | | |
| 3 | | |
| 4 | | |
| 5 | 4.8 | 0.73 |
| 6 | | 0.19 |
| 7 | | 0.18 |
| 8 | 0.12 | 1.06 |
| 9 | 0.13 | 0.52 |
| 10 | 10.73 | 1.45 |
| 11 | 0.2 | 0.42 |
| 12 | 0.48 | 0.26 |
| 13 | 0.13 | 0.36 |
| 14 | 0.41 | 0.3 |
| 15 | 0.1 | 0.59 |
| 16 | | 0.35 |
| 17 | | |
| 18 | | |
| 19 | 0.05 | |
| 20 | | 0.46 |
| 21 | 3.88 | 21.59 |
| 22 | 5.42 | 17.48 |
| 23 | 0.4 | 2.64 |
| 24 | 2.33 | 12.42 |
| 25 | | 0.38 |
| 26 | | 0.15 |
| 27 | | 0.36 |
| 28 | 3.3 | 15.38 |
| 29 | | 0.28 |
| 30 | | 0.27 |
| 31 | | 0.89 |
| 32 | 1.17 | |
| 33 | | 0.14 |
| 34 | | 0.45 |
| 35 | | |
| 36 | 0.1 | 0.66 |
| 37 | | 0.36 |
| 38 | | 0.46 |
| 39 | 33.56 | 5.95 |
| 40 | 0.23 | 0.63 |
| 41 | | 0.41 |
| 42 | 0.44 | 0.28 |
| 43 | 1.2 | 0.21 |
| 44 | 0.56 | 1 |
| 45 | | 0.11 |
| 46 | 2.55 | 1.53 |
| 47 | 0.29 | 0.17 |
| 48 | 2.04 | 0.93 |
| 49 | 0.2 | |
| 50 | 1.03 | 0.22 |
| 51 | 0.39 | 0.12 |
| 52 | 0.24 | |
| 53 | 0.05 | |
| 54 | 0.08 | 0.39 |
| 55 | | |
| 56 | | |
| 57 | | |
| 58 | | |
| 59 | | |
| 60 | | |
| 61 | 0.07 | 0.15 |
| 62 | 0.24 | 0.15 |
| 63 | 0.41 | 0.11 |
| 64 | 0.25 | 0.09 |
| 65 | 14.1 | 3.01 |
| 66 | 2.15 | |
| 67 | | 0.3 |
| 68 | 1.93 | 0.2 |
| 69 | 0.22 | |
| 70 | 2.37 | |
| Total | 98.35 | 96.74 |
| Monoterpene | 0.38 | 2.13 |
| Sesquiterpene | 15.58 | 74.17 |
| Oxygenated Sesquiterpene | 8.24 | 5.16 |
| Gingerol | 54.36 | 9.25 |

Compounds in Fractional SCCO₂ Separation of Ginger Essential Oil: Separator 1

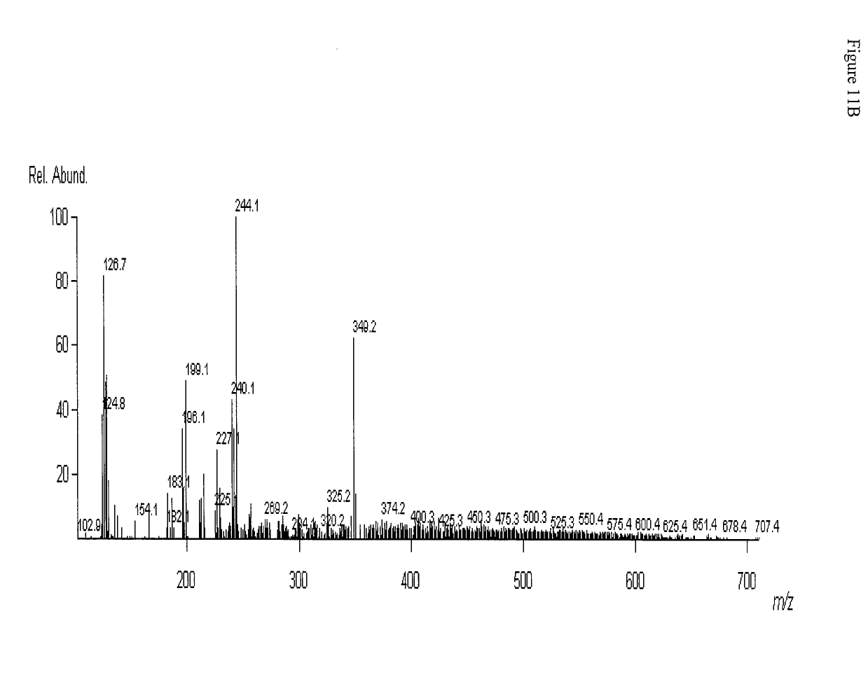

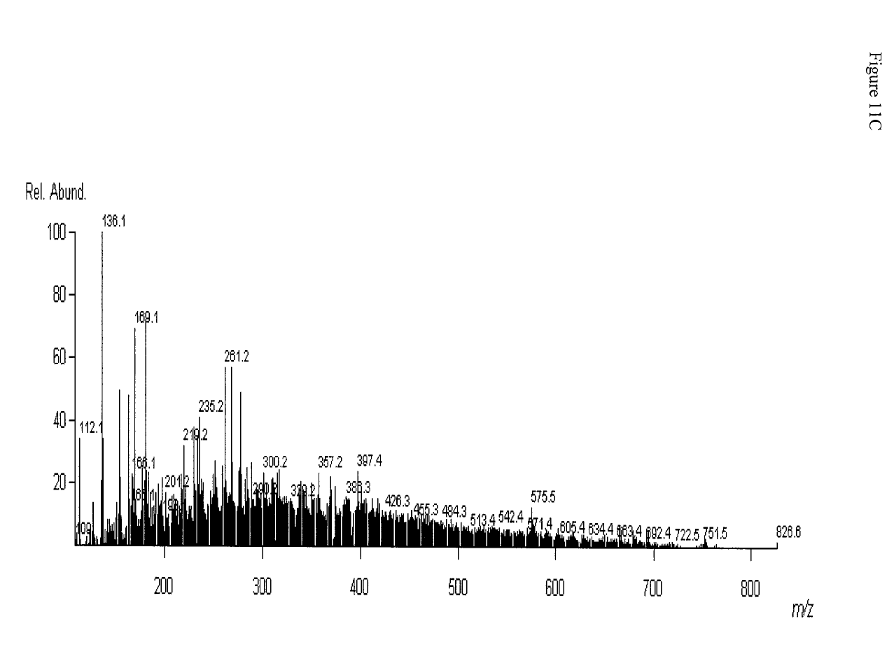

6-shogoal, 6 gingerol, and galanolactone were present in this extract in 97, 6.5 and 6.0% relative abundance, respectively. Other shogaols, paradols, gingerols, and gingerdiols were present in the extract. Amino acids, vitamins, fatty acids, saccharides, phenolic acids, phenols, sterols, capsaicins, gymnemagins, quinones, tumerones, ganoderols, xanthines, boswellic acids, and hydrocarbons were also present in this extract. 121 out of 524 (23%) unique chemicals have been directly identified in this extract using the DART TOF-MS. Table 23 shows the compounds identified in the extracts along with their relative abundance. FIG. 10A shows the DART Spectrum of this extract.

TABLE 23
Compounds in Fractional SCCO₂ separation of ginger essential oil: separator 1

| Compound | Meas. | Calc. | Diff(u) | Abund. |
|---|---|---|---|---|
| propyl sulfide | 119.0853 | 119.0894 | −0.0041 | 21.696 |
| pseudocumene/propynylcyclohexene | 121.1023 | 121.1017 | 0.0006 | 7.3109 |
| ornithine | 133.1004 | 133.0977 | 0.0027 | 8.2394 |
| dicyclopentadiene | 133.1004 | 133.1017 | −0.0013 | 8.2394 |
| p-cymene | 135.1192 | 135.1174 | 0.0018 | 6.9934 |
| anisaldehyde/formic acid benzoate | 137.0603 | 137.0602 | 0 | 36.304 |
| octalactone | 143.1064 | 143.1072 | −0.0009 | 0.8707 |
| lysine | 147.1174 | 147.1133 | 0.0041 | 6.5343 |
| nornicotine | 149.1068 | 149.1078 | −0.001 | 19.624 |
| 2-methoxy-4-vinylphenol | 151.0728 | 151.0759 | −0.0032 | 2.2009 |
| benzoic acid ethyl ester | 151.0728 | 151.0759 | −0.0032 | 2.2009 |
| cresyl acetate | 151.0728 | 151.0759 | −0.0032 | 2.2009 |
| hydrocinnamic acid | 151.0728 | 151.0759 | −0.0032 | 2.2009 |
| dihydroxyacetophenone | 153.052 | 153.0551 | −0.0032 | 1.157 |
| decadienal/santolina epoxide | 153.1285 | 153.1279 | 0.0006 | 5.0321 |
| pinene oxide | 153.1285 | 153.1279 | 0.0006 | 5.0321 |
| pseudopelletierine | 154.1317 | 154.1232 | 0.0084 | 0.3255 |
| methylcholine | 161.133 | 161.1416 | −0.0087 | 4.873 |
| methyl cinnamic acid | 163.0778 | 163.0759 | 0.0018 | 14.143 |
| safrole | 163.0778 | 163.0759 | 0.0018 | 14.143 |
| methoxycinnamaldehyde | 163.0778 | 163.0759 | 0.0018 | 14.143 |
| jasmone | 165.1215 | 165.1279 | −0.0064 | 1.197 |
| camphorquinone | 167.1029 | 167.1072 | −0.0043 | 0.8797 |
| perillic acid | 167.1029 | 167.1072 | −0.0043 | 0.8797 |
| 3-(phenylmethoxy)-1-propanol | 167.1029 | 167.1072 | −0.0043 | 0.8797 |
| 2-undecanal | 169.1642 | 169.1592 | 0.0049 | 0.2416 |
| undec-4-enal | 169.1642 | 169.1592 | 0.0049 | 0.2416 |
| cinnamyl acetate | 177.0907 | 177.0915 | −0.0008 | 36.862 |
| canavanine | 177.0907 | 177.0987 | −0.008 | 36.862 |
| coniferaldehyde | 179.0723 | 179.0708 | 0.0015 | 14.513 |
| methoxycinnamic acid | 179.0723 | 179.0708 | 0.0015 | 14.513 |
| D-mannosamine | 180.0895 | 180.0872 | 0.0023 | 4.1421 |
| galacgtosamine | 180.0895 | 180.0872 | 0.0023 | 4.1421 |
| glucosamine | 180.0895 | 180.0872 | 0.0023 | 4.1421 |
| stilbene | 181.1072 | 181.1017 | 0.0055 | 1.5466 |
| 1,3-di-tert-butylbenzene | 191.1837 | 191.18 | 0.0037 | 5.5631 |
| myristicin | 193.093 | 193.0864 | 0.0065 | 5.5942 |
| dehydrozingerone | 193.093 | 193.0865 | 0.0065 | 5.5942 |
| a-phenylindol | 194.0947 | 194.0969 | −0.0022 | 2.6837 |
| 1-tridenyn-4-ol | 197.1956 | 197.1905 | 0.0051 | 0.311 |
| dehydrocurcumene | 201.1646 | 201.1643 | 0.0003 | 12.468 |
| curcumene/cuparene/calamenene | 203.1795 | 203.18 | −0.0005 | 71.668 |
| zingiberene/farnesene/bisabolene | 205.1942 | 205.1956 | −0.0014 | 100 |
| alloaromadendrene/elemene | 205.1942 | 205.1956 | −0.0014 | 100 |
| cycloheptane, 4-methylene-1- | 205.1942 | 205.1956 | −0.0014 | 100 |
| aromadendrene, (+) | 205.1942 | 205.1956 | −0.0014 | 100 |
| caryophyllene | 205.1942 | 205.1956 | −0.0014 | 100 |
| cedrene | 205.1942 | 205.1956 | −0.0014 | 100 |
| farnesene | 205.1942 | 205.1956 | −0.0014 | 100 |
| humulene | 205.1942 | 205.1956 | −0.0014 | 100 |
| isocaryophyllene | 205.1942 | 205.1956 | −0.0014 | 100 |
| isolongifolene | 205.1942 | 205.1956 | −0.0014 | 100 |
| longicyclene/longifolene | 205.1942 | 205.1956 | −0.0014 | 100 |
| thujopsen | 205.1942 | 205.1956 | −0.0014 | 100 |
| valencene | 205.1942 | 205.1956 | −0.0014 | 100 |
| gualene/guainene | 205.1942 | 205.1956 | −0.0014 | 100 |
| copaene | 205.1942 | 205.1956 | −0.0014 | 100 |
| germacrene D | 205.1942 | 205.1956 | −0.0014 | 100 |
| a-cubebene | 205.1942 | 205.1956 | −0.0014 | 100 |
| a-muurolene | 205.1942 | 205.1956 | −0.0014 | 100 |
| (−)-a-panasinsen | 205.1942 | 205.1956 | −0.0014 | 100 |
| β-sesquiphellandrene | 205.1942 | 205.1956 | −0.0014 | 100 |
| 3,5-bis(1,1-dimethylethyl)-p | 207.173 | 207.1749 | −0.0019 | 8.2902 |
| hexylcinnamaldehyde | 217.1562 | 217.1592 | −0.003 | 9.3452 |
| ar-tumerone | 217.1562 | 217.1592 | −0.003 | 9.3452 |
| furanoeremophilane | 219.1747 | 219.1749 | −0.0002 | 27.159 |
| nootkatone | 219.1747 | 219.1749 | −0.0003 | 27.159 |
| valerenal | 219.1747 | 219.1749 | −0.0003 | 27.159 |
| xanthorrhizol | 219.1747 | 219.1749 | −0.0003 | 27.159 |
| curlone | 219.1747 | 219.1749 | −0.0003 | 27.159 |
| turmerone/ar-turmerol/xantho | 219.1747 | 219.1749 | −0.0003 | 27.159 |
| caryophellene oxide/bergamotol | 221.1915 | 221.1905 | 0.001 | 19.648 |
| spathulenol/9-cedranone/lanceol | 221.1915 | 221.1905 | 0.001 | 19.648 |
| 6-isopropenyl-4,8a-dimethyl- | 221.1915 | 221.1905 | 0.001 | 19.648 |
| caryophyllene oxide/(−)-spat | 221.1915 | 221.1905 | 0.001 | 19.648 |
| undec-2-ene-8,10-diynoic acid | 232.1651 | 232.1701 | −0.005 | 1.544 |
| costunolide | 233.1587 | 233.1541 | 0.0045 | 10.578 |
| panthenol | 234.1738 | 234.1705 | 0.0033 | 2.7337 |
| eremophilanlactone | 235.1688 | 235.1698 | −0.0011 | 13.273 |
| 2-octyl benzoate | 235.1688 | 235.1698 | −0.0011 | 13.273 |
| valerenic acid | 235.1688 | 235.1698 | −0.0011 | 13.273 |
| vellerdiol | 237.1835 | 237.1854 | −0.0019 | 12.762 |
| 3-methyl-but-2-enoic acid, 1 | 237.1835 | 237.1854 | −0.0019 | 12.762 |
| 2-pentenoic acid, 3-methyl-5 | 237.1835 | 237.1854 | −0.0019 | 12.762 |
| a-ionyl acetate | 237.1835 | 237.1854 | −0.0019 | 12.762 |
| 6-paradol | 251.1673 | 251.1647 | 0.0025 | 4.6708 |
| hydroxyvalerenic acid | 251.1673 | 251.1647 | 0.0025 | 4.6708 |
| palmitic acid | 257.2476 | 257.248 | −0.0004 | 0.4394 |
| panaxydol | 261.1885 | 261.1854 | 0.003 | 14.262 |
| oxymatrine | 265.1897 | 265.1916 | −0.002 | 1.6402 |
| eserine | 276.1737 | 276.1712 | 0.0025 | 17.491 |
| 6-shogaol | 277.179 | 277.1803 | −0.0014 | 97.039 |
| menthyl salicylate | 277.179 | 277.1803 | −0.0014 | 97.039 |
| cyclohexanecarboxylic acid, | 277.179 | 277.1803 | −0.0014 | 97.039 |
| 6-shogaol | 277.179 | 277.1804 | −0.0014 | 97.039 |
| linolenic acid | 279.2239 | 279.2324 | −0.0085 | 5.9227 |
| 9,12,15-octadecatrienoic acid | 279.2239 | 279.2324 | −0.0085 | 5.9227 |
| stearolic acid | 281.2465 | 281.248 | −0.0015 | 0.7788 |
| linoleic acid | 281.2465 | 281.248 | −0.0015 | 0.7788 |
| stearolic acid/linoelaidic a | 281.2465 | 281.248 | −0.0015 | 0.7788 |
| 9,12-octadecadienoic acid | 281.2465 | 281.248 | −0.0015 | 0.7788 |
| linoleic acid | 281.2465 | 281.248 | −0.0015 | 0.7788 |
| lynestrenol | 285.2182 | 285.2218 | −0.0036 | 2.7022 |
| vitamin A(retinol) | 287.231 | 287.2375 | −0.0065 | 3.3468 |
| abieta-8,11,13-trien-18-ol | 287.231 | 287.2375 | −0.0065 | 3.3468 |
| N-octyl-B-D-glucopyranoside | 293.1925 | 293.1964 | −0.0039 | 6.8357 |
| 6-gingerol | 295.1985 | 295.1909 | 0.0076 | 6.5307 |
| embelin | 295.1985 | 295.1909 | 0.0076 | 6.5307 |
| 6-gingerol | 295.1985 | 295.1909 | 0.0076 | 6.5307 |
| 6-gingerdiol | 297.209 | 297.2066 | 0.0025 | 1.3468 |
| 9,12-octadecadienoyl chloride | 299.2074 | 299.2141 | −0.0067 | 1.0009 |
| abietic acid | 303.2421 | 303.2324 | 0.0096 | 9.4454 |
| eicosapentaenoic acid | 303.2421 | 303.2324 | 0.0096 | 9.4454 |
| 8-shogaol | 305.2136 | 305.2117 | 0.0019 | 23.444 |
| 10-paradol | 307.2254 | 307.2273 | −0.0019 | 2.4769 |
| bioallethrin | 313.272 | 313.2742 | −0.0022 | 0.5011 |
| galanolactone/aframodial/galanal | 319.2237 | 319.2273 | −0.0036 | 6.0166 |
| homocapsaicin | 320.2258 | 320.2226 | 0.0032 | 3.2409 |
| 8-gingerdiol | 325.2289 | 325.2379 | −0.009 | 3.3346 |
| ajmaline | 327.2101 | 327.2072 | 0.0029 | 6.0516 |
| hydroxyprogesterone/DHEA acetate | 331.23 | 331.2273 | 0.0027 | 11.375 |
| 10-shogaol | 333.2453 | 333.243 | 0.0023 | 44.304 |
| lobelanidine | 340.2261 | 340.2276 | −0.0015 | 2.1477 |
| yohimbic acid | 341.191 | 341.1865 | 0.0045 | 7.9731 |
| menisperine | 341.191 | 341.1985 | −0.0075 | 7.9731 |
| 10-dehydrogingerdione | 347.2296 | 347.2222 | 0.0073 | 12.523 |
| calycanthine | 347.2296 | 347.2235 | 0.0061 | 12.523 |
| 10-gingerdiol | 353.2743 | 353.2692 | 0.0051 | 4.478 |
| corynanthine/vincamine/yohimbine | 355.1977 | 355.2021 | −0.0044 | 6.229 |
| kahweol acetate | 357.1984 | 357.2066 | −0.0082 | 12.191 |
| 12-shogaol | 361.2762 | 361.2743 | 0.0019 | 4.1202 |
| cinobufotalin | 363.2642 | 363.2688 | −0.0046 | 1.5059 |
| incensole oxide acetate | 365.2654 | 365.2692 | −0.0039 | 0.519 |
| vitexilactone | 381.2598 | 381.2641 | −0.0043 | 2.0628 |
| cholesteryl chloride | 405.3262 | 405.3288 | −0.0027 | 1.7948 |
| 4-methylumbelliferyl elaidate | 441.2956 | 441.3005 | −0.0049 | 13.017 |
| condelphine | 450.2872 | 450.2855 | 0.0017 | 5.9275 |
| Diepoxydammar diol | 459.3463 | 459.3474 | −0.0011 | 5.5302 |
| ganoderic acid D/M | 469.3301 | 469.3318 | −0.0017 | 7.2468 |
| psychosine | 478.3397 | 478.338 | 0.0017 | 9.4762 |
| 18-glycyrrhetinic acid methyl ester | 485.3569 | 485.3631 | −0.0062 | 11.987 |
| hovenolactone/trevoagenin D | 489.3674 | 489.358 | 0.0094 | 5.6947 |
| gymnemagenin | 492.3479 | 492.3451 | 0.0028 | 8.3876 |
| 3-O-acetyl-9,11-dehydro BA | 497.3595 | 497.3631 | −0.0036 | 14.012 |
| acetylboswellic acid/ganoderol | 499.3781 | 499.3787 | −0.0007 | 8.1177 |
| 3-O-acetyl-11-hydroxy boswellic acid | 515.369 | 515.3737 | −0.0047 | 5.6266 |
| 3-acetyl-a-boswellic acid | 541.4199 | 541.4257 | −0.0059 | 2.6112 |
| adhyperforin | 551.4186 | 551.41 | 0.0085 | 3.7405 |
| echinenone | 551.4186 | 551.4253 | −0.0067 | 3.7405 |
| kahweol palmitate | 553.4303 | 553.4257 | 0.0046 | 2.0327 |
| diatoxanthin | 567.4122 | 567.4202 | −0.0081 | 1.3546 |
| ganodermic acids R/S | 571.4077 | 571.3999 | 0.0078 | 0.4332 |

Compounds in Fractional SCCO₂ Separation of Ginger Essential Oil: Separator 2

6-shogoal was present in this extract in 10.5% relative abundance. Other shogaols, paradols, gingerols, and gingerdiols were present in the extract. Averionols were present in the extract in less than 1% relative abundance. Amino acids, vitamins, fatty acids, alkaloids, quinones, tumerones, phenolic acids, phenols, sterols, capsaicins, gymnemagins, boswellic acids, saponins and hydrocarbons were also present in this extract. 105 out of 214 (49%) unique chemicals have been directly identified in this extract using the DART TOF-MS. Table 24 shows the compounds identified in the extracts along with their relative abundance. FIG. 10A shows the DART Spectrum of this extract.

TABLE 24
Compounds in Fractional SCCO₂ separation
of ginger essential oil: separator 2

| Compound | Meas. | Calc. | Diff(u) | Abund. |
|---|---|---|---|---|
| ethylbenzene | 107.0871 | 107.0861 | 0.001 | 1.4285 |
| norcamphor/heptadienal | 111.0829 | 111.081 | 0.0019 | 0.276 |
| hexanoic acid/butyl acetate | 117.0897 | 117.0915 | −0.0018 | 0.9464 |
| propyl sulfide | 119.0861 | 119.0894 | −0.0034 | 19.214 |
| pseudocumene/propynylcyclohexene | 121.1018 | 121.1017 | 0.0001 | 16.041 |
| 2,6-dimethylanilene/conyrin | 122.1068 | 122.0969 | 0.0098 | 1.6346 |
| 5-hepten-2-one, 6-methyl- | 127.116 | 127.1123 | 0.0037 | 0.7999 |
| leucine | 132.0943 | 132.1024 | −0.0081 | 0.7863 |
| ornithine | 133.1024 | 133.0977 | 0.0047 | 5.4064 |
| dicyclopentadiene | 133.1024 | 133.1017 | 0.0007 | 5.4064 |
| p-cymene | 135.1175 | 135.1174 | 0.0002 | 6.0032 |
| ocimene/camphene/adamantane | 137.1314 | 137.133 | −0.0016 | 11.521 |
| octalactone | 143.1079 | 143.1072 | 0.0006 | 3.1128 |
| baogongteng B | 144.1102 | 144.1024 | 0.0077 | 0.2358 |
| crotonylbetaine | 145.1043 | 145.1103 | −0.006 | 2.4188 |
| lysine | 147.1174 | 147.1133 | 0.0041 | 5.359 |
| carvacrol/thymol/cymenol/myrtenol | 151.1135 | 151.1123 | 0.0012 | 4.4188 |
| norpseudophedrine | 152.1148 | 152.1075 | 0.0073 | 0.3701 |
| decadienal/santolina epoxide | 153.1276 | 153.1279 | −0.0004 | 6.0147 |
| pinene oxide | 153.1276 | 153.1279 | −0.0004 | 6.0147 |
| nonalactone | 157.1265 | 157.1228 | 0.0036 | 0.6421 |
| methylcholine | 161.1334 | 161.1416 | −0.0082 | 3.9843 |
| jasmone | 165.1239 | 165.1279 | −0.004 | 1.172 |
| ephedrine | 166.1273 | 166.1232 | 0.0041 | 0.1083 |
| hordenine | 166.1273 | 166.1232 | 0.0041 | 0.1083 |
| pseudoephedrine | 166.1273 | 166.1232 | 0.0041 | 0.1083 |
| camphorquinone | 167.1066 | 167.1072 | −0.0006 | 4.273 |
| perillic acid | 167.1066 | 167.1072 | −0.0006 | 4.273 |
| 3-(phenylmethoxy)-1-propanol | 167.1066 | 167.1072 | −0.0006 | 4.273 |
| (3Z)-3-hexenyl-2-butenoate | 169.123 | 169.1228 | 0.0001 | 1.6523 |
| chrysanthemolactone | 169.123 | 169.1228 | 0.0001 | 1.6523 |
| a-Limonene diepoxide | 169.123 | 169.1228 | 0.0001 | 1.6523 |
| arcaine | 173.1562 | 173.1514 | 0.0047 | 0.6846 |
| n-octyl acetate | 173.1562 | 173.1541 | 0.002 | 0.6846 |
| capric acid | 173.1562 | 173.1541 | 0.002 | 0.6846 |
| caprylic acid ethyl ester | 173.1562 | 173.1541 | 0.002 | 0.6846 |
| n-decanoic acid/1,3-dioxolane | 173.1562 | 173.1541 | 0.002 | 0.6846 |
| cinnamyl acetate | 177.0974 | 177.0915 | 0.0059 | 5.1643 |
| canavanine | 177.0974 | 177.0987 | −0.0013 | 5.1643 |
| cotinine | 177.0974 | 177.1028 | −0.0054 | 5.1643 |
| serotonin | 177.0974 | 177.1028 | −0.0054 | 5.1643 |
| 2(4H)-benzofuranone, 5,6,7,7 | 181.1257 | 181.1228 | 0.0029 | 0.8082 |
| pinonic acid | 185.1277 | 185.1177 | 0.01 | 0.549 |
| 3-methyl-2-butenoic acid, 2- | 185.1277 | 185.1177 | 0.01 | 0.549 |
| chamazulen | 185.1277 | 185.133 | −0.0053 | 0.549 |
| 1,3-di-tert-butylbenzene | 191.184 | 191.18 | 0.004 | 1.6182 |
| damascone | 193.155 | 193.1592 | −0.0042 | 0.6673 |
| ionone | 193.155 | 193.1592 | −0.0042 | 0.6673 |
| β-pinene, 3-(acetylmethyl)- | 193.155 | 193.1592 | −0.0042 | 0.6673 |
| sedanolide | 195.1302 | 195.1385 | −0.0083 | 1.8431 |
| trans-chrysanthenyl acetate | 195.1302 | 195.1385 | −0.0083 | 1.8431 |
| (−)-myrtenyl acetate | 195.1302 | 195.1385 | −0.0083 | 1.8431 |
| 2,6-octadien-1-ol, 3,7-dimethyl | 197.1506 | 197.1541 | −0.0035 | 0.2285 |
| dihydrocarvylacetate | 197.1506 | 197.1541 | −0.0035 | 0.2285 |
| geranyl acetate | 197.1506 | 197.1541 | −0.0035 | 0.2285 |
| isobornyl acetate | 197.1506 | 197.1541 | −0.0035 | 0.2285 |
| isopulegyl acetate | 197.1506 | 197.1541 | −0.0035 | 0.2285 |
| lavandulyl acetate | 197.1506 | 197.1541 | −0.0035 | 0.2285 |
| L-bornyl acetate | 197.1506 | 197.1541 | −0.0035 | 0.2285 |
| linalyl acetate | 197.1506 | 197.1541 | −0.0035 | 0.2285 |
| neryl acetate | 197.1506 | 197.1541 | −0.0035 | 0.2285 |
| terpinyl acetate | 197.1506 | 197.1541 | −0.0035 | 0.2285 |
| acetic acid, bornyl ester | 197.1506 | 197.1541 | −0.0035 | 0.2285 |
| butane, 1-cyclopropylidene-5 | 197.1506 | 197.1541 | −0.0035 | 0.2285 |
| bornyl acetate | 197.1506 | 197.1541 | −0.0035 | 0.2285 |
| bornyl acetate/linalyl acetate | 197.1506 | 197.1541 | −0.0035 | 0.2285 |
| guaiazulene | 199.1446 | 199.1487 | −0.0042 | 0.4886 |
| naphthalene, 1,6-dimethyl-4- | 199.1446 | 199.1487 | −0.0042 | 0.4886 |
| dehydrocurcumene | 201.1654 | 201.1643 | 0.0011 | 6.6375 |
| curcumene/cuparene/calamenene | 203.1799 | 203.18 | −0.0001 | 43.32 |
| zingiberene/farnesene/bisabolene | 205.1955 | 205.1956 | −0.0001 | 100 |
| alloaromadendrene/elemene | 205.1955 | 205.1956 | −0.0001 | 100 |
| cycloheptane, 4-methylene-1- | 205.1955 | 205.1956 | −0.0001 | 100 |
| aromadendrene, (+) | 205.1955 | 205.1956 | −0.0001 | 100 |
| caryophyllene | 205.1955 | 205.1956 | −0.0001 | 100 |
| cedrene | 205.1955 | 205.1956 | −0.0001 | 100 |
| farnesene | 205.1955 | 205.1956 | −0.0001 | 100 |
| humulene | 205.1955 | 205.1956 | −0.0001 | 100 |
| isocaryophyllene | 205.1955 | 205.1956 | −0.0001 | 100 |
| isolongifolene | 205.1955 | 205.1956 | −0.0001 | 100 |
| longicyclene/longifolene | 205.1955 | 205.1956 | −0.0001 | 100 |
| thujopsen | 205.1955 | 205.1956 | −0.0001 | 100 |
| valencene | 205.1955 | 205.1956 | −0.0001 | 100 |
| gualene/guianene | 205.1955 | 205.1956 | −0.0001 | 100 |
| copaene | 205.1955 | 205.1956 | −0.0001 | 100 |
| germacrene D | 205.1955 | 205.1956 | −0.0001 | 100 |
| a-cubebene | 205.1955 | 205.1956 | −0.0001 | 100 |
| a-muurolene | 205.1955 | 205.1956 | −0.0001 | 100 |
| trans-a-bergamotene | 205.1955 | 205.1956 | −0.0001 | 100 |
| (−)-a-panasinsen | 205.1955 | 205.1956 | −0.0001 | 100 |
| β-sesquiphellandrene | 205.1955 | 205.1956 | −0.0001 | 100 |
| 3,5-bis(1,1-dimethylethyl)-p | 207.1782 | 207.1749 | 0.0032 | 6.3086 |
| carvylacetate | 209.1622 | 209.1541 | 0.0081 | 0.2066 |
| hexylcinnamaldehyde | 217.1613 | 217.1592 | 0.0021 | 3.1588 |
| ar-tumerone | 217.1613 | 217.1592 | 0.0021 | 3.1588 |
| furanoeremophilane | 219.1757 | 219.1749 | 0.0008 | 10.394 |
| nootkatone | 219.1757 | 219.1749 | 0.0008 | 10.394 |
| valerenal | 219.1757 | 219.1749 | 0.0008 | 10.394 |
| xanthorrhizol | 219.1757 | 219.1749 | 0.0008 | 10.394 |
| curlone | 219.1757 | 219.1749 | 0.0008 | 10.394 |
| turmerone/ar-turmerol | 219.1757 | 219.1749 | 0.0008 | 10.394 |
| caryophellene oxide | 221.1916 | 221.1905 | 0.0011 | 8.8935 |
| spathulenol | 221.1916 | 221.1905 | 0.0011 | 8.8935 |
| bergamotol | 221.1916 | 221.1905 | 0.0011 | 8.8935 |
| 9-cedranone/lanceol | 221.1916 | 221.1905 | 0.0011 | 8.8935 |
| undec-2-ene-8,10-diynoic acid | 232.1753 | 232.1701 | 0.0052 | 0.2889 |
| costunolide | 233.1582 | 233.1541 | 0.004 | 1.4009 |
| panthenol | 234.1789 | 234.1705 | 0.0083 | 0.5173 |
| eremophilanlactone | 235.1702 | 235.1698 | 0.0004 | 2.7268 |
| 2-octyl benzoate | 235.1702 | 235.1698 | 0.0004 | 2.7268 |
| valerenic acid | 235.1702 | 235.1698 | 0.0004 | 2.7268 |
| vellerdiol | 237.184 | 237.1854 | −0.0014 | 3.4535 |
| 2-pentenoic acid, 3-methyl-5 | 237.184 | 237.1854 | −0.0014 | 3.4535 |
| bicyclo[4.4.0]dec-2-ene-4-ol | 237.184 | 237.1854 | −0.0014 | 3.4535 |
| a-ionyl acetate | 237.184 | 237.1854 | −0.0014 | 3.4535 |
| dodeca-2(E),4(E)-tetraenoic acid | 248.1934 | 248.2014 | −0.008 | 0.0805 |
| dodeca-2E-4E-8Z-10-tetraenoic acid | 248.1934 | 248.2014 | −0.008 | 0.0805 |
| atractylenolide III | 249.1576 | 249.149 | 0.0086 | 0.6627 |
| parthenolide | 249.1576 | 249.149 | 0.0086 | 0.6627 |
| 6-paradol | 251.164 | 251.1647 | −0.0008 | 0.807 |
| hydroxyvalerenic acid | 251.164 | 251.1647 | −0.0008 | 0.807 |
| palmitic acid | 257.2501 | 257.248 | 0.0021 | 0.9837 |
| C20H32/biformene/kaur-16-ene | 273.2574 | 273.2582 | −0.0008 | 1.4841 |
| 3,6-epoxy-1-(4-hydroxy-3-met | 275.1725 | 275.1647 | 0.0077 | 0.8016 |
| podocarpic acid | 275.1725 | 275.1647 | 0.0077 | 0.8016 |
| eserine | 276.1757 | 276.1712 | 0.0044 | 1.3512 |
| 6-shogaol | 277.18 | 277.1803 | −0.0003 | 10.459 |
| menthyl salicylate | 277.18 | 277.1803 | −0.0003 | 10.459 |
| cyclohexanecarboxylic acid, | 277.18 | 277.1803 | −0.0003 | 10.459 |
| stearolic acid | 281.2489 | 281.248 | 0.0009 | 0.255 |
| linoleic acid | 281.2489 | 281.248 | 0.0009 | 0.255 |
| 9,12-octadecadienoic acid | 281.2489 | 281.248 | 0.0009 | 0.255 |
| stearolic acid/linoelaidic acid | 281.2489 | 281.248 | 0.0009 | 0.255 |
| 16-oxokahweol | 283.1748 | 283.1698 | 0.005 | 5.0865 |
| miltirone | 283.1748 | 283.1698 | 0.005 | 5.0865 |
| vitamin A(retinol) | 287.2468 | 287.2375 | 0.0093 | 0.3358 |
| abieta-8,11,13-trien-18-ol | 287.2468 | 287.2375 | 0.0093 | 0.3358 |
| atropine | 291.185 | 291.1834 | 0.0015 | 0.5691 |
| N-octyl-B-D-glucopyranoside | 293.2006 | 293.1964 | 0.0041 | 0.1709 |
| averionol E | 303.2529 | 303.2609 | −0.008 | 0.2867 |
| aleuritic acid | 305.2241 | 305.2328 | −0.0087 | 0.9892 |
| sarpagine | 311.1783 | 311.1759 | 0.0024 | 0.6571 |
| ajmaline | 327.2021 | 327.2072 | −0.0052 | 3.7826 |
| averionol C | 331.2926 | 331.285 | 0.0076 | 0.0766 |
| 10-shogaol | 333.244 | 333.243 | 0.0011 | 0.494 |
| corynanthine/vincamine/yohii | 355.2017 | 355.2021 | −0.0005 | 0.1127 |

### Example 4

### Example of Step 2 (FIG. 3)

### Hydroalcoholic Leaching Extraction

A typical example of a three-stage solvent extraction of the phenolic chemical constituents of Ginger species is as follows: The feedstock was 25 gm of ground Ginger rhizome SFE residue from Step 1 SCCO₂ (40° C., 300 bar) extraction of the essential oil. The solvent was 80% aqueous ethanol. In this method, the feedstock material and 500 ml aqueous ethanol (solvent/feed ratio=20) were separately loaded into 1000 ml extraction vessel and mixed in a heated water bath at 40° C. for 2 hours. The extraction solution was filtered using Fisherbrand P4 filter paper having a particle retention size of 4-8 μm, centrifuged at 3000 rpm for 10 minutes, and the particulate residue used for further extraction. The filtrate (supernatant) was collected for yield calculation and HPLC analysis. The residue of Stage 1 was extracted for 2 hours (Stage 2) with 250 ml 80% ethanol (solvent/feed ratio=10) using the aforementioned methods. The two supernatants were collected and combined for mass balance, HPLC analysis, and total phenolic analysis (Folin-Ciocalteu assay) of the extract. The results are shown in Table 11 below.

### Example 5

### Example of Step 3 (FIG. 4)

### Affinity Adsorbent Extraction of Phenolic Fraction

In typical experiments, the working solution was the transparent hydroalcoholic solution of Ginger species aqueous ethanol leaching extract in Step 2. The ethanol in 400 ml of this solution (4.56 mg/ml) was removed using rotary evaporation to a final volume of 40 ml to which 150 ml of distilled water was added to make a final aqueous solution of 190 ml having a concentration of 10.78 mg/ml. The affinity adsorbent polymer resin was XAD7HP. 30 gm of affinity adsorbent was pre-washed with 95% ethanol (3 BV) and distilled water (3 BV) before and after packing into a column with an ID of 15 mm and length of 300 mm. The bed volume (BV) was 30 ml. 100 ml (10.78 mg/ml) mg/ml) aqueous solution (loading solution) was loading on the column at flow rate of 2.4 BV/hr (1.3 ml/min). The loading time was 75 minutes. The loaded column was washed with 100 ml of distilled water at a flow rate of 3.2 BV/hr (1.8 ml/min) with a washing time of 55 minutes. 100 ml of 75% aqueous ethanol was used to elute the loaded column at a flow rate of 7 BV/hr (3.8 ml/min) with an elution time 26 minutes. During the elution, 4 fractions were collected at 0.7, 1.3, 2.2, and 3.1 BV (F1-F4), respectively. Then 4-5 BV of 95% ethanol was used to clean out the remaining chemicals on the column at a flow rate of 5 BV/hr followed by washing with 4-5 BV distilled water at 5 BV/hr. The flow rate during whole process was controlled using a FPU 252 Omegaflex® variable speed (3-50 ml/min) peristaltic pump. Each elution fraction was collected and analyzed using HPLC and total phenolic assay methods and the results are shown in Table 25.

TABLE 25
Hydro-alcoholic leaching and PA purification yield and HPLC analysis results.

| | | | Purity (% mass weight) | | | | | | | |
|---|---|---|---|---|---|---|---|---|---|---|
| | Yield | Mass | | | | | Total | Total | Phenolic | 6-G |
| | (%) | (mg) | 6-G | 8-G | 10-G | 6-S | Stds¹ | Phenolics² | Mass (mg) | ratio³ |
| Crude extract | 12.4 | | 3.64 | 0.43 | 1.44 | 0.31 | 5.8 | 5.9 | | 62.5 |
| After | 10.7 | | 1.94 | 0.13 | 0.02 | 0.15 | 2.2 | 3.7 | | 86.4 |
| distillation | | | | | | | | | | |
| PA loading | 10.7 | 1055 | 1.94 | 0.13 | 0.02 | 0.15 | 2.2 | 3.7 | 23.7 | 86.4 |
| Effluent | 6.8 | 667 | 0.00 | 0.00 | 0.00 | 0.00 | 0.0 | 0.8 | 0.0 | |
| washing | 3.0 | 300 | 0.00 | 0.00 | 0.00 | 0.00 | 0.0 | 1.6 | 0.0 | |
| F1 | 0.1 | 7 | 0.00 | 0.00 | 0.00 | 0.00 | 0.0 | 2.6 | 0.0 | |
| F2 | 0.6 | 55 | 3.32 | 0.08 | 0.06 | 0.03 | 3.5 | 15.1 | 1.9 | 95.1 |
| F3 | 0.5 | 48 | 23.11 | 0.70 | 0.97 | 0.30 | 25.1 | 25.9 | 12.1 | 92.1 |
| F4 | 0.2 | 15 | 30.59 | 0.94 | 1.98 | 0.54 | 34.0 | 30.4 | 5.3 | 89.9 |

¹Purity of total stds = Purify of (6-G) + (8-G) + (10-G) + (6-S).
²Purity of total phenolic were analyzed by Folin-Ciocalteu method.
³6-G ratio = [purity of (6-G)]/(purity of total stds) × 100.

### Compounds in Phenolic Acids Fraction: 80% Ethanol Extract

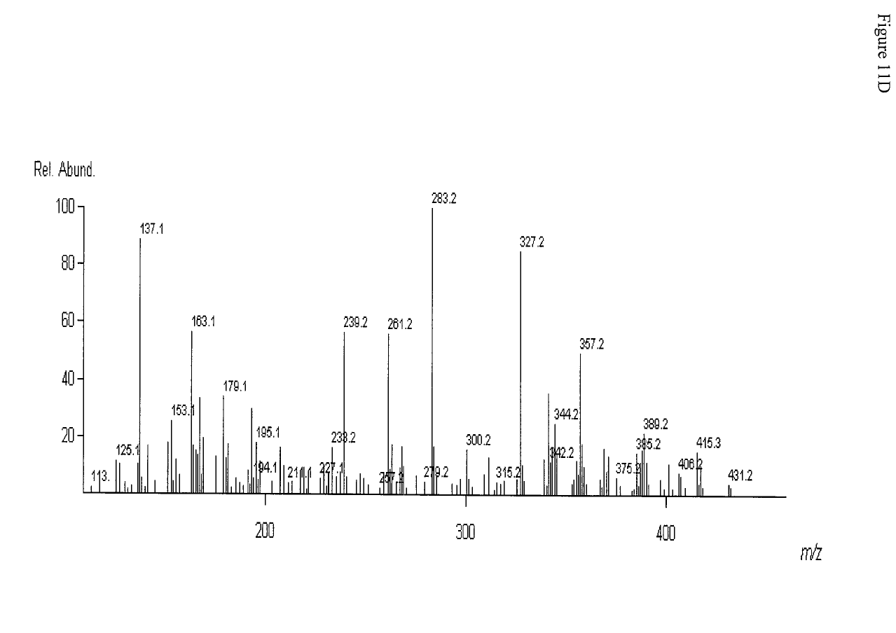

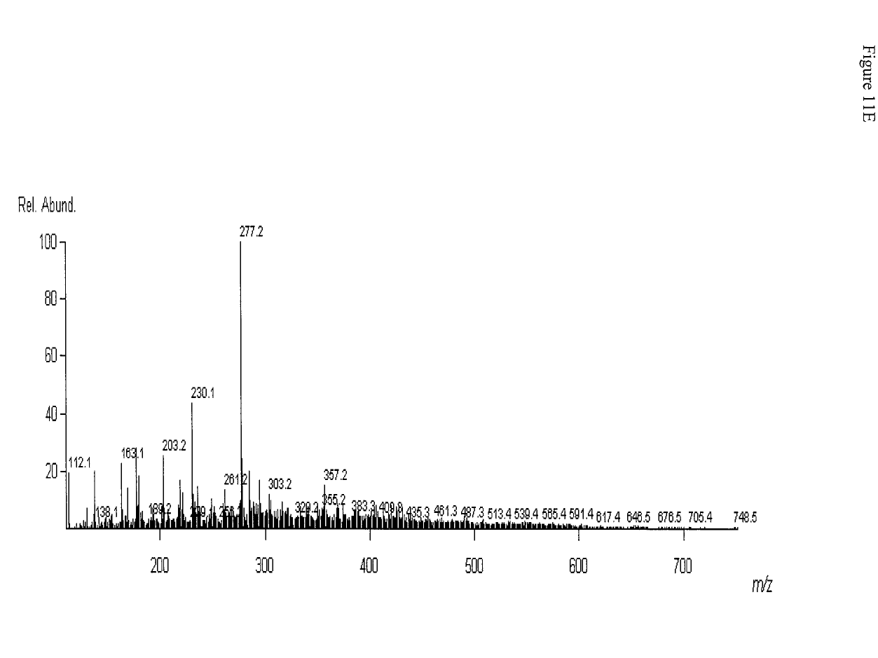

6-shogoal was present in this extract in 12.9% relative abundance. Other shogaols, paradols, gingerols, and gingerdiols were present in the extract. Averionols were present in the extract in less than 1% relative abundance. Amino acids, vitamins, fatty acids, tumerones, alkaloids, phenolic acids, phenols, sterols, capsaicins, gymnemagins, boswellic acids, saponins and hydrocarbons were also present in this extract. 112 out of 342 (33%) unique chemicals have been directly identified in this extract using the DART TOF-MS. Table 26 shows the compounds identified in the extracts along with their relative abundance. FIG. 11A shows the DART Spectrum of this extract.

TABLE 26
Compounds in Phenolic acids fraction: 80% ethanol extract

| Compounds | Meas. | Calc. | Diff(u) | Abund. |
|---|---|---|---|---|
| ethylbenzene | 107.0865 | 107.0861 | 0.0004 | 1.614 |
| hexanoic acid/butyl acetate | 117.086 | 117.0915 | −0.0055 | 0.4664 |
| propyl sulfide | 119.086 | 119.0894 | −0.0035 | 21.155 |
| pseudocumene/propynylcyclohexene | 121.1014 | 121.1017 | −0.0003 | 17.314 |
| cysteine | 122.0331 | 122.0275 | 0.0056 | 0.0299 |
| 2,6-dimethylanilene/conyrin | 122.1067 | 122.0969 | 0.0098 | 1.6995 |
| 5-hepten-2-one, 6-methyl- | 127.1131 | 127.1123 | 0.0008 | 0.4682 |
| leucine | 132.0948 | 132.1024 | −0.0077 | 0.4803 |
| ornithine | 133.1024 | 133.0977 | 0.0046 | 3.7892 |
| dicyclopentadiene | 133.1024 | 133.1017 | 0.0006 | 3.7892 |
| p-cymene | 135.1175 | 135.1174 | 0.0002 | 4.4914 |
| phenyl isothiocyanate | 136.0199 | 136.0221 | −0.0022 | 0.0561 |
| anisaldehyde/formic acid benzoate | 137.0601 | 137.0602 | −0.0001 | 6.4222 |
| trigonelline/vitamin H | 138.0628 | 138.0555 | 0.0073 | 0.096 |
| octalactone | 143.1104 | 143.1072 | 0.0032 | 1.6191 |
| crotonylbetaine | 145.1049 | 145.1103 | −0.0054 | 2.2061 |
| lysine | 147.1176 | 147.1133 | 0.0043 | 4.5077 |
| 1-methyl-3-phenylpropylamine | 150.1376 | 150.1282 | 0.0093 | 1.3092 |
| 4-phenylbutylamine | 150.1376 | 150.1282 | 0.0093 | 1.3092 |
| carvacrol/thymol/cymenol/myrtenol | 151.1156 | 151.1123 | 0.0033 | 2.0293 |
| 2-butyl-3-methylpyrazine | 151.1156 | 151.1235 | −0.0079 | 2.0293 |
| dihydroxyacetophenone | 153.0543 | 153.0551 | −0.0008 | 1.0111 |
| decadienal/santolina epoxide | 153.1279 | 153.1279 | 0 | 2.3946 |
| pinene oxide/piperitone pule | 153.1279 | 153.1279 | 0 | 2.3946 |
| cineole/borneol | 155.143 | 155.1436 | −0.0006 | 0.0754 |
| methone/pinocampheol/pulegol | 155.143 | 155.1436 | −0.0006 | 0.0754 |
| methylcholine | 161.1333 | 161.1416 | −0.0084 | 2.9952 |
| jasmone | 165.1354 | 165.1279 | 0.0074 | 0.4434 |
| camphorquinone | 167.1059 | 167.1072 | −0.0013 | 2.3518 |
| perillic acid | 167.1059 | 167.1072 | −0.0013 | 2.3518 |
| 3-(phenylmethoxy)-1-propanol | 167.1059 | 167.1072 | −0.0013 | 2.3518 |
| (3Z)-3-hexenyl-2-butenoate | 169.122 | 169.1228 | −0.0009 | 0.5062 |
| chrysanthemolactone | 169.122 | 169.1228 | −0.0009 | 0.5062 |
| a-Limonene diepoxide | 169.122 | 169.1228 | −0.0009 | 0.5062 |
| decalactone | 171.1471 | 171.1385 | 0.0086 | 0.8177 |
| linalool oxide | 171.1471 | 171.1385 | 0.0086 | 0.8177 |
| butanoic acid, 3-hexenyl ester | 171.1471 | 171.1385 | 0.0086 | 0.8177 |
| 3,7-octadiene-2,6-diol, 2,6- | 171.1471 | 171.1385 | 0.0086 | 0.8177 |
| 1,7-octadiene-3,6-diol, 2,6- | 171.1471 | 171.1385 | 0.0086 | 0.8177 |
| arcaine | 173.1472 | 173.1514 | −0.0042 | 0.7534 |
| n-octyl acetate | 173.1472 | 173.1541 | −0.0069 | 0.7534 |
| capric acid | 173.1472 | 173.1541 | −0.0069 | 0.7534 |
| caprylic acid ethyl ester | 173.1472 | 173.1541 | −0.0069 | 0.7534 |
| n-decanoic acid/1,3-dioxolane | 173.1472 | 173.1541 | −0.0069 | 0.7534 |
| cinnamyl acetate | 177.0927 | 177.0915 | 0.0011 | 7.9115 |
| canavanine | 177.0927 | 177.0987 | −0.0061 | 7.9115 |
| homophenylalanine | 180.1076 | 180.1024 | 0.0051 | 0.1867 |
| salsolinol | 180.1076 | 180.1024 | 0.0051 | 0.1867 |
| 2(4H)-benzofuranone, 5,6,7,7 | 181.1208 | 181.1228 | −0.0021 | 0.2355 |
| carbahcol | 183.0986 | 183.09 | 0.0086 | 0.1991 |
| difluoromethylornithine | 183.0986 | 183.0945 | 0.0041 | 0.1991 |
| dihydroconiferyl alcohol | 183.0986 | 183.1021 | −0.0035 | 0.1991 |
| chamazulen | 185.1344 | 185.133 | 0.0013 | 0.4252 |
| 1,3-di-tert-butylbenzene | 191.1814 | 191.18 | 0.0014 | 1.3191 |
| damascone | 193.1666 | 193.1592 | 0.0073 | 0.7174 |
| ionone | 193.1666 | 193.1592 | 0.0073 | 0.7174 |
| β-pinene, 3-(acetylmethyl)- | 193.1666 | 193.1592 | 0.0073 | 0.7174 |
| sedanolide | 195.1434 | 195.1385 | 0.0049 | 0.8538 |
| trans-chrysanthenyl acetate | 195.1434 | 195.1385 | 0.0049 | 0.8538 |
| (−)-myrtenyl acetate | 195.1434 | 195.1385 | 0.0049 | 0.8538 |
| 1-octen-3-yl butyrate | 199.1654 | 199.1698 | −0.0044 | 1.8393 |
| citronellyl acetate | 199.1654 | 199.1698 | −0.0044 | 1.8393 |
| dodecalactone | 199.1654 | 199.1698 | −0.0044 | 1.8393 |
| menthyl acetate | 199.1654 | 199.1698 | −0.0044 | 1.8393 |
| neomenthylacetate | 199.1654 | 199.1698 | −0.0044 | 1.8393 |
| dehydrocurcumene | 201.1654 | 201.1643 | 0.0011 | 6.399 |
| curcumene/cuparene/calamenene | 203.1796 | 203.18 | −0.0005 | 43.848 |
| zingiberene/farnesene/bisabolene | 205.1953 | 205.1956 | −0.0003 | 100 |
| alloaromadendrene/elemene | 205.1953 | 205.1956 | −0.0003 | 100 |
| cycloheptane, 4-methylene-1- | 205.1953 | 205.1956 | −0.0003 | 100 |
| aromadendrene | 205.1953 | 205.1956 | −0.0003 | 100 |
| caryophyllene | 205.1953 | 205.1956 | −0.0003 | 100 |
| cedrene | 205.1953 | 205.1956 | −0.0003 | 100 |
| farnesene | 205.1953 | 205.1956 | −0.0003 | 100 |
| humulene | 205.1953 | 205.1956 | −0.0003 | 100 |
| isocaryophyllene | 205.1953 | 205.1956 | −0.0003 | 100 |
| isolongifolene | 205.1953 | 205.1956 | −0.0003 | 100 |
| longicyclene/longifolene | 205.1953 | 205.1956 | −0.0003 | 100 |
| thujopsen | 205.1953 | 205.1956 | −0.0003 | 100 |
| valencene | 205.1953 | 205.1956 | −0.0003 | 100 |
| gualene/guainene | 205.1953 | 205.1956 | −0.0003 | 100 |
| copaene | 205.1953 | 205.1956 | −0.0003 | 100 |
| germacrene D | 205.1953 | 205.1956 | −0.0003 | 100 |
| a-cubebene | 205.1953 | 205.1956 | −0.0003 | 100 |
| a-muurolene | 205.1953 | 205.1956 | −0.0003 | 100 |
| trans-a-bergamotene | 205.1953 | 205.1956 | −0.0003 | 100 |
| (−)-a-panasinsen | 205.1953 | 205.1956 | −0.0003 | 100 |
| β-sesquiphellandrene | 205.1953 | 205.1956 | −0.0003 | 100 |
| 3,5-bis(1,1-dimethylethyl)-p | 207.1782 | 207.1749 | 0.0033 | 6.7522 |
| hexylcinnamaldehyde | 217.1627 | 217.1592 | 0.0035 | 3.6582 |
| ar-tumerone | 217.1627 | 217.1592 | 0.0035 | 3.6582 |
| furanoeremophilane | 219.1754 | 219.1749 | 0.0005 | 11.074 |
| nootkatone | 219.1754 | 219.1749 | 0.0005 | 11.074 |
| valerenal | 219.1754 | 219.1749 | 0.0005 | 11.074 |
| xanthorrhizol | 219.1754 | 219.1749 | 0.0005 | 11.074 |
| curlone | 219.1754 | 219.1749 | 0.0005 | 11.074 |
| turmerone/ar-turmerol | 219.1754 | 219.1749 | 0.0005 | 11.074 |
| caryophellene oxide | 221.1909 | 221.1905 | 0.0003 | 9.9821 |
| spathulenol | 221.1909 | 221.1905 | 0.0003 | 9.9821 |
| 6,10-dodecadien-1-yn-3-ol, 3 | 221.1909 | 221.1905 | 0.0003 | 9.9821 |
| bergamotol | 221.1909 | 221.1905 | 0.0003 | 9.9821 |
| spathulenol/9-cedranone/lanceol | 221.1909 | 221.1905 | 0.0003 | 9.9821 |
| methyl 2-hydroxydodecanoate | 231.199 | 231.196 | 0.0029 | 0.372 |
| undec-2-ene-8,10-diynoic acid | 232.1764 | 232.1701 | 0.0063 | 0.7327 |
| costunolide | 233.1627 | 233.1541 | 0.0086 | 1.3623 |
| eremophilanlactone | 235.1709 | 235.1698 | 0.0011 | 2.7972 |
| 2-octyl benzoate | 235.1709 | 235.1698 | 0.0011 | 2.7972 |
| valerenic acid | 235.1709 | 235.1698 | 0.0011 | 2.7972 |
| vellerdiol | 237.184 | 237.1854 | −0.0014 | 3.3682 |
| 3-methyl-but-2-enoic acid, 1 | 237.184 | 237.1854 | −0.0014 | 3.3682 |
| 2-pentenoic acid, 3-methyl-5 | 237.184 | 237.1854 | −0.0014 | 3.3682 |
| a-ionyl acetate | 237.184 | 237.1854 | −0.0014 | 3.3682 |
| menthyl isovalerate | 241.2118 | 241.2167 | −0.0049 | 0.1894 |
| dodecyl acrylate | 241.2118 | 241.2167 | −0.0049 | 0.1894 |
| 6-paradol | 251.1705 | 251.1647 | 0.0058 | 0.8079 |
| hydroxyvalerenic acid | 251.1705 | 251.1647 | 0.0057 | 0.8079 |
| palmitic acid | 257.2487 | 257.248 | 0.0007 | 1.1723 |
| C20H32/biformene/kaur-16-ene | 273.2565 | 273.2582 | −0.0017 | 2.1657 |
| podocarpic acid | 275.1708 | 275.1647 | 0.0061 | 1.2826 |
| eserine | 276.1748 | 276.1712 | 0.0036 | 1.2614 |
| 6-shogaol | 277.1798 | 277.1803 | −0.0005 | 12.939 |
| menthyl salicylate | 277.1798 | 277.1803 | −0.0005 | 12.939 |
| cyclohexanecarboxylic acid | 277.1798 | 277.1803 | −0.0005 | 12.939 |
| 6-shogaol | 277.1798 | 277.1804 | −0.0006 | 12.939 |
| 8-paradol | 279.2059 | 279.196 | 0.0099 | 1.1234 |
| stearolic acid | 281.2469 | 281.248 | −0.0011 | 0.4334 |
| 9,12-octadecadienoic acid | 281.2469 | 281.248 | −0.0011 | 0.4334 |
| stearolic acid/linoelaidic acid | 281.2469 | 281.248 | −0.0011 | 0.4334 |
| 9,12-octadecadienoic acid (Z | 281.2469 | 281.248 | −0.0011 | 0.4334 |
| linoleic acid | 281.2469 | 281.248 | −0.0011 | 0.4334 |
| 16-oxokahweol | 283.1793 | 283.1698 | 0.0094 | 1.5945 |
| miltirone | 283.1793 | 283.1698 | 0.0094 | 1.5945 |
| vitamin A(retinol) | 287.2442 | 287.2375 | 0.0066 | 1.0429 |
| abieta-8,11,13-trien-18-ol | 287.2442 | 287.2375 | 0.0066 | 1.0429 |
| 7-shogaol | 291.1942 | 291.196 | −0.0019 | 0.9892 |
| N-octyl-B-D-glucopyranoside | 293.2026 | 293.1964 | 0.0062 | 0.2779 |
| lauric acid, 2-butoxyethyl ester | 301.2701 | 301.2742 | −0.0041 | 0.3485 |
| averionol E | 303.2538 | 303.2609 | −0.0071 | 0.5579 |
| dihydrocapsaicin | 308.2307 | 308.2226 | 0.0081 | 0.0563 |
| linoleic acid, ethyl ester | 309.2754 | 309.2793 | −0.0039 | 0.357 |
| sclareol | 309.2754 | 309.2793 | −0.0039 | 0.357 |
| sclareol | 309.2754 | 309.2793 | −0.0039 | 0.357 |
| Z-8-octadecen-1-ol acetate | 311.2889 | 311.295 | −0.0061 | 0.2527 |
| 9-octadecenoic acid, ethyl ester | 311.2889 | 311.295 | −0.0061 | 0.2527 |
| 2-chloroethyl palmitate | 319.2442 | 319.2404 | 0.0038 | 0.1891 |
| ajmaline | 327.206 | 327.2072 | −0.0012 | 1.3566 |
| 10-shogaol | 333.2498 | 333.243 | 0.0069 | 1.3379 |
| 17a-hydroxypregnenolone | 335.2535 | 335.2586 | −0.0051 | 0.2951 |
| averionol B | 341.2987 | 341.307 | −0.0083 | 0.7783 |
| chenodeoxycholic acid | 345.3081 | 345.3005 | 0.0076 | 0.0643 |
| kauran-18-al, 17-(acetyloxy) | 347.2587 | 347.2586 | 0 | 0.0615 |
| averionol A | 359.3162 | 359.3149 | 0.0012 | 0.1837 |
| fraxin | 371.1037 | 371.0978 | 0.0059 | 3.7678 |
| octyl phthalate | 391.2873 | 391.2848 | 0.0025 | 0.9709 |
| mogroside backbone-4H2O | 405.3589 | 405.3522 | 0.0067 | 0.6202 |
| benzethonium | 413.3381 | 413.3294 | 0.0087 | 0.1432 |
| lupulone | 415.2917 | 415.2848 | 0.0069 | 0.4471 |
| amyrenone/lupenone | 425.3807 | 425.3783 | 0.0024 | 0.4208 |
| cholesteryl acetate | 429.3685 | 429.3732 | −0.0047 | 0.0399 |

### Compounds in Phenolic Acid Fraction: Polymer Adsorbent Processing: Loading Solution

6-shogoal, 6-gingerol and galanolactone were not identified in this extract. Amino acids, vitamins, flavonoids, alkaloids, phenolic acids, phenols, sterols, capsaicins, gymnemic acids and ganolucidenic acids were present in this extract. 54 out of 707 (8%) unique chemicals have been directly identified in this extract using the DART TOF-MS. Table 27 shows the compounds identified in the extracts along with their relative abundance. FIG. 11B shows the DART Spectrum of this extract.

TABLE 27
Compounds in Phenolic acid fraction: polymer adsorbent
processing loading solution

| Compound | Meas. | Calc. | Diff(u) | Abund. |
|---|---|---|---|---|
| 2-acetylpyrrole | 110.065 | 110.0606 | 0.0044 | 1.5153 |
| niacin | 124.0402 | 124.0398 | 0.0004 | 0.635 |
| vitamin B3 | 124.0402 | 124.0399 | 0.0003 | 0.635 |
| niacin | 124.0402 | 124.0399 | 0.0003 | 0.635 |
| tropine | 142.1304 | 142.1232 | 0.0071 | 2.8756 |
| ephedrine | 166.1272 | 166.1232 | 0.004 | 8.0736 |
| hordenine | 166.1272 | 166.1232 | 0.004 | 8.0736 |
| pseudoephedrine | 166.1272 | 166.1232 | 0.004 | 8.0736 |
| DL-a-methyl-m-tyrosine | 196.1037 | 196.0973 | 0.0064 | 33.6581 |
| 3-methoxy-l-tyrosine | 212.0957 | 212.0923 | 0.0034 | 4.5063 |
| nitrocyclopentanemethanol | 216.1407 | 216.1388 | 0.0018 | 3.2205 |
| 6-benzylaminopurine | 226.1131 | 226.1092 | 0.0039 | 1.4343 |
| terphenyl | 231.1208 | 231.1174 | 0.0034 | 2.2952 |
| flavanone hydrazone | 239.1232 | 239.1184 | 0.0048 | 1.5891 |
| huperazine A | 243.1466 | 243.1497 | −0.0031 | 12.5408 |
| lotaustralin | 262.1368 | 262.129 | 0.0078 | 1.5447 |
| abscisic acid | 265.1529 | 265.144 | 0.0089 | 3.3445 |
| sempervirine | 273.148 | 273.1391 | 0.0089 | 4.8922 |
| 16-oxocafestol | 285.1768 | 285.1854 | −0.0086 | 7.0716 |
| galanthamine | 288.1656 | 288.1599 | 0.0056 | 3.4845 |
| tanshinone IIA | 295.1432 | 295.1334 | 0.0098 | 1.0075 |
| 6-bromoflavone | 300.9864 | 300.9864 | 0 | 0.081 |
| evodiamine | 304.1505 | 304.145 | 0.0055 | 1.3138 |
| scopolamine | 304.1505 | 304.1549 | −0.0044 | 1.3138 |
| gelsemine | 323.1713 | 323.1759 | −0.0046 | 1.6378 |
| quinidine/quinine | 325.1875 | 325.1916 | −0.0042 | 9.42 |
| crocetin/geranoxy methoxycoumarin | 329.1743 | 329.1753 | −0.001 | 0.9301 |
| integerrimin/senecionine | 336.1841 | 336.1811 | 0.003 | 2.819 |
| lobeline | 338.2034 | 338.212 | −0.0086 | 3.9554 |
| esculin | 341.0861 | 341.0872 | −0.0011 | 0.1677 |
| linocinamarin | 341.1306 | 341.1236 | 0.007 | 0.1361 |
| rubrocyanin | 354.2173 | 354.2096 | 0.0076 | 4.4047 |
| aldosterone/cortisone/prednisone | 361.1942 | 361.2015 | −0.0073 | 1.5288 |
| hydrocortisone | 363.2102 | 363.2171 | −0.0069 | 2.4293 |
| tamoxifen | 372.2357 | 372.2327 | 0.003 | 3.4262 |
| diacetyl-6-gingerdiol | 381.2361 | 381.2277 | 0.0084 | 3.4722 |
| resibufogenin | 385.2341 | 385.2379 | −0.0038 | 2.004 |
| 1-deoxyforskolin | 395.2424 | 395.2433 | −0.001 | 2.5161 |
| dehydrocholic acid | 403.2408 | 403.2484 | −0.0076 | 3.3289 |
| pravastatin/strophanthidol | 407.2417 | 407.2433 | −0.0016 | 3.8085 |
| condelphine | 450.2883 | 450.2855 | 0.0027 | 3.0407 |
| celastrol | 451.2885 | 451.2848 | 0.0037 | 2.4729 |
| cytochalasin J | 452.2867 | 452.2801 | 0.0066 | 3.3552 |
| lucidenic acids A/N | 461.2993 | 461.2903 | 0.0089 | 2.1253 |
| deoxywithastramonolide/witha | 471.2813 | 471.2746 | 0.0066 | 2.2889 |
| emetine | 481.2982 | 481.3066 | −0.0084 | 1.7838 |
| ganolucidic acid A | 499.3133 | 499.306 | 0.0074 | 1.6635 |
| ganolucidic acid B | 501.3168 | 501.3216 | −0.0048 | 2.1161 |
| evomonoside | 521.317 | 521.3114 | 0.0056 | 1.7352 |
| acovenoside A | 554.3539 | 554.3455 | 0.0083 | 1.902 |
| carbenoxolone | 571.3559 | 571.3635 | −0.0076 | 1.2095 |
| 23-o-aetyl-shengmanol | 619.3997 | 619.3999 | −0.0001 | 0.5946 |
| cimiracemoside C | 621.3968 | 621.4002 | −0.0034 | 0.5169 |
| gymnemic acid I - GlcA | 631.4214 | 631.4209 | 0.0005 | 0.4419 |
| gymnemic acid XV - GlcA | 673.4596 | 673.468 | −0.0084 | 0.3294 |

### Compounds in Phenolic Acid Fraction: Polymer Adsorbent Processing: Collected Fraction 2

6-shogoal and galanolactone were present in this extract in 48.9 and 13.6% relative abundance, respectively. Other shagoals, paradols, gingerols and giongerdiols were also present in this extract. Amino acids, vitamins, fatty acids, saccharides, quinones, tumerones, alkaloids, xanthines, ganoderic acids, gymnemic acids, phenolic acids, phenols, sterols, capsaicins, gymnemagins, boswellic acids, saponins and hydrocarbons were also present in this extract. 138 out of 699 (20%) unique chemicals have been directly identified in this extract using the DART TOF-MS. Table 28 shows the compounds identified in the extracts along with their relative abundance. FIG. 11C shows the DART Spectrum of this extract.

TABLE 28
Compounds in polymer adsorbent processing: collected fraction 2

| Compound | Meas. | Calc. | Diff(u) | Abund. |
|---|---|---|---|---|
| 1,4-benzoquinone | 109.0341 | 109.0289 | 0.0052 | 1.3839 |
| 2-acetylpyrrole | 110.0624 | 110.0606 | 0.0018 | 3.5148 |
| histamine | 112.0874 | 112.0874 | 0 | 33.879 |
| 2-methylcyclohexanone | 113.0882 | 113.0966 | −0.0084 | 1.5435 |
| L-threonine | 120.0561 | 120.066 | −0.0099 | 2.6278 |
| pyrogallol/phlorglucinol/maltol | 127.0471 | 127.0395 | 0.0076 | 3.0403 |
| 6-methyluracil | 127.0471 | 127.0507 | −0.0036 | 3.0403 |
| p-cymene | 135.118 | 135.1174 | 0.0007 | 20.505 |
| adenine | 136.0628 | 136.0623 | 0.0005 | 100 |
| anisaldehyde/formic acid benzoate | 137.0614 | 137.0602 | 0.0011 | 33.782 |
| tyramine | 138.0918 | 138.0919 | −0.0001 | 4.944 |
| tropine | 142.1136 | 142.1232 | −0.0096 | 3.8071 |
| octalactone | 143.1081 | 143.1072 | 0.0009 | 7.8033 |
| 2-furanmethanol, tetrahydro- | 145.0788 | 145.0864 | −0.0076 | 3.916 |
| lysine | 147.1133 | 147.1133 | 0 | 4.1052 |
| 4-hydroxyisoleucine | 148.1054 | 148.0973 | 0.0081 | 6.6553 |
| 4-OH Ile | 148.1054 | 148.0974 | 0.008 | 6.6553 |
| anethole | 149.0948 | 149.0966 | −0.0019 | 1.3492 |
| cuminaldehyde | 149.0948 | 149.0966 | −0.0019 | 1.3492 |
| estragole | 149.0948 | 149.0966 | −0.0019 | 1.3492 |
| benzaldehyde, 4-propyl- | 149.0948 | 149.0966 | −0.0019 | 1.3492 |
| 2-acetyl-3-ethylpyrazine | 151.0956 | 151.0871 | 0.0084 | 13.289 |
| decadienal/santolina epoxide | 153.128 | 153.1279 | 0 | 49.49 |
| pinene oxide | 153.128 | 153.1279 | 0 | 49.49 |
| pseudopelletierine | 154.1181 | 154.1232 | −0.0051 | 21.325 |
| methyl cinnamic acid | 163.0765 | 163.0759 | 0.0006 | 47.704 |
| safrole | 163.0765 | 163.0759 | 0.0005 | 47.704 |
| methoxycinnamaldehyde | 163.0765 | 163.0759 | 0.0005 | 47.704 |
| camphorquinone | 167.1049 | 167.1072 | −0.0023 | 21.174 |
| perillic acid | 167.1049 | 167.1072 | −0.0023 | 21.174 |
| 3-(phenylmethoxy)-1-propanol | 167.1049 | 167.1072 | −0.0023 | 21.174 |
| norharman | 169.0772 | 169.0765 | 0.0007 | 69.625 |
| iridol | 169.0772 | 169.0864 | −0.0092 | 69.625 |
| vitamin B6 | 170.0858 | 170.0817 | 0.0041 | 14.214 |
| L-methylhistidine | 170.0858 | 170.0929 | −0.0071 | 14.214 |
| arginine | 175.1252 | 175.1195 | 0.0057 | 7.9928 |
| gramine | 175.1252 | 175.1235 | 0.0017 | 7.9928 |
| cinnamyl acetate | 177.092 | 177.0915 | 0.0005 | 25.067 |
| canavanine | 177.092 | 177.0987 | −0.0067 | 25.067 |
| coniferaldehyde | 179.0755 | 179.0708 | 0.0047 | 20.508 |
| methoxycinnamic acid | 179.0755 | 179.0708 | 0.0047 | 20.508 |
| D-mannosamine | 180.0947 | 180.0872 | 0.0075 | 71.465 |
| galacgtosamine | 180.0947 | 180.0872 | 0.0075 | 71.465 |
| glucosamine | 180.0947 | 180.0872 | 0.0075 | 71.465 |
| homophenylalanine | 180.0947 | 180.1024 | −0.0077 | 71.465 |
| salsolinol | 180.0947 | 180.1024 | −0.0077 | 71.465 |
| stilbene | 181.1051 | 181.1017 | 0.0034 | 23.706 |
| carbahcol | 183.0973 | 183.09 | 0.0073 | 23.277 |
| difluoromethylornithine | 183.0973 | 183.0945 | 0.0027 | 23.277 |
| dihydroconiferyl alcohol | 183.0973 | 183.1021 | −0.0048 | 23.277 |
| pinonic acid | 185.1191 | 185.1177 | 0.0013 | 16.717 |
| 3-methyl-2-butenoic acid, 2- | 185.1191 | 185.1177 | 0.0013 | 16.717 |
| baogongteng A | 186.1206 | 186.113 | 0.0075 | 5.7735 |
| proflavine | 188.1222 | 188.1187 | 0.0035 | 6.1099 |
| myristicin | 193.0959 | 193.0864 | 0.0094 | 19.432 |
| dehydrozingerone | 193.0959 | 193.0865 | 0.0094 | 19.432 |
| a-phenylindol | 194.1054 | 194.0969 | 0.0085 | 12.291 |
| dehydrocurcumene | 201.1632 | 201.1643 | −0.0011 | 16.445 |
| valeric acid phenylethylester | 207.1305 | 207.1385 | −0.008 | 15.471 |
| salsolidine | 208.1346 | 208.1337 | 0.0009 | 11.134 |
| isopilocarpine | 209.1353 | 209.129 | 0.0063 | 16.253 |
| philocarpine | 209.1353 | 209.129 | 0.0063 | 16.253 |
| (S)-(+)-carvone acetate | 211.1325 | 211.1334 | −0.0009 | 14.359 |
| hexylcinnamaldehyde | 217.1559 | 217.1592 | −0.0034 | 22.369 |
| ar-tumerone | 217.1559 | 217.1592 | −0.0034 | 22.369 |
| furanoeremophilane | 219.1748 | 219.1749 | −0.0001 | 31.416 |
| nootkatone | 219.1748 | 219.1749 | −0.0001 | 31.416 |
| valerenal | 219.1748 | 219.1749 | −0.0001 | 31.416 |
| xanthorrhizol | 219.1748 | 219.1749 | −0.0001 | 31.416 |
| curlone | 219.1748 | 219.1749 | −0.0001 | 31.416 |
| turmerone/ar-turmerol | 219.1748 | 219.1749 | −0.0001 | 31.416 |
| costunolide | 233.1548 | 233.1541 | 0.0007 | 34.224 |
| panthenol | 234.1713 | 234.1705 | 0.0007 | 19.135 |
| eremophilanlactone | 235.1677 | 235.1698 | −0.0021 | 40.793 |
| 2-octyl benzoate | 235.1677 | 235.1698 | −0.0021 | 40.793 |
| valerenic acid | 235.1677 | 235.1698 | −0.0021 | 40.793 |
| huperazine A | 243.1531 | 243.1497 | 0.0034 | 7.995 |
| dodec-2,4-diene-8,10-diynoic acid | 244.1606 | 244.1701 | −0.0095 | 12.683 |
| atractylenolide III | 249.158 | 249.149 | 0.0089 | 22.467 |
| parthenolide | 249.158 | 249.149 | 0.0089 | 22.467 |
| 6-paradol | 251.1645 | 251.1647 | −0.0002 | 27.036 |
| hydroxyvalerenic acid | 251.1645 | 251.1647 | −0.0002 | 27.036 |
| panaxydol | 261.1856 | 261.1854 | 0.0001 | 56.759 |
| adenosine | 268.1075 | 268.1046 | 0.0028 | 56.971 |
| estrone | 271.1658 | 271.1698 | −0.004 | 13.174 |
| 17-estradiol | 273.1829 | 273.1854 | −0.0026 | 10.592 |
| 19-nor-4-androstene-3,17-diol | 273.1829 | 273.1854 | −0.0026 | 10.592 |
| 1,6-octadien-3-ol, 3,7-dimethyl | 274.1852 | 274.1807 | 0.0044 | 12.167 |
| podocarpic acid | 275.1742 | 275.1647 | 0.0095 | 23.435 |
| 6-shogaol | 277.1808 | 277.1803 | 0.0005 | 48.923 |
| menthyl salicylate | 277.1808 | 277.1803 | 0.0005 | 48.923 |
| cyclohexanecarboxylic acid, | 277.1808 | 277.1803 | 0.0005 | 48.923 |
| 8-paradol | 279.2057 | 279.196 | 0.0096 | 11.949 |
| androstenedione | 287.1985 | 287.2011 | −0.0026 | 11.641 |
| 17a-methyl-19-nortestosteron | 289.2104 | 289.2167 | −0.0063 | 14.462 |
| androstanedione | 289.2104 | 289.2167 | −0.0063 | 14.462 |
| dehydroisoandosterone(DHEA) | 289.2104 | 289.2167 | −0.0063 | 14.462 |
| testosterone | 289.2104 | 289.2167 | −0.0063 | 14.462 |
| 7-shogaol | 291.1996 | 291.196 | 0.0036 | 12.292 |
| N-octyl-B-D-glucopyranoside | 293.2032 | 293.1964 | 0.0067 | 17.563 |
| 6-gingerdiol | 297.2 | 297.2066 | −0.0066 | 14.41 |
| retinoic acid | 301.2223 | 301.2167 | 0.0056 | 14.908 |
| C20H28O2 | 301.2223 | 301.2167 | 0.0056 | 14.908 |
| abietic acid | 303.2227 | 303.2324 | −0.0098 | 19.293 |
| eicosapentaenoic acid | 303.2227 | 303.2324 | −0.0098 | 19.293 |
| 8-shogaol | 305.2172 | 305.2117 | 0.0055 | 12.248 |
| 10-paradol | 307.222 | 307.2273 | −0.0053 | 13.321 |
| dihydrocapsaicin | 308.2193 | 308.2225 | −0.0032 | 12.858 |
| dihydrocapsaicin | 308.2193 | 308.2226 | −0.0033 | 12.858 |
| ethisterone | 313.2249 | 313.2167 | 0.0082 | 16.741 |
| guggulsterone | 313.2249 | 313.2167 | 0.0082 | 16.741 |
| kahweol | 315.202 | 315.196 | 0.006 | 14.769 |
| cafestol | 317.2166 | 317.2116 | 0.0049 | 15.569 |
| petasine | 317.2166 | 317.2117 | 0.0049 | 15.569 |
| galanolactone/aframodial/gal | 319.2205 | 319.2273 | −0.0068 | 13.606 |
| homocapsaicin | 320.2306 | 320.2226 | 0.008 | 15.125 |
| homodihydrocapsaicin | 322.2431 | 322.2382 | 0.0049 | 15.364 |
| 8-gingerdiol | 325.2281 | 325.2379 | −0.0098 | 13.119 |
| ajmaline | 327.2153 | 327.2072 | 0.0081 | 13.676 |
| deoxy-andrographolide | 335.2305 | 335.2222 | 0.0083 | 11.475 |
| lobelanidine | 340.2326 | 340.2276 | 0.005 | 17.261 |
| yohimbic acid | 341.195 | 341.1865 | 0.0085 | 17.417 |
| menisperine | 341.195 | 341.1985 | −0.0035 | 17.417 |
| 10-dehydrogingerdione | 347.2189 | 347.2222 | −0.0034 | 11.822 |
| calycanthine | 347.2189 | 347.2235 | −0.0047 | 11.822 |
| tetrahydrocorticosterone | 351.2473 | 351.2535 | −0.0062 | 14.536 |
| corynanthine/vincamine/yohimbine | 355.198 | 355.2021 | −0.0041 | 10.211 |
| cafestol acetate | 359.2206 | 359.2222 | −0.0016 | 11.071 |
| odorigenin/digitoxigenin | 375.2459 | 375.2535 | −0.0076 | 12.181 |
| resibufogenin | 385.2477 | 385.2379 | 0.0097 | 15.406 |
| mevinolin | 405.2607 | 405.2641 | −0.0035 | 10.042 |
| lupulone | 415.2912 | 415.2848 | 0.0064 | 8.9322 |
| neoruscogenin | 429.2978 | 429.3005 | −0.0027 | 9.4811 |
| 4-methylumbelliferyl elaidate | 441.2968 | 441.3005 | −0.0037 | 9.1414 |
| vitamin K2(menaquinone) | 445.3062 | 445.3106 | −0.0044 | 7.112 |
| condelphine | 450.2875 | 450.2855 | 0.0019 | 8.5232 |
| glycocholic acid | 466.3234 | 466.3168 | 0.0066 | 9.4874 |
| hovenolactone/trevoagenin D | 489.3494 | 489.358 | −0.0086 | 6.9963 |
| ganolucidic acid B | 501.324 | 501.3216 | 0.0024 | 4.6363 |
| acetylketoboswellic acid | 513.3602 | 513.358 | 0.0021 | 4.8801 |
| ganoderic acids A/B | 517.3183 | 517.3165 | 0.0018 | 4.1054 |
| alloxanthin | 565.395 | 565.4046 | −0.0096 | 4.0912 |
| canthaxanthin | 565.395 | 565.4046 | −0.0096 | 4.0912 |
| carbenoxolone | 571.3724 | 571.3635 | 0.0088 | 3.6649 |
| diadinoxanthin | 583.407 | 583.4151 | −0.0081 | 3.8055 |
| gymnemic acid IV/XIV - GlcA | 589.4152 | 589.4104 | 0.0047 | 3.9361 |
| gymnemic acid III/XIII - Glc | 591.4265 | 591.4261 | 0.0004 | 3.9858 |
| astaxanthin | 597.3858 | 597.3944 | −0.0086 | 1.9967 |
| cimiracemoside C | 621.4064 | 621.4002 | 0.0062 | 2.1688 |
| gymnemasaponin II - Glc | 637.4351 | 637.4315 | 0.0036 | 1.8635 |
| saponin H | 651.4089 | 651.4108 | −0.002 | 1.5592 |
| gymnemic acid V/XI/XVI - Glc | 671.4471 | 671.4523 | −0.0051 | 0.779 |

### Compounds in Phenolic Acid Fraction: Polymer Adsorbent Processing: Collected Fraction 3

6-gingerol was present in this extract in 2.9% relative abundance. Other shagoals, paradols, gingerols and giongerdiols were also present in this extract. Amino acids, vitamins, fatty acids, saccharides, phenolic acids, phenols, sterols, alkaloids, chalcones, coumarins and hydrocarbons were also present in this extract. 84 out of 159 (53%) unique chemicals have been directly identified in this extract using the DART TOF-MS. Table 29 shows the compounds identified in the extracts along with their relative abundance. FIG. 11D shows the DART Spectrum of this extract.

TABLE 29
Compounds in polymer adsorbent processing: collected fraction 3

| Compound | Meas. | Calc. | Diff(u) | Abund. |
|---|---|---|---|---|
| 1,4-benzoquinone | 109.0298 | 109.0289 | 0.0009 | 6.2008 |
| 6-azauracil | 113.0255 | 113.0225 | 0.003 | 2.0289 |
| uracil | 113.0255 | 113.0351 | −0.0096 | 2.0289 |
| levulinic acid | 117.0561 | 117.0551 | 0.001 | 3.4735 |
| guaiacol | 125.0611 | 125.0602 | 0.0008 | 11.03 |
| methoxyphenol | 125.0611 | 125.0602 | 0.0008 | 11.03 |
| methylcatechol | 125.0611 | 125.0602 | 0.0008 | 11.03 |
| salicyl alcohol | 125.0611 | 125.0602 | 0.0008 | 11.03 |
| 2-methoxyphenol | 125.0611 | 125.0603 | 0.0008 | 11.03 |
| pyrogallol/phlorglucinol/maltol | 127.042 | 127.0395 | 0.0025 | 10.086 |
| 6-methyluracil | 127.042 | 127.0507 | −0.0087 | 10.086 |
| adenine | 136.0641 | 136.0623 | 0.0018 | 10.017 |
| anisaldehyde/formic acid benzoate | 137.061 | 137.0602 | 0.0008 | 89.018 |
| trigonelline/vitamin H | 138.0633 | 138.0555 | 0.0078 | 5.3086 |
| furfuryl acetate | 141.0563 | 141.0551 | 0.0012 | 16.659 |
| 3-hydroxy-2,3 dihydromaltol | 145.0518 | 145.0501 | 0.0017 | 4.1047 |
| 2-methoxy-4-vinylphenol | 151.0781 | 151.0759 | 0.0022 | 17.381 |
| benzoic acid ethyl ester | 151.0781 | 151.0759 | 0.0022 | 17.381 |
| cresyl acetate | 151.0781 | 151.0759 | 0.0022 | 17.381 |
| hydrocinnamic acid | 151.0781 | 151.0759 | 0.0022 | 17.381 |
| 2-acetyl-3-ethylpyrazine | 151.0781 | 151.0871 | −0.009 | 17.381 |
| decadienal/santolina epoxide | 153.1281 | 153.1279 | 0.0001 | 25.384 |
| pinene oxide | 153.1281 | 153.1279 | 0.0001 | 25.384 |
| doederleinic acid | 157.0529 | 157.0501 | 0.0028 | 6.4024 |
| methyl cinnamic acid | 163.0773 | 163.0759 | 0.0014 | 56.301 |
| safrole | 163.0773 | 163.0759 | 0.0014 | 56.301 |
| methoxycinnamaldehyde | 163.0773 | 163.0759 | 0.0014 | 56.301 |
| 4-hydroxyphenyl-2-butanone | 165.0946 | 165.0915 | 0.0031 | 15.19 |
| acetic acid phenethyl ester | 165.0946 | 165.0915 | 0.0031 | 15.19 |
| eugenol | 165.0946 | 165.0915 | 0.0031 | 15.19 |
| isoeugenol | 165.0946 | 165.0915 | 0.0031 | 15.19 |
| phenylacetic acid ethylester | 165.0946 | 165.0915 | 0.0031 | 15.19 |
| eugenol | 165.0946 | 165.0916 | 0.0031 | 15.19 |
| phenylalanine | 166.0913 | 166.0868 | 0.0045 | 13.215 |
| norharman | 169.0845 | 169.0765 | 0.0079 | 19.469 |
| iridol | 169.0845 | 169.0864 | −0.002 | 19.469 |
| coniferaldehyde | 179.0718 | 179.0708 | 0.001 | 33.931 |
| methoxycinnamic acid | 179.0718 | 179.0708 | 0.001 | 33.931 |
| homophenylalanine | 180.1002 | 180.1024 | −0.0022 | 11.986 |
| salsolinol | 180.1002 | 180.1024 | −0.0022 | 11.986 |
| stilbene | 181.099 | 181.1017 | −0.0027 | 17.291 |
| 2,3-dimethoxy-5-methylbenzoquinone | 183.0636 | 183.0657 | −0.0021 | 2.1651 |
| 2,3-dimethoxybenzoic acid | 183.0636 | 183.0657 | −0.0021 | 2.1651 |
| dihydrocaffeic acid | 183.0636 | 183.0657 | −0.0021 | 2.1651 |
| veratric acid | 183.0636 | 183.0657 | −0.0021 | 2.1651 |
| homovanillic acid | 183.0636 | 183.0657 | −0.0021 | 2.1651 |
| harmane | 185.1177 | 185.1078 | 0.0099 | 5.3988 |
| pinonic acid | 185.1177 | 185.1177 | 0 | 5.3988 |
| n-acetyl-L-glutamine | 189.092 | 189.0875 | 0.0044 | 2.4378 |
| 2,6-diaminopimelic acid | 191.1065 | 191.1032 | 0.0033 | 8.0085 |
| ligustilide | 191.1065 | 191.1072 | −0.0007 | 8.0085 |
| 4-phenylbutylisothiocyanate | 192.0923 | 192.0847 | 0.0075 | 3.3038 |
| myristicin | 193.0887 | 193.0864 | 0.0022 | 29.297 |
| dehydrozingerone | 193.0887 | 193.0865 | 0.0022 | 29.297 |
| a-phenylindol | 194.0917 | 194.0969 | −0.0053 | 5.1849 |
| kynurenine | 209.0935 | 209.0926 | 0.0009 | 9.6683 |
| chalcone | 209.0935 | 209.0966 | −0.0031 | 9.6683 |
| flavan | 211.108 | 211.1123 | −0.0044 | 3.3649 |
| harmine | 213.1103 | 213.1028 | 0.0074 | 4.3617 |
| n-acetyl-DL-arginne | 217.1204 | 217.13 | −0.0096 | 7.8668 |
| abrine | 219.1201 | 219.1133 | 0.0068 | 9.2172 |
| homotryptophan | 219.1201 | 219.1133 | 0.0068 | 9.2172 |
| n-acetyl-serotonin | 219.1201 | 219.1133 | 0.0068 | 9.2172 |
| vitamin B5 | 220.1092 | 220.1185 | −0.0093 | 1.5386 |
| pantothenic acid | 220.1092 | 220.1185 | −0.0094 | 1.5386 |
| terphenyl | 231.1132 | 231.1174 | −0.0042 | 6.6829 |
| costunolide | 233.1525 | 233.1541 | −0.0016 | 15.903 |
| osthole | 245.1217 | 245.1177 | 0.0039 | 4.8234 |
| santonin | 247.1342 | 247.1334 | 0.0007 | 6.6885 |
| atractylenolide III | 249.1452 | 249.149 | −0.0038 | 5.1592 |
| parthenolide | 249.1452 | 249.149 | −0.0038 | 5.1592 |
| 6-paradol | 251.1617 | 251.1647 | −0.003 | 2.8793 |
| hydroxyvalerenic acid | 251.1617 | 251.1647 | −0.003 | 2.8793 |
| palmitic acid | 257.2497 | 257.248 | 0.0016 | 1.9664 |
| panaxydol | 261.189 | 261.1854 | 0.0036 | 56.201 |
| abscisic acid | 265.1513 | 265.144 | 0.0073 | 4.0288 |
| podocarpic acid | 275.1738 | 275.1647 | 0.0091 | 6.5138 |
| linolenic acid | 279.2355 | 279.2324 | 0.0031 | 4.042 |
| 9,12,15-octadecatrienoic acid | 279.2355 | 279.2324 | 0.0031 | 4.042 |
| 16-oxokahweol | 283.1761 | 283.1698 | 0.0063 | 100 |
| miltirone | 283.1761 | 283.1698 | 0.0063 | 100 |
| 16-oxocafestol | 285.1888 | 285.1854 | 0.0033 | 5.828 |
| embelin | 295.1916 | 295.1909 | 0.0006 | 2.9084 |
| 6-gingerol | 295.1916 | 295.1909 | 0.0006 | 2.9084 |
| 6-gingerdiol | 297.1997 | 297.2066 | −0.0069 | 5.4566 |
| retinoic acid | 301.2159 | 301.2167 | −0.0008 | 5.0919 |
| abietic acid | 303.2262 | 303.2324 | −0.0062 | 2.6172 |
| eicosapentaenoic acid | 303.2262 | 303.2324 | −0.0062 | 2.6172 |
| sarpagine | 311.1814 | 311.1759 | 0.0055 | 12.774 |
| kahweol | 315.1974 | 315.196 | 0.0013 | 4.1126 |
| cafestol | 317.2124 | 317.2116 | 0.0008 | 3.6671 |
| petasine | 317.2124 | 317.2117 | 0.0008 | 3.6671 |
| ajmaline | 327.202 | 327.2072 | −0.0052 | 85.288 |
| crocetin/geranoxy methoxycoumarin | 329.183 | 329.1753 | 0.0076 | 4.4891 |
| bavachinin A/bergamotin | 339.1674 | 339.1596 | 0.0078 | 12.214 |
| magnoflorine | 343.1821 | 343.1783 | 0.0038 | 16.541 |
| 10-gingerdiol | 353.2693 | 353.2692 | 0.0002 | 3.4199 |
| prednisone/myricanol | 359.1856 | 359.1858 | −0.0003 | 9.4463 |
| corydaline | 370.1996 | 370.2018 | −0.0022 | 7.815 |
| aricine | 383.1931 | 383.1971 | −0.0041 | 1.6424 |
| lucigenin | 387.1862 | 387.1861 | 0 | 11.322 |
| pravastatin/strophanthidol | 407.2359 | 407.2433 | −0.0075 | 6.205 |
| ascorbyl palmitate | 415.2648 | 415.2696 | −0.0049 | 15.167 |

### Compounds in Phenolic Acid Fraction: Polymer Adsorbent Processing: Collected Fraction 4

6-shogoal, 6-gingerol and galanolactone were present in this extract in 100, 8.3 and 5.5% relative abundance, respectively. Other shagoals, paradols, gingerols and giongerdiols were also present in this extract. Amino acids, vitamins, fatty acids, saccharides, phenolic acids, phenols, sterols, capsaicins, alkaloids, ganoderols, xanthines, gymnemagins, boswellic acids, saponins, and hydrocarbons were also present in this extract. 151 out of 628 (24%) unique chemicals have been directly identified in this extract using the DART TOF-MS. Table 30 shows the compounds identified in the extracts along with their relative abundance. FIG. 11E shows the DART Spectrum of this extract.

TABLE 30
Compounds in polymer adsorbent processing: collected fraction 4

| Compound | Meas. | Calc. | Diff(u) | Abund. |
|---|---|---|---|---|
| 2-acetylpyrrole | 110.0618 | 110.0606 | 0.0012 | 0.2532 |
| histamine | 112.0876 | 112.0874 | 0.0002 | 19.388 |
| 2-methylcyclohexanone | 113.0891 | 113.0966 | −0.0075 | 1.6028 |
| pyrogallol/phlorglucinol/maltol | 127.0477 | 127.0395 | 0.0081 | 0.4129 |
| 6-methyluracil | 127.0477 | 127.0507 | −0.0031 | 0.4129 |
| leucine | 132.1122 | 132.1024 | 0.0097 | 0.183 |
| p-cymene | 135.1198 | 135.1174 | 0.0024 | 1.8326 |
| adenine | 136.0657 | 136.0623 | 0.0033 | 4.8186 |
| anisaldehyde/formic acid benzoate | 137.0602 | 137.0602 | 0 | 19.756 |
| furfuryl acetate | 141.0636 | 141.0551 | 0.0085 | 0.557 |
| tropine | 142.1195 | 142.1232 | −0.0038 | 0.6169 |
| octalactone | 143.1064 | 143.1072 | −0.0008 | 1.5285 |
| baogongteng B | 144.1052 | 144.1024 | 0.0028 | 2.0389 |
| lysine | 147.117 | 147.1133 | 0.0036 | 2.952 |
| anethole | 149.0966 | 149.0966 | 0 | 0.2859 |
| cuminaldehyde | 149.0966 | 149.0966 | 0 | 0.2859 |
| estragole | 149.0966 | 149.0966 | 0 | 0.2859 |
| propylbenzaldehyde | 149.0966 | 149.0966 | 0 | 0.2859 |
| 2-acetyl-3-ethylpyrazine | 151.088 | 151.0871 | 0.0008 | 3.153 |
| decadienal/santolina epoxide | 153.1284 | 153.1279 | 0.0005 | 3.9299 |
| pinene oxide | 153.1284 | 153.1279 | 0.0005 | 3.9299 |
| diphenyl | 155.0913 | 155.0861 | 0.0052 | 1.2961 |
| arecoline/hydroxytropinone | 156.1069 | 156.1024 | 0.0045 | 0.7072 |
| betonicine/acetyl valine | 160.0967 | 160.0973 | −0.0007 | 1.4215 |
| methylcholine | 161.134 | 161.1416 | −0.0076 | 2.0386 |
| carnitine, L- | 162.1097 | 162.113 | −0.0033 | 2.1828 |
| methyl cinnamic acid | 163.0767 | 163.0759 | 0.0008 | 22.556 |
| safrole | 163.0767 | 163.0759 | 0.0008 | 22.556 |
| methoxycinnamaldehyde | 163.0767 | 163.0759 | 0.0008 | 22.556 |
| N-phenylmorpholine | 164.0981 | 164.1075 | −0.0095 | 6.2518 |
| 4-hydroxyphenyl-2-butanone | 165.0987 | 165.0915 | 0.0072 | 2.7417 |
| acetic acid phenethyl ester | 165.0987 | 165.0915 | 0.0072 | 2.7417 |
| tert-butyl-p-quinone | 165.0987 | 165.0915 | 0.0072 | 2.7417 |
| eugenol | 165.0987 | 165.0915 | 0.0072 | 2.7417 |
| isoeugenol | 165.0987 | 165.0915 | 0.0072 | 2.7417 |
| phenylacetic acid ethylester | 165.0987 | 165.0915 | 0.0072 | 2.7417 |
| eugenol | 165.0987 | 165.0916 | 0.0072 | 2.7417 |
| synephrine | 168.1077 | 168.1024 | 0.0053 | 2.0265 |
| norharman | 169.0773 | 169.0765 | 0.0008 | 13.627 |
| iridol | 169.0773 | 169.0864 | −0.0091 | 13.627 |
| vitamin B6 | 170.0866 | 170.0817 | 0.0049 | 2.4123 |
| L-methylhistidine | 170.0866 | 170.0929 | −0.0064 | 2.4123 |
| n-acetyl-DL-leucine | 174.1201 | 174.113 | 0.0071 | 2.9208 |
| swainsonine | 174.1201 | 174.113 | 0.0071 | 2.9208 |
| cinnamyl acetate | 177.0908 | 177.0915 | −0.0007 | 27.683 |
| canavanine | 177.0908 | 177.0987 | −0.0079 | 27.683 |
| coniferaldehyde | 179.0743 | 179.0708 | 0.0035 | 8.1787 |
| methoxycinnamic acid | 179.0743 | 179.0708 | 0.0035 | 8.1787 |
| homophenylalanine | 180.1017 | 180.1024 | −0.0007 | 18.073 |
| salsolinol | 180.1017 | 180.1024 | −0.0007 | 18.073 |
| stilbene | 181.1061 | 181.1017 | 0.0044 | 5.0821 |
| carbahcol | 183.097 | 183.09 | 0.007 | 5.683 |
| difluoromethylornithine | 183.097 | 183.0945 | 0.0025 | 5.683 |
| dihydroconiferyl alcohol | 183.097 | 183.1021 | −0.0051 | 5.683 |
| harmane | 185.1152 | 185.1078 | 0.0074 | 1.8445 |
| pinonic acid | 185.1152 | 185.1177 | −0.0025 | 1.8445 |
| DL-eleagnin | 187.1295 | 187.1235 | 0.0059 | 1.3965 |
| 10-hydroxy-2-decenoic acid | 187.1295 | 187.1334 | −0.0039 | 1.3965 |
| myristicin | 193.0907 | 193.0864 | 0.0043 | 6.8273 |
| dehydrozingerone | 193.0907 | 193.0865 | 0.0042 | 6.8273 |
| a-phenylindol | 194.1017 | 194.0969 | 0.0048 | 4.8303 |
| methyl-B-D-glucopyranoside | 195.0964 | 195.0868 | 0.0095 | 2.4894 |
| methylgalactopyranoside | 195.0964 | 195.0868 | 0.0095 | 2.4894 |
| caffeine | 195.0964 | 195.0882 | 0.0082 | 2.4894 |
| zingerone | 195.0964 | 195.1021 | −0.0057 | 2.4894 |
| dihydromyristicin | 195.0964 | 195.1021 | −0.0058 | 2.4894 |
| dehydrocurcumene (proposed c | 201.1649 | 201.1643 | 0.0006 | 7.5192 |
| curcumene/cuparene/calamenen | 203.1805 | 203.18 | 0.0005 | 25.237 |
| salsolidine | 208.1378 | 208.1337 | 0.004 | 4.0841 |
| asarone | 209.1266 | 209.1177 | 0.0089 | 2.9011 |
| isopilocarpine | 209.1266 | 209.129 | −0.0024 | 2.9011 |
| philocarpine | 209.1266 | 209.129 | −0.0024 | 2.9011 |
| (S)-(+)-carvone acetate | 211.1279 | 211.1334 | −0.0055 | 2.6539 |
| hexylcinnamaldehyde | 217.1589 | 217.1592 | −0.0003 | 7.8417 |
| ar-tumerone | 217.1589 | 217.1592 | −0.0003 | 7.8417 |
| furanoeremophilane | 219.1758 | 219.1749 | 0.0009 | 16.675 |
| nootkatone | 219.1758 | 219.1749 | 0.0008 | 16.675 |
| valerenal | 219.1758 | 219.1749 | 0.0008 | 16.675 |
| xanthorrhizol | 219.1758 | 219.1749 | 0.0008 | 16.675 |
| curlone | 219.1758 | 219.1749 | 0.0008 | 16.675 |
| turmerone/ar-turmerol | 219.1758 | 219.1749 | 0.0008 | 16.675 |
| caryophyllene oxide | 221.192 | 221.1905 | 0.0015 | 12.422 |
| bergamotol | 221.192 | 221.1905 | 0.0015 | 12.422 |
| spathulenol/9-cedranone/lanceol | 221.192 | 221.1905 | 0.0015 | 12.422 |
| terphenyl | 231.1253 | 231.1174 | 0.0079 | 11.952 |
| undec-2-ene-8,10-diynoic acid | 232.1673 | 232.1701 | −0.0028 | 6.6668 |
| costunolide | 233.1576 | 233.1541 | 0.0034 | 9.0214 |
| panthenol | 234.1709 | 234.1705 | 0.0004 | 5.0466 |
| eremophilanlactone | 235.1689 | 235.1698 | −0.0009 | 14.587 |
| 2-octyl benzoate | 235.1689 | 235.1698 | −0.0009 | 14.587 |
| valerenic acid | 235.1689 | 235.1698 | −0.0009 | 14.587 |
| vellerdiol | 237.1816 | 237.1854 | −0.0038 | 7.1431 |
| a-ionyl acetate | 237.1816 | 237.1854 | −0.0038 | 7.1431 |
| flavanone hydrazone | 239.1181 | 239.1184 | −0.0003 | 0.5586 |
| isobornyl isovalerate | 239.1983 | 239.2011 | −0.0028 | 0.5491 |
| linalyl iso-valerate | 239.1983 | 239.2011 | −0.0028 | 0.5491 |
| huperazine A | 243.1456 | 243.1497 | −0.0041 | 1.5184 |
| atractylenolide III | 249.1553 | 249.149 | 0.0063 | 10.303 |
| parthenolide | 249.1553 | 249.149 | 0.0063 | 10.303 |
| 6-paradol | 251.167 | 251.1647 | 0.0022 | 7.3163 |
| hydroxyvalerenic acid | 251.167 | 251.1647 | 0.0022 | 7.3163 |
| panaxydol | 261.1868 | 261.1854 | 0.0013 | 13.167 |
| 1,6-octadien-3-ol, 3,7-dimethyl | 274.1823 | 274.1807 | 0.0016 | 4.4175 |
| podocarpic acid | 275.1727 | 275.1647 | 0.008 | 8.717 |
| 6-shogaol | 277.1795 | 277.1803 | −0.0008 | 100 |
| menthyl salicylate | 277.1795 | 277.1803 | −0.0008 | 100 |
| cyclohexanecarboxylic acid | 277.1795 | 277.1803 | −0.0008 | 100 |
| 6-shogaol | 277.1795 | 277.1804 | −0.0008 | 100 |
| 8-paradol | 279.1914 | 279.196 | −0.0046 | 6.6254 |
| androstenedione | 287.1998 | 287.2011 | −0.0014 | 6.9021 |
| 17a-methyl-19-nortestosterone | 289.2242 | 289.2167 | 0.0074 | 7.1331 |
| androstanedione | 289.2242 | 289.2167 | 0.0074 | 7.1331 |
| dehydroisoandosterone(DHEA) | 289.2242 | 289.2167 | 0.0074 | 7.1331 |
| testosterone | 289.2242 | 289.2167 | 0.0074 | 7.1331 |
| atropine | 291.1881 | 291.1834 | 0.0047 | 8.5798 |
| 7-shogaol | 291.1881 | 291.196 | −0.0079 | 8.5798 |
| N-octyl-B-D-glucopyranoside | 293.1887 | 293.1964 | −0.0077 | 7.9377 |
| embelin | 295.1967 | 295.1909 | 0.0057 | 8.2909 |
| 6-gingerol | 295.1967 | 295.1909 | 0.0057 | 8.2909 |
| 6-gingerdiol | 297.2071 | 297.2066 | 0.0005 | 5.1095 |
| 9,12-octadecadienoyl chloride | 299.2052 | 299.2141 | −0.009 | 4.0511 |
| abietic acid | 303.2296 | 303.2324 | −0.0028 | 11.801 |
| eicosapentaenoic acid | 303.2296 | 303.2324 | −0.0028 | 11.801 |
| 8-shogaol | 305.2211 | 305.2117 | 0.0095 | 9.3321 |
| dihydrocapsaicin | 308.2132 | 308.2225 | −0.0093 | 5.7902 |
| ethisterone | 313.2123 | 313.2167 | −0.0044 | 2.7706 |
| guggulsterone | 313.2123 | 313.2167 | −0.0044 | 2.7706 |
| kahweol | 315.2045 | 315.196 | 0.0084 | 4.3348 |
| cafestol | 317.2193 | 317.2116 | 0.0077 | 5.9874 |
| petasine | 317.2193 | 317.2117 | 0.0076 | 5.9874 |
| galanolactone/aframodial/galanal | 319.2276 | 319.2273 | 0.0003 | 5.53 |
| homodihydrocapsaicin | 322.2398 | 322.2382 | 0.0016 | 6.5653 |
| 10-shogaol | 333.2403 | 333.243 | −0.0026 | 7.1283 |
| lobelanidine | 340.2282 | 340.2276 | 0.0006 | 4.7736 |
| yohimbic acid | 341.1854 | 341.1865 | −0.0011 | 7.4786 |
| 10-dehydrogingerdione | 347.2315 | 347.2222 | 0.0093 | 2.2657 |
| calycanthine | 347.2315 | 347.2235 | 0.008 | 2.2657 |
| 10-gingerdione | 349.2388 | 349.2379 | 0.0009 | 2.9499 |
| tetrahydrocorticosterone | 351.2504 | 351.2535 | −0.0032 | 4.3562 |
| laudanosine | 358.2067 | 358.2018 | 0.0049 | 6.1404 |
| cafestol acetate | 359.2208 | 359.2222 | −0.0014 | 4.4943 |
| uncarine/mitraphylline | 369.1877 | 369.1814 | 0.0062 | 8.2697 |
| tamoxifen | 372.2411 | 372.2327 | 0.0084 | 3.1651 |
| diacetyl-6-gingerdiol | 381.2358 | 381.2277 | 0.0081 | 2.2763 |
| resibufogenin | 385.2345 | 385.2379 | −0.0035 | 5.5455 |
| octyl phthalate | 391.2837 | 391.2848 | −0.0011 | 4.2528 |
| dehydrocholic acid | 403.2546 | 403.2484 | 0.0062 | 4.6957 |
| mevinolin | 405.2558 | 405.2641 | −0.0083 | 3.4105 |
| cholic acid | 409.2921 | 409.2954 | −0.0033 | 3.5847 |
| lupulone | 415.2892 | 415.2848 | 0.0044 | 1.959 |
| jervine | 426.2923 | 426.3008 | −0.0085 | 5.5861 |
| neoruscogenin | 429.3027 | 429.3005 | 0.0022 | 3.2756 |
| 4-methylumbelliferyl elaidate | 441.2975 | 441.3005 | −0.003 | 3.2866 |
| vitamin K2(menaquinone) | 445.3066 | 445.3106 | −0.004 | 2.0333 |
| condelphine | 450.2857 | 450.2855 | 0.0002 | 2.7028 |
| glycocholic acid | 466.3239 | 466.3168 | 0.0071 | 2.8887 |
| hovenolactone/trevoagenin D | 489.3513 | 489.358 | −0.0066 | 2.3028 |
| gymnemagenin | 492.3371 | 492.3451 | −0.0081 | 3.4641 |
| acetylboswellic acid/ganoderol | 499.371 | 499.3787 | −0.0078 | 1.4751 |
| ganoderic acids A/B | 517.3206 | 517.3165 | 0.004 | 1.2687 |
| belladonin hydrogen sulfate | 543.3298 | 543.3223 | 0.0074 | 1.6598 |
| gymnemic acid X - GlcA | 549.3816 | 549.3791 | 0.0025 | 1.9842 |
| carbenoxolone | 571.3723 | 571.3635 | 0.0088 | 1.7038 |
| cimiracemoside C | 621.41 | 621.4002 | 0.0098 | 0.3105 |
| gymnemasaponin II - Glc | 637.4269 | 637.4315 | −0.0047 | 0.2645 |
| fucoxanthin | 659.4294 | 659.4311 | −0.0017 | 0.3448 |

### Example 6

### Example of Step 4 (FIG. 5)

### Polysaccharide Fraction Extraction

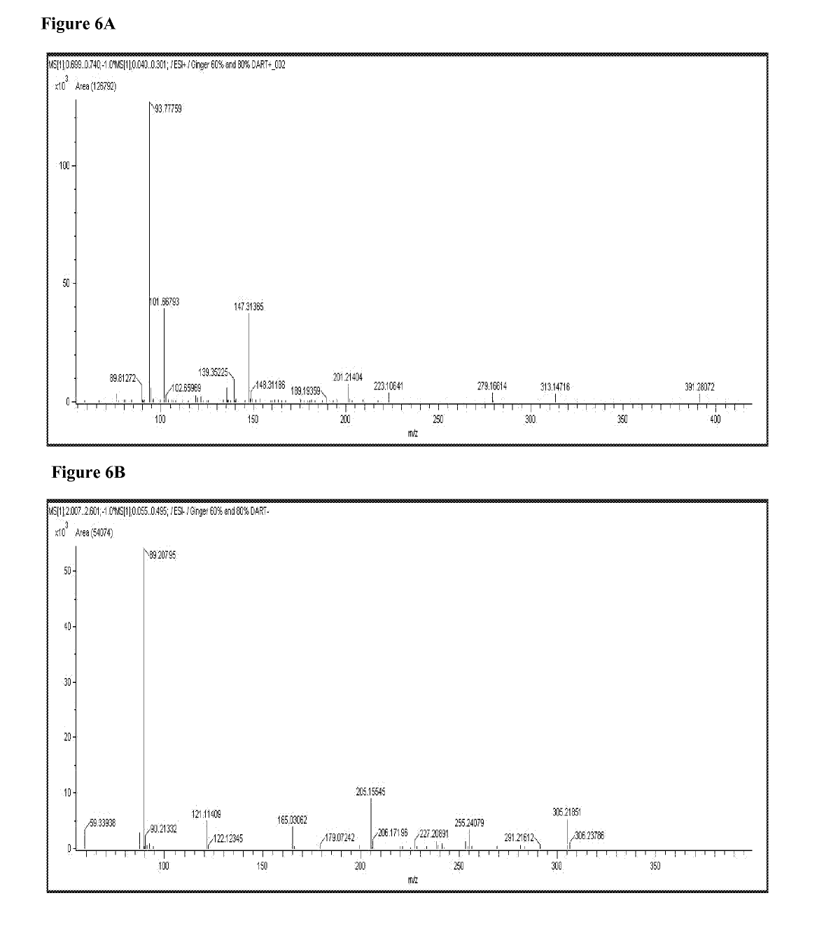

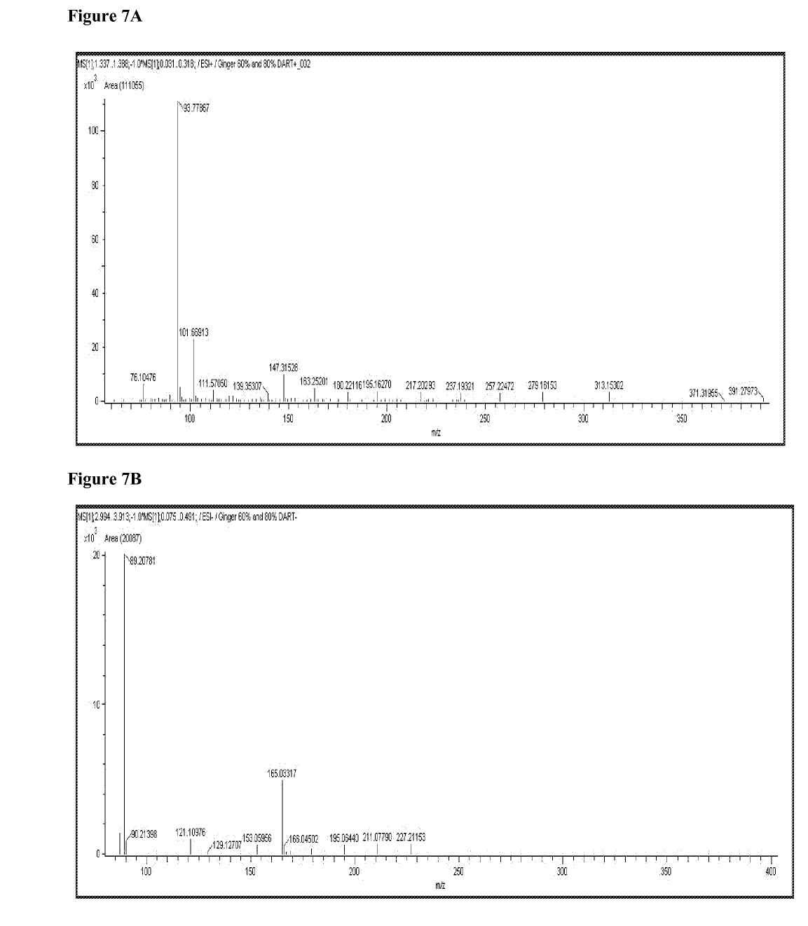

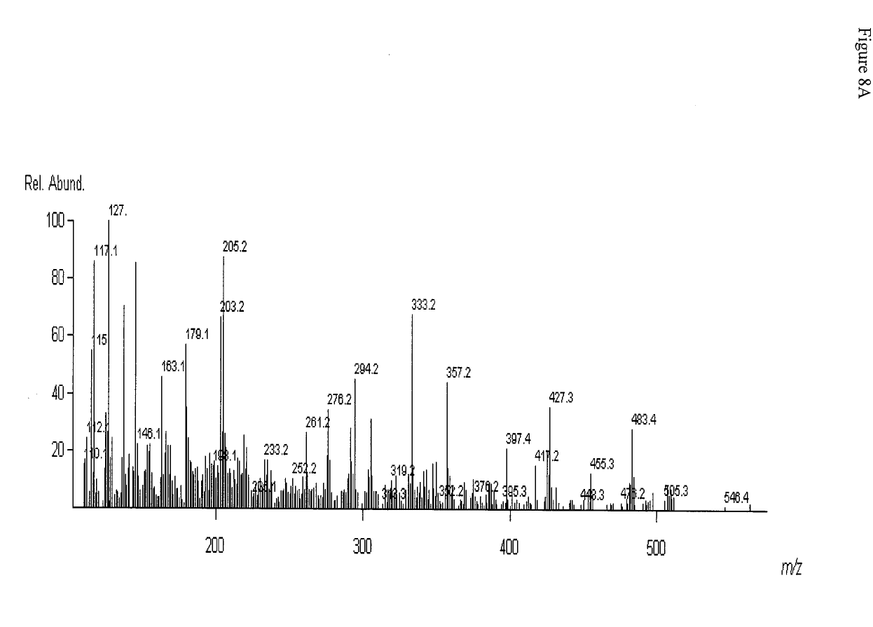

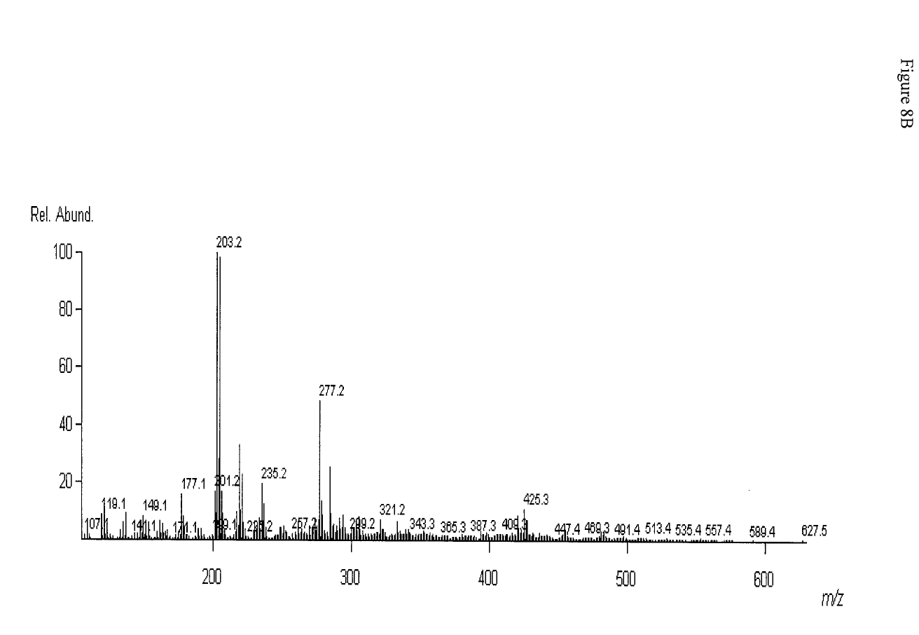

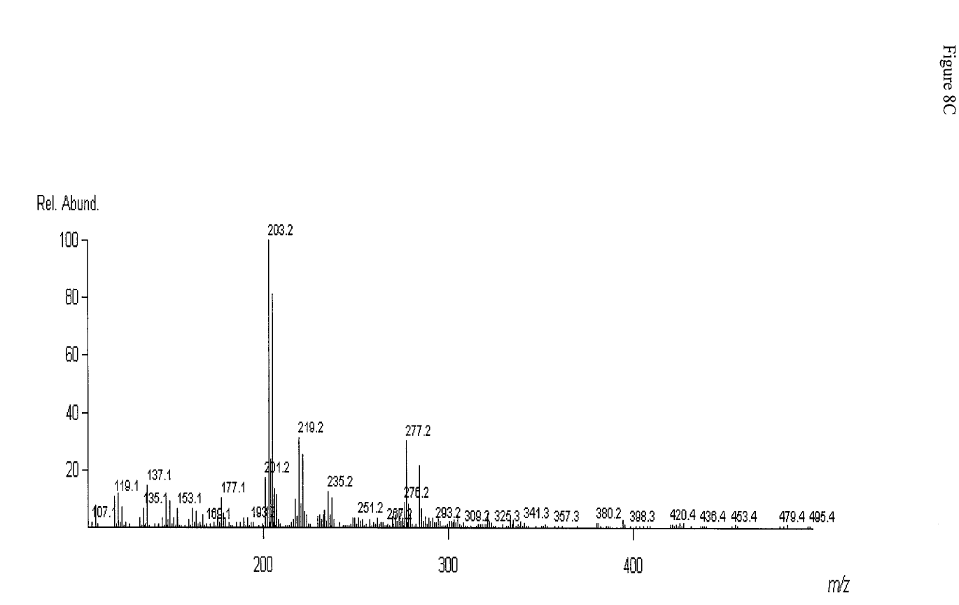

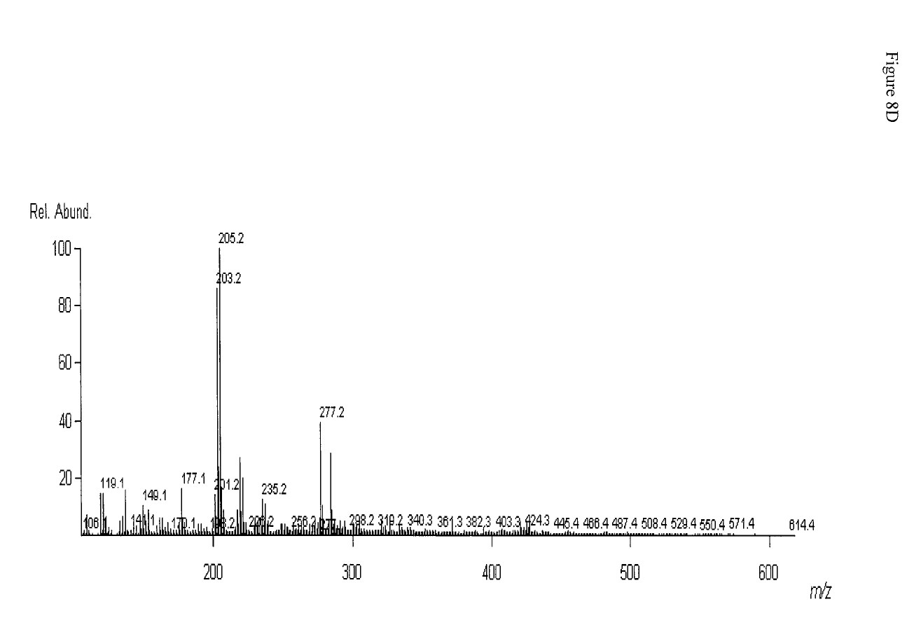

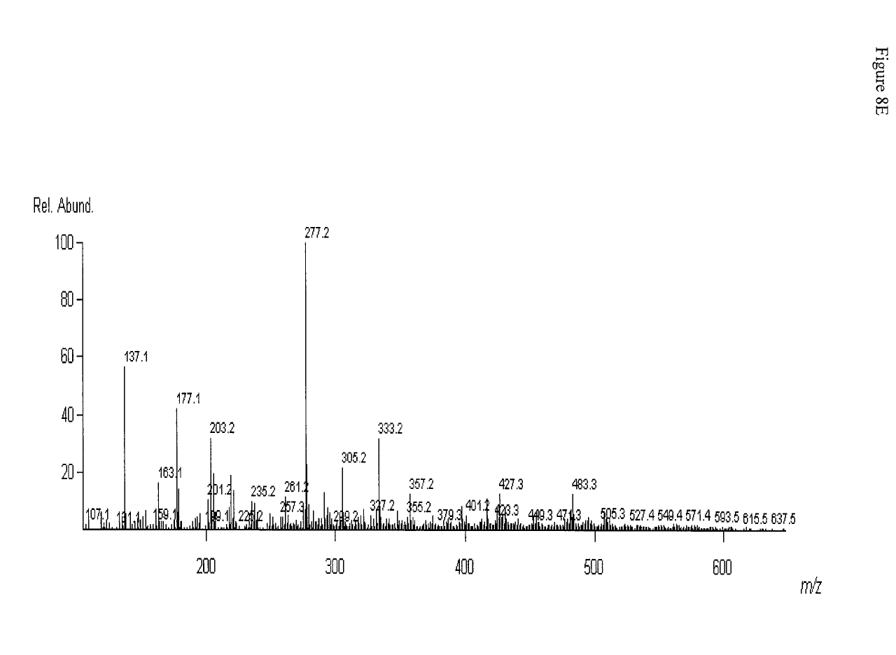

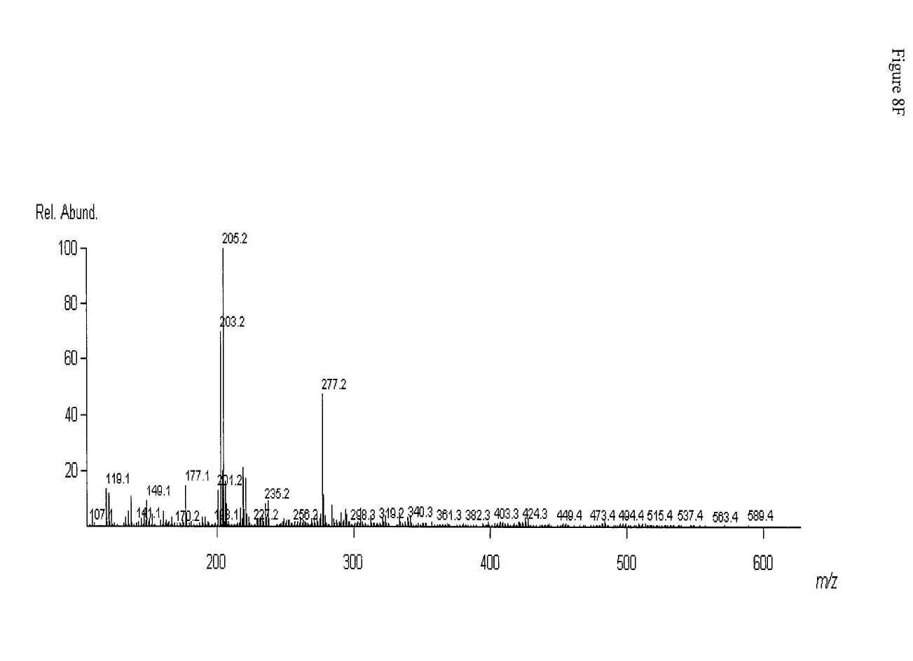

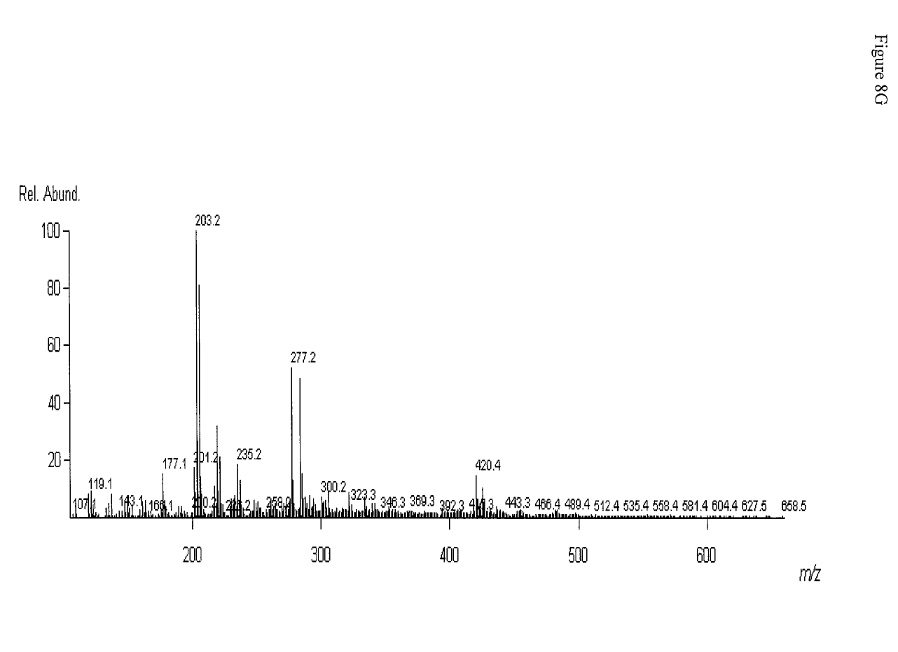

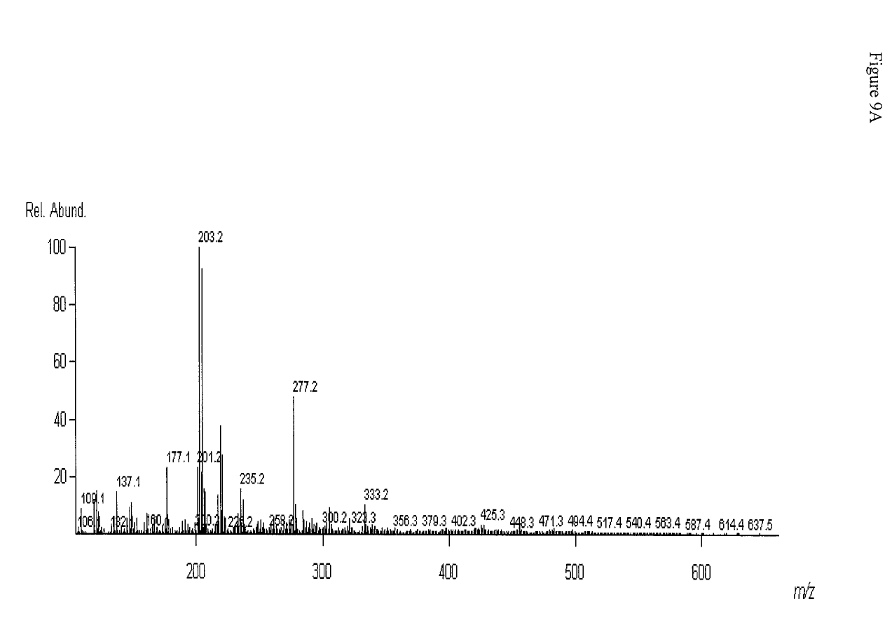

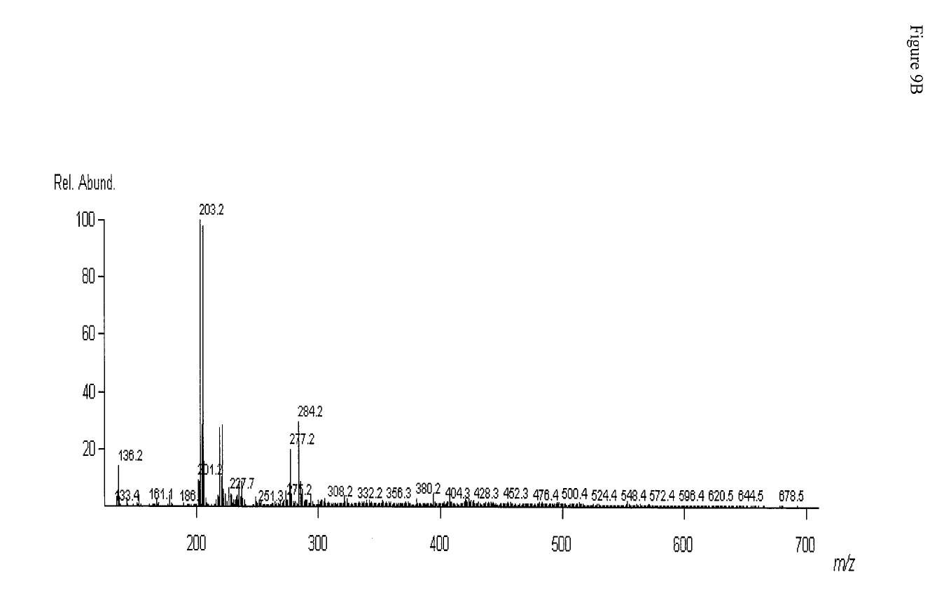

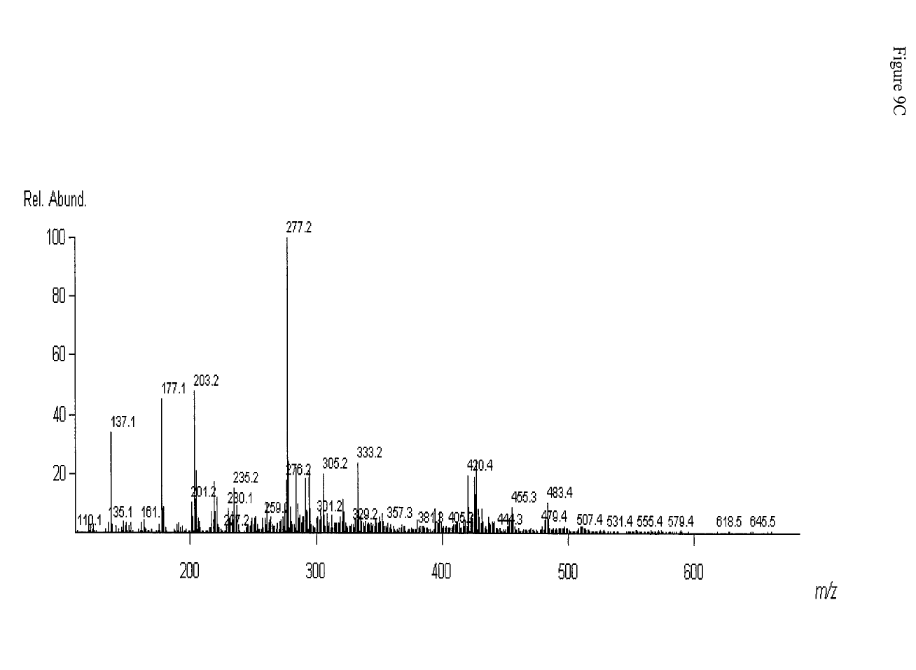

A typical experimental example of solvent extraction and precipitation of the water soluble, ethanol insoluble purified polysaccharide fraction chemical constituents of Ginger species is as follows: 25 gm of the solid residue from the 2 stage hydro-alcoholic leaching extraction of Step 2 was extracted using 750 ml of distilled water for three hour at 80° C. in two stages. The solvent (500 ml) to feedstock ratio was 20:1 for the first stage and 10:1 (250 ml) for the second stage. The two extraction solutions were combined and the slurry was filtered using Fisherbrand P4 filter paper (pore size 4-8 μm) and centrifuged at 3,000 rpm for 10 minutes. The supernatant was collected. The weight of solid extract was 3.74 gm and the yield was 15% by mass weight. To 25 ml of the clear supernatant extract solution, 100 ml of anhydrous ethanol was added to make up a final concentration of either 60% or 80% ethanol. A precipitate was observed in each sample. The polysaccharide extraction solutions were centrifuged at 3,000 rpm for 10 minutes and the supernatant decanted and discarded. The precipitates were collected, dried in an oven at 50° C. for 12 hours, and labeled as PS60 (60% ethanol precipitation and PS80 (80% ethanol precipitation). The dried polysaccharide fraction was weighed and dissolved in water for analysis of polysaccharide purity with the colormetric method using dextran as reference standards. The results are shown in Table 31. AccuTOF-DART mass spectrums of both purified polysaccharide fractions are shown in FIGS. 6 and 7, A peak data table is presented in Table 32.

TABLE 31
Ginger polysaccharide extraction yield and purity.

| | | Purity (g/g) | | |
|---|---|---|---|---|
| Sample | Yield (%) | Dextran 5K | Dextran 50K | Dextran 410K |
| PS60 | 1.15 | 0.59 | 0.43 | 0.37 |
| PS80 | 1.16 | 0.35 | 0.26 | 0.22 |

TABLE 32
Peak data of AccuTOF-DART mass spectrums for purified polysaccharide
fractions for PS60 positive ion mode, PS60 negative ion mode, PS80 positive ion mode,
and PS80 negative ion mode.
Purified Polysaccharide Fraction

| PS60 (+) ion mode | | PS60 (−) ion mode | | PS80 (+) ion mode | | PS80 (−) ion mode | |
|---|---|---|---|---|---|---|---|
| (m + H)/z | rel. inten. | (m − H)/z | rel. inten. | (m + H)/z | rel. inten. | (m − H)/z | rel. inten. |
| 58.56 | 103.53 | 59.33 | 3256.27 | 58.56 | 103.53 | 87.20 | 1327.72 |
| 66.32 | 105.28 | 87.20 | 2633.43 | 66.32 | 105.28 | 89.20 | 20087.08 |
| 76.10 | 2862.40 | 89.20 | 54073.64 | 76.10 | 2862.40 | 89.38 | 137.08 |
| 77.08 | 149.12 | 89.38 | 351.52 | 77.08 | 149.12 | 90.213 | 764.37 |
| 80.01 | 597.45 | 89.69 | 124.19 | 80.01 | 597.45 | 121.10 | 428.12 |
| 80.96 | 399.28 | 89.74 | 137.73 | 80.96 | 399.28 | 129.12 | 52.88 |
| 83.94 | 606.98 | 90.21 | 2081.80 | 83.94 | 606.98 | 153.05 | 90.4 |
| 89.81 | 6545.53 | 91.20 | 368.84 | 89.81 | 6545.53 | 165.03 | 4399.27 |
| 90.05 | 211.95 | 92.19 | 624.23 | 90.05 | 211.95 | 166.04 | 434.08 |
| 90.09 | 131.70 | 94.17 | 132.24 | 90.09 | 131.70 | 167.06 | 84.82 |
| 90.40 | 148.07 | 121.11 | 3509.17 | 90.40 | 148.07 | 169.06 | 101.66 |
| 90.79 | 421.96 | 122.12 | 421.36 | 90.79 | 421.96 | 179.07 | 261.14 |
| 93.77 | 126792.3 | 165.03 | 2465.50 | 93.77 | 126792.3 | 195.06 | 72.75 |
| 94.76 | 5249.60 | 166.04 | 204.00 | 94.76 | 5249.60 | 211.07 | 121.04 |
| 95.50 | 76.79 | 179.07 | 518.63 | 95.50 | 76.79 | 227.21 | 119.72 |
| 95.75 | 770.79 | 199.18 | 428.59 | 95.75 | 770.79 | | |
| 99.68 | 385.26 | 205.15 | 7500.63 | 99.68 | 385.26 | | |
| 101.66 | 36097.33 | 205.67 | 77.48 | 101.66 | 36097.33 | | |
| 102.65 | 2581.32 | 206.17 | 1447.27 | 102.65 | 2581.32 | | |
| 103.65 | 419.59 | 220.15 | 102.00 | 103.65 | 419.59 | | |
| 105.61 | 295.68 | 221.15 | 143.16 | 105.61 | 295.68 | | |
| 106.59 | 163.53 | 225.19 | 81.11 | 106.59 | 163.53 | | |
| 107.60 | 105.34 | 227.20 | 1280.91 | 107.60 | 105.34 | | |
| 111.55 | 369.20 | 228.21 | 93.34 | 111.55 | 369.20 | | |
| 114.59 | 158.5 | 233.17 | 167.51 | 114.59 | 158.5 | | |
| 118.50 | 2089.43 | 238.15 | 1018.25 | 118.50 | 2089.43 | | |
| 119.47 | 1276.56 | 239.20 | 335.28 | 119.47 | 1276.56 | | |
| 121.47 | 1657.44 | 241.22 | 727.00 | 121.47 | 1657.44 | | |
| 122.46 | 108.95 | 242.23 | 72.81 | 122.46 | 108.95 | | |
| 124.42 | 141.96 | 253.22 | 1074.64 | 124.42 | 141.96 | | |
| 125.43 | 82.73 | 254.23 | 91.16 | 125.43 | 82.73 | | |
| 133.38 | 608.01 | 255.24 | 1927.08 | 133.38 | 608.01 | | |
| 135.34 | 5256.85 | 256.24 | 203.15 | 135.34 | 5256.85 | | |
| 135.54 | 400.56 | 269.26 | 208.10 | 135.54 | 400.56 | | |
| 135.96 | 72.68 | 281.25 | 312.28 | 135.96 | 72.68 | | |
| 136.27 | 110.45 | 283.28 | 174.47 | 136.27 | 110.45 | | |
| 136.34 | 337.14 | 291.21 | 450.98 | 136.34 | 337.14 | | |
| 137.35 | 127.09 | 305.21 | 3957.51 | 137.35 | 127.09 | | |
| 139.35 | 9169.85 | 306.23 | 927.40 | 139.35 | 9169.85 | | |
| 139.82 | 99.12 | | | 139.82 | 99.12 | | |
| 139.99 | 145.40 | | | 139.99 | 145.40 | | |
| 140.34 | 838.65 | | | 140.34 | 838.65 | | |
| 145.30 | 168.68 | | | 145.30 | 168.68 | | |
| 147.31 | 34072.66 | | | 147.31 | 34072.66 | | |
| 147.93 | 702.46 | | | 147.93 | 702.46 | | |
| 148.08 | 684.39 | | | 148.08 | 684.39 | | |
| 148.31 | 3830.21 | | | 148.31 | 3830.21 | | |
| 149.31 | 700.26 | | | 149.31 | 700.26 | | |
| 149.43 | 223.84 | | | 149.43 | 223.84 | | |
| 151.26 | 393.87 | | | 151.26 | 393.87 | | |
| 153.26 | 921.31 | | | 153.26 | 921.31 | | |
| 159.22 | 76.67 | | | 159.22 | 76.67 | | |
| 160.27 | 48.75 | | | 160.27 | 48.75 | | |
| 161.25 | 256.93 | | | 161.25 | 256.93 | | |
| 163.24 | 415.31 | | | 163.24 | 415.31 | | |
| 165.25 | 81.87 | | | 165.25 | 81.87 | | |
| 167.22 | 91.73 | | | 167.22 | 91.73 | | |
| 175.25 | 439.14 | | | 175.25 | 439.14 | | |
| 177.17 | 79.68 | | | 177.17 | 79.68 | | |
| 179.19 | 117.62 | | | 179.19 | 117.62 | | |
| 180.22 | 110.85 | | | 180.22 | 110.85 | | |
| 181.19 | 360.57 | | | 181.19 | 360.57 | | |
| 183.23 | 162.23 | | | 183.23 | 162.23 | | |
| 187.19 | 132.32 | | | 187.19 | 132.32 | | |
| 189.19 | 1297.96 | | | 189.19 | 1297.96 | | |
| 193.21 | 194.23 | | | 193.21 | 194.23 | | |
| 195.15 | 460.04 | | | 195.15 | 460.04 | | |
| 201.21 | 3939.98 | | | 201.21 | 3939.98 | | |
| 202.21 | 624.73 | | | 202.21 | 624.73 | | |
| 203.21 | 198.60 | | | 203.21 | 198.60 | | |
| 209.19 | 255.01 | | | 209.19 | 255.01 | | |
| 217.20 | 164.84 | | | 217.20 | 164.84 | | |
| 223.10 | 217.54 | | | 223.10 | 217.54 | | |
| 279.16 | 418.25 | | | 279.16 | 418.25 | | |
| 279.26 | 140.51 | | | 279.26 | 140.515 | | |
| 313.14 | 158.14 | | | 313.14 | 158.14 | | |
| 391.28 | 113.70 | | | 391.28 | 113.70 | | |

### Example 7

The following ingredients are mixed for the formulation:

| | |
|---|---|
| Extract of Ginger root | 150.0 mg |
| Volatile Oil Fraction (20 mg, 13.3% dry weight) | |
| Gingerol Fraction (100 mg, 66.7% dry weight) | |
| Phenolic Fraction (20 mg, 13.3% dry weight) | |
| Polysaccharides (10 mg, 6.7% dry weight) | |
| Stevioside (Extract of Stevia) | 12.5 mg |
| Carboxymethylcellulose | 35.5 mg |
| Lactose | 77.0 mg |
| Total | 275.0 mg |

The novel extract of Ginger species comprises an essential oil fraction, a triterpene glycoside fraction, a phenolic acid fraction, and a polysaccharide fraction by % mass weight greater than that found in the natural rhizome material or convention extraction products. The formulations can be made into any oral dosage form and administered daily or to 15 times per day as needed.

### Example 8

The following ingredients were mixed for the following formulation:

| | |
|---|---|
| Extract of Ginger root | 300.0 mg |
| Volatile Oil Fraction (15 mg, 5% dry weight) | |
| Gingerol Fraction (150 mg, 50% dry weight) | |
| Phenolic Fraction (60 mg, 20% dry weight) | |
| Polysaccharides (75 mg, 25% dry weight) | |
| Vitamin C | 15.0 mg |
| Sucralose | 35.0 mg |
| Mung Bean Powder 10:1 | 70.0 mg |
| Mocha Flavor | 60.0 mg |
| Chocolate Flavor | 20.0 mg |
| Total | 500.0 mg |

The novel extract composition of Ginger species comprises an essential oil, triterpene glycoside, phenolic acid, and polysaccharide chemical constituent fractions by % mass weight greater than that found in the natural plant material or conventional extraction products. The formulation can be made into any oral dosage form and administered safely up to 15 times per day as needed.

### REFERENCES

U.S. Pat. Nos. 5,298,261; 5,407,339; 5,464,632; 6,106,861; 6,221,392; and 6,200,604.
1. Smith C et al. Obstet Gynecol 103(4): 639-645, 2004.
2. Lien H C et al. Am J Physiol Gastrointest Liver Physiol. 284(3): G481-489, 2003.
3. Prongrojpaw D & Chiamchanya C. J Med Assoc Thai 86(3): 244-250, 2003.
4. Willetts K E et al. Aust N Z J Obstet Gynaecol 43(2): 139-144, 2003.
5. Portnoi G et al. Am J Obstet Gynecol 189(5): 1374-1377.
6. Borelli F et al. Obstet Gynecol 105(4): 849-856, 2005.
7. Abdel-Aziz H et al. Eur J Pharmacol 530(1-2): 136-143, 2006.
8. Abdel-Aziz H et al. Planta Med 71(7): 609-616, 2005.
9. Holtmann S et al. Acta Otolaryngol (Stockh) 108: 168-174, 1989.
10. Arfeen Z et al. Anaesthesia 23: 449-452, 1995.
11. Altman R D et al. Arthritis Rheum 44:2531-2538, 2001.
12. Wigler I et al. Osteoarthritis Cartilage 11(11): 783-789, 2003.
13. Penna S C et al. Phytomedicine 10(5): 381-385, 2003.
14. Shen C L et al. J Med Food 8(2): 149-153, 2005.
15. Grzanna R et al. J Med Food 8(2): 125-132, 2005.
16. Phan P V et al. J Altern Complement Med 11 (1): 149-154, 2005.
17. Kim H W et al. Antioxid Redox Signal 7(11-12): 1621-1629, 2005.
18. Young H Y et al. J Ethnopharmacol 96(1-2): 207-10, 2005.
19. Shin S G et al. J Agric Food Chem 53(19): 7617-7622, 2005.
20. Chrubasik S et al. Phytomedicine 12(9): 684-701, 2005.
21. Masuda Y et al. Biofactors 21(1-4): 293-296, 2004.
22. Lu P et al. Zhongguo Zhong Yao Za Zhi 28(9): 873-875, 2003.
23. Kuo P C et al. Arch Pharm Res 28(5): 518-528, 2005.
24. Kadnur S V & Goyal R K. Indian J Exp Biol 43(12): 1161-1164, 2005.
25. Bhandari U et al. J Ethnopharmacol 97(2): 227-230, 2005.
26. Sekiya K et al. Biofactors 22(1-4): 153-156, 2004.
27. Akhani S P et al. J Pharm Pharmacol 56(1): 101-105, 2004.
28. Nurtjahja-Tjendraputra E et al. Thromb Res 111(4-5): 259-265, 2003.
29. Ghayur M N et al. Vascul Pharmacol 43(4): 234-241, 2005.
30. Ghayur M N & Gilani A H. J Cardiovasc Pharmacol 45(1): 74-80, 2005.
31. Verma S K et al. Indian J Exp Biol 42(7): 736-738, 2004.
32. Han L K et al. Yakugaku Zasshi 125(2): 213-217, 2005.
33. Grzanna R et al. J Altern Complement Med 10(6): 1009-1013, 2004.
34. Kabuto H et al. Neurochem Res 30(3): 325-332, 2005.
35. Cady R K et al. Med Sci Monit 11(9): P165-169,2005.
36. Zhou H L et al. J Ethnopharmacol (in press), 2006.
37. Jagetia G et al. Cancer Biother Radiopharm 19(4): 422-435, 2004.
38. Borrelli F et al. Life Sci 74(23): 2889-2896, 2004.
39. Ghayur M N & Gilani A H. Dig Dis Sci 50(10): 1889-1897, 2005.
40. Lopez P et al. J Agric Food Chem 53(7): 6939-6946, 2005.
41. Mahady G B et al. Phytother Res 19(11): 988-991, 2005.
42. Thongson C et al. Lett Appl Microbiol 39(5): 401-406, 2004.
43. Wei O Y et al. J Ethnopharmacol 102(2): 177-184, 2005.
44. Wang G et al. Cell Mol Life Sci 62(7-8): 881-893, 2005.
45. Kim E C et al. Biochem Biophys Res Commun 335(2): 300-308, 2005.
46. Manju V & Nalini N. Clin Chim Acta 358(1-2): 60-67, 2005.
47. Kirana C et al. Nutr Cancer 45(2): 218-225, 2003.
48. Leal P F et al. J Agric Food Chem 51(9): 2550-2555, 2003.
49. Williamson E M. Phytomedicine 8: 401-409, 2001.
50. Dubois M et al. Analytical Chem 28:350-356, 1956.
51. Chen C C & Ho C T. J Agric Food Chem 36:322-328, 1988.
52. Chen C C et al. J Agric Food Chem 34:477-480, 1986.
53. Roy B C et al. Ind Eng Chem Res 35:607-612, 1996.
54. Yonei Y et al. J Supercritical Fluids 8:156-161, 1995.

We claim:

1. A composition comprising a gingerol in an amount greater than 2% by weight.

2. The composition of claim 1, wherein the gingerol comprises 6-gingerol, 8-gingerol, 10-gingerol, 6-shagaol, or combinations thereof.

3. The composition of claim 2, wherein the amount of gingerol is greater than 5, 10, 15, 20, 25, 30, 35, 40, 45, 50, 55, 60, 65, or 70% by weight.

4. The composition of claim 2, wherein the amount of gingerol is 50% to 70% by weight.

5. The composition of claim 2, wherein the amount of gingerol is 50% by weight.

6. The composition of claim 2, wherein the amount of gingerol is greater than 65% weight.

7. The composition of claim 2, further comprising an essential oil, wherein said essential oil comprises beta-bisabolene, zingiberene, beta-sesquinhellandrene, arcurcumene, geranial, neral, champhene, phellandrene, cineol, citral, borneol, citronellol, linalool, limonene, zingiberol, betpinene, 2-undecanone, beta-elemene, beta-farnesene, cariophilene, cis-trans-alpha-farnesene, beta-sesquifel, elemol, nerolidol, beta-eudesmol, octanol, decenal, α-terpineol, or combinations thereof.

8. The composition of claim 7, wherein the amount of essential oil is 5% to 20% by weight.

9. The composition of claim 8, wherein the essential oil is zingiberene.

10. The composition of claim 7, wherein the amount of gingerol is greater than 5, 10, 15, 20, 25, 30, 35, 40, 45, 50, 55, 60, 65, or 70% by weight.

11. The composition of claim 7, wherein the amount of gingerol is 50% to 70% by weight.

12. The composition of claim 7, wherein the amount of gingerol is 50% by weight.

13. The composition of claim 7, wherein the amount of gingerol is greater than 65% by weight.

14. The composition of claim 7, wherein the amount of gingerol is 50% to 70% by weight, and the amount of essential oil is 5% to 20% by weight.

15. The composition of claim 7, wherein the amount of gingerol is greater than 65% by weight, and the amount of essential oil is greater than 10% by weight.

16. The composition of claim 7, wherein the amount of gingerol is 50% by weight, and the amount of essential oil is 5% by weight.

17. The composition of claim 2, further comprising a polysaccharide.

18. The composition of claim 17, wherein the amount of polysaccharide is greater than 5% to 30% by weight.

19. The composition of claim 18, wherein the polysaccharide comprises glucose, arabinose, galactose, rhamnose, xylose, uronic acid, or combinations thereof.

20. The composition of claim 17, wherein the amount of gingerol is greater than 5, 10, 15, 20, 25, 30, 35, 40, 45, 50, 55, 60, 65, or 70% by weight.

21. The composition of claim 17, wherein the amount of gingerol is 50% to 70% by weight.

22. The composition of claim 17, wherein the amount of gingerol is 50% by weight.

23. The composition of claim 17, wherein the amount of gingerol is greater than 65% by weight.

24. The composition of claim 17, wherein the amount of gingerol is 50% to 70% by weight, and the amount of polysaccharide is greater than 5% to 30% by weight.

25. The composition of claim 17, wherein the amount of gingerol is greater than 65% by weight, and the amount of polysaccharide is greater than 5% by weight.

26. The composition of claim 17, wherein the amount of gingerol is 50% by weight, and the amount of polysaccharide is 25% by weight.

27. The composition of claim 17, further comprising an essential oil, wherein said essential oil comprises beta-bisabolene, zingiberene, beta-sesquinhellandrene, arcurcumene, geranial, neral, champhene, phellandrene, cineol, citral, borneol, citronellol, linalool, limonene, zingiberol, betpinene, 2-undecanone, beta-elemene, beta-farnesene, cariophilene, cis-trans-alpha-farnesene, beta-sesquifel, elemol, nerolidol, beta-eudesmol, octanol, decenal, α-terpineol, or combinations thereof.

28. The composition of claim 27, wherein the amount of essential oil is 5% to 20% by weight.

29. The composition of claim 17, further comprising the essential oil zingiberene.

30. The composition of claim 29, wherein the amount of essential oil is 5% to 20% by weight.

31. The composition of claim 28, further comprising phenolics.

32. The composition of claim 31, wherein the amount of phenolics is greater than 1% to 25% by weight.

33. The composition of claim 2, further comprising a pharmaceutical carrier.

34. The composition of claim 27 further comprising a pharmaceutical carrier.

35. The composition of claim 29 further comprising a pharmaceutical carrier.

36. The composition of claim 17, wherein the polysaccharide has the DART profile of FIG. 6, FIG. 7, or Table 13.

37. A method for extracting a ginger species comprising, sequentially extracting a ginger species plant material to yield an essential oil fraction, a gingerol fraction, a phenolic fraction, and a polysaccharide fraction, wherein the essential oil and gingerol fractions are derived by extracting plant feedstock material by supercritical carbon dioxide extraction, the phenolic fraction is extracted from the plant feedstock material or from the remainder of the essential oil and gingerol extractions by hydroalcoholic extraction, and the polysaccharide fraction is derived by water extraction of the remainder of the phenolic extraction.

38. The method of claim 37, wherein the supercritical carbon dioxide extraction comprises:
a) placing ginger bark in an extraction vessel;
b) extracting the ginger bark with supercritical carbon dioxide at between 60 bar and 800 bar and between 35 degrees C. and 90 degrees C. for a time sufficient to extract essential oil and gingerol; and
c) collecting the essential oil and gingerol fractions.

39. The method of claim 38, wherein the ginger bark is dried and ground.

40. The method of claim 38, wherein step b) is conducted at between 60 bar and 500 bar and between 40 degrees C. and 80 degrees C.

41. The method of claim 38, wherein the time sufficient to extract essential oil and gingerol is between 30 minutes and 2.5 hours.

42. The method of claim 37, wherein phenolic extraction comprises:
(a) contacting a plant feedstock material, or remainder thereof from an extraction of essential oil and gingerol fractions by supercritical carbon dioxide, with a hydroalcoholic mixture for a time sufficient to extract phenolics to form an aqueous solution of extracted phenolics;
(b) passing the aqueous solution of extracted phenolics through an adsorbent resin column wherein the phenolics are adsorbed; and
(c) eluting phenolics from adsorbent resin.

43. The method of claim 42, wherein hydroalcoholic mixture comprises water and ethanol.

44. The method of claim 43, wherein the amount of ethanol is from 10% to 95% by weight.

45. The method of claim 43, wherein the amount of ethanol is 25% by weight.

46. The method of claim 42, wherein step a) comprises heating and stirring the plant feedstock material or remainder between about 30 degrees C. and 100 degrees C. for about 1 to 10 hours.

47. The method of claim 42, wherein in step C) eluting phenolics from adsorbent resin is done with methanol, ethanol, or propanol.

48. The method of claim 37, wherein the water extraction of the remainder of the phenolic extraction to obtain the polysaccharide fraction comprises:
a) mixing the remainder of the phenolic extraction with water;
b) heating and stirring the mixture for a time effective for extracting the polysaccharides;
c) separating solids from the mixture of step b) to form a solution; and
d) adding alcohol to the solution to precipitate the polysaccharide.

49. The method of claim 48, wherein step b) is conducted between 60 degrees C. and 100 degrees C. for 1 to 5 hours.

50. The method of claim 48, wherein the alcohol is ethanol.
